(19) United States
(12) Patent Application Publication
(10) Pub. No.: US 2025/0388902 A1
(43) Pub. Date: Dec. 25, 2025

(54) ANTISENSE OLIGOMERS FOR TREATMENT OF NON-SENSE MEDIATED RNA DECAY BASED CONDITIONS AND DISEASES

(71) Applicant: Stoke Therapeutics, Inc., Bedford, MA (US)

(72) Inventors: Isabel Aznarez, Boston, MA (US); Jacob Kach, Boston, MA (US); Pavitra Ramachandran, Winchester, MA (US); Ana Corrionero Saiz, Cambridge, MA (US)

(21) Appl. No.: 19/187,338

(22) Filed: Apr. 23, 2025

Related U.S. Application Data
(63) Continuation of application No. PCT/US2023/036297, filed on Oct. 30, 2023.
(60) Provisional application No. 63/381,640, filed on Oct. 31, 2022.

(51) Int. Cl. C12N 15/113

(52) U.S. Cl. CPC C12N 15/113; C12N 2310/14; C12N 2310/313; C12N 2310/321; C12N 2320/33

(57) ABSTRACT

Alternative splicing events in genes can lead to non-productive mRNA transcripts which in turn can lead to aberrant or reduced protein expression, and therapeutic agents which can target the alternative splicing events in the genes can modulate the expression level of functional proteins in patients and/or inhibit aberrant protein expression. Such therapeutic agents can be used to treat a condition or disease caused by protein deficiency.

### CROSS-REFERENCE

This application is a continuation of International Application No. PCT/US2023/036297, filed Oct. 30, 2023, which claims the benefit of U.S. Provisional Application No. 63/381,640, filed Oct. 31, 2022, each of which is incorporated herein by reference in its entirety.

### SEQUENCE LISTING

The instant application contained a Sequence Listing which has been submitted electronically in XML format and is hereby incorporated by reference in its entirety. Said XML copy, created on Apr. 23, 2025, is named 47991_737_301_SL.xml and is 1,936,504 bytes in size.

### BACKGROUND

Alternative splicing events in genes can lead to non-productive mRNA transcripts which in turn can lead to aberrant protein expression, and therapeutic agents which can target the alternative splicing events in genes can modulate the expression level of functional proteins in patients and/or inhibit aberrant protein expression. Such therapeutic agents can be used to treat a condition or disease caused by protein deficiency.

### SUMMARY

Provided herein, in some aspects, is a method of modulating expression of a target protein in a cell comprising a pre-mRNA that is transcribed from a target gene and that encodes the target protein, the pre-mRNA comprising an alternatively-spliced coding exon (ASCE), wherein an alternative processed mRNA that is produced by splicing out of the ASCE during processing of the pre-mRNA undergoes non-sense mediated RNA decay, the method comprising contacting a therapeutic agent or a vector encoding the therapeutic agent to the cell, wherein the therapeutic agent promotes inclusion of the ASCE during the processing of the pre-mRNA, thereby increasing a level of a processed mRNA that is processed from the pre-mRNA and comprises the ASCE.

Provided herein, in some aspects, is a method of treating or reducing the likelihood of developing a disease or condition in a subject in need thereof by modulating expression of a target protein in a cell of the subject, the method comprising: contacting the cell of the subject with a therapeutic agent or a vector encoding the therapeutic agent, wherein the cell comprises a pre-mRNA that is transcribed from a target gene and that encodes the target protein, the pre-mRNA comprising an alternatively-spliced coding exon (ASCE), wherein an alternative processed mRNA that is produced by splicing out of the ASCE during processing of the pre-mRNA undergoes non-sense mediated RNA decay, wherein the therapeutic agent promotes inclusion of the ASCE during the processing of the pre-mRNA, thereby increasing a level of a processed mRNA that is processed from the pre-mRNA and comprises the ASCE.

In some embodiments, the expression of the target protein is increased in the cell.

In some embodiments, the target gene is selected from the group consisting of: PKD1, ABCA4, FUS, CEL, and NSD1.

In some embodiments, the target protein is selected from the group consisting of: polycystin-1, ATP binding cassette subfamily A member 4, FUS RNA binding protein, carboxyl ester lipase, and nuclear receptor binding SET domain protein 1.

In some embodiments, the therapeutic agent
(a) binds to a targeted portion of the mRNA encoding the target protein;
(b) modulates binding of a factor involved in splicing of the ASCE; or
(c) a combination of (a) and (b).

In some embodiments, the therapeutic agent interferes with binding of the factor involved in splicing of the ASCE to a region of the targeted portion.

In some embodiments, the targeted portion is proximal to the ASCE.

In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the ASCE.

In some embodiments, the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the ASCE.

In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the ASCE.

In some embodiments, the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the ASCE.

In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site selected from the group consisting of: GRCh38/hg38: chr16 2092954; GRCh38/hg38: chr1 94111438; GRCh38/hg38: chr16 31186802; GRCh38/hg38: chr9 133066530; and GRCh38/hg38: chr5 177238237.

In some embodiments, the targeted portion is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site selected from the group consisting of: GRCh38/hg38: chr16 2092954; GRCh38/hg38: chr1 94111438; GRCh38/hg38: chr16 31186802; GRCh38/hg38: chr9 133066530; and GRCh38/hg38: chr5 177238237.

In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr16 2093093; GRCh38/hg38: chr1 94111579; GRCh38/hg38: chr16 31186836; GRCh38/hg38: chr9 133066660; and GRCh38/hg38: chr5 177238507.

In some embodiments, the targeted portion is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr16 2093093; GRCh38/hg38: chr1 94111579; GRCh38/hg38: chr16 31186836; GRCh38/hg38: chr9 133066660; and GRCh38/hg38: chr5 177238507.

In some embodiments, the targeted portion is located in an intronic region between the ASCE and a canonical exonic region upstream of the ASCE of the mRNA encoding the target protein.

In some embodiments, the targeted portion is located in an intronic region between the ASCE and a canonical exonic region downstream of the ASCE of the mRNA encoding the target protein.

In some embodiments, the targeted portion at least partially overlaps with the ASCE.

In some embodiments, the targeted portion at least partially overlaps with an intron upstream or downstream of the ASCE.

In some embodiments, the targeted portion does not comprise a 5′ exon-intron junction or a 3′ exon-intron junction.

In some embodiments, the targeted portion is within the ASCE.

In some embodiments, the targeted portion comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the ASCE.

In some embodiments, the mRNA encoding the target protein comprises a sequence with at least about 80%, 85%, 90%, 95%, 97%, or 100% sequence identity to a sequence selected from the group consisting of SEQ ID NOS: 6-10.

In some embodiments, the mRNA encoding the target protein is encoded by a genetic sequence with at least about 80%, 85%, 90%, 95%, 97%, or 100% sequence identity to a sequence selected from the group consisting of SEQ ID NOS: 1-5.

In some embodiments, the targeted portion of the mRNA comprises a sequence with at least 80%, 85%, 90%, 95%, 97%, or 100% sequence identity to a region comprising at least 8 contiguous nucleic acids of a sequence selected from the group consisting of SEQ ID NOS: 6-10.

In some embodiments, the targeted portion of the mRNA is within the ASCE selected from the group consisting of: GRCh38/hg38: chr16 2092954 2093093; GRCh38/hg38: chr1 94111438 94111579; GRCh38/hg38: chr16 31186802 31186836; GRCh38/hg38: chr9 133066530 133066660; and GRCh38/hg38: chr5 177238237 177238507.

In some embodiments, the targeted portion of the mRNA is upstream or downstream of the ASCE selected from the group consisting of: GRCh38/hg38: chr16 2092954 2093093; GRCh38/hg38: chr1 94111438 94111579; GRCh38/hg38: chr16 31186802 31186836; GRCh38/hg38: chr9 133066530 133066660; and GRCh38/hg38: chr5 177238237 177238507.

In some embodiments, the targeted portion of the mRNA does not comprise an exon-intron junction of an ASCE selected from the group consisting of: GRCh38/hg38: chr16 2092954 2093093; GRCh38/hg38: chr1 94111438 94111579; GRCh38/hg38: chr16 31186802 31186836; GRCh38/hg38: chr9 133066530 133066660; and GRCh38/hg38: chr5 177238237 177238507.

In some embodiments, the target protein produced is a full-length protein or a wild-type protein.

In some embodiments, inclusion of the ASCE during the processing of the pre-mRNA in the cell contacted with the therapeutic agent or the vector encoding the therapeutic agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to inclusion of the ASCE during the processing of the pre-mRNA in a corresponding cell that is not contacted with the therapeutic agent or the vector encoding the therapeutic agent.

In some embodiments, the level of the processed mRNA produced in the cell contacted with the therapeutic agent or the vector encoding the therapeutic agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA in a corresponding cell that is not contacted with the therapeutic agent or the vector encoding the therapeutic agent.

In some embodiments, a level of the target protein produced in the cell contacted with the therapeutic agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein produced in a corresponding cell that is not contacted with the therapeutic agent or the vector encoding the therapeutic agent.

In some embodiments, exclusion of the ASCE during the processing of the pre-mRNA in the cell contacted with the therapeutic agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the ASCE during the processing of the pre-mRNA in a corresponding cell that is not contacted with the therapeutic agent or the vector encoding the therapeutic agent.

In some embodiments, the target protein is NSD1, and the method causes a modification of a histone protein in the cell.

In some embodiments, the histone protein is Histone H3.

In some embodiments, the modification comprises acetylation, methylation, phosphorylation, or ubiquitination.

In some embodiments, the modification is methylation.

In some embodiments, the methylation of the histone protein is increased in the cell.

In some embodiments, the methylation of the histone protein in the cell contacted with the therapeutic agent or the vector encoding the therapeutic agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the methylation of the histone protein in a corresponding cell that is not contacted with the therapeutic agent or the vector encoding the therapeutic agent.

In some embodiments, the method further comprises assessing mRNA level or expression level of the target protein.

In some embodiments, the disease or condition is induced by a loss-of-function mutation in the target gene.

In some embodiments, the disease or condition is associated with haploinsufficiency of a gene encoding the target protein, and the subject has a first allele encoding a functional target protein, and a second allele from which the target protein is not produced or produced at a reduced level, or a second allele encoding a nonfunctional target protein or a partially functional target protein.

In some embodiments, the disease or condition is selected from the group consisting of: Polycystic Kidney Disease 1 with or without Polycystic Liver Disease; Autosomal Dominant Polycystic Kidney Disease; Age-related macular degeneration-2; Stargardt Disease 1; Amyotrophic Lateral Sclerosis; Amyotrophic Lateral Sclerosis 6 with or without Frontotemporal Dementia; Tremor, Hereditary Essential, 4; Frontotemporal Dementia; Maturity-Onset Diabetes Of The Young, Type 8, with Exocrine Dysfunction; Maturity-Onset Diabetes Of The Young; Sotos Syndrome 1; and Beckwith-Wiedemann Syndrome.

In some embodiments, the disease or condition is associated with an autosomal recessive mutation of a gene encoding the target protein, wherein the subject has a first allele encoding from which: (i) the target protein is not produced or produced at a reduced level compared to a wild-type allele; or (ii) the target protein produced is nonfunctional or partially functional compared to a wild-type allele, and a second allele from which: (iii) the target protein is produced at a reduced level compared to a wild-type allele and the target protein produced is at least partially functional compared to a wild-type allele; or (iv) the target protein produced is partially functional compared to a wild-type allele.

In some embodiments, the disease or condition is induced by a gain-of-function mutation in the target protein.

In some embodiments, the subject has an allele from which the target protein is produced at an increased level, or an allele encoding a mutant target protein that exhibits increased activity in the cell.

In some embodiments, the subject is a human.

In some embodiments, the subject is a non-human animal.

In some embodiments, the subject is a fetus, an embryo, or a child.

In some embodiments, the cell or the cells is ex vivo, or in a tissue, or organ ex vivo.

In some embodiments, the therapeutic agent is administered by intrathecal injection, intracerebroventricular injection, intraperitoneal injection, intramuscular injection, subcutaneous injection, intravitreal, or intravenous injection of the subject.

In some embodiments, the method further comprises administering a second therapeutic agent to the subject.

In some embodiments, the second therapeutic agent is a small molecule.

In some embodiments, the second therapeutic agent is an antisense oligomer.

In some embodiments, the second therapeutic agent corrects intron retention.

In some embodiments, the disease or condition is a disease or condition associated with a deficiency in amount or activity of the target protein.

In some embodiments, the disease or condition is a disease or condition associated with a deficiency in amount or activity of a protein that the target protein functionally augments, compensates for, replaces or functionally interacts with.

In some embodiments, the disease or the condition is caused by a deficient amount or activity of the target protein.

In some embodiments, the method further comprises assessing the subject's genome for at least one genetic mutation associated with the disease.

In some embodiments, at least one genetic mutation is within a locus of a gene associated with the disease.

In some embodiments, at least one genetic mutation is within a locus associated with expression of a gene associated with the disease.

In some embodiments, at least one genetic mutation is within the locus of the gene encoding the target protein.

In some embodiments, at least one genetic mutation is within a locus associated with expression of the gene encoding the target protein.

In some embodiments, the method treats the disease or condition.

In some embodiments, the target protein is the canonical isoform of the protein.

In some embodiments, the alternative processed mRNA that is produced by splicing out of the ASCE comprises a premature termination codon (PTC).

In some embodiments, the agent is an antisense oligomer (ASO).

In some embodiments, the ASO is at least 80%, at least 85%, at least 90%, at least 95%, at least 98%, at least 99%, or 100%, complementary to the targeted portion of the mRNA.

In some embodiments, the ASO comprises a sequence that is at least about 80%, 85%, 90%, 95%, 97%, or 100% complementary to at least 8 contiguous nucleic acids of a sequence selected from the group consisting of SEQ ID NOS: 6-10.

In some embodiments, the ASO comprises a backbone modification comprising a phosphorothioate linkage or a phosphorodiamidate linkage.

In some embodiments, the ASO comprises a phosphorodiamidate morpholino, a locked nucleic acid, a peptide nucleic acid, a 2′-O-methyl, a 2′-Fluoro, or a 2′-O-methoxyethyl moiety.

In some embodiments, the ASO comprises at least one modified sugar moiety.

In some embodiments, each sugar moiety is a modified sugar moiety.

In some embodiments, the ASO consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some embodiments, the target gene is NSD1, and the ASO comprises a sequence with at least about 80%, 85%, 90%, 95%, 97%, or 100% sequence identity to any one of SEQ ID NOS: 16-1748.

In some embodiments, the target gene is NSD1, and the vector encoding the agent encodes a polynucleotide comprising a sequence with at least about 80%, 85%, 90%, 95%, 97%, or 100% sequence identity to any one of the ASO sequences listed in Table 4, Table 5A, Table 5A-1, Table 5B, Table 5B-1, Table 5D, Table 5E, Table 5G, and Table 5G-1.

In some embodiments, the vector encoding the agent is a viral vector.

In some embodiments, the viral vector is an adenovirus-associated viral vector.

In some embodiments, the vector encoding the agent encodes a polynucleotide comprising an ASO sequence and an snRNA.

In some embodiments, the snRNA comprises a modified snRNA.

In some embodiments, the modified snRNA is a modified U1 snRNA or a modified U7 snRNA.

In some embodiments, the snRNA comprises a U1 snRNA.

In some embodiments, the target gene is NSD1, and the ASO sequence comprises a sequence with at least about 80%, 85%, 90%, 95%, 97%, or 100% sequence identity to any one of the ASO sequences listed in Table 4, Table 5A, Table 5B, Table 5D, Table 5E, and Table 5G.

In some embodiments, the snRNA comprises a U7 snRNA.

In some embodiments, the target gene is NSD1, and the ASO comprises a sequence with at least about 80%, 85%, 90%, 95%, 97%, or 100% sequence identity to any one of the ASO sequences listed in Table 4, Table 5A, Table 5A-1, Table 5B, Table 5B-1, Table 5G, and Table 5G-1.

Provided herein, in some aspects, is a composition comprising an agent or a vector encoding the agent, wherein the agent modulates splicing of a pre-mRNA in a cell that is transcribed from a target gene and that encodes the target protein, wherein the pre-mRNA comprises an alternatively-spliced coding exon (ASCE), wherein an alternative processed mRNA that is produced by splicing out of the ASCE during processing of the pre-mRNA undergoes non-sense mediated RNA decay, wherein the agent promotes inclusion of the ASCE during the processing of the pre-mRNA, thereby increasing a level of a processed mRNA that is processed from the pre-mRNA and comprises the ASCE.

In some embodiments, the agent increases expression of the target protein in the cell.

In some embodiments, the target gene is selected from the group consisting of: PKD1, ABCA4, FUS, CEL, and NSD1.

In some embodiments, the target protein is selected from the group consisting of: polycystin-1, ATP binding cassette subfamily A member 4, FUS RNA binding protein, carboxyl ester lipase, and nuclear receptor binding SET domain protein 1.

In some embodiments, the agent
(a) binds to a targeted portion of the mRNA encoding the target protein;
(b) modulates binding of a factor involved in splicing of the ASCE; or
(c) a combination of (a) and (b).

In some embodiments, the agent interferes with binding of the factor involved in splicing of the ASCE to a region of the targeted portion.

In some embodiments, the targeted portion is proximal to the ASCE.

In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the ASCE.

In some embodiments, the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotide(s) upstream of 5′ end of the ASCE.

In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the ASCE.

In some embodiments, the targeted portion is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the ASCE.

In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site selected from the group consisting of: GRCh38/hg38: chr16 2092954; GRCh38/hg38: chr1 94111438; GRCh38/hg38: chr16 31186802; GRCh38/hg38: chr9 133066530; and GRCh38/hg38: chr5 177238237.

In some embodiments, the targeted portion is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site selected from the group consisting of: GRCh38/hg38: chr16 2092954; GRCh38/hg38: chr1 94111438; GRCh38/hg38: chr16 31186802; GRCh38/hg38: chr9 133066530; and GRCh38/hg38: chr5 177238237.

In some embodiments, the targeted portion is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr16 2093093; GRCh38/hg38: chr1 94111579; GRCh38/hg38: chr16 31186836; GRCh38/hg38: chr9 133066660; and GRCh38/hg38: chr5 177238507.

In some embodiments, the targeted portion is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site selected from the group consisting of: GRCh38/hg38: chr16 2093093; GRCh38/hg38: chr1 94111579; GRCh38/hg38: chr16 31186836; GRCh38/hg38: chr9 133066660; and GRCh38/hg38: chr5 177238507.

In some embodiments, the targeted portion is located in an intronic region between the ASCE and a canonical exonic region upstream of the ASCE of the mRNA encoding the target protein.

In some embodiments, the targeted portion is located in an intronic region between the ASCE and a canonical exonic region downstream of the ASCE of the mRNA encoding the target protein.

In some embodiments, the targeted portion at least partially overlaps with the ASCE.

In some embodiments, the targeted portion at least partially overlaps with an intron upstream or downstream of the ASCE.

In some embodiments, the targeted portion does not comprise a 5′ exon-intron junction or a 3′ exon-intron junction.

In some embodiments, the targeted portion is within the ASCE.

In some embodiments, the targeted portion comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the ASCE.

In some embodiments, the mRNA encoding the target protein comprises a sequence with at least about 80%, 85%, 90%, 95%, 97%, or 100% sequence identity to a sequence selected from the group consisting of SEQ ID NOS: 6-10.

In some embodiments, the mRNA encoding the target protein is encoded by a genetic sequence with at least about 80%, 85%, 90%, 95%, 97%, or 100% sequence identity to a sequence selected from the group consisting of SEQ ID NOS: 1-5.

In some embodiments, the targeted portion of the mRNA comprises a sequence with at least 80%, 85%, 90%, 95%, 97%, or 100% sequence identity to a region comprising at least 8 contiguous nucleic acids of a sequence selected from the group consisting of SEQ ID NOS: 6-10.

In some embodiments, the targeted portion of the mRNA is within the ASCE selected from the group consisting of: GRCh38/hg38: chr16 2092954 2093093; GRCh38/hg38: chr1 94111438 94111579; GRCh38/hg38: chr16 31186802 31186836; GRCh38/hg38: chr9 133066530 133066660; and GRCh38/hg38: chr5 177238237 177238507.

In some embodiments, the targeted portion of the mRNA is upstream or downstream of the ASCE selected from the group consisting of: GRCh38/hg38: chr16 2092954 2093093; GRCh38/hg38: chr1 94111438 94111579; GRCh38/hg38: chr16 31186802 31186836; GRCh38/hg38: chr9 133066530 133066660; and GRCh38/hg38: chr5 177238237 177238507.

In some embodiments, the targeted portion of the mRNA does not comprise an exon-intron junction of an ASCE selected from the group consisting of: GRCh38/hg38: chr16 2092954 2093093; GRCh38/hg38: chr1 94111438 94111579; GRCh38/hg38: chr16 31186802 31186836; GRCh38/hg38: chr9 133066530 133066660; and GRCh38/hg38: chr5 177238237 177238507.

In some embodiments, the target protein produced is a full-length protein or a wild-type protein.

In some embodiments, inclusion of the ASCE during the processing of the pre-mRNA in the cell contacted with the therapeutic agent or the vector encoding the therapeutic agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to inclusion of the ASCE during the processing of the pre-mRNA in a corresponding cell that is not contacted with the therapeutic agent or the vector encoding the therapeutic agent.

In some embodiments, the level of the processed mRNA produced in the cell contacted with the therapeutic agent or the vector encoding the therapeutic agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA in a corresponding cell that is not contacted with the therapeutic agent or the vector encoding the therapeutic agent.

In some embodiments, a level of the target protein produced in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein produced in a corresponding cell that is not contacted with the therapeutic agent or the vector encoding the therapeutic agent.

In some embodiments, exclusion of the ASCE during the processing of the pre-mRNA in the cell contacted with the therapeutic agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the ASCE during the processing of the pre-mRNA in a corresponding cell that is not contacted with the therapeutic agent or the vector encoding the therapeutic agent.

In some embodiments, the target protein is NSD1, and the method causes a modification of a histone protein in the cell.

In some embodiments, the histone protein is Histone H3.

In some embodiments, the modification comprises acetylation, methylation, phosphorylation, or ubiquitination.

In some embodiments, the modification is methylation.

In some embodiments, the methylation of the histone protein is increased in the cell.

In some embodiments, the methylation of the histone protein in the cell contacted with the therapeutic agent or the vector encoding the therapeutic agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the methylation of the histone protein in a corresponding cell that is not contacted with the therapeutic agent or the vector encoding the therapeutic agent.

In some embodiments, the target protein is the canonical isoform of the protein.

In some embodiments, the alternative processed mRNA that is produced by splicing out of the ASCE comprises a premature termination codon (PTC).

In some embodiments, the agent is an antisense oligomer (ASO).

In some embodiments, the ASO is at least 80%, at least 85%, at least 90%, at least 95%, at least 98%, at least 99%, or 100%, complementary to the targeted portion of the mRNA.

In some embodiments, the ASO comprises a sequence that is at least about 80%, 85%, 90%, 95%, 97%, or 100% complementary to at least 8 contiguous nucleic acids of a sequence selected from the group consisting of SEQ ID NOS: 6-10.

In some embodiments, the ASO comprises a backbone modification comprising a phosphorothioate linkage or a phosphorodiamidate linkage.

In some embodiments, the ASO comprises a phosphorodiamidate morpholino, a locked nucleic acid, a peptide nucleic acid, a 2′-O-methyl, a 2′-Fluoro, or a 2′-O-methoxyethyl moiety.

In some embodiments, the ASO comprises at least one modified sugar moiety.

In some embodiments, each sugar moiety is a modified sugar moiety.

In some embodiments, the ASO consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some embodiments, the ASO comprises a sequence with at least about 80%, 85%, 90%, 95%, 97%, or 100% sequence identity to any one of SEQ ID NOS: 16-1748.

In some embodiments, the target gene is NSD1, and the vector encoding the agent encodes a polynucleotide comprising a sequence with at least about 80%, 85%, 90%, 95%, 97%, or 100% sequence identity to any one of the ASO sequences listed in Table 4, Table 5A, Table 5A-1, Table 5B, Table 5B-1, Table 5D, Table 5E, Table 5G, and Table 5G-1.

In some embodiments, the vector encoding the agent is a viral vector.

In some embodiments, the viral vector is an adenovirus-associated viral vector.

In some embodiments, the vector encoding the agent encodes a polynucleotide comprising an ASO sequence and an snRNA.

In some embodiments, the snRNA comprises a modified snRNA.

In some embodiments, the modified snRNA is a modified U1 snRNA or a modified U7 snRNA.

In some embodiments, the snRNA comprises a U1 snRNA.

In some embodiments, the target gene is NSD1, and the ASO sequence comprises a sequence with at least about 80%, 85%, 90%, 95%, 97%, or 100% sequence identity to any one of the ASO sequences listed in Table 4, Table 5A, Table 5B, Table 5D, Table 5E, and Table 5G.

In some embodiments, the snRNA comprises a U7 snRNA.

In some embodiments, the target gene is NSD1, and the ASO comprises a sequence with at least about 80%, 85%, 90%, 95%, 97%, or 100% sequence identity to any one of the ASO sequences listed in Table 4, Table 5A, Table 5A-1, Table 5B, Table 5B-1, Table 5G, and Table 5G-1.

Provided herein, in some aspects, is a composition comprising an ASO that comprises a sequence with at least about 80%, 85%, 90%, 95%, 97%, or 100% sequence identity to any one of SEQ ID NOS: 16-1748.

In some embodiments, the ASO comprises a backbone modification comprising a phosphorothioate linkage or a phosphorodiamidate linkage.

In some embodiments, the ASO comprises a phosphorodiamidate morpholino, a locked nucleic acid, a peptide nucleic acid, a 2′-O-methyl, a 2′-Fluoro, or a 2′-O-methoxyethyl moiety.

In some embodiments, the ASO comprises at least one modified sugar moiety.

In some embodiments, each sugar moiety is a modified sugar moiety.

In some embodiments, the ASO consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Provided herein, in some aspects, is a composition comprising a vector encoding a polynucleotide comprising a sequence with at least about 80%, 85%, 90%, 95%, 97%, or 100% sequence identity to any one of the ASO sequences listed in Table 4, Table 5A, Table 5A-1, Table 5B, Table 5B-1, Table 5D, Table 5E, Table 5G, and Table 5G-1.

In some embodiments, the vector encoding the agent is a viral vector.

In some embodiments, the viral vector is an adenovirus-associated viral vector.

In some embodiments, the vector encoding the agent encodes a polynucleotide comprising an ASO sequence and an snRNA.

In some embodiments, the snRNA comprises a modified snRNA.

In some embodiments, the modified snRNA is a modified U1 snRNA or a modified U7 snRNA.

In some embodiments, the snRNA comprises a U1 snRNA.

In some embodiments, the ASO sequence comprises a sequence with at least about 80%, 85%, 90%, 95%, 97%, or 100% sequence identity to any one of the ASO sequences listed in Table 4, Table 5A, Table 5B, Table 5D, Table 5E, and Table 5G.

In some embodiments, the snRNA comprises a U7 snRNA.

In some embodiments, the ASO comprises a sequence with at least about 80%, 85%, 90%, 95%, 97%, or 100% sequence identity to any one of the ASO sequences listed in Table 4, Table 5A, Table 5A-1, Table 5B, Table 5B-1, Table 5G, and Table 5G-1.

Provided herein, in some aspects, is a pharmaceutical composition comprising the composition described herein; and a pharmaceutically acceptable excipient and/or a delivery vehicle.

Provided herein, in some aspects, is a method of treating or reducing the likelihood of developing a disease or condition in a subject in need thereof, the method comprising: administering to the subject a pharmaceutical composition described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B depict schematic representations of a target pre-mRNA that contains an alternatively-spliced coding exon (ASCE) which may be alternatively-spliced to produce a non-productive mRNA that undergoes nonsense mediated RNA decay (NMD) and therapeutic agent-mediated promotion of canonical splicing to increase expression of functional mRNA or the full-length target protein target mRNA.

FIG. 1A shows a cell divided into nuclear and cytoplasmic compartments. In the nucleus, a pre-mRNA transcript of a target gene undergoes splicing to generate mRNA, and this mRNA is exported to the cytoplasm and translated into target protein. For this target gene, some fraction of the pre-mRNA is alternatively-spliced leading to formation of a processed mRNA lacking the ASCE (non-productive mRNA) that undergoes NMD and is degraded in the cytoplasm, thus leading to no target protein production from the non-productive mRNA.

FIG. 1B shows an example of the same cell divided into nuclear and cytoplasmic compartments. Treatment with a therapeutic agent, such as an antisense oligomer (ASO), promotes inclusion of the ASCE in an mRNA processed from the pre-mRNA resulting in an increase in functional (productive) mRNA containing the ASCE, which is in turn translated into higher levels of target proteins.

FIG. 1C shows the difference between two alternative splicing events of a pre-mRNA transcript where one of the alternative splicing events leads to the formation of a non-productive mRNA lacking an ASCE (bottom) and where the other alternative splicing events leads to the formation of a productive mRNA containing the ASCE (top).

FIG. 1D shows the difference between two alternative splicing events of a NSD1 pre-mRNA transcript where one of the alternative splicing events leads to the formation of a non-productive mRNA lacking an ASCE (exon 8) (bottom) and where the other alternative splicing events leads to the formation of a productive mRNA containing the ASCE (exon 8) (top).

FIGS. 2A-2C depict confirmation of exemplary alternative splicing events of an ASCE in the NSD1 gene via cycloheximide treatment in astrocytes, Schwann cells and cynomolgus monkey brain cells.

FIG. 2A depicts a schematic in which peaks corresponding to RNA sequencing reads were identified in exon 8 of NSD1 (GRCh38/hg38: chr5 177238237:177238507).

FIG. 2B depicts gel images and a graph showing that cycloheximide treatment led to increase in the amount of non-productive mature NSD1 mRNA transcripts (processed NSD1 mRNA containing a premature termination codon rendering the transcript a target of NMD) in various human cells, including astrocytes, Schwann cells, HEK293 cells, SH-SY-5Y (neuroblastoma cell line) cells, and SK-N-AS (neuroblastoma cell line) cells.

FIG. 2C depicts a gel image and a graph showing the existence of non-productive mature NSD1 mRNA transcript in various cynomolgus brain regions, including cortex, brain stem, hippocampus, and cerebellum.

FIG. 2D depicts a gel image and a graph showing the existence of non-productive mature NSD1 mRNA transcript in human cortex.

FIGS. 3A-3D depict confirmation of inclusion or exclusion an ASCE of mouse NSD1 (mouse exon 7, corresponding to human exon 8) in NSD1 mRNA products processed from NSD1 pre-mRNA in mouse brains via in vivo or ex vivo cycloheximide treatment.

FIG. 3A depicts gel images showing that, in ex vivo cycloheximide-treated mouse brains, exclusion of an ASCE of mouse NSD1 (mouse exon 7, corresponding to human exon 8) leads to formation of a processed mRNA containing a premature termination codon rendering the transcript a target of NMD.

FIG. 3B depicts graphs of the percentage of NMD (top) and fold-change (bottom) of the NMD event of the non-productive NSD1 mRNA product relative to NSD1 productive NSD1 mRNA product from the gel images of FIG. 3A.

FIG. 3C depicts gel images showing that, in in vivo cycloheximide-treated mouse brains, exclusion of an ASCE of mouse NSD1 (mouse exon 7, corresponding to human exon 8) leads to formation of a processed mRNA containing a premature termination codon rendering the transcript a target of NMD.

FIG. 3D depicts graphs of the percentage of NMD (left) and fold-change (right) of the NMD event of the non-productive NSD1 mRNA product relative to NSD1 productive NSD1 mRNA product from the gel images of FIG. 3C.

FIGS. 4A-4B depict confirmation of inclusion or exclusion an ASCE of mouse NSD1 (mouse exon 7, corresponding to human exon 8) in NSD1 mRNA products processed from NSD1 pre-mRNA in mouse brains via in vivo cycloheximide treatment.

FIG. 4A depicts a gel image showing that, in in vivo cycloheximide-treated mouse brains, exclusion of an ASCE of mouse NSD1 (mouse exon 7, corresponding to human exon 8) leads to formation of a processed mRNA containing a premature termination codon rendering the transcript a target of NMD.

FIG. 4B depicts graphs of the percentage of NMD (left) and fold-change (right) of the NMD event of the non-productive NSD1 mRNA product relative to NSD1 productive NSD1 mRNA product from the gel images of FIG. 4A.

FIG. 5 depicts an exemplary ASO walk around the human NSD1 exon 8 (GRCh38/hg38: chr5 177238237:177238507) region. The underlined nucleotides correspond to the exon skipping event and arrows point to canonical 5′ or 3′ splice sites. Figure discloses SEQ ID NOS: 1775-1780, respectively, in order of appearance.

FIGS. 6A-6B show graphs summarizing the changes in the level of productive NSD1 mRNA (FIG. 6A) and non-productive NSD1 mRNA (FIG. 6B) in one ASO walk around exon 8 in HEK293 cells.

FIGS. 7A-7B show graphs summarizing the changes in the level of productive NSD1 mRNA (FIG. 7A) and non-productive NSD1 mRNA (FIG. 7B) in one ASO walk around exon 8.

FIG. 8 depicts an exemplary ASO walk around the human NSD1 exon 8 (GRCh38/hg38: chr5 177238237:177238507) region for an ASO vectorization approach using U7 snRNA.

FIG. 9 depicts an exemplary ASO walk around the human NSD1 exon 8 (GRCh38/hg38: chr5 177238237:177238507) region for an ASO vectorization approach using U1 snRNA.

FIG. 10 shows representative histograms of non-productive NSD1 mRNA levels when different cell lines are treated with alternative NMD inhibitors. SH-SY5Y, U-87 MG, HEK293, and SK-N-AS cell lines were each treated with one of three conditions: a mock control (vehicle only), NMD inhibitor cycloheximide (CHX), or NMD inhibitor SMG1i. Treatment with SMG1i resulted in ˜28% NSD1 non-productive mRNA (percentage of the level of non-productive NSD1 mRNA transcript in the total level of all NSD1 mRNA transcripts) in U-87 MG cells, ˜19% NSD1 non-productive mRNA levels in SH-SY5Y cells, and <˜18% NSD1 non-productive mRNA levels in HEK293 and SK-N-AS cells. Treatment with CHX resulted in ˜23% NSD1 non-productive mRNA in SH-SY5Y cells, ˜15% NSD1 non-productive mRNA in U-87 MG cells, and ˜13% NSD1 non-productive mRNA in HEK293 and SK-N-AS cells. In cells treated only with vehicle (mock), the percentage of non-productive RNA remained low.

FIGS. 11A-11C show data demonstrating that exemplary ASOs with alternative backbone modifications have similar effects on NSD1 pre-mRNA splicing. FIG. 11A is a table showing the ASO names, their backbone chemistries, sequences, and lengths. Figure discloses SEQ ID NOS: 1768-1774, respectively, in order of appearance. FIG. 11B is a scatterplot showing the fold change in productive and non-productive NSD1 mRNA when various ASOs with either PMO or 2′MOE-PS backbone modifications were nucleofected into U-87 MG cells, relative to cells treated with mock control. FIG. 11C is a histogram of the NSD1 protein levels present in U-87 MG cells after treatment with the ASOs of the various backbones (see FIG. 11A), relative to cells treated with mock control. Data from both FIG. 11B and FIG. 11C are normalized to the mock controls.

FIGS. 12A-12B depict representative data illustrating the effects of exemplary ASOs on NSD1 protein expression and H3K36me2 levels in U-87 MG cells. FIG. 12A is a histogram showing the fold change of NSD1 protein in U-87 MG cells treated with various ASOs, relative to cells treated with water only. FIG. 12B is a histogram showing the fold change in cellular H3K36me2 levels in U-87 MG cells treated with various ASOs, relative to cells treated with water only. U-87 cells were nucleofected with 1 μM of each ASO and cells were harvested 72 hours after nucleofection. NSD1 protein levels were measured by immuno-capillary electrophoresis (JESS), and H3K36me2 levels were measured by AlphaLISA®. The data present in FIGS. 12A-12B are the sum of 2-3 independent experiments; mean±SEM.

FIGS. 13A-13C depict representative data illustrating the dose-dependent effect of an exemplary ASO on NSD1 protein expression and H3K36me2 levels in U-87 MG cells. FIG. 13A is a histogram showing the fold change of NSD1 protein in U-87 MG cells treated with ASO 211 at various dosage concentrations (0.25 μM, 0.5 μM, 1.0 μM, or 2.0 μM), relative to cells treated with water only. FIG. 13B is a histogram showing the fold change in cellular H3K36me2 levels in U-87 MG cells treated ASO 211 at various dosage concentrations (0.25 μM, 0.5 μM, 1.0 μM, or 2.0 μM), relative to cells treated with water only. FIG. 13C is a histogram showing the total Histone H3 levels present in U-87 cells treated with ASO 211 at various dosage concentrations, as compared to cells treated with water only. U-87 cells were nucleofected with ASO 211 at four tested dosages (0.25 μM, 0.5 μM, 1.0 μM, or 2.0 μM) and cells were harvested 72 hours after nucleofection. NSD1 protein measured by immuno-capillary electrophoresis (JESS), and H3K36me2 levels were measured by AlphaLISA®. Total cellular Histone H3 levels were also measured by AlphaLISA®. The data present in FIGS. 13A-13C are the sum of 2-3 independent experiments; mean±SEM; one-way ANOVA; * pval<0.05, ***pval<0.01; ****pval<0.001.

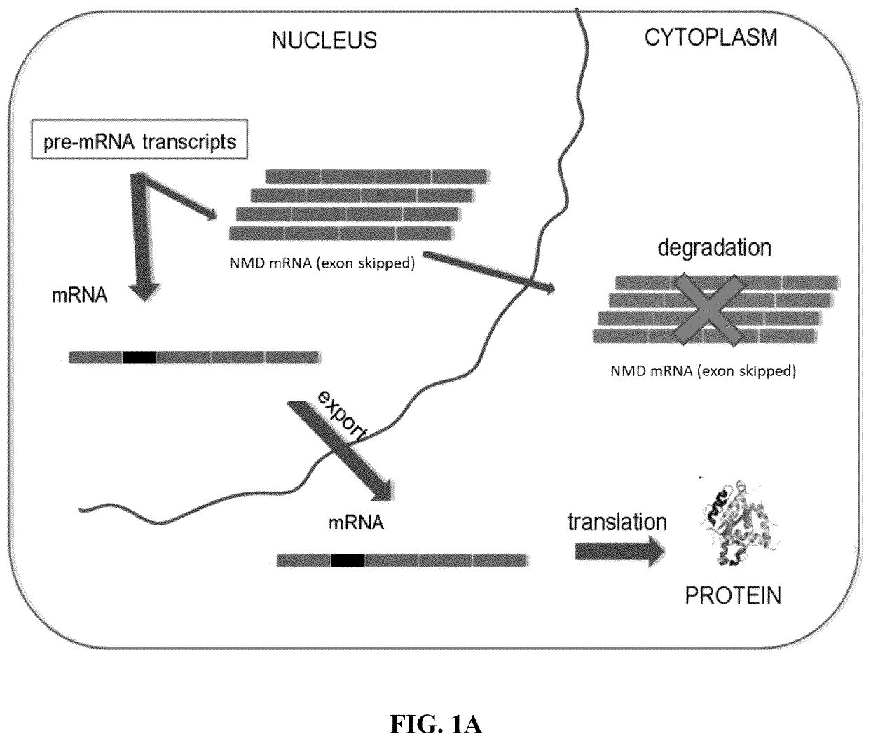

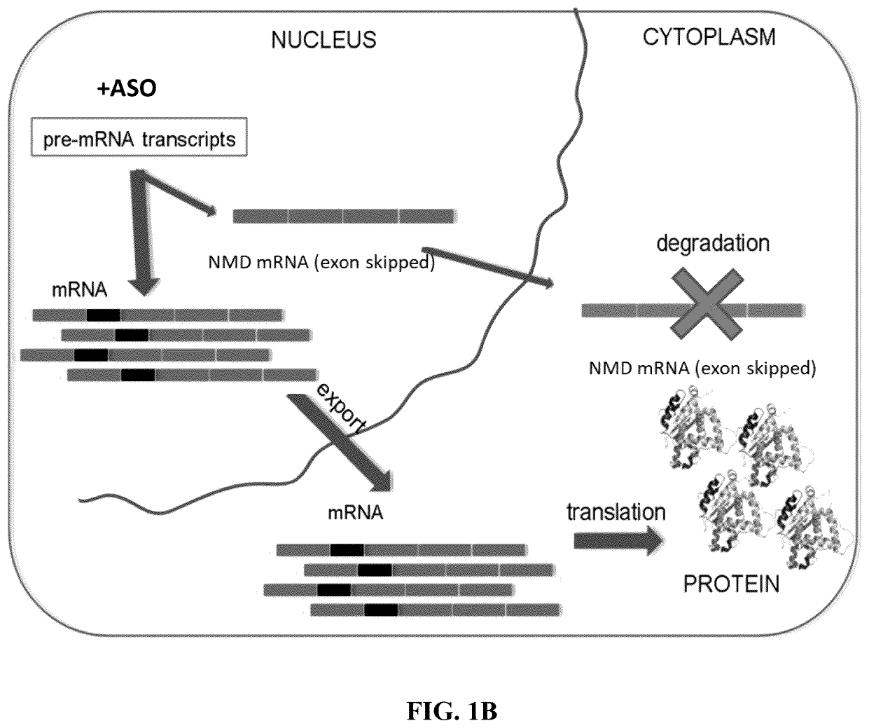

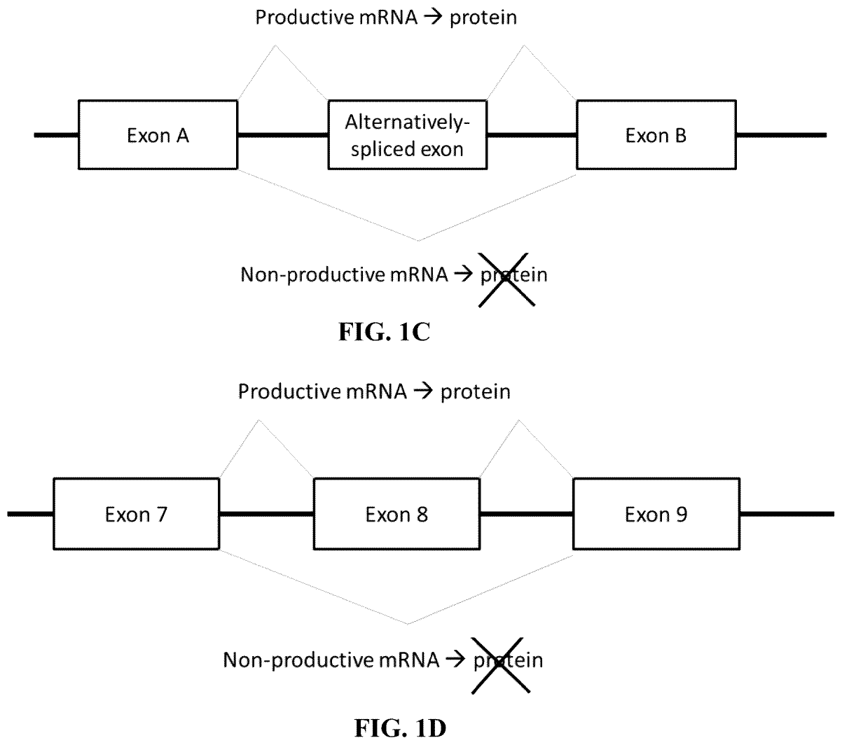

### DETAILED DESCRIPTION

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The coordinate as used herein refers to the coordinate of the genome reference assembly GRCh38 (Genome Research Consortium human build 38), also known as Hg38 (Human genome build 38).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

Alternative splicing events in PKD1, ABCA4, FUS, CEL or NSD1 gene can lead to non-productive mRNA transcripts which in turn can lead to reduced protein expression, and therapeutic agents which can target the alternative splicing events in PKD1, ABCA4, FUS, CEL or NSD1 gene can modulate (e.g., increase) the expression level of functional proteins in patients. Such therapeutic agents can be used to treat a condition caused by deficiency in amount or activity of polycystin-1, retinal-specific phospholipid-transporting ATPase ABCA4, RNA-binding protein FUS, bile salt-activated lipase, or Histone-lysine N-methyltransferase, H3 lysine-36 specific.

One alternative splicing event that can lead to non-productive mRNA transcripts is an alternatively-spliced coding exon (ASCE) event. For example, exclusion of an alternatively-spliced coding exon can result in a processed mRNA that is shorter than a corresponding processed mRNA in which the ASCE is included (the shorter processed mRNA is also termed “alternative processed mRNA” herein). For example, skipping of an alternatively-spliced coding exon can result in a processed mRNA that is shorter than a corresponding processed mRNA in which the ASCE is included. For example, exclusion of an alternatively-spliced coding exon resulting from the reduced or inhibited splicing of a 3′ splice-site of the ASCE (e.g., the canonical 3′ ss) and/or reduced or inhibited splicing of a 5′ splice-site of the ASCE (e.g., the canonical 5′ ss) can result in a processed mRNA that is shorter than a corresponding processed mRNA in which the ASCE is included. The present disclosure provides compositions and methods for modulating alternative splicing of PKD1, ABCA4, FUS, CEL or NSD1 pre-mRNA to increase the production of protein-coding mature mRNA, and thus, translated functional polycystin-1, retinal-specific phospholipid-transporting ATPase ABCA4, RNA-binding protein FUS, bile salt-activated lipase, or Histone-lysine N-methyltransferase, H3 lysine-36 specific. For example, the compositions and methods provided herein can modulate processing of PKD1, ABCA4, FUS, CEL or NSD1 pre-mRNA by promoting or increasing splicing of a 3′ splice-site of the ASCE (e.g., the canonical 3′ ss) and/or promoting or increasing splicing of a 5′ splice-site of the ASCE (e.g., the canonical 5′ ss). For example, the compositions and methods provided herein can modulate processing of PKD1, ABCA4, FUS, CEL or NSD1 pre-mRNA by promoting or increasing splicing of a 3′ splice-site of the intron upstream of the ASCE and/or by promoting or increasing splicing of a 5′ splice-site of the intron downstream of the ASCE.

These compositions and methods include antisense oligomers (ASOs) or vectors encoding ASOs that can promote constitutive splicing of PKD1, ABCA4, FUS, CEL or NSD1 pre-mRNA. For example, these compositions and methods include ASOs or vectors encoding ASOs that can promote inclusion of an ASCE in a processed mRNA that is processed from a PKD1, ABCA4, FUS, CEL or NSD1 pre-mRNA. In various embodiments, functional polycystin-1, retinal-specific phospholipid-transporting ATPase ABCA4, RNA-binding protein FUS, bile salt-activated lipase, or Histone-lysine N-methyltransferase, H3 lysine-36 specific can be increased using the methods of the disclosure to treat a condition caused by deficient amount or activity of polycystin-1, retinal-specific phospholipid-transporting ATPase ABCA4, RNA-binding protein FUS, bile salt-activated lipase, or Histone-lysine N-methyltransferase, H3 lysine-36 specific protein.

“Polycystin-1” or “PC1,” also known as Autosomal dominant polycystic kidney disease 1 protein, as referred to herein, can be encoded by a PKD1 gene and can be a membrane protein involved in cell-to-cell or cell-matrix interactions that can be a component of a heteromeric calcium-permeable ion channel formed with polycystin-2 (encoded by a PKD2 gene) that is activated by interaction with a Wnt family member, such as WNT3A and WNT9B, and that regulates multiple signaling pathways to maintain normal renal tubular structure and function, includes any of the recombinant or naturally-occurring forms of polycystin-1 or variants or homologs thereof that have or maintain polycystin-1 activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99%, or 100% amino acid sequence identity across the whole sequence or a portion of the sequence (e.g., a 50, 100, 150 or 200 continuous amino acid portion) compared to a naturally occurring polycystin-1. In some embodiments, polycystin-1 is substantially identical to the protein identified by the UniProt reference number P98161 or a variant or homolog having substantial identity thereto.

“Retinal-specific phospholipid-transporting ATPase ABCA4,” also known as ATP binding cassette subfamily A member 4, RIM ABC transporter (RIM protein or RmP), Retinal-specific ATP-binding cassette transporter, or Stargardt disease protein, as referred to herein, can be encoded by a ABCA4 gene (also known as ABCR) and can be a membrane-associated protein that is a member of the superfamily of ATP-binding cassette (ABC) transporters that can be a retina-specific ABC transporter with N-retinylidene-PE as a substrate, and can be expressed exclusively in retina photoreceptor cells and can mediate transport of an essential molecule, all-trans-retinal aldehyde (atRAL), across the photoreceptor cell membrane, includes any of the recombinant or naturally-occurring forms of Retinal-specific phospholipid-transporting ATPase ABCA4 or variants or homologs thereof that have or maintain Retinal-specific phospholipid-transporting ATPase ABCA4 activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99%, or 100% amino acid sequence identity across the whole sequence or a portion of the sequence (e.g., a 50, 100, 150 or 200 continuous amino acid portion) compared to a naturally occurring Retinal-specific phospholipid-transporting ATPase ABCA4. In some embodiments, Retinal-specific phospholipid-transporting ATPase ABCA4 is substantially identical to the protein identified by the UniProt reference number P78363 or a variant or homolog having substantial identity thereto.

“RNA-binding protein FUS,” also known as FUS RNA binding protein, 75 kDa DNA-pairing protein, Oncogene FUS, Oncogene TLS, POMp75, or Translocated in liposarcoma protein, as referred to herein, can be encoded by a FUS gene (also known as TLS) and can be a DNA/RNA-binding protein that plays a role in various cellular processes such as transcription regulation, RNA splicing, RNA transport, DNA repair and damage response, includes any of the recombinant or naturally-occurring forms of RNA-binding protein FUS or variants or homologs thereof that have or maintain RNA-binding protein FUS activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99%, or 100% amino acid sequence identity across the whole sequence or a portion of the sequence (e.g., a 50, 100, 150 or 200 continuous amino acid portion) compared to a naturally occurring RNA-binding protein FUS. In some embodiments, RNA-binding protein FUS is substantially identical to the protein identified by the UniProt reference number P35637 or a variant or homolog having substantial identity thereto.

“Bile salt-activated lipase,” also known as Carboxyl ester lipase, Bile salt-stimulated lipase (BSSL), Bucelipase, Cholesterol esterase, Pancreatic lysophospholipase, or Sterol esterase, as referred to herein, can be encoded by a CEL gene (also known as BAL) and can catalyzes the hydrolysis of a wide range of substrates including cholesteryl esters, phospholipids, lysophospholipids, di- and tri-acylglycerols, and fatty acid esters of hydroxy fatty acids (FAHFAs), includes any of the recombinant or naturally-occurring forms of Bile salt-activated lipase or variants or homologs thereof that have or maintain Bile salt-activated lipase activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Bile salt-activated lipase In some embodiments, Bile salt-activated lipase is substantially identical to the protein identified by the UniProt reference number P19835 or a variant or homolog having substantial identity thereto.

“Histone-lysine N-methyltransferase, H3 lysine-36 specific,” also known as Androgen receptor coactivator 267 kDa protein, Androgen receptor-associated protein of 267 kDa, H3-K36-HMTase, Lysine N-methyltransferase 3B, Nuclear receptor-binding SET domain-containing protein 1 (NR-binding SET domain-containing protein), as referred to herein, can be encoded by a NSD1 gene (also known as ARA267 and KMT3B) and can be a histone methyltransferase that dimethylates Lys-36 of histone H3 (H3K36me2) and can be a transcriptional intermediary factor capable of both negatively or positively influencing transcription, depending on the cellular context, includes any of the recombinant or naturally-occurring forms of Histone-lysine N-methyltransferase, H3 lysine-36 specific or variants or homologs thereof that have or maintain Histone-lysine N-methyltransferase, H3 lysine-36 specific activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Histone-lysine N-methyltransferase, H3 lysine-36 specific In some embodiments, Histone-lysine N-methyltransferase, H3 lysine-36 specific is substantially identical to the protein identified by the UniProt reference number Q96L73 or a variant or homolog having substantial identity thereto.

The terms “alternatively-spliced coding exon” or “ASCE” are used interchangeably and can refer to a coding exon (e.g., a canonical exon) that can prevent activation of the nonsense-mediated mRNA decay (NMD) pathway if present in a mature RNA transcript or promote activation of the NMD pathway if absent in a mature RNA transcript. In constitutive splicing events, the ASCE is usually not spliced out, but the ASCE may be excluded during alternative or aberrant splicing events. Mature mRNA transcripts lacking an ASCE may be non-productive, for example, due to frame shifts which induce the NMD pathway. In some embodiments, an ASCE is a skipped exon. In some embodiments, an ASCE is an exon that leads to an alteration of reading frame when the ASCE is not included in a mature or processed mRNA. In some embodiments, an ASCE is an exon containing a number of nucleotides that is not evenly divisible by 3. In some embodiments, a mature or processed mRNA in which the ASCE has been excluded contains a premature stop codon (or premature termination codon (PTC)) or other sequences that facilitate degradation of a mature RNA transcript in which the ASCE has been excluded. Exclusion of an ASCE in mature or processed RNA transcripts may downregulate gene expression. In some embodiments, a mature or processed mRNA in which the ASCE has been excluded is created from alternative splicing events. For example, a mature or processed mRNA in which the ASCE has been excluded can be created from an alternative 3′ splice site event. For example, a mature or processed mRNA in which the ASCE has been excluded can be created from an alternative 5′ splice site event. For example, a mature or processed mRNA in which the ASCE has been excluded can be created from an alternative 5′ splice site event and an alternative 3′ splice site event. For example, a mature or processed mRNA in which the ASCE has been excluded can be created from an exon skipping event. For example, an ASCE can be a canonical exon. For example, only exons that are evenly divisible by 3 can be skipped or included in the mRNA without any alteration of reading frame.

Alternative splicing can result in exclusion of at least one ASCE in the mature mRNA transcripts. The terms “mature mRNA,” and “fully spliced mRNA,” are used interchangeably herein to describe a fully processed mRNA. A mature mRNA that lacks an ASCE can be non-productive mRNA and lead to NMD of the mature mRNA. Mature mRNA lacking an ASCE may sometimes lead to reduced protein expression compared to protein expression from a corresponding mature mRNA that contains the ASCE.

Pseudo splice sites have the same splicing recognition sequences as genuine splice sites but are not used in splicing reactions. They outnumber genuine splice sites in the human genome by an order of a magnitude and are normally repressed by thus far poorly understood molecular mechanisms. Cryptic 5′ splice sites have the consensus NNN/GUNNNN or NNN/GCNNNN where N is any nucleotide and/is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where M is C or A, R is G or A, and Y is C or U.

Splice sites and their regulatory sequences can be readily identified by a skilled person using suitable algorithms publicly available, listed for example in Kralovicova, J. and Vorechovsky, I. (2007) Global control of aberrant splice site activation by auxiliary splicing sequences: evidence for a gradient in exon and intron definition. Nucleic Acids Res., 35, 6399-6413, (ncbi.nlm.nih.gov/pmc/articles/PMC2095810/pdf/gkm680.pdf).

Splicing and Nonsense-Mediated mRNA Decay

Intervening sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of the 5′ splice site (5′ss) by U1 snRNA or the 3′ splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) or premature stop codons at least 50 bp upstream of an exon junction complex are likely to be targeted for degradation by the nonsense-mediated mRNA decay (NMD) pathway. Mutations in traditional (BPS/PPT/3′ss/5′ss) and auxiliary splicing motifs can cause aberrant splicing, such as exon skipping or cryptic (or pseudo-) exon inclusion or splice-site activation and contribute significantly to human morbidity and mortality. Both aberrant and alternative splicing patterns can be influenced by natural DNA variants in exons and introns.

Given that exon-intron boundaries can occur at any of the three positions of a codon, it is clear that only a subset of alternative splicing events can maintain the canonical open reading frame. For example, only exons that are evenly divisible by 3 can be skipped or included in the mRNA without any alteration of reading frame. Splicing events that do not have compatible phases will induce a frameshift. Unless reversed by downstream events, frameshifts can certainly lead to one or more PTCs, probably resulting in subsequent degradation by NMD. NMD is a translation-coupled mechanism that eliminates mRNAs containing PTCs. NMD can function as a surveillance pathway that exists in all eukaryotes. NMD can reduce errors in gene expression by eliminating mRNA transcripts that contain premature stop codons or PTCs. Translation of these aberrant mRNAs could, in some cases, lead to deleterious gain-of-function or dominant-negative activity of the resulting proteins. NMD targets not only transcripts with PTCs but also a broad array of mRNA isoforms expressed from many endogenous genes, suggesting that NMD is a master regulator that drives both fine and coarse adjustments in steady-state RNA levels in the cell.

In some cases, a therapeutic agent comprises a modified snRNA, such as a modified human or murine snRNA. In some cases, a therapeutic agent comprises a vector, such as a viral vector, that encodes a modified snRNA. In some embodiments, the modified snRNA is a modified U1 snRNA (see, e.g., Alanis et al., Human Molecular Genetics, 2012, Vol. 21, No. 11 2389-2398). In some embodiments, the modified snRNA is a modified U7 snRNA (see, e.g., Gadgil et al., J Gene Med. 2021; 23:e3321). Modified U7 snRNAs can be made by any method known in the art including the methods described in Meyer, K.; Schümperli, Daniel (2012), Antisense Derivatives of U7 Small Nuclear RNA as Modulators of Pre-mRNA Splicing. In: Stamm, Stefan; Smith, Christopher W. J.; Lührmann, Reinhard (eds.) Alternative pre-mRNA Splicing: Theory and Protocols (pp. 481-494), Chichester: John Wiley & Sons 10.1002/9783527636778.ch45, incorporated by reference herein in its entirety. In some embodiments, a modified U7 (smOPT) does not compete with WT U7 (Stefanovic et al., 1995).

In some embodiments, the modified snRNA comprises an smOPT modification. For example, the modified snRNA can comprise a sequence AAUUUUUGGAG (SEQ ID NO: 1749). For example, the sequence AAUUUUUGGAG (SEQ ID NO: 1749) can replace a sequence AAUUUGUCUAG (SEQ ID NO: 1750) in a wild-type U7 snRNA to generate the modified U7 snRNA (smOPT). In some embodiments, a smOPT modification of a U7 snRNA renders the particle functionally inactive in histone pre-mRNA processing (Stefanovic et al., 1995). In some embodiments, a modified U7 (smOPT) is expressed stably in the nucleus and at higher levels than WT U7 (Stefanovic et al., 1995). In some embodiments, the snRNA comprises a U1 snRNP-targeted sequence. In some embodiments, the snRNA comprises a U7 snRNP-targeted sequence. In some embodiments, the snRNA comprises a modified U7 snRNP-targeted sequence and wherein the modified U7 snRNP-targeted sequence comprises smOPT. In some embodiments, the modified snRNA has been modified to comprise a single-stranded nucleotide sequence that hybridizes to a pre-mRNA, such as an ASCE-containing pre-mRNA. For example, the modified snRNA can be modified to comprise a single-stranded nucleotide sequence that hybridizes to PKD1, ABCA4, FUS, CEL or NSD1 pre-mRNA. In some embodiments, the modified snRNA is designed according to the format described in Table 5C or Table 5F. In some cases, the modified snRNA that comprises U7 snRNP-targeted sequence is designed according to the format described in Table 5C. In some cases, the modified snRNA that comprises U1 snRNP-targeted sequence is designed according to the format described in Table 5F. In some embodiments, a U7 snRNP-targeted sequence comprises a single-stranded nucleotide sequence that hybridizes to PKD1, ABCA4, FUS, CEL or NSD1 pre-mRNA, where the single-stranded nucleotide sequence starts with dinucleotides AA, such as the sequences in Table 5A-1, Table 5B-1, and Table 5G-1. In some of these embodiments, when designing a single-stranded nucleotide sequence that is complementary to a target sequence in the target pre-mRNA (e.g., PKD1, ABCA4, FUS, CEL or NSD1 pre-mRNA), if the sequence complementary to the target sequence starts with nucleotides other than dinucleotides AA on the 5′ end, dinucleotides AA will be added to its 5′ end; if the sequence complementary to the target sequence starts with one A nucleotide on the 5′ end that is followed by a non-A nucleotide, then one A will be added to its 5′ end. In some other cases, if the sequence complementary to the target sequence starts with dinucleotides AA on the 5′ end, then no additional A nucleotides will be added.

In some embodiments, the modified snRNA has been modified to comprise a single-stranded nucleotide sequence that hybridizes to a PKD1, ABCA4, FUS, CEL or NSD1 ASCE-containing pre-mRNA. In some embodiments, the modified snRNA has been modified to comprise a single-stranded nucleotide sequence that comprises one or two or more sequences of the ASOs disclosed herein. In some embodiments, the modified snRNA has been modified to comprise a single-stranded nucleotide sequence that hybridizes to sequence of a pre-mRNA with a mutation, such as a PKD1, ABCA4, FUS, CEL or NSD1 ASCE-containing pre-mRNA with a mutation. In some embodiments, the modified snRNA has been modified to comprise a single-stranded nucleotide sequence that comprises two or more sequences that hybridize to two or more target regions of an ASCE-containing pre-mRNA, such as a PKD1, ABCA4, FUS, CEL or NSD1 ASCE-containing pre-mRNA. For example, a modified snRNA can be modified to comprise a single-stranded nucleotide sequence that hybridizes to at least 8 contiguous nucleic acids of an ASCE-containing pre-mRNA, such as a PKD1, ABCA4, FUS, CEL or NSD1 ASCE-containing pre-mRNA. In some embodiments, the modified snRNA has been modified to comprise a single-stranded nucleotide sequence that hybridizes to any of the target regions of an ASCE-containing pre-mRNA, such as a PKD1, ABCA4, FUS, CEL or NSD1 ASCE-containing pre-mRNA disclosed herein. In some embodiments, the modified snRNA has been modified to comprise a single-stranded nucleotide sequence that comprises two or more sequences that hybridize to two or more target regions of an ASCE-containing pre-mRNA, such as a PKD1, ABCA4, FUS, CEL or NSD1 ASCE-containing pre-mRNA. For example, a modified snRNA can be modified to comprise a single-stranded nucleotide sequence that hybridizes to one or two or more sequences of an intron upstream of the ASCE, an intron downstream of the ASCE, an exon upstream of the ASCE, an exon downstream of the ASCE or within the ASCE of an ASCE-containing pre-mRNA, such as a PKD1, ABCA4, FUS, CEL or NSD1 ASCE-containing pre-mRNA, or to an ASCE-skipping regulatory sequence in the ASCE-containing pre-mRNA. For example, a modified snRNA can be modified to comprise a single-stranded nucleotide sequence that hybridizes to one or two or more sequences of an intron upstream of the ASCE. For example, a modified snRNA can be modified to comprise a single-stranded nucleotide sequence that hybridizes to one or two or more sequences of an intron downstream of the ASCE. For example, a modified snRNA can be modified to comprise a single-stranded nucleotide sequence that hybridizes to one or two or more sequences of an exon upstream of the ASCE. For example, a modified snRNA can be modified to comprise a single-stranded nucleotide sequence that hybridizes to one or two or more sequences of an exon downstream of the ASCE. For example, a modified snRNA can be modified to comprise a single-stranded nucleotide sequence that hybridizes to one or two or more sequences within the ASCE. For example, a modified snRNA can be modified to comprise a single-stranded nucleotide sequence that hybridizes to one or two or more sequences of an intron upstream of the ASCE, an intron downstream of the ASCE, an exon upstream of the ASCE, an exon downstream of the ASCE or within the ASCE of a PKD1, ABCA4, FUS, CEL or NSD1 ASCE-containing pre-mRNA (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1). For example, a modified snRNA can be modified to comprise a single-stranded nucleotide sequence that hybridizes to a region within an ASCE or upstream or downstream of an ASCE (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1). In some embodiments, the modified snRNA has a 5′ region that has been modified to comprise a single-stranded nucleotide sequence that hybridizes to an ASCE-containing pre-mRNA, such as a PKD1, ABCA4, FUS, CEL or NSD1 ASCE-containing pre-mRNA. In some embodiments, the modified snRNA has a 3′ region that has been modified to comprise a single-stranded nucleotide sequence that hybridizes to an ASCE-containing pre-mRNA, such as a PKD1, ABCA4, FUS, CEL or NSD1 ASCE-containing pre-mRNA.

For example, a modified snRNA can be modified to comprise a single-stranded nucleotide sequence that hybridizes to a region that does not overlap with an ASCE and an intron upstream of the ASCE (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1). For example, a modified snRNA can be modified to comprise a single-stranded nucleotide sequence that does not hybridize to a region that overlaps with an ASCE and an intron downstream of the ASCE (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1).

For example, a modified snRNA can be modified to comprise a single-stranded nucleotide sequence that is complementary to an exon sequence or an intron sequence that is downstream of an ASCE (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1). For example, a modified snRNA can be modified to comprise a single-stranded nucleotide sequence that is not complementary to a 3′ splice site of an intron sequence that is downstream of an ASCE (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1). For example, a modified snRNA can be modified to comprise a single-stranded nucleotide sequence that is not complementary to a 5′ splice site of an intron sequence that is downstream of an ASCE (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1).

For example, a modified snRNA can be modified to comprise a single-stranded nucleotide sequence that is complementary to an intron sequence that is upstream of an ASCE (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1). For example, a modified snRNA can be modified to comprise a single-stranded nucleotide sequence that is not complementary to a splice site of an intron sequence that is upstream of an ASCE (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1). For example, a modified snRNA can be modified to comprise a single-stranded nucleotide sequence that is not complementary to a 3′ splice site of an intron sequence that is upstream of an ASCE (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1). For example, a modified snRNA can be modified to comprise a single-stranded nucleotide sequence that is not complementary to a 5′ splice site of an intron sequence that is upstream of an ASCE (e.g., exon 38 of PKD1 (e.g., exon (GRCh38/hg38: chr16 2092954 2093093) of PKD1), e.g., exon 3 of ABCA4 (e.g., exon (GRCh38/hg38: chr1 94111438 94111579) of ABCA4), e.g., exon 7 of FUS (e.g., exon (GRCh38/hg38: chr16 31186802 31186836) of FUS), e.g., exon 5 of CEL (e.g., exon (GRCh38/hg38: chr9 133066530 133066660) of CEL), e.g., exon 8 of NSD1 (e.g., exon (GRCh38/hg38: chr5 177238237 177238507)) of NSD1).

Methods of Identifying Additional ASOs that Promote Splicing at a Canonical 3′ Splice Site and/or to Promote Splicing at a Canonical 5′ Splice Site

Also within the scope of the present disclosure are methods for identifying or determining therapeutic agents, such as ASOs, that promote splicing at a canonical 3′ splice site of an ASCE, that promote splicing at a canonical 3′ splice site of the intron upstream of an ASCE, that promote splicing at a canonical 5′ splice site of an ASCE and/or that promote splicing at a canonical 5′ splice site of the intron downstream of an ASCE of an ASCE-containing pre-mRNA, such as a PKD1, ABCA4, FUS, CEL or NSD1 ASCE-containing pre-mRNA. For example, a method can comprise identifying or determining ASOs that inhibit or reduce ASCE skipping of an ASCE-containing pre-mRNA, such as a PKD1, ABCA4, FUS, CEL or NSD1 ASCE-containing pre-mRNA. ASOs that specifically hybridize to different nucleotides within the target region of the pre-mRNA may be screened to identify or determine ASOs that improve the rate and/or extent of splicing at a canonical 3′ splice site of an ASCE, a canonical 3′ splice site of the intron upstream of an ASCE, a canonical 5′ splice site of an ASCE and/or that a canonical 5′ splice site of the intron downstream of an ASCE, and/or reduce the rate and/or extent of splicing at an alternative 3′ splice site and/or alternative 5′ splice site of an ASCE. In some embodiments, the ASO may block or interfere with the binding site(s) of a splicing repressor(s)/silencer. Any method known in the art may be used to identify (determine) an ASO that when hybridized to the target region results in the desired effect (e.g., promoting splicing at a canonical 3′ splice site of an ASCE, promoting splicing at a canonical 3′ splice site of the intron upstream of an ASCE, promoting splicing at a canonical 5′ splice site of an ASCE, promoting splicing at a canonical 5′ splice site of the intron downstream of an ASCE, protein production, or functional RNA production). These methods also can be used for identifying ASOs that promote or increase inclusion of an ASCE by binding to a target region flanking the ASCE, or in the ASCE. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ or 5′ splice site of the ASCE to approximately 100 nucleotides downstream of the 3′ or 5′ splice site of the ASCE. For example, the ASOs used in the ASO walk can be tiled every 5 nucleotides from approximately 100 nucleotides upstream of the 5′ splice site of the intron following the ASCE to approximately 100 nucleotides downstream of the 3′ splice site of the intron following the ASCE. For example, the ASOs used in the ASO walk can be tiled every 5 nucleotides from approximately 100 nucleotides upstream of the 3′ splice site of the intron preceding the ASCE to approximately 100 nucleotides downstream of the 5′ splice site of the intron preceding the ASCE. For example, the ASOs used in the ASO walk can be tiled every 5 nucleotides from approximately 100 nucleotides upstream of the 5′ splice site of the intron following the ASCE to approximately 100 nucleotides downstream of the 5′ splice site of the intron following the ASCE. For example, the ASOs used in the ASO walk can be tiled every 5 nucleotides from approximately 100 nucleotides upstream of the 3′ splice site of the intron following the ASCE to approximately 100 nucleotides downstream of the 3′ splice site of the intron following the ASCE. For example, the ASOs used in the ASO walk can be tiled every 5 nucleotides from approximately 100 nucleotides upstream of the 3′ splice site of the intron preceding the ASCE to approximately 100 nucleotides downstream of the 3′ splice site of the intron preceding the ASCE. For example, the ASOs used in the ASO walk can be tiled every 5 nucleotides from approximately 100 nucleotides upstream of the 5′ splice site of the intron preceding the ASCE to approximately 100 nucleotides downstream of the 5′ splice site of the intron preceding the ASCE. For example, the ASOs used in the ASO walk can be tiled every 5 nucleotides from approximately 100 nucleotides upstream of the 3′ or 5′ splice site of the ASCE to approximately 100 nucleotides downstream of the 3′ or 5′ splice site of the ASCE. For example, a first ASO of 15 nucleotides in length may be designed to specifically hybridize to nucleotides +6 to +20 relative to the 3′ splice site of the intron preceding of the ASCE. A second ASO may be designed to specifically hybridize to nucleotides +11 to +25 relative to the 3′ splice site of the intron preceding the ASCE. ASOs are designed as such spanning the target region of the pre-mRNA. In embodiments, the ASOs can be tiled more closely, e.g., every 1, 7, 8, or 9 nucleotides. Further, the ASOs can be tiled from 100 nucleotides downstream of the 5′ splice site, to 100 nucleotides upstream of the 3′ splice site. In some embodiments, the ASOs can be tiled from about 500 nucleotides upstream of the 3′ splice site, to about 500 nucleotides downstream of the 5′ splice site. In some embodiments, the ASOs can be tiled from about 500 nucleotides upstream of the 3′ splice site, to about 500 nucleotides downstream of the 3′ splice site.

One or more ASOs, or a control ASO (an ASO with a scrambled sequence, sequence that is not expected to hybridize to the target region) can be delivered, for example by transfection, into a disease-relevant cell line that expresses the target pre-mRNA (e.g., a ASCE-containing pre-mRNA described herein). The exon skipping inhibition or ASCE inclusion promotion effects of each of the ASOs may be assessed by any method known in the art, for example by reverse transcriptase (RT)-PCR using primers that span the splice junction. An increase or presence of a longer RT-PCR product produced using the primers spanning the region containing the ASCE (e.g., including the exons flanking the ASCE) in ASO-treated cells as compared to in control ASO-treated cells indicates that splicing out of the target ASCE has been inhibited. In some embodiments, the exon skipping inhibition efficiency, the ratio of unspliced to spliced pre-mRNA, the decrease in rate of splicing, or the reduction in extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in promotion of inclusion of an ASCE in a mature RNA transcript, and/or inhibition or reduction of skipping of an ASCE from an ASCE-containing pre-mRNA transcript.

Regions defined by ASOs that promote inclusion of an ASCE in a mature RNA transcript are explored in greater detail by means of an ASO “micro-walk,” involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the ASCE, as described herein. An increase or presence of a longer RT-PCR product produced using the primers spanning the region containing the ASCE (e.g., including the exons flanking the ASCE) in ASO-treated cells as compared to in control ASO-treated cells indicates that splicing out of the target ASCE has been inhibited. In some embodiments, the exon skipping inhibition efficiency, the ratio of unspliced to spliced pre-mRNA, the decrease in rate of splicing, or the reduction in extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

ASOs that when hybridized to a region of a pre-mRNA result in promotion of inclusion of an ASCE in a mature RNA transcript, and/or inhibition or reduction of skipping of an ASCE from an ASCE-containing pre-mRNA transcript, and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intrathecal injection, intracerebroventricular injection, intraperitoneal injection, intramuscular injection, subcutaneous injection, or intravenous injection. Following administration, the cells, tissues, and/or organs of the model animals may be assessed to determine the effect of the ASO treatment by for example evaluating splicing (e.g., efficiency, rate, extent) and protein production by methods known in the art and described herein. The animal models may also be any phenotypic or behavioral indication of the disease or disease severity.

Also within the scope of the present disclosure is a method to identify or validate an ASCE in the presence of an NMD inhibitor, for example, cycloheximide. An exemplary method is provided in Example 2.

Exemplary genes encoding ASCE-containing pre-mRNAs and ASCE sequences are summarized in Table 1 and Table 2 (SEQ ID NOS indicate the corresponding nucleotide sequences represented by the Gene ID Nos (NCBI Entrez Gene No.). Sequences of exemplary target sequences in pre-mRNA transcripts are shown in Table 3. Exemplary ASO sequences are shown in Table 4.

TABLE 1
List of Exemplary Target Genes Encoding ASCE-Containing Pre-mRNAs

| | | NMD | | | Human | CHX |
|---|---|---|---|---|---|---|
| Gene | | Event | | | tissue event | responsiveness |
| Symbol | Disease | type | Event | Organ | abundance | in vitro |
| PKD1 | PKD | Exon | chr16 | Kidney | 5.5% | 2-3X (PCR) |
| | | skipping | 2092954 | | | |
| | | | 2093093 | | | |
| ABCA4 | Age-related | Exon | chr1 | Ocular | 9.6% | 18X |
| | macular | skipping | 94111438 | | (40/53) | |
| | degeneration-2 | | 94111579 | | | |
| FUS | ALS/FTD | Exon | chr16 | CNS | 5.9% | 1.9-9.1X |
| | | skipping | 31186802 | | (15/52) | |
| | | | 31186836 | | | |
| CEL | MODY 8 | Exon | chr9 | Pancreas | No pancreas | 1.5X |
| | (<1% of MODY, | skipping | 133066530 | | data | |
| | MODY 1:1000) | | 133066660 | | | |
| NSD1 | Sotos syndrome | Exon | chr5 | CNS | 12.6% | 2-11X |
| | (1:10,000- | skipping | 177238237 | | (22/52) | |
| | 14,000) | | 177238507 | | | |

TABLE 2
List of Exemplary Gene and ASCE Sequences

| | Gene | | | | | |
|---|---|---|---|---|---|---|
| Gene | ID | | | | | ASCE |
| Symbol | No. | SEQ ID NO. | Disease | OMIM | Genetics | Sequences |
| PKD1 | 5310 | chr16: 2088708-2135898 | PKD | 601313 | Autosomal | chr16 |
| | | (GRCh38/hg38) | | | dominant | 2092954 |
| | | Size: 47,191 bases | | | | 2093093 |
| | | Orientation: Minus strand | | | | |
| ABCA4 | 24 | chr1: 93992834-94121148 | Age-related | 601691 | Autosomal | chr1 |
| | | (GRCh38/hg38) | macular | | dominant | 94111438 |
| | | Size: 128,315 bases | degeneration- | | | 94111579 |
| | | Orientation: Minus strand | 2 | | | |
| FUS | 2521 | chr16: 31180110-31194871 | ALS/FTD | 137070 | Autosomal | chr16 |
| | | (GRCh38/hg38) | | | dominant | 31186802 |
| | | Size: 14,762 bases | | | | 31186836 |
| | | Orientation: Plus strand | | | | |
| CEL | 1056 | chr9: 133061981- | MODY 8 | 114840 | Autosomal | chr9 |
| | | 133071861 | | | dominant | 133066530 |
| | | (GRCh38/hg38) | | | | 133066660 |
| | | Size: 9,881 bases | | | | |
| | | Orientation: Plus strand | | | | |
| NSD1 | 64324 | chr5: 177131835- | Sotos | 606681 | Autosomal | chr5 |
| | | 177300213 | syndrome | | dominant | 177238237 |
| | | (GRCh38/hg38) | | | | 177238507 |
| | | Size: 168,379 bases | | | | |
| | | Orientation: Plus strand | | | | |

TABLE 3
Sequences of Exemplary Target Sequences in Human Pre-mRNA Transcripts.

| Gene/pre- | | | |
|---|---|---|---|
| mRNA | | | |
| symbol | Gene | Pre-mRNA | Sequence of ASCE |
| PKD1 | ENSG00000008710 | ENST00000262304.8 | chr16 |
| | (SEQ ID NO: 1) | | 2092954 2093093 |
| ABCA4 | ENSG00000198691 | ENST00000370225.4 | chr1 |
| | (SEQ ID NO: 2) | | 94111438 94111579 |
| FUS | ENSG00000089280 | ENST00000254108.11 | chr16 |
| | (SEQ ID NO: 3) | | 31186802 31186836 |
| CEL | ENSG00000170835 | ENST00000372080.6 | chr9 |
| | (SEQ ID NO: 4) | | 133066530 133066660 |
| NSD1 | ENSG00000165671 | ENST00000354179.8 | chr5 |
| | (SEQ ID NO: 5) | | 177238237 177238507 |

TABLE 4
Exemplary ASO Sequences

| | | | | Oligo Start | Oligo End | | ASO |
|---|---|---|---|---|---|---|---|
| | | | | (Chr | Chr | | SEQ ID |
| Event | Sequence | Chr | Strand | coordinate) | coordinate) | Oligo Names | NO: |
| Exon | ACAAAGGCT | chr5 | + | 177238123 | 177238141 | NSD1:NM_ | 16 |
| skipping | ACAAAAAGT | | | | | 172349:IVS7:−96 | |
| Exon | TTCTGACAA | chr5 | + | 177238128 | 177238146 | NSD1:NM_ | 17 |
| skipping | AGGCTACAA | | | | | 172349:IVS7:−91 | |
| Exon | TGAAATTCT | chr5 | + | 177238133 | 177238151 | NSD1:NM_ | 18 |
| skipping | GACAAAGGC | | | | | 172349:IVS7:−86 | |
| Exon | AGGAATGAA | chr5 | + | 177238138 | 177238156 | NSD1:NM_ | 19 |
| skipping | ATTCTGACA | | | | | 172349:IVS7:−81 | |
| Exon | TTAAAAGGA | chr5 | + | 177238143 | 177238161 | NSD1:NM_ | 20 |
| skipping | ATGAAATTC | | | | | 172349:IVS7:−76 | |
| Exon | ACACTTTAA | chr5 | + | 177238148 | 177238166 | NSD1:NM_ | 21 |
| skipping | AAGGAATGA | | | | | 172349:IVS7:−71 | |
| Exon | ATAACACAC | chr5 | + | 177238153 | 177238171 | NSD1:NM_ | 22 |
| skipping | TTTAAAAGG | | | | | 172349:IVS7:−66 | |
| Exon | AAAGAATAA | chr5 | + | 177238158 | 177238176 | NSD1:NM_ | 23 |
| skipping | CACACTTTA | | | | | 172349:IVS7:−61 | |
| Exon | GTCAAAAAG | chr5 | + | 177238163 | 177238181 | NSD1:NM_ | 24 |
| skipping | AATAACACA | | | | | 172349:IVS7:−56 | |
| Exon | TAAGTGTCA | chr5 | + | 177238168 | 177238186 | NSD1:NM_ | 25 |
| skipping | AAAAGAATA | | | | | 172349:IVS7:−51 | |
| Exon | TAATTTAAG | chr5 | + | 177238173 | 177238191 | NSD1:NM_ | 26 |
| skipping | TGTCAAAAA | | | | | 172349:IVS7:−46 | |
| Exon | TGTTGTAAT | chr5 | + | 177238178 | 177238196 | NSD1:NM_ | 27 |
| skipping | TTAAGTGTC | | | | | 172349:IVS7:−41 | |
| Exon | AAAATTGTT | chr5 | + | 177238183 | 177238201 | NSD1:NM_ | 28 |
| skipping | GTAATTTAA | | | | | 172349:IVS7:−36 | |
| Exon | AGGCCAAAA | chr5 | + | 177238188 | 177238206 | NSD1:NM_ | 29 |
| skipping | TTGTTGTAA | | | | | 172349:IVS7:−31 | |
| Exon | TCCACAGGC | chr5 | + | 177238193 | 177238211 | NSD1:NM_ | 30 |
| skipping | CAAAATTGT | | | | | 172349:IVS7:−26 | |
| Exon | TAGAGTCCA | chr5 | + | 177238198 | 177238216 | NSD1:NM_ | 31 |
| skipping | CAGGCCAAA | | | | | 172349:IVS7:−21 | |
| Exon | AAAAATAGA | chr5 | + | 177238203 | 177238221 | NSD1:NM_ | 32 |
| skipping | GTCCACAGG | | | | | 172349:IVS7:−16 | |
| Exon | CTTCACAGC | chr5 | + | 177238237 | 177238255 | NSD1:NM_ | 33 |
| skipping | GGGAACTTA | | | | | 172349:EX8:+2 | |
| Exon | TTCCTCTTCA | chr5 | + | 177238242 | 177238260 | NSD1:NM_ | 34 |
| skipping | CAGCGGGA | | | | | 172349:EX8:+7 | |
| Exon | AGGCTTTCC | chr5 | + | 177238247 | 177238265 | NSD1:NM_ | 35 |
| skipping | TCTTCACAG | | | | | 172349:EX8:+12 | |
| Exon | CTAGAAGGC | chr5 | + | 177238252 | 177238270 | NSD1:NM_ | 36 |
| skipping | TTTCCTCTT | | | | | 172349:EX8:+17 | |
| Exon | TCGGGCTAG | chr5 | + | 177238257 | 177238275 | NSD1:NM_ | 37 |
| skipping | AAGGCTTTC | | | | | 172349:EX8:+22 | |
| Exon | CGACCTCGG | chr5 | + | 177238262 | 177238280 | NSD1:NM_ | 38 |
| skipping | GCTAGAAGG | | | | | 172349:EX8:+27 | |
| Exon | TAGATCGAC | chr5 | + | 177238267 | 177238285 | NSD1:NM_ | 39 |
| skipping | CTCGGGCTA | | | | | 172349:EX8:+32 | |
| Exon | AGCACTAGA | chr5 | + | 177238272 | 177238290 | NSD1:NM_ | 40 |
| skipping | TCGACCTCG | | | | | 172349:EX8:+37 | |
| Exon | TTCTGAGCA | chr5 | + | 177238277 | 177238295 | NSD1:NM_ | 41 |
| skipping | CTAGATCGA | | | | | 172349:EX8:+42 | |
| Exon | GCTTGTTCT | chr5 | + | 177238282 | 177238300 | NSD1:NM_ | 42 |
| skipping | GAGCACTAG | | | | | 172349:EX8:+47 | |
| Exon | CACCTGCTT | chr5 | + | 177238287 | 177238305 | NSD1:NM_ | 43 |
| skipping | GTTCTGAGC | | | | | 172349:EX8:+52 | |
| Exon | TCGTCCACC | chr5 | + | 177238292 | 177238310 | NSD1:NM_ | 44 |
| skipping | TGCTTGTTC | | | | | 172349:EX8:+57 | |
| Exon | AATTCTCGT | chr5 | + | 177238297 | 177238315 | NSD1:NM_ | 45 |
| skipping | CCACCTGCT | | | | | 172349:EX8:+62 | |
| Exon | CAAAGAATT | chr5 | + | 177238302 | 177238320 | NSD1:NM_ | 46 |
| skipping | CTCGTCCAC | | | | | 172349:EX8:+67 | |
| Exon | GAAATCAAA | chr5 | + | 177238307 | 177238325 | NSD1:NM_ | 47 |
| skipping | GAATTCTCG | | | | | 172349:EX8:+72 | |
| Exon | TGGTTGAAA | chr5 | + | 177238312 | 177238330 | NSD1:NM_ | 48 |
| skipping | TCAAAGAAT | | | | | 172349:EX8:+77 | |
| Exon | TTCTTTGGTT | chr5 | + | 177238317 | 177238335 | NSD1:NM_ | 49 |
| skipping | GAAATCAA | | | | | 172349:EX8:+82 | |
| Exon | GGCTCTTCT | chr5 | + | 177238322 | 177238340 | NSD1:NM_ | 50 |
| skipping | TTGGTTGAA | | | | | 172349:EX8:+87 | |
| Exon | CTGGAGGCT | chr5 | + | 177238327 | 177238345 | NSD1:NM_ | 51 |
| skipping | CTTCTTTGG | | | | | 172349:EX8:+92 | |
| Exon | AAGAACTGG | chr5 | + | 177238332 | 177238350 | NSD1:NM_ | 52 |
| skipping | AGGCTCTTC | | | | | 172349:EX8:+97 | |
| Exon | CTTTCAAGA | chr5 | + | 177238337 | 177238355 | NSD1:NM_ | 53 |
| skipping | ACTGGAGGC | | | | | 172349:EX8:+102 | |
| Exon | CCTCCCTTTC | chr5 | + | 177238342 | 177238360 | NSD1:NM_ | 54 |
| skipping | AAGAACTG | | | | | 172349:EX8:+107 | |
| Exon | CGGAGCCTC | chr5 | + | 177238347 | 177238365 | NSD1:NM_ | 55 |
| skipping | CCTTTCAAG | | | | | 172349:EX8:+112 | |
| Exon | AAAAACGGA | chr5 | + | 177238352 | 177238370 | NSD1:NM_ | 56 |
| skipping | GCCTCCCTT | | | | | 172349:EX8:+117 | |
| Exon | CCTCCAAAA | chr5 | + | 177238357 | 177238375 | NSD1:NM_ | 57 |
| skipping | ACGGAGCCT | | | | | 172349:EX8:+122 | |
| Exon | GGGGCCCTC | chr5 | + | 177238362 | 177238380 | NSD1:NM_ | 58 |
| skipping | CAAAAACGG | | | | | 172349:EX8:+127 | |
| Exon | GCCAAGGGG | chr5 | + | 177238367 | 177238385 | NSD1:NM_ | 59 |
| skipping | CCCTCCAAA | | | | | 172349:EX8:+132 | |
| Exon | ACTGAGCCA | chr5 | + | 177238372 | 177238390 | NSD1:NM_ | 60 |
| skipping | AGGGGCCCT | | | | | 172349:EX8:−118 | |
| Exon | TTCTGACTG | chr5 | + | 177238377 | 177238395 | NSD1:NM_ | 61 |
| skipping | AGCCAAGGG | | | | | 172349:EX8:−113 | |
| Exon | CCAAGTTCT | chr5 | + | 177238382 | 177238400 | NSD1:NM_ | 62 |
| skipping | GACTGAGCC | | | | | 172349:EX8:−108 | |
| Exon | CACCTCCAA | chr5 | + | 177238387 | 177238405 | NSD1:NM_ | 63 |
| skipping | GTTCTGACT | | | | | 172349:EX8:−103 | |
| Exon | ATGTCCACC | chr5 | + | 177238392 | 177238410 | NSD1:NM_ | 64 |
| skipping | TCCAAGTTC | | | | | 172349:EX8:−98 | |
| Exon | TCAGCATGT | chr5 | + | 177238397 | 177238415 | NSD1:NM_ | 65 |
| skipping | CCACCTCCA | | | | | 172349:EX8:−93 | |
| Exon | GCAACTCAG | chr5 | + | 177238402 | 177238420 | NSD1:NM_ | 66 |
| skipping | CATGTCCAC | | | | | 172349:EX8:−88 | |
| Exon | CTGCGGCAA | chr5 | + | 177238407 | 177238425 | NSD1:NM_ | 67 |
| skipping | CTCAGCATG | | | | | 172349:EX8:−83 | |
| Exon | GTCAGCTGC | chr5 | + | 177238412 | 177238430 | NSD1:NM_ | 68 |
| skipping | GGCAACTCA | | | | | 172349:EX8:−78 | |
| Exon | ACAAGGTCA | chr5 | + | 177238417 | 177238435 | NSD1:NM_ | 69 |
| skipping | GCTGCGGCA | | | | | 172349:EX8:−73 | |
| Exon | CACAGACAA | chr5 | + | 177238422 | 177238440 | NSD1:NM | 70 |
| skipping | GGTCAGCTG | | | | | 172349:EX8:−68 | |
| Exon | ACAGGCACA | chr5 | + | 177238427 | 177238445 | NSD1:NM_ | 71 |
| skipping | GACAAGGTC | | | | | 172349:EX8:−63 | |
| Exon | GAGCCACAG | chr5 | + | 177238432 | 177238450 | NSD1:NM_ | 72 |
| skipping | GCACAGACA | | | | | 172349:EX8:−58 | |
| Exon | TTCCGGAGC | chr5 | + | 177238437 | 177238455 | NSD1:NM_ | 73 |
| skipping | CACAGGCAC | | | | | 172349:EX8:−53 | |
| Exon | GAGACTTCC | chr5 | + | 177238442 | 177238460 | NSD1:NM_ | 74 |
| skipping | GGAGCCACA | | | | | 172349:EX8:−48 | |
| Exon | GTGGAGAGA | chr5 | + | 177238447 | 177238465 | NSD1:NM_ | 75 |
| skipping | CTTCCGGAG | | | | | 172349:EX8:−43 | |
| Exon | AGGCCGTGG | chr5 | + | 177238452 | 177238470 | NSD1:NM_ | 76 |
| skipping | AGAGACTTC | | | | | 172349:EX8:−38 | |
| Exon | AGGGCAGGC | chr5 | + | 177238457 | 177238475 | NSD1:NM_ | 77 |
| skipping | CGTGGAGAG | | | | | 172349:EX8:−33 | |
| Exon | ACTCAAGGG | chr5 | + | 177238462 | 177238480 | NSD1:NM_ | 78 |
| skipping | CAGGCCGTG | | | | | 172349:EX8:−28 | |
| Exon | CTCAGACTC | chr5 | + | 177238467 | 177238485 | NSD1:NM_ | 79 |
| skipping | AAGGGCAGG | | | | | 172349:EX8:−23 | |
| Exon | AATTCCTCA | chr5 | + | 177238472 | 177238490 | NSD1:NM_ | 80 |
| skipping | GACTCAAGG | | | | | 172349:EX8:−18 | |
| Exon | CTAGCAATT | chr5 | + | 177238477 | 177238495 | NSD1:NM_ | 81 |
| skipping | CCTCAGACT | | | | | 172349:EX8:−13 | |
| Exon | TTTAACTAG | chr5 | + | 177238482 | 177238500 | NSD1:NM_ | 82 |
| skipping | CAATTCCTC | | | | | 172349:EX8:−8 | |
| Exon | GGCGTTTTA | chr5 | + | 177238487 | 177238505 | NSD1:NM_ | 83 |
| skipping | ACTAGCAAT | | | | | 172349:EX8:−3 | |
| Exon | CTGAGACCC | chr5 | + | 177238512 | 177238530 | NSD1:NM_ | 84 |
| skipping | CAACCCCAC | | | | | 172349:IVS8:+6 | |
| Exon | AAATACTGA | chr5 | + | 177238517 | 177238535 | NSD1:NM_ | 85 |
| skipping | GACCCCAAC | | | | | 172349:IVS8:+11 | |
| Exon | TGCTCAAAT | chr5 | + | 177238522 | 177238540 | NSD1:NM_ | 86 |
| skipping | ACTGAGACC | | | | | 172349:IVS8:+16 | |
| Exon | ATATCTGCT | chr5 | + | 177238527 | 177238545 | NSD1:NM_ | 87 |
| skipping | CAAATACTG | | | | | 172349:IVS8:+21 | |
| Exon | TAATCATAT | chr5 | + | 177238532 | 177238550 | NSD1:NM_ | 88 |
| skipping | CTGCTCAAA | | | | | 172349:IVS8:+26 | |
| Exon | TCCTCTAAT | chr5 | + | 177238537 | 177238555 | NSD1:NM_ | 89 |
| skipping | CATATCTGC | | | | | 172349:IVS8:+31 | |
| Exon | CTGCTTCCT | chr5 | + | 177238542 | 177238560 | NSD1:NM_ | 90 |
| skipping | CTAATCATA | | | | | 172349:IVS8:+36 | |
| Exon | ATCTCCTGC | chr5 | + | 177238547 | 177238565 | NSD1:NM_ | 91 |
| skipping | TTCCTCTAA | | | | | 172349:IVS8:+41 | |
| Exon | CTAAAATCT | chr5 | + | 177238552 | 177238570 | NSD1:NM_ | 92 |
| skipping | CCTGCTTCC | | | | | 172349:IVS8:+46 | |
| Exon | ACATACTAA | chr5 | + | 177238557 | 177238575 | NSD1:NM_ | 93 |
| skipping | AATCTCCTG | | | | | 172349:IVS8:+51 | |
| Exon | TCAAAACAT | chr5 | + | 177238562 | 177238580 | NSD1:NM_ | 94 |
| skipping | ACTAAAATC | | | | | 172349:IVS8:+56 | |
| Exon | TTACATCAA | chr5 | + | 177238567 | 177238585 | NSD1:NM_ | 95 |
| skipping | AACATACTA | | | | | 172349:IVS8:+61 | |
| Exon | TGGCTTTAC | chr5 | + | 177238572 | 177238590 | NSD1:NM_ | 96 |
| skipping | ATCAAAACA | | | | | 172349:IVS8:+66 | |
| Exon | AATGTTGGC | chr5 | + | 177238577 | 177238595 | NSD1:NM_ | 97 |
| skipping | TTTACATCA | | | | | 172349:IVS8:+71 | |
| Exon | GATACAATG | chr5 | + | 177238582 | 177238600 | NSD1:NM_ | 98 |
| skipping | TTGGCTTTA | | | | | 172349:IVS8:+76 | |
| Exon | ATATAGATA | chr5 | + | 177238587 | 177238605 | NSD1:NM_ | 99 |
| skipping | CAATGTTGG | | | | | 172349:IVS8:+81 | |
| Exon | ATTGTATAT | chr5 | + | 177238592 | 177238610 | NSD1:NM_ | 100 |
| skipping | AGATACAAT | | | | | 172349:IVS8:+86 | |
| Exon | AGTTTATTG | chr5 | + | 177238597 | 177238615 | NSD1:NM_ | 101 |
| skipping | TATATAGAT | | | | | 172349:IVS8:+91 | |
| Exon | GGGGTAGTT | chr5 | + | 177238602 | 177238620 | NSD1:NM_ | 102 |
| skipping | TATTGTATA | | | | | 172349:IVS8:+96 | |
| Exon | AGTCCACAG | chr5 | + | 177238195 | 177238213 | NSD1:NM_ | 103 |
| skipping | GCCAAAATT | | | | | 172349:IVS7:−24 | |
| Exon | GAGTCCACA | chr5 | + | 177238196 | 177238214 | NSD1:NM_ | 104 |
| skipping | GGCCAAAAT | | | | | 172349:IVS7:−23 | |
| Exon | AGAGTCCAC | chr5 | + | 177238197 | 177238215 | NSD1:NM_ | 105 |
| skipping | AGGCCAAAA | | | | | 172349:IVS7:−22 | |
| Exon | ATAGAGTCC | chr5 | + | 177238199 | 177238217 | NSD1:NM_ | 106 |
| skipping | ACAGGCCAA | | | | | 172349:IVS7:−20 | |
| Exon | AATAGAGTC | chr5 | + | 177238200 | 177238218 | NSD1:NM_ | 107 |
| skipping | CACAGGCCA | | | | | 172349:IVS7:−19 | |
| Exon | AAATAGAGT | chr5 | + | 177238201 | 177238219 | NSD1:NM_ | 108 |
| skipping | CCACAGGCC | | | | | 172349:IVS7:−18 | |
| Exon | AAAATAGAG | chr5 | + | 177238202 | 177238220 | NSD1:NM_ | 109 |
| skipping | TCCACAGGC | | | | | 172349:IVS7:−17 | |

TABLE 5A
Exemplary ASO Sequences

| | | | | | SEQ | |
|---|---|---|---|---|---|---|
| chr | | | | | ID | |
| # | Start | End | Name | Strand | NO: | ASO sequence |
| chr5 | 177238119 | 177238137 | NSD1:NM_ | + | 322 | AGGCTACAAAAAGTGGAT |
| | | | 001365684:U7: | | | |
| | | | IVS7:−100 | | | |
| chr5 | 177238124 | 177238142 | NSD1:NM_ | + | 323 | GACAAAGGCTACAAAAAG |
| | | | 001365684:U7: | | | |
| | | | IVS7:−95 | | | |
| chr5 | 177238129 | 177238147 | NSD1:NM_ | + | 324 | ATTCTGACAAAGGCTACA |
| | | | 001365684:U7: | | | |
| | | | IVS7:−90 | | | |
| chr5 | 177238134 | 177238152 | NSD1:NM_ | + | 325 | ATGAAATTCTGACAAAGG |
| | | | 001365684:U7: | | | |
| | | | IVS7:−85 | | | |
| chr5 | 177238139 | 177238157 | NSD1:NM_ | + | 326 | AAGGAATGAAATTCTGAC |
| | | | 001365684:U7: | | | |
| | | | IVS7:−80 | | | |
| chr5 | 177238144 | 177238162 | NSD1:NM_ | + | 327 | TTTAAAAGGAATGAAATT |
| | | | 001365684:U7: | | | |
| | | | IVS7:−75 | | | |
| chr5 | 177238149 | 177238167 | NSD1:NM_ | + | 328 | CACACTTTAAAAGGAATG |
| | | | 001365684:U7: | | | |
| | | | IVS7:−70 | | | |
| chr5 | 177238154 | 177238172 | NSD1:NM_ | + | 329 | AATAACACACTTTAAAAG |
| | | | 001365684:U7: | | | |
| | | | IVS7:−65 | | | |
| chr5 | 177238159 | 177238177 | NSD1:NM_ | + | 330 | AAAAGAATAACACACTTT |
| | | | 001365684:U7: | | | |
| | | | VIS7:−60 | | | |
| chr5 | 177238164 | 177238182 | NSD1:NM_ | + | 331 | TGTCAAAAAGAATAACAC |
| | | | 001365684:U7: | | | |
| | | | IVS7:−55 | | | |
| chr5 | 177238169 | 177238187 | NSD1:NM_ | + | 332 | TTAAGTGTCAAAAAGAAT |
| | | | 001365684:U7: | | | |
| | | | IVS7:−50 | | | |
| chr5 | 177238174 | 177238192 | NSD1:NM_ | + | 333 | GTAATTTAAGTGTCAAAA |
| | | | 001365684:U7: | | | |
| | | | IVS7:−45 | | | |
| chr5 | 177238179 | 177238197 | NSD1:NM_ | + | 334 | TTGTTGTAATTTAAGTGT |
| | | | 001365684:U7: | | | |
| | | | IVS7:−40 | | | |
| chr5 | 177238184 | 177238202 | NSD1:NM_ | + | 335 | CAAAATTGTTGTAATTTA |
| | | | 001365684:U7: | | | |
| | | | IVS7:−35 | | | |
| chr5 | 177238189 | 177238207 | NSD1:NM_ | + | 336 | CAGGCCAAAATTGTTGTA |
| | | | 001365684:U7: | | | |
| | | | IVS7:−30 | | | |
| chr5 | 177238194 | 177238212 | NSD1:NM_ | + | 337 | GTCCACAGGCCAAAATTG |
| | | | 001365684:U7: | | | |
| | | | IVS7:−25 | | | |
| chr5 | 177238199 | 177238217 | NSD1:NM_ | + | 338 | ATAGAGTCCACAGGCCAA |
| | | | 001365684:U7: | | | |
| | | | IVS7:−20 | | | |
| chr5 | 177238237 | 177238255 | NSD1:NM_ | + | 339 | CTTCACAGCGGGAACTTA |
| | | | 001365684:U7: | | | |
| | | | Ex8:2 | | | |
| chr5 | 177238241 | 177238259 | NSD1:NM_ | + | 340 | TCCTCTTCACAGCGGGAA |
| | | | 001365684:U7: | | | |
| | | | Ex8:6 | | | |
| chr5 | 177238246 | 177238264 | NSD1:NM_ | + | 341 | GGCTTTCCTCTTCACAGC |
| | | | 001365684:U7: | | | |
| | | | Ex8:11 | | | |
| chr5 | 177238251 | 177238269 | NSD1:NM_ | + | 342 | TAGAAGGCTTTCCTCTTC |
| | | | 001365684:U7: | | | |
| | | | Ex8:16 | | | |
| chr5 | 177238256 | 177238274 | NSD1:NM_ | + | 343 | CGGGCTAGAAGGCTTTCC |
| | | | 001365684:U7: | | | |
| | | | Ex8:21 | | | |
| chr5 | 177238261 | 177238279 | NSD1:NM_ | + | 344 | GACCTCGGGCTAGAAGGC |
| | | | 001365684:U7: | | | |
| | | | Ex8:26 | | | |
| chr5 | 177238266 | 177238284 | NSD1:NM_ | + | 345 | AGATCGACCTCGGGCTAG |
| | | | 001365684:U7: | | | |
| | | | Ex8:31 | | | |
| chr5 | 177238271 | 177238289 | NSD1:NM_ | + | 346 | GCACTAGATCGACCTCGG |
| | | | 001365684:U7: | | | |
| | | | Ex8:36 | | | |
| chr5 | 177238276 | 177238294 | NSD1:NM_ | + | 347 | TCTGAGCACTAGATCGAC |
| | | | 001365684:U7: | | | |
| | | | Ex8:41 | | | |
| chr5 | 177238281 | 177238299 | NSD1:NM_ | + | 348 | CTTGTTCTGAGCACTAGA |
| | | | 100365684:U7: | | | |
| | | | Ex8:46 | | | |
| chr5 | 177238286 | 177238304 | NSD1:NM_ | + | 349 | ACCTGCTTGTTCTGAGCA |
| | | | 001365684:U7: | | | |
| | | | Ex8:51 | | | |
| chr5 | 177238291 | 177238309 | NSD1:NM_ | + | 350 | CGTCCACCTGCTTGTTCT |
| | | | 001365684:U7: | | | |
| | | | Ex8:56 | | | |
| chr5 | 177238296 | 177238314 | NSD1:NM_ | + | 35 | ATTCTCGTCCACCTGCTT |
| | | | 001365684:U7: | | | |
| | | | Ex8:61 | | | |
| chr5 | 177238301 | 177238319 | NSD1:NM_ | + | 352 | AAAGAATTCTCGTCCACC |
| | | | 001365684:U7: | | | |
| | | | Ex8:66 | | | |
| chr5 | 177238306 | 177238324 | NSD1:NM_ | + | 353 | AAATCAAAGAATTCTCGT |
| | | | 001365684:U7: | | | |
| | | | Ex8:71 | | | |
| chr5 | 177238311 | 177238329 | NSD1:NM_ | + | 354 | GGTTGAAATCAAAGAATT |
| | | | 001365684:U7: | | | |
| | | | Ex8:76 | | | |
| chr5 | 177238316 | 177238334 | NSD1:NM_ | + | 355 | TCTTTGGTTGAAATCAAA |
| | | | 001365684:U7: | | | |
| | | | Ex8:81 | | | |
| chr5 | 177238321 | 177238339 | NSD1:NM_ | + | 356 | GCTCTTCTTTGGTTGAAA |
| | | | 001365684:U7: | | | |
| | | | Ex8:86 | | | |
| chr5 | 177238326 | 177238344 | NSD1:NM_ | + | 357 | TGGAGGCTCTTCTTTGGT |
| | | | 001365684:U7: | | | |
| | | | Ex8:91 | | | |
| chr5 | 177238331 | 177238349 | NSD1:NM_ | + | 358 | AGAACTGGAGGCTCTTCT |
| | | | 001365684:U7: | | | |
| | | | Ex8:96 | | | |
| chr5 | 177238336 | 177238354 | NSD1:NM_ | + | 359 | TTTCAAGAACTGGAGGCT |
| | | | 001365684:U7: | | | |
| | | | Ex8:101 | | | |
| chr5 | 177238341 | 177238359 | NSD1:NM_ | + | 360 | CTCCCTTTCAAGAACTGG |
| | | | 001365684:U7: | | | |
| | | | Ex8:106 | | | |
| chr5 | 177238346 | 177238364 | NSD1:NM_ | + | 361 | GGAGCCTCCCTTTCAAGA |
| | | | E001365684:U7: | | | |
| | | | x8:111 | | | |
| chr5 | 177238351 | 177238369 | NSD1:NM_ | + | 362 | AAAACGGAGCCTCCCTTT |
| | | | 001365684:U7: | | | |
| | | | Ex8:116 | | | |
| chr5 | 177238356 | 177238374 | NSD1:NM_ | + | 363 | CTCCAAAAACGGAGCCTC |
| | | | 001365684:U7: | | | |
| | | | Ex8:121 | | | |
| chr5 | 177238361 | 177238379 | NSD1:NM_ | + | 364 | GGGCCCTCCAAAAACGGA |
| | | | 001365684:U7: | | | |
| | | | Ex8:126 | | | |
| chr5 | 177238366 | 177238384 | NSD1:NM_ | + | 365 | CCAAGGGGCCCTCCAAAA |
| | | | 001365684:U7: | | | |
| | | | Ex8:131 | | | |
| chr5 | 177238371 | 177238389 | NSD1:NM_ | + | 366 | CTGAGCCAAGGGGCCCTC |
| | | | 001365684:U7: | | | |
| | | | Ex8:−119 | | | |
| chr5 | 177238376 | 177238394 | NSD1:NM_ | + | 367 | TCTGACTGAGCCAAGGGG |
| | | | 001365684:U7: | | | |
| | | | Ex8:−114 | | | |
| chr5 | 177238381 | 177238399 | NSD1:NM_ | + | 368 | CAAGTTCTGACTGAGCCA |
| | | | 001365684:U7: | | | |
| | | | Ex8:−109 | | | |
| chr5 | 177238386 | 177238404 | NSD1:NM_ | + | 369 | ACCTCCAAGTTCTGACTG |
| | | | 001365684:U7: | | | |
| | | | Ex8:−104 | | | |
| chr5 | 177238391 | 177238409 | NSD1:NM_ | + | 370 | TGTCCACCTCCAAGTTCT |
| | | | 001365684:U7: | | | |
| | | | Ex8:−99 | | | |
| chr5 | 177238396 | 177238414 | NSD1:NM_ | + | 371 | CAGCATGTCCACCTCCAA |
| | | | 001365684:U7: | | | |
| | | | Ex8:−94 | | | |
| chr5 | 177238401 | 177238419 | NSD1:NM_ | + | 372 | CAACTCAGCATGTCCACC |
| | | | 001365684:U7: | | | |
| | | | Ex8:−89 | | | |
| chr5 | 177238406 | 177238424 | NSD1:NM_ | + | 373 | TGCGGCAACTCAGCATGT |
| | | | 001365684:U7: | | | |
| | | | Ex8:−84 | | | |
| chr5 | 177238411 | 177238429 | NSD1:NM_ | + | 374 | TCAGCTGCGGCAACTCAG |
| | | | 001365684:U7: | | | |
| | | | xE8:−79 | | | |
| chr5 | 177238416 | 177238434 | NSD1:NM_ | + | 375 | CAAGGTCAGCTGCGGCAA |
| | | | 001365684:U7: | | | |
| | | | Ex8:−74 | | | |
| chr5 | 177238421 | 177238439 | NSD1:NM_ | + | 376 | ACAGACAAGGTCAGCTGC |
| | | | 001365684:U7: | | | |
| | | | Ex8:−69 | | | |
| chr5 | 177238426 | 177238444 | NSD1:NM_ | + | 377 | CAGGCACAGACAAGGTCA |
| | | | 001365684:U7: | | | |
| | | | Ex8:−64 | | | |
| chr5 | 177238431 | 177238449 | NSD1:NM_ | + | 378 | AGCCACAGGCACAGACAA |
| | | | 001365684:U7: | | | |
| | | | Ex8:−59 | | | |
| chr5 | 177238436 | 177238454 | NSD1:NM_ | + | 379 | TCCGGAGCCACAGGCACA |
| | | | 001365684:U7: | | | |
| | | | Ex8:−54 | | | |
| chr5 | 177238441 | 177238459 | NSD1:NM_ | + | 380 | AGACTTCCGGAGCCACAG |
| | | | 001365684:U7: | | | |
| | | | Ex8:−49 | | | |
| chr5 | 177238446 | 177238464 | NSD1:NM_ | + | 381 | TGGAGAGACTTCCGGAGC |
| | | | 001365684:U7: | | | |
| | | | Ex8:−44 | | | |
| chr5 | 177238451 | 177238469 | NSD1:NM_ | + | 382 | GGCCGTGGAGAGACTTCC |
| | | | 001365684:U7: | | | |
| | | | Ex8:−39 | | | |
| chr5 | 177238456 | 177238474 | NSD1:NM_ | + | 383 | GGGCAGGCCGTGGAGAGA |
| | | | 001365684:U7: | | | |
| | | | xE8:−34 | | | |
| chr5 | 177238461 | 177238479 | NSD1:NM_ | + | 384 | CTCAAGGGCAGGCCGTGG |
| | | | 100365684:U7: | | | |
| | | | Ex8:−29 | | | |
| chr5 | 177238466 | 177238484 | NSD1:NM_ | + | 385 | TCAGACTCAAGGGCAGGC |
| | | | 001365684:U7: | | | |
| | | | Ex8:−24 | | | |
| chr5 | 177238471 | 177238489 | NSD1:NM_ | + | 386 | ATTCCTCAGACTCAAGGG |
| | | | 100365684:U7: | | | |
| | | | Ex8:−19 | | | |
| chr5 | 177238476 | 177238494 | NSD1:NM_ | + | 387 | TAGCAATTCCTCAGACTC |
| | | | 100365684:U7: | | | |
| | | | Ex8:−14 | | | |
| chr5 | 177238481 | 177238499 | NSD1:NM_ | + | 388 | TAACTAGCAATTCCTCAG |
| | | | 001365684:U7: | | | |
| | | | Ex8:−9 | | | |
| chr5 | 177238514 | 177238532 | NSD1:NM_ | + | 389 | TTAACTAGCAATTCCTCA |
| | | | 001365684:U7: | | | |
| | | | IVS8:8 | | | |
| chr5 | 177238519 | 177238537 | NSD1:NM_ | + | 390 | TACTGAGACCCCAACCCC |
| | | | 001365684:U7: | | | |
| | | | VIS8:13 | | | |
| chr5 | 177238524 | 177238542 | NSD1:NM_ | + | 391 | TCAAATACTGAGACCCCA |
| | | | 100365684:U7: | | | |
| | | | IVS8:18 | | | |
| chr5 | 177238529 | 177238547 | NSD1:NM_ | + | 392 | TCTGCTCAAATACTGAGA |
| | | | 001365684:U7: | | | |
| | | | IVS8:23 | | | |
| chr5 | 177238534 | 177238552 | NSD1:NM_ | + | 393 | TCATATCTGCTCAAATAC |
| | | | 001365684:U7: | | | |
| | | | IVS8:28 | | | |
| chr5 | 177238539 | 177238557 | NSD1:NM_ | + | 394 | TCTAATCATATCTGCTCA |
| | | | 001365684:U7: | | | |
| | | | IVS8:33 | | | |
| chr5 | 177238544 | 177238562 | NSD1:NM_ | + | 395 | CTTCCTCTAATCATATCT |
| | | | 100365684:U7: | | | |
| | | | IVS8:38 | | | |
| chr5 | 177238549 | 177238567 | NSD1:NM_ | + | 396 | TCCTGCTTCCTCTAATCA |
| | | | 100365684:U7: | | | |
| | | | IVS8:43 | | | |
| chr5 | 177238554 | 177238572 | NSD1:NM_ | + | 397 | AAATCTCCTGCTTCCTCT |
| | | | 001365684:U7: | | | |
| | | | IVS8:48 | | | |
| chr5 | 177238559 | 177238577 | NSD1:NM_ | + | 398 | TACTAAAATCTCCTGCTT |
| | | | 001365684:U7: | | | |
| | | | VIS8:53 | | | |
| chr5 | 177238564 | 177238582 | NSD1:NM_ | + | 399 | AAACATACTAAAATCTCC |
| | | | 001365684:U7: | | | |
| | | | VIS8:58 | | | |
| chr5 | 177238569 | 177238587 | NSD1:NM_ | + | 400 | CATCAAAACATACTAAAA |
| | | | 001365684:U7: | | | |
| | | | VSI8:63 | | | |
| chr5 | 177238574 | 177238592 | NSD1:NM_ | + | 401 | CTTTACATCAAAACATAC |
| | | | 100365684:U7: | | | |
| | | | IVS8:68 | | | |
| chr5 | 177238579 | 177238597 | NSD1:NM_ | + | 402 | GTTGGCTTTACATCAAAA |
| | | | 001365684:U7: | | | |
| | | | VIS8:73 | | | |
| chr5 | 177238584 | 177238602 | NSD1:NM_ | + | 403 | ACAATGTTGGCTTTACAT |
| | | | 001365684:U7: | | | |
| | | | IVS8:78 | | | |
| chr5 | 177238589 | 177238607 | NSD1:NM_ | + | 404 | TAGATACAATGTTGGCTT |
| | | | 001365684:U7: | | | |
| | | | IVS8:83 | | | |
| chr5 | 177238594 | 177238612 | NSD1:NM_ | + | 405 | GTATATAGATACAATGTT |
| | | | 001365684:U7: | | | |
| | | | IVS8:88 | | | |
| chr5 | 177238599 | 177238617 | NSD1:NM_ | + | 406 | TTATTGTATATAGATACA |
| | | | 001365684:U7: | | | |
| | | | IVS8:93 | | | |
| chr5 | 177238604 | 177238622 | NSD1:NM_ | + | 407 | GTAGTTTATTGTATATAG |
| | | | 100365684:U7: | | | |
| | | | IVS8:98 | | | |
| chr5 | 177238609 | 177238627 | NSD1:NM_ | + | 408 | AGGGGGTAGTTTATTGTA |
| | | | 001365684:U7: | | | |
| | | | IVS8:103 | | | |

TABLE 5A-1
Exemplary ASO Sequences

| chr # | Start | End | Strand | SEQ ID NO: | ASO sequence |
|---|---|---|---|---|---|
| chr5 | 177238119 | 177238137 | + | 409 | AAGGCTACAAAAAGTGGAT |
| chr5 | 177238124 | 177238142 | + | 410 | AAGACAAAGGCTACAAAAAG |
| chr5 | 177238129 | 177238147 | + | 411 | AATTCTGACAAAGGCTACA |
| chr5 | 177238134 | 177238152 | + | 412 | AATGAAATTCTGACAAAGG |
| chr5 | 177238139 | 177238157 | + | 413 | AAGGAATGAAATTCTGAC |
| chr5 | 177238144 | 177238162 | + | 414 | AATTTAAAAGGAATGAAATT |
| chr5 | 177238149 | 177238167 | + | 415 | AACACACTTTAAAAGGAATG |
| chr5 | 177238154 | 177238172 | + | 416 | AATAACACACTTTAAAAG |
| chr5 | 177238159 | 177238177 | + | 417 | AAAAGAATAACACACTTT |
| chr5 | 177238164 | 177238182 | + | 418 | AATGTCAAAAAGAATAACAC |
| chr5 | 177238169 | 177238187 | + | 419 | AATTAAGTGTCAAAAAGAAT |
| chr5 | 177238174 | 177238192 | + | 420 | AAGTAATTTAAGTGTCAAAA |
| chr5 | 177238179 | 177238197 | + | 421 | AATTGTTGTAATTTAAGTGT |
| chr5 | 177238184 | 177238202 | + | 422 | AACAAAATTGTTGTAATTTA |
| chr5 | 177238189 | 177238207 | + | 423 | AACAGGCCAAAATTGTTGTA |
| chr5 | 177238194 | 177238212 | + | 424 | AAGTCCACAGGCCAAAATTG |
| chr5 | 177238199 | 177238217 | + | 425 | AATAGAGTCCACAGGCCAA |
| chr5 | 177238237 | 177238255 | + | 426 | AACTTCACAGCGGGAACTTA |
| chr5 | 177238241 | 177238259 | + | 427 | AATCCTCTTCACAGCGGGAA |
| chr5 | 177238246 | 177238264 | + | 428 | AAGGCTTTCCTCTTCACAGC |
| chr5 | 177238251 | 177238269 | + | 429 | AATAGAAGGCTTTCCTCTTC |
| chr5 | 177238256 | 177238274 | + | 430 | AACGGGCTAGAAGGCTTTCC |
| chr5 | 177238261 | 177238279 | + | 431 | AAGACCTCGGGCTAGAAGGC |
| chr5 | 177238266 | 177238284 | + | 432 | AAGATCGACCTCGGGCTAG |
| chr5 | 177238271 | 177238289 | + | 433 | AAGCACTAGATCGACCTCGG |
| chr5 | 177238276 | 177238294 | + | 434 | AATCTGAGCACTAGATCGAC |
| chr5 | 177238281 | 177238299 | + | 435 | AACTTGTTCTGAGCACTAGA |
| chr5 | 177238286 | 177238304 | + | 436 | AACCTGCTTGTTCTGAGCA |
| chr5 | 177238291 | 177238309 | + | 437 | AACGTCCACCTGCTTGTTCT |
| chr5 | 177238296 | 177238314 | + | 438 | AATTCTCGTCCACCTGCTT |
| chr5 | 177238301 | 177238319 | + | 439 | AAAGAATTCTCGTCCACC |
| chr5 | 177238306 | 177238324 | + | 440 | AAATCAAAGAATTCTCGT |
| chr5 | 177238311 | 177238329 | + | 441 | AAGGTTGAAATCAAAGAATT |
| chr5 | 177238316 | 177238334 | + | 442 | AATCTTTGGTTGAAATCAAA |
| chr5 | 177238321 | 177238339 | + | 443 | AAGCTCTTCTTTGGTTGAAA |
| chr5 | 177238326 | 177238344 | + | 444 | AATGGAGGCTCTTCTTTGGT |
| chr5 | 177238331 | 177238349 | + | 445 | AAGAACTGGAGGCTCTTCT |
| chr5 | 177238336 | 177238354 | + | 446 | AATTTCAAGAACTGGAGGCT |
| chr5 | 177238341 | 177238359 | + | 447 | AACTCCCTTTCAAGAACTGG |
| chr5 | 177238346 | 177238364 | + | 448 | AAGGAGCCTCCCTTTCAAGA |
| chr5 | 177238351 | 177238369 | + | 449 | AAAACGGAGCCTCCCTTT |
| chr5 | 177238356 | 177238374 | + | 450 | AACTCCAAAAACGGAGCCTC |
| chr5 | 177238361 | 177238379 | + | 451 | AAGGGCCCTCCAAAAACGGA |
| chr5 | 177238366 | 177238384 | + | 452 | AACCAAGGGGCCCTCCAAAA |
| chr5 | 177238371 | 177238389 | + | 453 | AACTGAGCCAAGGGGCCCTC |
| chr5 | 177238376 | 177238394 | + | 454 | AATCTGACTGAGCCAAGGGG |
| chr5 | 177238381 | 177238399 | + | 455 | AACAAGTTCTGACTGAGCCA |
| chr5 | 177238386 | 177238404 | + | 456 | AACCTCCAAGTTCTGACTG |
| chr5 | 177238391 | 177238409 | + | 457 | AATGTCCACCTCCAAGTTCT |
| chr5 | 177238396 | 177238414 | + | 458 | AACAGCATGTCCACCTCCAA |
| chr5 | 177238401 | 177238419 | + | 459 | AACAACTCAGCATGTCCACC |
| chr5 | 177238406 | 177238424 | + | 460 | AATGCGGCAACTCAGCATGT |
| chr5 | 177238411 | 177238429 | + | 461 | AATCAGCTGCGGCAACTCAG |
| chr5 | 177238416 | 177238434 | + | 462 | AACAAGGTCAGCTGCGGCAA |
| chr5 | 177238421 | 177238439 | + | 463 | AACAGACAAGGTCAGCTGC |
| chr5 | 177238426 | 177238444 | + | 464 | AACAGGCACAGACAAGGTCA |
| chr5 | 177238431 | 177238449 | + | 465 | AAGCCACAGGCACAGACAA |
| chr5 | 177238436 | 177238454 | + | 466 | AATCCGGAGCCACAGGCACA |
| chr5 | 177238441 | 177238459 | + | 467 | AAGACTTCCGGAGCCACAG |
| chr5 | 177238446 | 177238464 | + | 468 | AATGGAGAGACTTCCGGAGC |
| chr5 | 177238451 | 177238469 | + | 469 | AAGGCCGTGGAGAGACTTCC |
| chr5 | 177238456 | 177238474 | + | 470 | AAGGGCAGGCCGTGGAGAGA |
| chr5 | 177238461 | 177238479 | + | 471 | AACTCAAGGGCAGGCCGTGG |
| chr5 | 177238466 | 177238484 | + | 472 | AATCAGACTCAAGGGCAGGC |
| chr5 | 177238471 | 177238489 | + | 473 | AATTCCTCAGACTCAAGGG |
| chr5 | 177238476 | 177238494 | + | 474 | AATAGCAATTCCTCAGACTC |
| chr5 | 177238481 | 177238499 | + | 475 | AATAACTAGCAATTCCTCAG |
| chr5 | 177238514 | 177238532 | + | 476 | AATTAACTAGCAATTCCTCA |
| chr5 | 177238519 | 177238537 | + | 477 | AATACTGAGACCCCAACCCC |
| chr5 | 177238524 | 177238542 | + | 478 | AATCAAATACTGAGACCCCA |
| chr5 | 177238529 | 177238547 | + | 479 | AATCTGCTCAAATACTGAGA |
| chr5 | 177238534 | 177238552 | + | 480 | AATCATATCTGCTCAAATAC |
| chr5 | 177238539 | 177238557 | + | 481 | AATCTAATCATATCTGCTCA |
| chr5 | 177238544 | 177238562 | + | 482 | AACTTCCTCTAATCATATCT |
| chr5 | 177238549 | 177238567 | + | 483 | AATCCTGCTTCCTCTAATCA |
| chr5 | 177238554 | 177238572 | + | 484 | AAATCTCCTGCTTCCTCT |
| chr5 | 177238559 | 177238577 | + | 485 | AATACTAAAATCTCCTGCTT |
| chr5 | 177238564 | 177238582 | + | 486 | AAACATACTAAAATCTCC |
| chr5 | 177238569 | 177238587 | + | 487 | AACATCAAAACATACTAAAA |
| chr5 | 177238574 | 177238592 | + | 488 | AACTTTACATCAAAACATAC |
| chr5 | 177238579 | 177238597 | + | 489 | AAGTTGGCTTTACATCAAAA |
| chr5 | 177238584 | 177238602 | + | 490 | AACAATGTTGGCTTTACAT |
| chr5 | 177238589 | 177238607 | + | 491 | AATAGATACAATGTTGGCTT |
| chr5 | 177238594 | 177238612 | + | 492 | AAGTATATAGATACAATGTT |
| chr5 | 177238599 | 177238617 | + | 493 | AATTATTGTATATAGATACA |
| chr5 | 177238604 | 177238622 | + | 494 | AAGTAGTTTATTGTATATAG |
| chr5 | 177238609 | 177238627 | + | 495 | AAGGGGGTAGTTTATTGTA |

TABLE 5B
Exemplary ASO Sequences

| | | | | | SEQ | |
|---|---|---|---|---|---|---|
| | | | | | ID | |
| chr | Start | End | Name | Strand | NO: | ASO sequence |
| chr5 | 177238118 | 177238136 | NSD1:NM_001365684: | + | 496 | GGCTACAAAAAGTGGATG |
| | | | U7:IVS7:−101 | | | |
| chr5 | 177238119 | 177238137 | NSD1:NM_001365684: | + | 497 | AGGCTACAAAAAGTGGAT |
| | | | U7:IVS7:−100 | | | |
| chr5 | 177238120 | 177238138 | NSD1:NM_001365684: | + | 498 | AAGGCTACAAAAAGTGGA |
| | | | U7:IVS7:−99 | | | |
| chr5 | 177238121 | 177238139 | NSD1:NM_001365684: | + | 499 | AAAGGCTACAAAAAGTGG |
| | | | U7:IVS7:−98 | | | |
| chr5 | 177238122 | 177238140 | NSD1:NM_001365684: | + | 500 | CAAAGGCTACAAAAAGTG |
| | | | U7:IVS7:−97 | | | |
| chr5 | 177238123 | 177238141 | NSD1:NM_001365684: | + | 501 | ACAAAGGCTACAAAAAGT |
| | | | U7:IVS7:−96 | | | |
| chr5 | 177238124 | 177238142 | NSD1:NM_001365684: | + | 502 | GACAAAGGCTACAAAAAG |
| | | | U7:IVS7:−95 | | | |
| chr5 | 177238125 | 177238143 | NSD1:NM_001365684: | + | 503 | TGACAAAGGCTACAAAAA |
| | | | U7:IVS7:−94 | | | |
| chr5 | 177238126 | 177238144 | NSD1:NM_001365684: | + | 504 | CTGACAAAGGCTACAAAA |
| | | | U7:IVS7:−93 | | | |
| chr5 | 177238127 | 177238145 | NSD1:NM_001365684: | + | 505 | TCTGACAAAGGCTACAAA |
| | | | U7:IVS7:−92 | | | |
| chr5 | 177238128 | 177238146 | NSD1:NM_001365684: | + | 506 | TTCTGACAAAGGCTACAA |
| | | | U7:IVS7:−91 | | | |
| chr5 | 177238129 | 177238147 | NSD1:NM_001365684: | + | 507 | ATTCTGACAAAGGCTACA |
| | | | U7:IVS7:−90 | | | |
| chr5 | 177238130 | 177238148 | NSD1:NM_001365684: | + | 508 | AATTCTGACAAAGGCTAC |
| | | | U7:IVS7:−89 | | | |
| chr5 | 177238131 | 177238149 | NSD1:NM_001365684: | + | 509 | AAATTCTGACAAAGGCTA |
| | | | U7:IVS7:−88 | | | |
| chr5 | 177238132 | 177238150 | NSD1:NM_001365684: | + | 510 | GAAATTCTGACAAAGGCT |
| | | | U7:IVS7:−87 | | | |
| chr5 | 177238133 | 177238151 | NSD1:NM_001365684: | + | 511 | TGAAATTCTGACAAAGGC |
| | | | U7:IVS7:−86 | | | |
| chr5 | 177238134 | 177238152 | NSD1:NM_001365684: | + | 512 | ATGAAATTCTGACAAAGG |
| | | | U7:IVS7:−85 | | | |
| chr5 | 177238135 | 177238153 | NSD1:NM_001365684: | + | 513 | AATGAAATTCTGACAAAG |
| | | | U7:IVS7:−84 | | | |
| chr5 | 177238136 | 177238154 | NSD1:NM_001365684: | + | 514 | GAATGAAATTCTGACAAA |
| | | | U7:IVS7:−83 | | | |
| chr5 | 177238137 | 177238155 | NSD1:NM_001365684: | + | 515 | GGAATGAAATTCTGACAA |
| | | | U7:IVS7:−82 | | | |
| chr5 | 177238138 | 177238156 | NSD1:NM_001365684: | + | 516 | AGGAATGAAATTCTGACA |
| | | | U7:IVS7:−81 | | | |
| chr5 | 177238139 | 177238157 | NSD1:NM_001365684: | + | 517 | AAGGAATGAAATTCTGAC |
| | | | U7:IVS7:−80 | | | |
| chr5 | 177238140 | 177238158 | NSD1:NM_001365684: | + | 518 | AAAGGAATGAAATTCTGA |
| | | | U7:IVS7:−79 | | | |
| chr5 | 177238141 | 177238159 | NSD1:NM_001365684: | + | 519 | AAAAGGAATGAAATTCTG |
| | | | U7:IVS7:−78 | | | |
| chr5 | 177238142 | 177238160 | NSD1:NM_001365684: | + | 520 | TAAAAGGAATGAAATTCT |
| | | | U7:IVS7:−77 | | | |
| chr5 | 177238143 | 177238161 | NSD1:NM_001365684: | + | 521 | TTAAAAGGAATGAAATTC |
| | | | U7:IVS7:−76 | | | |
| chr5 | 177238144 | 177238162 | NSD1:NM_001365684: | + | 522 | TTTAAAAGGAATGAAATT |
| | | | U7:IVS7:−75 | | | |
| chr5 | 177238145 | 177238163 | NSD1:NM_001365684: | + | 523 | CTTTAAAAGGAATGAAAT |
| | | | U7:IVS7:−74 | | | |
| chr5 | 177238146 | 177238164 | NSD1:NM_001365684: | + | 524 | ACTTTAAAAGGAATGAAA |
| | | | U7:IVS7:−73 | | | |
| chr5 | 177238147 | 177238165 | NSD1:NM_001365684: | + | 525 | CACTTTAAAAGGAATGAA |
| | | | U7:IVS7:−72 | | | |
| chr5 | 177238148 | 177238166 | NSD1:NM_001365684: | + | 526 | ACACTTTAAAAGGAATGA |
| | | | U7:IVS7:−71 | | | |
| chr5 | 177238149 | 177238167 | NSD1:NM_001365684: | + | 527 | CACACTTTAAAAGGAATG |
| | | | U7:IVS7:−70 | | | |
| chr5 | 177238150 | 177238168 | NSD1:NM_001365684: | + | 528 | ACACACTTTAAAAGGAAT |
| | | | U7:IVS7:−69 | | | |
| chr5 | 177238151 | 177238169 | NSD1:NM_001365684: | + | 529 | AACACACTTTAAAAGGAA |
| | | | U7:IVS7:−68 | | | |
| chr5 | 177238152 | 177238170 | NSD1:NM_001365684: | + | 530 | TAACACACTTTAAAAGGA |
| | | | U7:IVS7:−67 | | | |
| chr5 | 177238153 | 177238171 | NSD1:NM_001365684: | + | 531 | ATAACACACTTTAAAAGG |
| | | | U7:IVS7:−66 | | | |
| chr5 | 177238154 | 177238172 | NSD1:NM_001365684: | + | 532 | AATAACACACTTTAAAAG |
| | | | U7:IVS7:−65 | | | |
| chr5 | 177238155 | 177238173 | NSD1:NM_001365684: | + | 533 | GAATAACACACTTTAAAA |
| | | | U7:IVS7:−64 | | | |
| chr5 | 177238156 | 177238174 | NSD1:NM_001365684: | + | 534 | AGAATAACACACTTTAAA |
| | | | U7:IVS7:−63 | | | |
| chr5 | 177238157 | 177238175 | NSD1:NM_001365684: | + | 535 | AAGAATAACACACTTTAA |
| | | | U7:IVS7:−62 | | | |
| chr5 | 177238158 | 177238176 | NSD1:NM_001365684: | + | 536 | AAAGAATAACACACTTTA |
| | | | U7:IVS7:−61 | | | |
| chr5 | 177238159 | 177238177 | NSD1:NM_001365684: | + | 537 | AAAAGAATAACACACTTT |
| | | | U7:IVS7:−60 | | | |
| chr5 | 177238160 | 177238178 | NSD1:NM_001365684: | + | 538 | AAAAAGAATAACACACTT |
| | | | U7:IVS7:−59 | | | |
| chr5 | 177238161 | 177238179 | NSD1:NM_001365684: | + | 539 | CAAAAAGAATAACACACT |
| | | | U7:IVS7:−58 | | | |
| chr5 | 177238162 | 177238180 | NSD1:NM_001365684: | + | 540 | TCAAAAAGAATAACACAC |
| | | | U7:IVS7:−57 | | | |
| chr5 | 177238163 | 177238181 | NSD1:NM_001365684: | + | 541 | GTCAAAAAGAATAACACA |
| | | | U7:IVS7:−56 | | | |
| chr5 | 177238164 | 177238182 | NSD1:NM_001365684: | + | 542 | TGTCAAAAAGAATAACAC |
| | | | U7:IVS7:−55 | | | |
| chr5 | 177238165 | 177238183 | NSD1:NM_001365684: | + | 543 | GTGTCAAAAAGAATAACA |
| | | | U7:IVS7:−54 | | | |
| chr5 | 177238166 | 177238184 | NSD1:NM_001365684: | + | 544 | AGTGTCAAAAAGAATAAC |
| | | | U7:IVS7:−53 | | | |
| chr5 | 177238167 | 177238185 | NSD1:NM_001365684: | + | 545 | AAGTGTCAAAAAGAATAA |
| | | | U7:IVS7:−52 | | | |
| chr5 | 177238168 | 177238186 | NSD1:NM_001365684: | + | 546 | TAAGTGTCAAAAAGAATA |
| | | | U7:IVS7:−51 | | | |
| chr5 | 177238169 | 177238187 | NSD1:NM_001365684: | + | 547 | TTAAGTGTCAAAAAGAAT |
| | | | U7:IVS7:−50 | | | |
| chr5 | 177238170 | 177238188 | NSD1:NM_001365684: | + | 548 | TTTAAGTGTCAAAAAGAA |
| | | | U7:IVS7:−49 | | | |
| chr5 | 177238171 | 177238189 | NSD1:NM_001365684: | + | 549 | ATTTAAGTGTCAAAAAGA |
| | | | U7:IVS7:−48 | | | |
| chr5 | 177238172 | 177238190 | NSD1:NM_001365684: | + | 550 | AATTTAAGTGTCAAAAAG |
| | | | U7:IVS7:−47 | | | |
| chr5 | 177238173 | 177238191 | NSD1:NM_001365684: | + | 551 | TAATTTAAGTGTCAAAAA |
| | | | U7:IVS7:−46 | | | |
| chr5 | 177238174 | 177238192 | NSD1:NM_001365684: | + | 552 | GTAATTTAAGTGTCAAAA |
| | | | U7:IVS7:−45 | | | |
| chr5 | 177238175 | 177238193 | NSD1:NM_001365684: | + | 553 | TGTAATTTAAGTGTCAAA |
| | | | U7:IVS7:−44 | | | |
| chr5 | 177238176 | 177238194 | NSD1:NM_001365684: | + | 554 | TTGTAATTTAAGTGTCAA |
| | | | U7:IVS7:−43 | | | |
| chr5 | 177238177 | 177238195 | NSD1:NM_001365684: | + | 555 | GTTGTAATTTAAGTGTCA |
| | | | U7:IVS7:−42 | | | |
| chr5 | 177238178 | 177238196 | NSD1:NM_001365684: | + | 556 | TGTTGTAATTTAAGTGTC |
| | | | U7:IVS7:−41 | | | |
| chr5 | 177238179 | 177238197 | NSD1:NM_001365684: | + | 557 | TTGTTGTAATTTAAGTGT |
| | | | U7:IVS7:−40 | | | |
| chr5 | 177238180 | 177238198 | NSD1:NM_001365684: | + | 558 | ATTGTTGTAATTTAAGTG |
| | | | U7:IVS7:−39 | | | |
| chr5 | 177238181 | 177238199 | NSD1:NM_001365684: | + | 559 | AATTGTTGTAATTTAAGT |
| | | | U7:IVS7:−38 | | | |
| chr5 | 177238182 | 177238200 | NSD1:NM_001365684: | + | 560 | AAATTGTTGTAATTTAAG |
| | | | U7:IVS7:−37 | | | |
| chr5 | 177238183 | 177238201 | NSD1:NM_001365684: | + | 561 | AAAATTGTTGTAATTTAA |
| | | | U7:IVS7:−36 | | | |
| chr5 | 177238184 | 177238202 | NSD1:NM_001365684: | + | 562 | CAAAATTGTTGTAATTTA |
| | | | U7:IVS7:−35 | | | |
| chr5 | 177238185 | 177238203 | NSD1:NM_001365684: | + | 563 | CCAAAATTGTTGTAATTT |
| | | | U7:IVS7:−34 | | | |
| chr5 | 177238186 | 177238204 | NSD1:NM_001365684: | + | 564 | GCCAAAATTGTTGTAATT |
| | | | U7:IVS7:−33 | | | |
| chr5 | 177238187 | 177238205 | NSD1:NM_001365684: | + | 565 | GGCCAAAATTGTTGTAAT |
| | | | U7:IVS7:−32 | | | |
| chr5 | 177238188 | 177238206 | NSD1:NM_001365684: | + | 566 | AGGCCAAAATTGTTGTAA |
| | | | U7:IVS7:−31 | | | |
| chr5 | 177238189 | 177238207 | NSD1:NM_001365684: | + | 567 | CAGGCCAAAATTGTTGTA |
| | | | U7:IVS7:−30 | | | |
| chr5 | 177238190 | 177238208 | NSD1:NM_001365684: | + | 568 | ACAGGCCAAAATTGTTGT |
| | | | U7:IVS7:−29 | | | |
| chr5 | 177238191 | 177238209 | NSD1:NM_001365684: | + | 569 | CACAGGCCAAAATTGTTG |
| | | | U7:IVS7:−28 | | | |
| chr5 | 177238192 | 177238210 | NSD1:NM_001365684: | + | 570 | CCACAGGCCAAAATTGTT |
| | | | U7:IVS7:−27 | | | |
| chr5 | 177238193 | 177238211 | NSD1:NM_001365684: | + | 571 | TCCACAGGCCAAAATTGT |
| | | | U7:IVS7:−26 | | | |
| chr5 | 177238194 | 177238212 | NSD1:NM_001365684: | + | 572 | GTCCACAGGCCAAAATTG |
| | | | U7:IVS7:−25 | | | |
| chr5 | 177238195 | 177238213 | NSD1:NM_001365684: | + | 573 | AGTCCACAGGCCAAAATT |
| | | | U7:IVS7:−24 | | | |
| chr5 | 177238196 | 177238214 | NSD1:NM_001365684: | + | 574 | GAGTCCACAGGCCAAAAT |
| | | | U7:IVS7:−23 | | | |
| chr5 | 177238197 | 177238215 | NSD1:NM_001365684: | + | 575 | AGAGTCCACAGGCCAAAA |
| | | | U7:IVS7:−22 | | | |
| chr5 | 177238198 | 177238216 | NSD1:NM_001365684: | + | 576 | TAGAGTCCACAGGCCAAA |
| | | | U7:IVS7:−21 | | | |
| chr5 | 177238199 | 177238217 | NSD1:NM_001365684: | + | 577 | ATAGAGTCCACAGGCCAA |
| | | | U7:IVS7:−20 | | | |
| chr5 | 177238200 | 177238218 | NSD1:NM_001365684: | + | 578 | AATAGAGTCCACAGGCCA |
| | | | U7:IVS7:−19 | | | |
| chr5 | 177238201 | 177238219 | NSD1:NM_001365684: | + | 579 | AAATAGAGTCCACAGGCC |
| | | | U7:IVS7:−18 | | | |
| chr5 | 177238202 | 177238220 | NSD1:NM_001365684: | + | 580 | AAAATAGAGTCCACAGGC |
| | | | U7:IVS7:−17 | | | |
| chr5 | 177238237 | 177238255 | NSD1:NM_001365684: | + | 581 | CTTCACAGCGGGAACTTA |
| | | | U7:Ex8:2 | | | |
| chr5 | 177238238 | 177238256 | NSD1:NM_001365684: | + | 582 | TCTTCACAGCGGGAACTT |
| | | | U7:Ex8:3 | | | |
| chr5 | 177238239 | 177238257 | NSD1:NM_001365684: | + | 583 | CTCTTCACAGCGGGAACT |
| | | | U7:Ex8:4 | | | |
| chr5 | 177238240 | 177238258 | NSD1:NM_001365684: | + | 584 | CCTCTTCACAGCGGGAAC |
| | | | U7:Ex8:5 | | | |
| chr5 | 177238241 | 177238259 | NSD1:NM_001365684: | + | 585 | TCCTCTTCACAGCGGGAA |
| | | | U7:Ex8:6 | | | |
| chr5 | 177238242 | 177238260 | NSD1:NM_001365684: | + | 586 | TTCCTCTTCACAGCGGGA |
| | | | U7:Ex8:7 | | | |
| chr5 | 177238243 | 177238261 | NSD1:NM_001365684: | + | 587 | TTTCCTCTTCACAGCGGG |
| | | | U7:Ex8:8 | | | |
| chr5 | 177238244 | 177238262 | NSD1:NM_001365684: | + | 588 | CTTTCCTCTTCACAGCGG |
| | | | U7:Ex8:9 | | | |
| chr5 | 177238245 | 177238263 | NSD1:NM_001365684: | + | 589 | GCTTTCCTCTTCACAGCG |
| | | | U7:Ex8:10 | | | |
| chr5 | 177238246 | 177238264 | NSD1:NM_001365684: | + | 590 | GGCTTTCCTCTTCACAGC |
| | | | U7:Ex8:11 | | | |
| chr5 | 177238247 | 177238265 | NSD1:NM_001365684: | + | 591 | AGGCTTTCCTCTTCACAG |
| | | | U7:Ex8:12 | | | |
| chr5 | 177238248 | 177238266 | NSD1:NM_001365684: | + | 592 | AAGGCTTTCCTCTTCACA |
| | | | U7:Ex8:13 | | | |
| chr5 | 177238249 | 177238267 | NSD1:NM_001365684: | + | 593 | GAAGGCTTTCCTCTTCAC |
| | | | U7:Ex8:14 | | | |
| chr5 | 177238250 | 177238268 | NSD1:NM_001365684: | + | 594 | AGAAGGCTTTCCTCTTCA |
| | | | U7:Ex8:15 | | | |
| chr5 | 177238251 | 177238269 | NSD1:NM_001365684: | + | 595 | TAGAAGGCTTTCCTCTTC |
| | | | U7:Ex8:16 | | | |
| chr5 | 177238252 | 177238270 | NSD1:NM_001365684: | + | 596 | CTAGAAGGCTTTCCTCTT |
| | | | U7:Ex8:17 | | | |
| chr5 | 177238253 | 177238271 | NSD1:NM_001365684: | + | 597 | GCTAGAAGGCTTTCCTCT |
| | | | U7:Ex8:18 | | | |
| chr5 | 177238254 | 177238272 | NSD1:NM_001365684: | + | 598 | GGCTAGAAGGCTTTCCTC |
| | | | U7:Ex8:19 | | | |
| chr5 | 177238255 | 177238273 | NSD1:NM_001365684: | + | 599 | GGGCTAGAAGGCTTTCCT |
| | | | U7:Ex8:20 | | | |
| chr5 | 177238256 | 177238274 | NSD1:NM_001365684: | + | 600 | CGGGCTAGAAGGCTTTCC |
| | | | U7:Ex8:21 | | | |
| chr5 | 177238257 | 177238275 | NSD1:NM_001365684: | + | 601 | TCGGGCTAGAAGGCTTTC |
| | | | U7:Ex8:22 | | | |
| chr5 | 177238258 | 177238276 | NSD1:NM_001365684: | + | 602 | CTCGGGCTAGAAGGCTTT |
| | | | U7:Ex8:23 | | | |
| chr5 | 177238259 | 177238277 | NSD1:NM_001365684: | + | 603 | CCTCGGGCTAGAAGGCTT |
| | | | U7:Ex8:24 | | | |
| chr5 | 177238260 | 177238278 | NSD1:NM_001365684: | + | 604 | ACCTCGGGCTAGAAGGCT |
| | | | U7:Ex8:25 | | | |
| chr5 | 177238261 | 177238279 | NSD1:NM_001365684: | + | 605 | GACCTCGGGCTAGAAGGC |
| | | | U7:Ex8:26 | | | |
| chr5 | 177238262 | 177238280 | NSD1:NM_001365684: | + | 606 | CGACCTCGGGCTAGAAGG |
| | | | U7:Ex8:27 | | | |
| chr5 | 177238263 | 177238281 | NSD1:NM_001365684: | + | 607 | TCGACCTCGGGCTAGAAG |
| | | | U7:Ex8:28 | | | |
| chr5 | 177238264 | 177238282 | NSD1:NM_001365684: | + | 608 | ATCGACCTCGGGCTAGAA |
| | | | U7:Ex8:29 | | | |
| chr5 | 177238265 | 177238283 | NSD1:NM_001365684: | + | 609 | GATCGACCTCGGGCTAGA |
| | | | U7:Ex8:30 | | | |
| chr5 | 177238266 | 177238284 | NSD1:NM_001365684: | + | 610 | AGATCGACCTCGGGCTAG |
| | | | U7:Ex8:31 | | | |
| chr5 | 177238267 | 177238285 | NSD1:NM_001365684: | + | 611 | TAGATCGACCTCGGGCTA |
| | | | U7:Ex8:32 | | | |
| chr5 | 177238268 | 177238286 | NSD1:NM_001365684: | + | 612 | CTAGATCGACCTCGGGCT |
| | | | U7:Ex8:33 | | | |
| chr5 | 177238269 | 177238287 | NSD1:NM_001365684: | + | 613 | ACTAGATCGACCTCGGGC |
| | | | U7:Ex8:34 | | | |
| chr5 | 177238270 | 177238288 | NSD1:NM_001365684: | + | 614 | CACTAGATCGACCTCGGG |
| | | | U7:Ex8:35 | | | |
| chr5 | 177238271 | 177238289 | NSD1:NM_001365684: | + | 615 | GCACTAGATCGACCTCGG |
| | | | U7:Ex8:36 | | | |
| chr5 | 177238272 | 177238290 | NSD1:NM_001365684: | + | 616 | AGCACTAGATCGACCTCG |
| | | | U7:Ex8:37 | | | |
| chr5 | 177238273 | 177238291 | NSD1:NM_001365684: | + | 617 | GAGCACTAGATCGACCTC |
| | | | U7:Ex8:38 | | | |
| chr5 | 177238274 | 177238292 | NSD1:NM_001365684: | + | 618 | TGAGCACTAGATCGACCT |
| | | | U7:Ex8:39 | | | |
| chr5 | 177238275 | 177238293 | NSD1:NM_001365684: | + | 619 | CTGAGCACTAGATCGACC |
| | | | U7:Ex8:40 | | | |
| chr5 | 177238276 | 177238294 | NSD1:NM_001365684: | + | 620 | TCTGAGCACTAGATCGAC |
| | | | U7:Ex8:41 | | | |
| chr5 | 177238277 | 177238295 | NSD1:NM_001365684: | + | 621 | TTCTGAGCACTAGATCGA |
| | | | U7:Ex8:42 | | | |
| chr5 | 177238278 | 177238296 | NSD1:NM_001365684: | + | 622 | GTTCTGAGCACTAGATCG |
| | | | U7:Ex8:43 | | | |
| chr5 | 177238279 | 177238297 | NSD1:NM_001365684: | + | 623 | TGTTCTGAGCACTAGATC |
| | | | U7:Ex8:44 | | | |
| chr5 | 177238280 | 177238298 | NSD1:NM_001365684: | + | 624 | TTGTTCTGAGCACTAGAT |
| | | | U7:Ex8:45 | | | |
| chr5 | 177238281 | 177238299 | NSD1:NM_001365684: | + | 625 | CTTGTTCTGAGCACTAGA |
| | | | U7:Ex8:46 | | | |
| chr5 | 177238282 | 177238300 | NSD1:NM_001365684: | + | 626 | GCTTGTTCTGAGCACTAG |
| | | | U7:Ex8:47 | | | |
| chr5 | 177238283 | 177238301 | NSD1:NM_001365684: | + | 627 | TGCTTGTTCTGAGCACTA |
| | | | U7:Ex8:48 | | | |
| chr5 | 177238284 | 177238302 | NSD1:NM_001365684: | + | 628 | CTGCTTGTTCTGAGCACT |
| | | | U7:Ex8:49 | | | |
| chr5 | 177238285 | 177238303 | NSD1:NM_001365684: | + | 629 | CCTGCTTGTTCTGAGCAC |
| | | | U7:Ex8:50 | | | |
| chr5 | 177238286 | 177238304 | NSD1:NM_001365684: | + | 630 | ACCTGCTTGTTCTGAGCA |
| | | | U7:Ex8:51 | | | |
| chr5 | 177238287 | 177238305 | NSD1:NM_001365684: | + | 631 | CACCTGCTTGTTCTGAGC |
| | | | U7:Ex8:52 | | | |
| chr5 | 177238288 | 177238306 | NSD1:NM_001365684: | + | 632 | CCACCTGCTTGTTCTGAG |
| | | | U7:Ex8:53 | | | |
| chr5 | 177238289 | 177238307 | NSD1:NM_001365684: | + | 633 | TCCACCTGCTTGTTCTGA |
| | | | U7:Ex8:54 | | | |
| chr5 | 177238290 | 177238308 | NSD1:NM_001365684: | + | 634 | GTCCACCTGCTTGTTCTG |
| | | | U7:Ex8:55 | | | |
| chr5 | 177238291 | 177238309 | NSD1:NM_001365684: | + | 635 | CGTCCACCTGCTTGTTCT |
| | | | U7:Ex8:56 | | | |
| chr5 | 177238292 | 177238310 | NSD1:NM_001365684: | + | 636 | TCGTCCACCTGCTTGTTC |
| | | | U7:Ex8:57 | | | |
| chr5 | 177238293 | 177238311 | NSD1:NM_001365684: | + | 637 | CTCGTCCACCTGCTTGTT |
| | | | U7:Ex8:58 | | | |
| chr5 | 177238294 | 177238312 | NSD1:NM_001365684: | + | 638 | TCTCGTCCACCTGCTTGT |
| | | | U7:Ex8:59 | | | |
| chr5 | 177238295 | 177238313 | NSD1:NM_001365684: | + | 639 | TTCTCGTCCACCTGCTTG |
| | | | U7:Ex8:60 | | | |
| chr5 | 177238296 | 177238314 | NSD1:NM_001365684: | + | 640 | ATTCTCGTCCACCTGCTT |
| | | | U7:Ex8:61 | | | |
| chr5 | 177238297 | 177238315 | NSD1:NM_001365684: | + | 641 | AATTCTCGTCCACCTGCT |
| | | | U7:Ex8:62 | | | |
| chr5 | 177238298 | 177238316 | NSD1:NM_001365684: | + | 642 | GAATTCTCGTCCACCTGC |
| | | | U7:Ex8:63 | | | |
| chr5 | 177238299 | 177238317 | NSD1:NM_001365684: | + | 643 | AGAATTCTCGTCCACCTG |
| | | | U7:Ex8:64 | | | |
| chr5 | 177238300 | 177238318 | NSD1:NM_001365684: | + | 644 | AAGAATTCTCGTCCACCT |
| | | | U7:Ex8:65 | | | |
| chr5 | 177238301 | 177238319 | NSD1:NM_001365684: | + | 645 | AAAGAATTCTCGTCCACC |
| | | | U7:Ex8:66 | | | |
| chr5 | 177238302 | 177238320 | NSD1:NM_001365684: | + | 646 | CAAAGAATTCTCGTCCAC |
| | | | U7:Ex8:67 | | | |
| chr5 | 177238303 | 177238321 | NSD1:NM_001365684: | + | 647 | TCAAAGAATTCTCGTCCA |
| | | | U7:Ex8:68 | | | |
| chr5 | 177238304 | 177238322 | NSD1:NM_001365684: | + | 648 | ATCAAAGAATTCTCGTCC |
| | | | U7:Ex8:69 | | | |
| chr5 | 177238305 | 177238323 | NSD1:NM_001365684: | + | 649 | AATCAAAGAATTCTCGTC |
| | | | U7:Ex8:70 | | | |
| chr5 | 177238306 | 177238324 | NSD1:NM_001365684: | + | 650 | AAATCAAAGAATTCTCGT |
| | | | U7:Ex8:71 | | | |
| chr5 | 177238307 | 177238325 | NSD1:NM_001365684: | + | 651 | GAAATCAAAGAATTCTCG |
| | | | U7:Ex8:72 | | | |
| chr5 | 177238308 | 177238326 | NSD1:NM_001365684: | + | 652 | TGAAATCAAAGAATTCTC |
| | | | U7:Ex8:73 | | | |
| chr5 | 177238309 | 177238327 | NSD1:NM_001365684: | + | 653 | TTGAAATCAAAGAATTCT |
| | | | U7:Ex8:74 | | | |
| chr5 | 177238310 | 177238328 | NSD1:NM_001365684: | + | 654 | GTTGAAATCAAAGAATTC |
| | | | U7:Ex8:75 | | | |
| chr5 | 177238311 | 177238329 | NSD1:NM_001365684: | + | 655 | GGTTGAAATCAAAGAATT |
| | | | U7:Ex8:76 | | | |
| chr5 | 177238312 | 177238330 | NSD1:NM_001365684: | + | 656 | TGGTTGAAATCAAAGAAT |
| | | | U7:Ex8:77 | | | |
| chr5 | 177238313 | 177238331 | NSD1:NM_001365684: | + | 657 | TTGGTTGAAATCAAAGAA |
| | | | U7:Ex8:78 | | | |
| chr5 | 177238314 | 177238332 | NSD1:NM_001365684: | + | 658 | TTTGGTTGAAATCAAAGA |
| | | | U7:Ex8:79 | | | |
| chr5 | 177238315 | 177238333 | NSD1:NM_001365684: | + | 659 | CTTTGGTTGAAATCAAAG |
| | | | U7:Ex8:80 | | | |
| chr5 | 177238316 | 177238334 | NSD1:NM_001365684: | + | 660 | TCTTTGGTTGAAATCAAA |
| | | | U7:Ex8:81 | | | |
| chr5 | 177238317 | 177238335 | NSD1:NM_001365684: | + | 661 | TTCTTTGGTTGAAATCAA |
| | | | U7:Ex8:82 | | | |
| chr5 | 177238318 | 177238336 | NSD1:NM_001365684: | + | 662 | CTTCTTTGGTTGAAATCA |
| | | | U7:Ex8:83 | | | |
| chr5 | 177238319 | 177238337 | NSD1:NM_001365684: | + | 663 | TCTTCTTTGGTTGAAATC |
| | | | U7:Ex8:84 | | | |
| chr5 | 177238320 | 177238338 | NSD1:NM_001365684: | + | 664 | CTCTTCTTTGGTTGAAAT |
| | | | U7:Ex8:85 | | | |
| chr5 | 177238321 | 177238339 | NSD1:NM_001365684: | + | 665 | GCTCTTCTTTGGTTGAAA |
| | | | U7:Ex8:86 | | | |
| chr5 | 177238322 | 177238340 | NSD1:NM_001365684: | + | 666 | GGCTCTTCTTTGGTTGAA |
| | | | U7:Ex8:87 | | | |
| chr5 | 177238323 | 177238341 | NSD1:NM_001365684: | + | 667 | AGGCTCTTCTTTGGTTGA |
| | | | U7:Ex8:88 | | | |
| chr5 | 177238324 | 177238342 | NSD1:NM_001365684: | + | 668 | GAGGCTCTTCTTTGGTTG |
| | | | U7:Ex8:89 | | | |
| chr5 | 177238325 | 177238343 | NSD1:NM_001365684: | + | 669 | GGAGGCTCTTCTTTGGTT |
| | | | U7:Ex8:90 | | | |
| chr5 | 177238326 | 177238344 | NSD1:NM_001365684: | + | 670 | TGGAGGCTCTTCTTTGGT |
| | | | U7:Ex8:91 | | | |
| chr5 | 177238327 | 177238345 | NSD1:NM_001365684: | + | 671 | CTGGAGGCTCTTCTTTGG |
| | | | U7:Ex8:92 | | | |
| chr5 | 177238328 | 177238346 | NSD1:NM_001365684: | + | 672 | ACTGGAGGCTCTTCTTTG |
| | | | U7:Ex8:93 | | | |
| chr5 | 177238329 | 177238347 | NSD1:NM_001365684: | + | 673 | AACTGGAGGCTCTTCTTT |
| | | | U7:Ex8:94 | | | |
| chr5 | 177238330 | 177238348 | NSD1:NM_001365684: | + | 674 | GAACTGGAGGCTCTTCTT |
| | | | U7:Ex8:95 | | | |
| chr5 | 177238331 | 177238349 | NSD1:NM_001365684: | + | 675 | AGAACTGGAGGCTCTTCT |
| | | | U7:Ex8:96 | | | |
| chr5 | 177238332 | 177238350 | NSD1:NM_001365684: | + | 676 | AAGAACTGGAGGCTCTTC |
| | | | U7:Ex8:97 | | | |
| chr5 | 177238333 | 177238351 | NSD1:NM_001365684: | + | 677 | CAAGAACTGGAGGCTCTT |
| | | | U7:Ex8:98 | | | |
| chr5 | 177238334 | 177238352 | NSD1:NM_001365684: | + | 678 | TCAAGAACTGGAGGCTCT |
| | | | U7:Ex8:99 | | | |
| chr5 | 177238335 | 177238353 | NSD1:NM_001365684: | + | 679 | TTCAAGAACTGGAGGCTC |
| | | | U7:Ex8:100 | | | |
| chr5 | 177238336 | 177238354 | NSD1:NM_001365684: | + | 680 | TTTCAAGAACTGGAGGCT |
| | | | U7:Ex8:101 | | | |
| chr5 | 177238337 | 177238355 | NSD1:NM_001365684: | + | 681 | CTTTCAAGAACTGGAGGC |
| | | | U7:Ex8:102 | | | |
| chr5 | 177238338 | 177238356 | NSD1:NM_001365684: | + | 682 | CCTTTCAAGAACTGGAGG |
| | | | U7:Ex8:103 | | | |
| chr5 | 177238339 | 177238357 | NSD1:NM_001365684: | + | 683 | CCCTTTCAAGAACTGGAG |
| | | | U7:Ex8:104 | | | |
| chr5 | 177238340 | 177238358 | NSD1:NM_001365684: | + | 684 | TCCCTTTCAAGAACTGGA |
| | | | U7:Ex8:105 | | | |
| chr5 | 177238341 | 177238359 | NSD1:NM_001365684: | + | 685 | CTCCCTTTCAAGAACTGG |
| | | | U7:Ex8:106 | | | |
| chr5 | 177238342 | 177238360 | NSD1:NM_001365684: | + | 686 | CCTCCCTTTCAAGAACTG |
| | | | U7:Ex8:107 | | | |
| chr5 | 177238343 | 177238361 | NSD1:NM_001365684: | + | 687 | GCCTCCCTTTCAAGAACT |
| | | | U7:Ex8:108 | | | |
| chr5 | 177238344 | 177238362 | NSD1:NM_001365684: | + | 688 | AGCCTCCCTTTCAAGAAC |
| | | | U7:Ex8:109 | | | |
| chr5 | 177238345 | 177238363 | NSD1:NM_001365684: | + | 689 | GAGCCTCCCTTTCAAGAA |
| | | | U7:Ex8:110 | | | |
| chr5 | 177238346 | 177238364 | NSD1:NM_001365684: | + | 690 | GGAGCCTCCCTTTCAAGA |
| | | | U7:Ex8:111 | | | |
| chr5 | 177238347 | 177238365 | NSD1:NM_001365684: | + | 691 | CGGAGCCTCCCTTTCAAG |
| | | | U7:Ex8:112 | | | |
| chr5 | 177238348 | 177238366 | NSD1:NM_001365684: | + | 692 | ACGGAGCCTCCCTTTCAA |
| | | | U7:Ex8:113 | | | |
| chr5 | 177238349 | 177238367 | NSD1:NM_001365684: | + | 693 | AACGGAGCCTCCCTTTCA |
| | | | U7:Ex8:114 | | | |
| chr5 | 177238350 | 177238368 | NSD1:NM_001365684: | + | 694 | AAACGGAGCCTCCCTTTC |
| | | | U7:Ex8:115 | | | |
| chr5 | 177238351 | 177238369 | NSD1:NM_001365684: | + | 695 | AAAACGGAGCCTCCCTTT |
| | | | U7:Ex8:116 | | | |
| chr5 | 177238352 | 177238370 | NSD1:NM_001365684: | + | 696 | AAAAACGGAGCCTCCCTT |
| | | | U7:Ex8:117 | | | |
| chr5 | 177238353 | 177238371 | NSD1:NM_001365684: | + | 697 | CAAAAACGGAGCCTCCCT |
| | | | U7:Ex8:118 | | | |
| chr5 | 177238354 | 177238372 | NSD1:NM_001365684: | + | 698 | CCAAAAACGGAGCCTCCC |
| | | | U7:Ex8:119 | | | |
| chr5 | 177238355 | 177238373 | NSD1:NM_001365684: | + | 699 | TCCAAAAACGGAGCCTCC |
| | | | U7:Ex8:120 | | | |
| chr5 | 177238356 | 177238374 | NSD1:NM_001365684: | + | 700 | CTCCAAAAACGGAGCCTC |
| | | | U7:Ex8:121 | | | |
| chr5 | 177238357 | 177238375 | NSD1:NM_001365684: | + | 701 | CCTCCAAAAACGGAGCCT |
| | | | U7:Ex8:122 | | | |
| chr5 | 177238358 | 177238376 | NSD1:NM_001365684: | + | 702 | CCCTCCAAAAACGGAGCC |
| | | | U7:Ex8:123 | | | |
| chr5 | 177238359 | 177238377 | NSD1:NM_001365684: | + | 703 | GCCCTCCAAAAACGGAGC |
| | | | U7:Ex8:124 | | | |
| chr5 | 177238360 | 177238378 | NSD1:NM_001365684: | + | 704 | GGCCCTCCAAAAACGGAG |
| | | | U7:Ex8:125 | | | |
| chr5 | 177238361 | 177238379 | NSD1:NM_001365684: | + | 705 | GGGCCCTCCAAAAACGGA |
| | | | U7:Ex8:126 | | | |
| chr5 | 177238362 | 177238380 | NSD1:NM_001365684: | + | 706 | GGGGCCCTCCAAAAACGG |
| | | | U7:Ex8:127 | | | |
| chr5 | 177238363 | 177238381 | NSD1:NM_001365684: | + | 707 | AGGGGCCCTCCAAAAACG |
| | | | U7:Ex8:128 | | | |
| chr5 | 177238364 | 177238382 | NSD1:NM_001365684: | + | 708 | AAGGGGCCCTCCAAAAAC |
| | | | U7:Ex8:129 | | | |
| chr5 | 177238365 | 177238383 | NSD1:NM_001365684: | + | 709 | CAAGGGGCCCTCCAAAAA |
| | | | U7:Ex8:130 | | | |
| chr5 | 177238366 | 177238384 | NSD1:NM_001365684: | + | 710 | CCAAGGGGCCCTCCAAAA |
| | | | U7:Ex8:131 | | | |
| chr5 | 177238367 | 177238385 | NSD1:NM_001365684: | + | 711 | GCCAAGGGGCCCTCCAAA |
| | | | U7:Ex8:132 | | | |
| chr5 | 177238368 | 177238386 | NSD1:NM_001365684: | + | 712 | AGCCAAGGGGCCCTCCAA |
| | | | U7:Ex8:133 | | | |
| chr5 | 177238369 | 177238387 | NSD1:NM_001365684: | + | 713 | GAGCCAAGGGGCCCTCCA |
| | | | U7:Ex8:134 | | | |
| chr5 | 177238370 | 177238388 | NSD1:NM_001365684: | + | 714 | TGAGCCAAGGGGCCCTCC |
| | | | U7:Ex8:135 | | | |
| chr5 | 177238371 | 177238389 | NSD1:NM_001365684: | + | 715 | CTGAGCCAAGGGGCCCTC |
| | | | U7:Ex8:−119 | | | |
| chr5 | 177238372 | 177238390 | NSD1:NM_001365684: | + | 716 | ACTGAGCCAAGGGGCCCT |
| | | | U7:Ex8:−118 | | | |
| chr5 | 177238373 | 177238391 | NSD1:NM_001365684: | + | 717 | GACTGAGCCAAGGGGCCC |
| | | | U7:Ex8:−117 | | | |
| chr5 | 177238374 | 177238392 | NSD1:NM_001365684: | + | 718 | TGACTGAGCCAAGGGGCC |
| | | | U7:Ex8:−116 | | | |
| chr5 | 177238375 | 177238393 | NSD1:NM_001365684: | + | 719 | CTGACTGAGCCAAGGGGC |
| | | | U7:Ex8:−115 | | | |
| chr5 | 177238376 | 177238394 | NSD1:NM_001365684: | + | 720 | TCTGACTGAGCCAAGGGG |
| | | | U7:Ex8:−114 | | | |
| chr5 | 177238377 | 177238395 | NSD1:NM_001365684: | + | 721 | TTCTGACTGAGCCAAGGG |
| | | | U7:Ex8:−113 | | | |
| chr5 | 177238378 | 177238396 | NSD1:NM_001365684: | + | 722 | GTTCTGACTGAGCCAAGG |
| | | | U7:Ex8:−112 | | | |
| chr5 | 177238379 | 177238397 | NSD1:NM_001365684: | + | 723 | AGTTCTGACTGAGCCAAG |
| | | | U7:Ex8:−111 | | | |
| chr5 | 177238380 | 177238398 | NSD1:NM_001365684: | + | 724 | AAGTTCTGACTGAGCCAA |
| | | | U7:Ex8:−110 | | | |
| chr5 | 177238381 | 177238399 | NSD1:NM_001365684: | + | 725 | CAAGTTCTGACTGAGCCA |
| | | | U7:Ex8:−109 | | | |
| chr5 | 177238382 | 177238400 | NSD1:NM_001365684: | + | 726 | CCAAGTTCTGACTGAGCC |
| | | | U7:Ex8:−108 | | | |
| chr5 | 177238383 | 177238401 | NSD1:NM_001365684: | + | 727 | TCCAAGTTCTGACTGAGC |
| | | | U7:Ex8:−107 | | | |
| chr5 | 177238384 | 177238402 | NSD1:NM_001365684: | + | 728 | CTCCAAGTTCTGACTGAG |
| | | | U7:Ex8:−106 | | | |
| chr5 | 177238385 | 177238403 | NSD1:NM_001365684: | + | 729 | CCTCCAAGTTCTGACTGA |
| | | | U7:Ex8:−105 | | | |
| chr5 | 177238386 | 177238404 | NSD1:NM_001365684: | + | 730 | ACCTCCAAGTTCTGACTG |
| | | | U7:Ex8:−104 | | | |
| chr5 | 177238387 | 177238405 | NSD1:NM_001365684: | + | 731 | CACCTCCAAGTTCTGACT |
| | | | U7:Ex8:−103 | | | |
| chr5 | 177238388 | 177238406 | NSD1:NM_001365684: | + | 732 | CCACCTCCAAGTTCTGAC |
| | | | U7:Ex8:−102 | | | |
| chr5 | 177238389 | 177238407 | NSD1:NM_001365684: | + | 733 | TCCACCTCCAAGTTCTGA |
| | | | U7:Ex8:−101 | | | |
| chr5 | 177238390 | 177238408 | NSD1:NM_001365684: | + | 734 | GTCCACCTCCAAGTTCTG |
| | | | U7:Ex8:−100 | | | |
| chr5 | 177238391 | 177238409 | NSD1:NM_001365684: | + | 735 | TGTCCACCTCCAAGTTCT |
| | | | U7:Ex8:−99 | | | |
| chr5 | 177238392 | 177238410 | NSD1:NM_001365684: | + | 736 | ATGTCCACCTCCAAGTTC |
| | | | U7:Ex8:−98 | | | |
| chr5 | 177238393 | 177238411 | NSD1:NM_001365684: | + | 737 | CATGTCCACCTCCAAGTT |
| | | | U7:Ex8:−97 | | | |
| chr5 | 177238394 | 177238412 | NSD1:NM_001365684: | + | 738 | GCATGTCCACCTCCAAGT |
| | | | U7:Ex8:−96 | | | |
| chr5 | 177238395 | 177238413 | NSD1:NM_001365684: | + | 739 | AGCATGTCCACCTCCAAG |
| | | | U7:Ex8:−95 | | | |
| chr5 | 177238396 | 177238414 | NSD1:NM_001365684: | + | 740 | CAGCATGTCCACCTCCAA |
| | | | U7:Ex8:−94 | | | |
| chr5 | 177238397 | 177238415 | NSD1:NM_001365684: | + | 741 | TCAGCATGTCCACCTCCA |
| | | | U7:Ex8:−93 | | | |
| chr5 | 177238398 | 177238416 | NSD1:NM_001365684: | + | 742 | CTCAGCATGTCCACCTCC |
| | | | U7:Ex8:−92 | | | |
| chr5 | 177238399 | 177238417 | NSD1:NM_001365684: | + | 743 | ACTCAGCATGTCCACCTC |
| | | | U7:Ex8:−91 | | | |
| chr5 | 177238400 | 177238418 | NSD1:NM_001365684: | + | 744 | AACTCAGCATGTCCACCT |
| | | | U7:Ex8:−90 | | | |
| chr5 | 177238401 | 177238419 | NSD1:NM_001365684: | + | 745 | CAACTCAGCATGTCCACC |
| | | | U7:Ex8:−89 | | | |
| chr5 | 177238402 | 177238420 | NSD1:NM_001365684: | + | 746 | GCAACTCAGCATGTCCAC |
| | | | U7:Ex8:−88 | | | |
| chr5 | 177238403 | 177238421 | NSD1:NM_001365684: | + | 747 | GGCAACTCAGCATGTCCA |
| | | | U7:Ex8:−87 | | | |
| chr5 | 177238404 | 177238422 | NSD1:NM_001365684: | + | 748 | CGGCAACTCAGCATGTCC |
| | | | U7:Ex8:−86 | | | |
| chr5 | 177238405 | 177238423 | NSD1:NM_001365684: | + | 749 | GCGGCAACTCAGCATGTC |
| | | | U7:Ex8:−85 | | | |
| chr5 | 177238406 | 177238424 | NSD1:NM_001365684: | + | 750 | TGCGGCAACTCAGCATGT |
| | | | U7:Ex8:−84 | | | |
| chr5 | 177238407 | 177238425 | NSD1:NM_001365684: | + | 751 | CTGCGGCAACTCAGCATG |
| | | | U7:Ex8:−83 | | | |
| chr5 | 177238408 | 177238426 | NSD1:NM_001365684: | + | 752 | GCTGCGGCAACTCAGCAT |
| | | | U7:Ex8:−82 | | | |
| chr5 | 177238409 | 177238427 | NSD1:NM_001365684: | + | 753 | AGCTGCGGCAACTCAGCA |
| | | | U7:Ex8:−81 | | | |
| chr5 | 177238410 | 177238428 | NSD1:NM_001365684: | + | 754 | CAGCTGCGGCAACTCAGC |
| | | | U7:Ex8:−80 | | | |
| chr5 | 177238411 | 177238429 | NSD1:NM_001365684: | + | 755 | TCAGCTGCGGCAACTCAG |
| | | | U7:Ex8:−79 | | | |
| chr5 | 177238412 | 177238430 | NSD1:NM_001365684: | + | 756 | GTCAGCTGCGGCAACTCA |
| | | | U7:Ex8:−78 | | | |
| chr5 | 177238413 | 177238431 | NSD1:NM_001365684: | + | 757 | GGTCAGCTGCGGCAACTC |
| | | | U7:Ex8:−77 | | | |
| chr5 | 177238414 | 177238432 | NSD1:NM_001365684: | + | 758 | AGGTCAGCTGCGGCAACT |
| | | | U7:Ex8:−76 | | | |
| chr5 | 177238415 | 177238433 | NSD1:NM_001365684: | + | 759 | AAGGTCAGCTGCGGCAAC |
| | | | U7:Ex8:−75 | | | |
| chr5 | 177238416 | 177238434 | NSD1:NM_001365684: | + | 760 | CAAGGTCAGCTGCGGCAA |
| | | | U7:Ex8:−74 | | | |
| chr5 | 177238417 | 177238435 | NSD1:NM_001365684: | + | 761 | ACAAGGTCAGCTGCGGCA |
| | | | U7:Ex8:−73 | | | |
| chr5 | 177238418 | 177238436 | NSD1:NM_001365684: | + | 762 | GACAAGGTCAGCTGCGGC |
| | | | U7:Ex8:−72 | | | |
| chr5 | 177238419 | 177238437 | NSD1:NM_001365684: | + | 763 | AGACAAGGTCAGCTGCGG |
| | | | U7:Ex8:−71 | | | |
| chr5 | 177238420 | 177238438 | NSD1:NM_001365684: | + | 764 | CAGACAAGGTCAGCTGCG |
| | | | U7:Ex8:−70 | | | |
| chr5 | 177238421 | 177238439 | NSD1:NM_001365684: | + | 765 | ACAGACAAGGTCAGCTGC |
| | | | U7:Ex8:−69 | | | |
| chr5 | 177238422 | 177238440 | NSD1:NM_001365684: | + | 766 | CACAGACAAGGTCAGCTG |
| | | | U7:Ex8:−68 | | | |
| chr5 | 177238423 | 177238441 | NSD1:NM_001365684: | + | 767 | GCACAGACAAGGTCAGCT |
| | | | U7:Ex8:−67 | | | |
| chr5 | 177238424 | 177238442 | NSD1:NM_001365684: | + | 768 | GGCACAGACAAGGTCAGC |
| | | | U7:Ex8:−66 | | | |
| chr5 | 177238425 | 177238443 | NSD1:NM_001365684: | + | 769 | AGGCACAGACAAGGTCAG |
| | | | U7:Ex8:−65 | | | |
| chr5 | 177238426 | 177238444 | NSD1:NM_001365684: | + | 770 | CAGGCACAGACAAGGTCA |
| | | | U7:Ex8:−64 | | | |
| chr5 | 177238427 | 177238445 | NSD1:NM_001365684: | + | 771 | ACAGGCACAGACAAGGTC |
| | | | U7:Ex8:−63 | | | |
| chr5 | 177238428 | 177238446 | NSD1:NM_001365684: | + | 772 | CACAGGCACAGACAAGGT |
| | | | U7:Ex8:−62 | | | |
| chr5 | 177238429 | 177238447 | NSD1:NM_001365684: | + | 773 | CCACAGGCACAGACAAGG |
| | | | U7:Ex8:−61 | | | |
| chr5 | 177238430 | 177238448 | NSD1:NM_001365684: | + | 774 | GCCACAGGCACAGACAAG |
| | | | U7:Ex8:−60 | | | |
| chr5 | 177238431 | 177238449 | NSD1:NM_001365684: | + | 775 | AGCCACAGGCACAGACAA |
| | | | U7:Ex8:−59 | | | |
| chr5 | 177238432 | 177238450 | NSD1:NM_001365684: | + | 776 | GAGCCACAGGCACAGACA |
| | | | U7:Ex8:−58 | | | |
| chr5 | 177238433 | 177238451 | NSD1:NM_001365684: | + | 777 | GGAGCCACAGGCACAGAC |
| | | | U7:Ex8:−57 | | | |
| chr5 | 177238434 | 177238452 | NSD1:NM_001365684: | + | 778 | CGGAGCCACAGGCACAGA |
| | | | U7:Ex8:−56 | | | |
| chr5 | 177238435 | 177238453 | NSD1:NM_001365684: | + | 779 | CCGGAGCCACAGGCACAG |
| | | | U7:Ex8:−55 | | | |
| chr5 | 177238436 | 177238454 | NSD1:NM_001365684: | + | 780 | TCCGGAGCCACAGGCACA |
| | | | U7:Ex8:−54 | | | |
| chr5 | 177238437 | 177238455 | NSD1:NM_001365684: | + | 781 | TTCCGGAGCCACAGGCAC |
| | | | U7:Ex8:−53 | | | |
| chr5 | 177238438 | 177238456 | NSD1:NM_001365684: | + | 782 | CTTCCGGAGCCACAGGCA |
| | | | U7:Ex8:−52 | | | |
| chr5 | 177238439 | 177238457 | NSD1:NM_001365684: | + | 783 | ACTTCCGGAGCCACAGGC |
| | | | U7:Ex8:−51 | | | |
| chr5 | 177238440 | 177238458 | NSD1:NM_001365684: | + | 784 | GACTTCCGGAGCCACAGG |
| | | | U7:Ex8:−50 | | | |
| chr5 | 177238441 | 177238459 | NSD1:NM_001365684: | + | 785 | AGACTTCCGGAGCCACAG |
| | | | U7:Ex8:−49 | | | |
| chr5 | 177238442 | 177238460 | NSD1:NM_001365684: | + | 786 | GAGACTTCCGGAGCCACA |
| | | | U7:Ex8:−48 | | | |
| chr5 | 177238443 | 177238461 | NSD1:NM_001365684: | + | 787 | AGAGACTTCCGGAGCCAC |
| | | | U7:Ex8:−47 | | | |
| chr5 | 177238444 | 177238462 | NSD1:NM_001365684: | + | 788 | GAGAGACTTCCGGAGCCA |
| | | | U7:Ex8:−46 | | | |
| chr5 | 177238445 | 177238463 | NSD1:NM_001365684: | + | 789 | GGAGAGACTTCCGGAGCC |
| | | | U7:Ex8:−45 | | | |
| chr5 | 177238446 | 177238464 | NSD1:NM_001365684: | + | 790 | TGGAGAGACTTCCGGAGC |
| | | | U7:Ex8:−44 | | | |
| chr5 | 177238447 | 177238465 | NSD1:NM_001365684: | + | 791 | GTGGAGAGACTTCCGGAG |
| | | | U7:Ex8:−43 | | | |
| chr5 | 177238448 | 177238466 | NSD1:NM_001365684: | + | 792 | CGTGGAGAGACTTCCGGA |
| | | | U7:Ex8:−42 | | | |
| chr5 | 177238449 | 177238467 | NSD1:NM_001365684: | + | 793 | CCGTGGAGAGACTTCCGG |
| | | | U7:Ex8:−41 | | | |
| chr5 | 177238450 | 177238468 | NSD1:NM_001365684: | + | 794 | GCCGTGGAGAGACTTCCG |
| | | | U7:Ex8:−40 | | | |
| chr5 | 177238451 | 177238469 | NSD1:NM_001365684: | + | 795 | GGCCGTGGAGAGACTTCC |
| | | | U7:Ex8:−39 | | | |
| chr5 | 177238452 | 177238470 | NSD1:NM_001365684: | + | 796 | AGGCCGTGGAGAGACTTC |
| | | | U7:Ex8:−38 | | | |
| chr5 | 177238453 | 177238471 | NSD1:NM_001365684: | + | 797 | CAGGCCGTGGAGAGACTT |
| | | | U7:Ex8:−37 | | | |
| chr5 | 177238454 | 177238472 | NSD1:NM_001365684: | + | 798 | GCAGGCCGTGGAGAGACT |
| | | | U7:Ex8:−36 | | | |
| chr5 | 177238455 | 177238473 | NSD1:NM_001365684: | + | 799 | GGCAGGCCGTGGAGAGAC |
| | | | U7:Ex8:−35 | | | |
| chr5 | 177238456 | 177238474 | NSD1:NM_001365684: | + | 800 | GGGCAGGCCGTGGAGAGA |
| | | | U7:Ex8:−34 | | | |
| chr5 | 177238457 | 177238475 | NSD1:NM_001365684: | + | 801 | AGGGCAGGCCGTGGAGAG |
| | | | U7:Ex8:−33 | | | |
| chr5 | 177238458 | 177238476 | NSD1:NM_001365684: | + | 802 | AAGGGCAGGCCGTGGAGA |
| | | | U7:Ex8:−32 | | | |
| chr5 | 177238459 | 177238477 | NSD1:NM_001365684: | + | 803 | CAAGGGCAGGCCGTGGAG |
| | | | U7:Ex8:−31 | | | |
| chr5 | 177238460 | 177238478 | NSD1:NM_001365684: | + | 804 | TCAAGGGCAGGCCGTGGA |
| | | | U7:Ex8:−30 | | | |
| chr5 | 177238461 | 177238479 | NSD1:NM_001365684: | + | 805 | CTCAAGGGCAGGCCGTGG |
| | | | U7:Ex8:−29 | | | |
| chr5 | 177238462 | 177238480 | NSD1:NM_001365684: | + | 806 | ACTCAAGGGCAGGCCGTG |
| | | | U7:Ex8:−28 | | | |
| chr5 | 177238463 | 177238481 | NSD1:NM_001365684: | + | 807 | GACTCAAGGGCAGGCCGT |
| | | | U7:Ex8:−27 | | | |
| chr5 | 177238464 | 177238482 | NSD1:NM_001365684: | + | 808 | AGACTCAAGGGCAGGCCG |
| | | | U7:Ex8:−26 | | | |
| chr5 | 177238465 | 177238483 | NSD1:NM_001365684: | + | 809 | CAGACTCAAGGGCAGGCC |
| | | | U7:Ex8:−25 | | | |
| chr5 | 177238466 | 177238484 | NSD1:NM_001365684: | + | 810 | TCAGACTCAAGGGCAGGC |
| | | | U7:Ex8:−24 | | | |
| chr5 | 177238467 | 177238485 | NSD1:NM_001365684: | + | 811 | CTCAGACTCAAGGGCAGG |
| | | | U7:Ex8:−23 | | | |
| chr5 | 177238468 | 177238486 | NSD1:NM_001365684: | + | 812 | CCTCAGACTCAAGGGCAG |
| | | | U7:Ex8:−22 | | | |
| chr5 | 177238469 | 177238487 | NSD1:NM_001365684: | + | 813 | TCCTCAGACTCAAGGGCA |
| | | | U7:Ex8:−21 | | | |
| chr5 | 177238470 | 177238488 | NSD1:NM_001365684: | + | 814 | TTCCTCAGACTCAAGGGC |
| | | | U7:Ex8:−20 | | | |
| chr5 | 177238471 | 177238489 | NSD1:NM_001365684: | + | 815 | ATTCCTCAGACTCAAGGG |
| | | | U7:Ex8:−19 | | | |
| chr5 | 177238472 | 177238490 | NSD1:NM_001365684: | + | 816 | AATTCCTCAGACTCAAGG |
| | | | U7:Ex8:−18 | | | |
| chr5 | 177238473 | 177238491 | NSD1:NM_001365684: | + | 817 | CAATTCCTCAGACTCAAG |
| | | | U7:Ex8:−17 | | | |
| chr5 | 177238474 | 177238492 | NSD1:NM_001365684: | + | 818 | GCAATTCCTCAGACTCAA |
| | | | U7:Ex8:−16 | | | |
| chr5 | 177238475 | 177238493 | NSD1:NM_001365684: | + | 819 | AGCAATTCCTCAGACTCA |
| | | | U7:Ex8:−15 | | | |
| chr5 | 177238476 | 177238494 | NSD1:NM_001365684: | + | 820 | TAGCAATTCCTCAGACTC |
| | | | U7:Ex8:−14 | | | |
| chr5 | 177238477 | 177238495 | NSD1:NM_001365684: | + | 821 | CTAGCAATTCCTCAGACT |
| | | | U7:Ex8:−13 | | | |
| chr5 | 177238478 | 177238496 | NSD1:NM_001365684: | + | 822 | ACTAGCAATTCCTCAGAC |
| | | | U7:Ex8:−12 | | | |
| chr5 | 177238479 | 177238497 | NSD1:NM_001365684: | + | 823 | AACTAGCAATTCCTCAGA |
| | | | U7:Ex8:−11 | | | |
| chr5 | 177238480 | 177238498 | NSD1:NM_001365684: | + | 824 | TAACTAGCAATTCCTCAG |
| | | | U7:Ex8:−10 | | | |
| chr5 | 177238481 | 177238499 | NSD1:NM_001365684: | + | 825 | TTAACTAGCAATTCCTCA |
| | | | U7:Ex8:−9 | | | |
| chr5 | 177238482 | 177238500 | NSD1:NM_001365684: | + | 826 | TTTAACTAGCAATTCCTC |
| | | | U7:Ex8:−8 | | | |
| chr5 | 177238483 | 177238501 | NSD1:NM_001365684: | + | 827 | TTTTAACTAGCAATTCCT |
| | | | U7:Ex8:−7 | | | |
| chr5 | 177238484 | 177238502 | NSD1:NM_001365684: | + | 828 | GTTTTAACTAGCAATTCC |
| | | | U7:Ex8:−6 | | | |
| chr5 | 177238485 | 177238503 | NSD1:NM_001365684: | + | 829 | CGTTTTAACTAGCAATTC |
| | | | U7:Ex8:−5 | | | |
| chr5 | 177238514 | 177238532 | NSD1:NM_001365684: | + | 830 | TACTGAGACCCCAACCCC |
| | | | U7:IVS8:8 | | | |
| chr5 | 177238515 | 177238533 | NSD1:NM_001365684: | + | 831 | ATACTGAGACCCCAACCC |
| | | | U7:IVS8:9 | | | |
| chr5 | 177238516 | 177238534 | NSD1:NM_001365684: | + | 832 | AATACTGAGACCCCAACC |
| | | | U7:IVS8:10 | | | |
| chr5 | 177238517 | 177238535 | NSD1:NM_001365684: | + | 833 | AAATACTGAGACCCCAAC |
| | | | U7:IVS8:11 | | | |
| chr5 | 177238518 | 177238536 | NSD1:NM_001365684: | + | 834 | CAAATACTGAGACCCCAA |
| | | | U7:IVS8:12 | | | |
| chr5 | 177238519 | 177238537 | NSD1:NM_001365684: | + | 835 | TCAAATACTGAGACCCCA |
| | | | U7:IVS8:13 | | | |
| chr5 | 177238520 | 177238538 | NSD1:NM_001365684: | + | 836 | CTCAAATACTGAGACCCC |
| | | | U7:IVS8:14 | | | |
| chr5 | 177238521 | 177238539 | NSD1:NM_001365684: | + | 837 | GCTCAAATACTGAGACCC |
| | | | U7:IVS8:15 | | | |
| chr5 | 177238522 | 177238540 | NSD1:NM_001365684: | + | 838 | TGCTCAAATACTGAGACC |
| | | | U7:IVS8:16 | | | |
| chr5 | 177238523 | 177238541 | NSD1:NM_001365684: | + | 839 | CTGCTCAAATACTGAGAC |
| | | | U7:IVS8:17 | | | |
| chr5 | 177238524 | 177238542 | NSD1:NM_001365684: | + | 840 | TCTGCTCAAATACTGAGA |
| | | | U7:IVS8:18 | | | |
| chr5 | 177238525 | 177238543 | NSD1:NM_001365684: | + | 841 | ATCTGCTCAAATACTGAG |
| | | | U7:IVS8:19 | | | |
| chr5 | 177238526 | 177238544 | NSD1:NM_001365684: | + | 842 | TATCTGCTCAAATACTGA |
| | | | U7:IVS8:20 | | | |
| chr5 | 177238527 | 177238545 | NSD1:NM_001365684: | + | 843 | ATATCTGCTCAAATACTG |
| | | | U7:IVS8:21 | | | |
| chr5 | 177238528 | 177238546 | NSD1:NM_001365684: | + | 844 | CATATCTGCTCAAATACT |
| | | | U7:IVS8:22 | | | |
| chr5 | 177238529 | 177238547 | NSD1:NM_001365684: | + | 845 | TCATATCTGCTCAAATAC |
| | | | U7:IVS8:23 | | | |
| chr5 | 177238530 | 177238548 | NSD1:NM_001365684: | + | 846 | ATCATATCTGCTCAAATA |
| | | | U7:IVS8:24 | | | |
| chr5 | 177238531 | 177238549 | NSD1:NM_001365684: | + | 847 | AATCATATCTGCTCAAAT |
| | | | U7:IVS8:25 | | | |
| chr5 | 177238532 | 177238550 | NSD1:NM_001365684: | + | 848 | TAATCATATCTGCTCAAA |
| | | | U7:IVS8:26 | | | |
| chr5 | 177238533 | 177238551 | NSD1:NM_001365684: | + | 849 | CTAATCATATCTGCTCAA |
| | | | U7:IVS8:27 | | | |
| chr5 | 177238534 | 177238552 | NSD1:NM_001365684: | + | 850 | TCTAATCATATCTGCTCA |
| | | | U7:IVS8:28 | | | |
| chr5 | 177238535 | 177238553 | NSD1:NM_001365684: | + | 851 | CTCTAATCATATCTGCTC |
| | | | U7:IVS8:29 | | | |
| chr5 | 177238536 | 177238554 | NSD1:NM_001365684: | + | 852 | CCTCTAATCATATCTGCT |
| | | | U7:IVS8:30 | | | |
| chr5 | 177238537 | 177238555 | NSD1:NM_001365684: | + | 853 | TCCTCTAATCATATCTGC |
| | | | U7:IVS8:31 | | | |
| chr5 | 177238538 | 177238556 | NSD1:NM_001365684: | + | 854 | TTCCTCTAATCATATCTG |
| | | | U7:IVS8:32 | | | |
| chr5 | 177238539 | 177238557 | NSD1:NM_001365684: | + | 855 | CTTCCTCTAATCATATCT |
| | | | U7:IVS8:33 | | | |
| chr5 | 177238540 | 177238558 | NSD1:NM_001365684: | + | 856 | GCTTCCTCTAATCATATC |
| | | | U7:IVS8:34 | | | |
| chr5 | 177238541 | 177238559 | NSD1:NM_001365684: | + | 857 | TGCTTCCTCTAATCATAT |
| | | | U7:IVS8:35 | | | |
| chr5 | 177238542 | 177238560 | NSD1:NM_001365684: | + | 858 | CTGCTTCCTCTAATCATA |
| | | | U7:IVS8:36 | | | |
| chr5 | 177238543 | 177238561 | NSD1:NM_001365684: | + | 859 | CCTGCTTCCTCTAATCAT |
| | | | U7:IVS8:37 | | | |
| chr5 | 177238544 | 177238562 | NSD1:NM_001365684: | + | 860 | TCCTGCTTCCTCTAATCA |
| | | | U7:IVS8:38 | | | |
| chr5 | 177238545 | 177238563 | NSD1:NM_001365684: | + | 861 | CTCCTGCTTCCTCTAATC |
| | | | U7:IVS8:39 | | | |
| chr5 | 177238546 | 177238564 | NSD1:NM_001365684: | + | 862 | TCTCCTGCTTCCTCTAAT |
| | | | U7:IVS8:40 | | | |
| chr5 | 177238547 | 177238565 | NSD1:NM_001365684: | + | 863 | ATCTCCTGCTTCCTCTAA |
| | | | U7:IVS8:41 | | | |
| chr5 | 177238548 | 177238566 | NSD1:NM_001365684: | + | 864 | AATCTCCTGCTTCCTCTA |
| | | | U7:IVS8:42 | | | |
| chr5 | 177238549 | 177238567 | NSD1:NM_001365684: | + | 865 | AAATCTCCTGCTTCCTCT |
| | | | U7:IVS8:43 | | | |
| chr5 | 177238550 | 177238568 | NSD1:NM_001365684: | + | 866 | AAAATCTCCTGCTTCCTC |
| | | | U7:IVS8:44 | | | |
| chr5 | 177238551 | 177238569 | NSD1:NM_001365684: | + | 867 | TAAAATCTCCTGCTTCCT |
| | | | U7:IVS8:45 | | | |
| chr5 | 177238552 | 177238570 | NSD1:NM_001365684: | + | 868 | CTAAAATCTCCTGCTTCC |
| | | | U7:IVS8:46 | | | |
| chr5 | 177238553 | 177238571 | NSD1:NM_001365684: | + | 869 | ACTAAAATCTCCTGCTTC |
| | | | U7:IVS8:47 | | | |
| chr5 | 177238554 | 177238572 | NSD1:NM_001365684: | + | 870 | TACTAAAATCTCCTGCTT |
| | | | U7:IVS8:48 | | | |
| chr5 | 177238555 | 177238573 | NSD1:NM_001365684: | + | 871 | ATACTAAAATCTCCTGCT |
| | | | U7:IVS8:49 | | | |
| chr5 | 177238556 | 177238574 | NSD1:NM_001365684: | + | 872 | CATACTAAAATCTCCTGC |
| | | | U7:IVS8:50 | | | |
| chr5 | 177238557 | 177238575 | NSD1:NM_001365684: | + | 873 | ACATACTAAAATCTCCTG |
| | | | U7:IVS8:51 | | | |
| chr5 | 177238558 | 177238576 | NSD1:NM_001365684: | + | 874 | AACATACTAAAATCTCCT |
| | | | U7:IVS8:52 | | | |
| chr5 | 177238559 | 177238577 | NSD1:NM_001365684: | + | 875 | AAACATACTAAAATCTCC |
| | | | U7:IVS8:53 | | | |
| chr5 | 177238560 | 177238578 | NSD1:NM_001365684: | + | 876 | AAAACATACTAAAATCTC |
| | | | U7:IVS8:54 | | | |
| chr5 | 177238561 | 177238579 | NSD1:NM_001365684: | + | 877 | CAAAACATACTAAAATCT |
| | | | U7:IVS8:55 | | | |
| chr5 | 177238562 | 177238580 | NSD1:NM_001365684: | + | 878 | TCAAAACATACTAAAATC |
| | | | U7:IVS8:56 | | | |
| chr5 | 177238563 | 177238581 | NSD1:NM_001365684: | + | 879 | ATCAAAACATACTAAAAT |
| | | | U7:IVS8:57 | | | |
| chr5 | 177238564 | 177238582 | NSD1:NM_001365684: | + | 880 | CATCAAAACATACTAAAA |
| | | | U7:IVS8:58 | | | |
| chr5 | 177238565 | 177238583 | NSD1:NM_001365684: | + | 881 | ACATCAAAACATACTAAA |
| | | | U7:IVS8:59 | | | |
| chr5 | 177238566 | 177238584 | NSD1:NM_001365684: | + | 882 | TACATCAAAACATACTAA |
| | | | U7:IVS8:60 | | | |
| chr5 | 177238567 | 177238585 | NSD1:NM_001365684: | + | 883 | TTACATCAAAACATACTA |
| | | | U7:IVS8:61 | | | |
| chr5 | 177238568 | 177238586 | NSD1:NM_001365684: | + | 884 | TTTACATCAAAACATACT |
| | | | U7:IVS8:62 | | | |
| chr5 | 177238569 | 177238587 | NSD1:NM_001365684: | + | 885 | CTTTACATCAAAACATAC |
| | | | U7:IVS8:63 | | | |
| chr5 | 177238570 | 177238588 | NSD1:NM_001365684: | + | 886 | GCTTTACATCAAAACATA |
| | | | U7:IVS8:64 | | | |
| chr5 | 177238571 | 177238589 | NSD1:NM_001365684: | + | 887 | GGCTTTACATCAAAACAT |
| | | | U7:IVS8:65 | | | |
| chr5 | 177238572 | 177238590 | NSD1:NM_001365684: | + | 888 | TGGCTTTACATCAAAACA |
| | | | U7:IVS8:66 | | | |
| chr5 | 177238573 | 177238591 | NSD1:NM_001365684: | + | 889 | TTGGCTTTACATCAAAAC |
| | | | U7:IVS8:67 | | | |
| chr5 | 177238574 | 177238592 | NSD1:NM_001365684: | + | 890 | GTTGGCTTTACATCAAAA |
| | | | U7:IVS8:68 | | | |
| chr5 | 177238575 | 177238593 | NSD1:NM_001365684: | + | 891 | TGTTGGCTTTACATCAAA |
| | | | U7:IVS8:69 | | | |
| chr5 | 177238576 | 177238594 | NSD1:NM_001365684: | + | 892 | ATGTTGGCTTTACATCAA |
| | | | U7:IVS8:70 | | | |
| chr5 | 177238577 | 177238595 | NSD1:NM_001365684: | + | 893 | AATGTTGGCTTTACATCA |
| | | | U7:IVS8:71 | | | |
| chr5 | 177238578 | 177238596 | NSD1:NM_001365684: | + | 894 | CAATGTTGGCTTTACATC |
| | | | U7:IVS8:72 | | | |
| chr5 | 177238579 | 177238597 | NSD1:NM_001365684: | + | 895 | ACAATGTTGGCTTTACAT |
| | | | U7:IVS8:73 | | | |
| chr5 | 177238580 | 177238598 | NSD1:NM_001365684: | + | 896 | TACAATGTTGGCTTTACA |
| | | | U7:IVS8:74 | | | |
| chr5 | 177238581 | 177238599 | NSD1:NM_001365684: | + | 897 | ATACAATGTTGGCTTTAC |
| | | | U7:IVS8:75 | | | |
| chr5 | 177238582 | 177238600 | NSD1:NM_001365684: | + | 898 | GATACAATGTTGGCTTTA |
| | | | U7:IVS8:76 | | | |
| chr5 | 177238583 | 177238601 | NSD1:NM_001365684: | + | 899 | AGATACAATGTTGGCTTT |
| | | | U7:IVS8:77 | | | |
| chr5 | 177238584 | 177238602 | NSD1:NM_001365684: | + | 900 | TAGATACAATGTTGGCTT |
| | | | U7:IVS8:78 | | | |
| chr5 | 177238585 | 177238603 | NSD1:NM_001365684: | + | 901 | ATAGATACAATGTTGGCT |
| | | | U7:IVS8:79 | | | |
| chr5 | 177238586 | 177238604 | NSD1:NM_001365684: | + | 902 | TATAGATACAATGTTGGC |
| | | | U7:IVS8:80 | | | |
| chr5 | 177238587 | 177238605 | NSD1:NM_001365684: | + | 903 | ATATAGATACAATGTTGG |
| | | | U7:IVS8:81 | | | |
| chr5 | 177238588 | 177238606 | NSD1:NM_001365684: | + | 904 | TATATAGATACAATGTTG |
| | | | U7:IVS8:82 | | | |
| chr5 | 177238589 | 177238607 | NSD1:NM_001365684: | + | 905 | GTATATAGATACAATGTT |
| | | | U7:IVS8:83 | | | |
| chr5 | 177238590 | 177238608 | NSD1:NM_001365684: | + | 906 | TGTATATAGATACAATGT |
| | | | U7:IVS8:84 | | | |
| chr5 | 177238591 | 177238609 | NSD1:NM_001365684: | + | 907 | TTGTATATAGATACAATG |
| | | | U7:IVS8:85 | | | |
| chr5 | 177238592 | 177238610 | NSD1:NM_001365684: | + | 908 | ATTGTATATAGATACAAT |
| | | | U7:IVS8:86 | | | |
| chr5 | 177238593 | 177238611 | NSD1:NM_001365684: | + | 909 | TATTGTATATAGATACAA |
| | | | U7:IVS8:87 | | | |
| chr5 | 177238594 | 177238612 | NSD1:NM_001365684: | + | 910 | TTATTGTATATAGATACA |
| | | | U7:IVS8:88 | | | |
| chr5 | 177238595 | 177238613 | NSD1:NM_001365684: | + | 911 | TTTATTGTATATAGATAC |
| | | | U7:IVS8:89 | | | |
| chr5 | 177238596 | 177238614 | NSD1:NM_001365684: | + | 912 | GTTTATTGTATATAGATA |
| | | | U7:IVS8:90 | | | |
| chr5 | 177238597 | 177238615 | NSD1:NM_001365684: | + | 913 | AGTTTATTGTATATAGAT |
| | | | U7:IVS8:91 | | | |
| chr5 | 177238598 | 177238616 | NSD1:NM_001365684: | + | 914 | TAGTTTATTGTATATAGA |
| | | | U7:IVS8:92 | | | |
| chr5 | 177238599 | 177238617 | NSD1:NM_001365684: | + | 915 | GTAGTTTATTGTATATAG |
| | | | U7:IVS8:93 | | | |
| chr5 | 177238600 | 177238618 | NSD1:NM_001365684: | + | 916 | GGTAGTTTATTGTATATA |
| | | | U7:IVS8:94 | | | |
| chr5 | 177238601 | 177238619 | NSD1:NM_001365684: | + | 917 | GGGTAGTTTATTGTATAT |
| | | | U7:IVS8:95 | | | |
| chr5 | 177238602 | 177238620 | NSD1:NM_001365684: | + | 918 | GGGGTAGTTTATTGTATA |
| | | | U7:IVS8:96 | | | |
| chr5 | 177238603 | 177238621 | NSD1:NM_001365684: | + | 919 | GGGGGTAGTTTATTGTAT |
| | | | U7:IVS8:97 | | | |
| chr5 | 177238604 | 177238622 | NSD1:NM_001365684: | + | 920 | AGGGGGTAGTTTATTGTA |
| | | | U7:IVS8:98 | | | |
| chr5 | 177238605 | 177238623 | NSD1:NM_001365684: | + | 921 | AAGGGGGTAGTTTATTGT |
| | | | U7:IVS8:99 | | | |
| chr5 | 177238606 | 177238624 | NSD1:NM_001365684: | + | 922 | AAAGGGGGTAGTTTATTG |
| | | | U7:IVS8:100 | | | |
| chr5 | 177238607 | 177238625 | NSD1:NM_001365684: | + | 923 | AAAAGGGGGTAGTTTATT |
| | | | U7:IVS8:101 | | | |
| chr5 | 177238608 | 177238626 | NSD1:NM_001365684: | + | 924 | CAAAAGGGGGTAGTTTAT |
| | | | U7:IVS8:102 | | | |
| chr5 | 177238609 | 177238627 | NSD1:NM_001365684: | + | 925 | ACAAAAGGGGGTAGTTTA |
| | | | U7:IVS8:103 | | | |

TABLE 5B-1
Exemplary ASO Sequences

| | | | | SEQ ID | |
|---|---|---|---|---|---|
| chr | Start | End | Strand | NO: | ASO sequence |
| chr5 | 177238118 | 177238136 | + | 926 | AAGGCTACAAAAAGTGGATG |
| chr5 | 177238119 | 177238137 | + | 927 | AAGGCTACAAAAAGTGGAT |
| chr5 | 177238120 | 177238138 | + | 928 | AAGGCTACAAAAAGTGGA |
| chr5 | 177238121 | 177238139 | + | 929 | AAAGGCTACAAAAAGTGG |
| chr5 | 177238122 | 177238140 | + | 930 | AACAAAGGCTACAAAAAGTG |
| chr5 | 177238123 | 177238141 | + | 931 | AACAAAGGCTACAAAAAGT |
| chr5 | 177238124 | 177238142 | + | 932 | AAGACAAAGGCTACAAAAAG |
| chr5 | 177238125 | 177238143 | + | 933 | AATGACAAAGGCTACAAAAA |
| chr5 | 177238126 | 177238144 | + | 934 | AACTGACAAAGGCTACAAAA |
| chr5 | 177238127 | 177238145 | + | 935 | AATCTGACAAAGGCTACAAA |
| chr5 | 177238128 | 177238146 | + | 936 | AATTCTGACAAAGGCTACAA |
| chr5 | 177238129 | 177238147 | + | 937 | AATTCTGACAAAGGCTACA |
| chr5 | 177238130 | 177238148 | + | 938 | AATTCTGACAAAGGCTAC |
| chr5 | 177238131 | 177238149 | + | 939 | AAATTCTGACAAAGGCTA |
| chr5 | 177238132 | 177238150 | + | 940 | AAGAAATTCTGACAAAGGCT |
| chr5 | 177238133 | 177238151 | + | 941 | AATGAAATTCTGACAAAGGC |
| chr5 | 177238134 | 177238152 | + | 942 | AATGAAATTCTGACAAAGG |
| chr5 | 177238135 | 177238153 | + | 943 | AATGAAATTCTGACAAAG |
| chr5 | 177238136 | 177238154 | + | 944 | AAGAATGAAATTCTGACAAA |
| chr5 | 177238137 | 177238155 | + | 945 | AAGGAATGAAATTCTGACAA |
| chr5 | 177238138 | 177238156 | + | 946 | AAGGAATGAAATTCTGACA |
| chr5 | 177238139 | 177238157 | + | 947 | AAGGAATGAAATTCTGAC |
| chr5 | 177238140 | 177238158 | + | 948 | AAAGGAATGAAATTCTGA |
| chr5 | 177238141 | 177238159 | + | 949 | AAAAGGAATGAAATTCTG |
| chr5 | 177238142 | 177238160 | + | 950 | AATAAAAGGAATGAAATTCT |
| chr5 | 177238143 | 177238161 | + | 951 | AATTAAAAGGAATGAAATTC |
| chr5 | 177238144 | 177238162 | + | 952 | AATTTAAAAGGAATGAAATT |
| chr5 | 177238145 | 177238163 | + | 953 | AACTTTAAAAGGAATGAAAT |
| chr5 | 177238146 | 177238164 | + | 954 | AACTTTAAAAGGAATGAAA |
| chr5 | 177238147 | 177238165 | + | 955 | CACTTTAAAAGGAATGAA |
| chr5 | 177238148 | 177238166 | + | 956 | AACACTTTAAAAGGAATGA |
| chr5 | 177238149 | 177238167 | + | 957 | AACACACTTTAAAAGGAATG |
| chr5 | 177238150 | 177238168 | + | 958 | AACACACTTTAAAAGGAAT |
| chr5 | 177238151 | 177238169 | + | 959 | AACACACTTTAAAAGGAA |
| chr5 | 177238152 | 177238170 | + | 960 | AATAACACACTTTAAAAGGA |
| chr5 | 177238153 | 177238171 | + | 961 | AATAACACACTTTAAAAGG |
| chr5 | 177238154 | 177238172 | + | 962 | AATAACACACTTTAAAAG |
| chr5 | 177238155 | 177238173 | + | 963 | AAGAATAACACACTTTAAAA |
| chr5 | 177238156 | 177238174 | + | 964 | AAGAATAACACACTTTAAA |
| chr5 | 177238157 | 177238175 | + | 965 | AAGAATAACACACTTTAA |
| chr5 | 177238158 | 177238176 | + | 966 | AAAGAATAACACACTTTA |
| chr5 | 177238159 | 177238177 | + | 967 | AAAAGAATAACACACTTT |
| chr5 | 177238160 | 177238178 | + | 968 | AAAAAGAATAACACACTT |
| chr5 | 177238161 | 177238179 | + | 969 | AACAAAAAGAATAACACACT |
| chr5 | 177238162 | 177238180 | + | 970 | AATCAAAAAGAATAACACAC |
| chr5 | 177238163 | 177238181 | + | 971 | AAGTCAAAAAGAATAACACA |
| chr5 | 177238164 | 177238182 | + | 972 | AATGTCAAAAAGAATAACAC |
| chr5 | 177238165 | 177238183 | + | 973 | AAGTGTCAAAAAGAATAACA |
| chr5 | 177238166 | 177238184 | + | 974 | AAGTGTCAAAAAGAATAAC |
| chr5 | 177238167 | 177238185 | + | 975 | AAGTGTCAAAAAGAATAA |
| chr5 | 177238168 | 177238186 | + | 976 | AATAAGTGTCAAAAAGAATA |
| chr5 | 177238169 | 177238187 | + | 977 | AATTAAGTGTCAAAAAGAAT |
| chr5 | 177238170 | 177238188 | + | 978 | AATTTAAGTGTCAAAAAGAA |
| chr5 | 177238171 | 177238189 | + | 979 | AATTTAAGTGTCAAAAAGA |
| chr5 | 177238172 | 177238190 | + | 980 | AATTTAAGTGTCAAAAAG |
| chr5 | 177238173 | 177238191 | + | 981 | AATAATTTAAGTGTCAAAAA |
| chr5 | 177238174 | 177238192 | + | 982 | AAGTAATTTAAGTGTCAAAA |
| chr5 | 177238175 | 177238193 | + | 983 | AATGTAATTTAAGTGTCAAA |
| chr5 | 177238176 | 177238194 | + | 984 | AATTGTAATTTAAGTGTCAA |
| chr5 | 177238177 | 177238195 | + | 985 | AAGTTGTAATTTAAGTGTCA |
| chr5 | 177238178 | 177238196 | + | 986 | AATGTTGTAATTTAAGTGTC |
| chr5 | 177238179 | 177238197 | + | 987 | AATTGTTGTAATTTAAGTGT |
| chr5 | 177238180 | 177238198 | + | 988 | AATTGTTGTAATTTAAGTG |
| chr5 | 177238181 | 177238199 | + | 989 | AATTGTTGTAATTTAAGT |
| chr5 | 177238182 | 177238200 | + | 990 | AAATTGTTGTAATTTAAG |
| chr5 | 177238183 | 177238201 | + | 991 | AAAATTGTTGTAATTTAA |
| chr5 | 177238184 | 177238202 | + | 992 | AACAAAATTGTTGTAATTTA |
| chr5 | 177238185 | 177238203 | + | 993 | AACCAAAATTGTTGTAATTT |
| chr5 | 177238186 | 177238204 | + | 994 | AAGCCAAAATTGTTGTAATT |
| chr5 | 177238187 | 177238205 | + | 995 | AAGGCCAAAATTGTTGTAAT |
| chr5 | 177238188 | 177238206 | + | 996 | AAGGCCAAAATTGTTGTAA |
| chr5 | 177238189 | 177238207 | + | 997 | AACAGGCCAAAATTGTTGTA |
| chr5 | 177238190 | 177238208 | + | 998 | AACAGGCCAAAATTGTTGT |
| chr5 | 177238191 | 177238209 | + | 999 | AACACAGGCCAAAATTGTTG |
| chr5 | 177238192 | 177238210 | + | 1000 | AACCACAGGCCAAAATTGTT |
| chr5 | 177238193 | 177238211 | + | 1001 | AATCCACAGGCCAAAATTGT |
| chr5 | 177238194 | 177238212 | + | 1002 | AAGTCCACAGGCCAAAATTG |
| chr5 | 177238195 | 177238213 | + | 1003 | AAGTCCACAGGCCAAAATT |
| chr5 | 177238196 | 177238214 | + | 1004 | AAGAGTCCACAGGCCAAAAT |
| chr5 | 177238197 | 177238215 | + | 1005 | AAGAGTCCACAGGCCAAAA |
| chr5 | 177238198 | 177238216 | + | 1006 | AATAGAGTCCACAGGCCAAA |
| chr5 | 177238199 | 177238217 | + | 1007 | AATAGAGTCCACAGGCCAA |
| chr5 | 177238200 | 177238218 | + | 1008 | AATAGAGTCCACAGGCCA |
| chr5 | 177238201 | 177238219 | + | 1009 | AAATAGAGTCCACAGGCC |
| chr5 | 177238202 | 177238220 | + | 1010 | AAAATAGAGTCCACAGGC |
| chr5 | 177238237 | 177238255 | + | 1011 | AACTTCACAGCGGGAACTTA |
| chr5 | 177238238 | 177238256 | + | 1012 | AATCTTCACAGCGGGAACTT |
| chr5 | 177238239 | 177238257 | + | 1013 | AACTCTTCACAGCGGGAACT |
| chr5 | 177238240 | 177238258 | + | 1014 | AACCTCTTCACAGCGGGAAC |
| chr5 | 177238241 | 177238259 | + | 1015 | AATCCTCTTCACAGCGGGAA |
| chr5 | 177238242 | 177238260 | + | 1016 | AATTCCTCTTCACAGCGGGA |
| chr5 | 177238243 | 177238261 | + | 1017 | AATTTCCTCTTCACAGCGGG |
| chr5 | 177238244 | 177238262 | + | 1018 | AACTTTCCTCTTCACAGCGG |
| chr5 | 177238245 | 177238263 | + | 1019 | AAGCTTTCCTCTTCACAGCG |
| chr5 | 177238246 | 177238264 | + | 1020 | AAGGCTTTCCTCTTCACAGC |
| chr5 | 177238247 | 177238265 | + | 1021 | AAGGCTTTCCTCTTCACAG |
| chr5 | 177238248 | 177238266 | + | 1022 | AAGGCTTTCCTCTTCACA |
| chr5 | 177238249 | 177238267 | + | 1023 | AAGAAGGCTTTCCTCTTCAC |
| chr5 | 177238250 | 177238268 | + | 1024 | AAGAAGGCTTTCCTCTTCA |
| chr5 | 177238251 | 177238269 | + | 1025 | AATAGAAGGCTTTCCTCTTC |
| chr5 | 177238252 | 177238270 | + | 1026 | AACTAGAAGGCTTTCCTCTT |
| chr5 | 177238253 | 177238271 | + | 1027 | AAGCTAGAAGGCTTTCCTCT |
| chr5 | 177238254 | 177238272 | + | 1028 | AAGGCTAGAAGGCTTTCCTC |
| chr5 | 177238255 | 177238273 | + | 1029 | AAGGGCTAGAAGGCTTTCCT |
| chr5 | 177238256 | 177238274 | + | 1030 | AACGGGCTAGAAGGCTTTCC |
| chr5 | 177238257 | 177238275 | + | 1031 | AATCGGGCTAGAAGGCTTTC |
| chr5 | 177238258 | 177238276 | + | 1032 | AACTCGGGCTAGAAGGCTTT |
| chr5 | 177238259 | 177238277 | + | 1033 | AACCTCGGGCTAGAAGGCTT |
| chr5 | 177238260 | 177238278 | + | 1034 | AACCTCGGGCTAGAAGGCT |
| chr5 | 177238261 | 177238279 | + | 1035 | AAGACCTCGGGCTAGAAGGC |
| chr5 | 177238262 | 177238280 | + | 1036 | AACGACCTCGGGCTAGAAGG |
| chr5 | 177238263 | 177238281 | + | 1037 | AATCGACCTCGGGCTAGAAG |
| chr5 | 177238264 | 177238282 | + | 1038 | AATCGACCTCGGGCTAGAA |
| chr5 | 177238265 | 177238283 | + | 1039 | AAGATCGACCTCGGGCTAGA |
| chr5 | 177238266 | 177238284 | + | 1040 | AAGATCGACCTCGGGCTAG |
| chr5 | 177238267 | 177238285 | + | 1041 | AATAGATCGACCTCGGGCTA |
| chr5 | 177238268 | 177238286 | + | 1042 | AACTAGATCGACCTCGGGCT |
| chr5 | 177238269 | 177238287 | + | 1043 | AACTAGATCGACCTCGGGC |
| chr5 | 177238270 | 177238288 | + | 1044 | AACACTAGATCGACCTCGGG |
| chr5 | 177238271 | 177238289 | + | 1045 | AAGCACTAGATCGACCTCGG |
| chr5 | 177238272 | 177238290 | + | 1046 | AAGCACTAGATCGACCTCG |
| chr5 | 177238273 | 177238291 | + | 1047 | AAGAGCACTAGATCGACCTC |
| chr5 | 177238274 | 177238292 | + | 1048 | AATGAGCACTAGATCGACCT |
| chr5 | 177238275 | 177238293 | + | 1049 | AACTGAGCACTAGATCGACC |
| chr5 | 177238276 | 177238294 | + | 1050 | AATCTGAGCACTAGATCGAC |
| chr5 | 177238277 | 177238295 | + | 1051 | AATTCTGAGCACTAGATCGA |
| chr5 | 177238278 | 177238296 | + | 1052 | AAGTTCTGAGCACTAGATCG |
| chr5 | 177238279 | 177238297 | + | 1053 | AATGTTCTGAGCACTAGATC |
| chr5 | 177238280 | 177238298 | + | 1054 | AATTGTTCTGAGCACTAGAT |
| chr5 | 177238281 | 177238299 | + | 1055 | AACTTGTTCTGAGCACTAGA |
| chr5 | 177238282 | 177238300 | + | 1056 | AAGCTTGTTCTGAGCACTAG |
| chr5 | 177238283 | 177238301 | + | 1057 | AATGCTTGTTCTGAGCACTA |
| chr5 | 177238284 | 177238302 | + | 1058 | AACTGCTTGTTCTGAGCACT |
| chr5 | 177238285 | 177238303 | + | 1059 | AACCTGCTTGTTCTGAGCAC |
| chr5 | 177238286 | 177238304 | + | 1060 | AACCTGCTTGTTCTGAGCA |
| chr5 | 177238287 | 177238305 | + | 1061 | AACACCTGCTTGTTCTGAGC |
| chr5 | 177238288 | 177238306 | + | 1062 | AACCACCTGCTTGTTCTGAG |
| chr5 | 177238289 | 177238307 | + | 1063 | AATCCACCTGCTTGTTCTGA |
| chr5 | 177238290 | 177238308 | + | 1064 | AAGTCCACCTGCTTGTTCTG |
| chr5 | 177238291 | 177238309 | + | 1065 | AACGTCCACCTGCTTGTTCT |
| chr5 | 177238292 | 177238310 | + | 1066 | AATCGTCCACCTGCTTGTTC |
| chr5 | 177238293 | 177238311 | + | 1067 | AACTCGTCCACCTGCTTGTT |
| chr5 | 177238294 | 177238312 | + | 1068 | AATCTCGTCCACCTGCTTGT |
| chr5 | 177238295 | 177238313 | + | 1069 | AATTCTCGTCCACCTGCTTG |
| chr5 | 177238296 | 177238314 | + | 1070 | AATTCTCGTCCACCTGCTT |
| chr5 | 177238297 | 177238315 | + | 1071 | AATTCTCGTCCACCTGCT |
| chr5 | 177238298 | 177238316 | + | 1072 | AAGAATTCTCGTCCACCTGC |
| chr5 | 177238299 | 177238317 | + | 1073 | AAGAATTCTCGTCCACCTG |
| chr5 | 177238300 | 177238318 | + | 1074 | AAGAATTCTCGTCCACCT |
| chr5 | 177238301 | 177238319 | + | 1075 | AAAGAATTCTCGTCCACC |
| chr5 | 177238302 | 177238320 | + | 1076 | AACAAAGAATTCTCGTCCAC |
| chr5 | 177238303 | 177238321 | + | 1077 | AATCAAAGAATTCTCGTCCA |
| chr5 | 177238304 | 177238322 | + | 1078 | AATCAAAGAATTCTCGTCC |
| chr5 | 177238305 | 177238323 | + | 1079 | AATCAAAGAATTCTCGTC |
| chr5 | 177238306 | 177238324 | + | 1080 | AAATCAAAGAATTCTCGT |
| chr5 | 177238307 | 177238325 | + | 1081 | AAGAAATCAAAGAATTCTCG |
| chr5 | 177238308 | 177238326 | + | 1082 | AATGAAATCAAAGAATTCTC |
| chr5 | 177238309 | 177238327 | + | 1083 | AATTGAAATCAAAGAATTCT |
| chr5 | 177238310 | 177238328 | + | 1084 | AAGTTGAAATCAAAGAATTC |
| chr5 | 177238311 | 177238329 | + | 1085 | AAGGTTGAAATCAAAGAATT |
| chr5 | 177238312 | 177238330 | + | 1086 | AATGGTTGAAATCAAAGAAT |
| chr5 | 177238313 | 177238331 | + | 1087 | AATTGGTTGAAATCAAAGAA |
| chr5 | 177238314 | 177238332 | + | 1088 | AATTTGGTTGAAATCAAAGA |
| chr5 | 177238315 | 177238333 | + | 1089 | AACTTTGGTTGAAATCAAAG |
| chr5 | 177238316 | 177238334 | + | 1090 | AATCTTTGGTTGAAATCAAA |
| chr5 | 177238317 | 177238335 | + | 1091 | AATTCTTTGGTTGAAATCAA |
| chr5 | 177238318 | 177238336 | + | 1092 | AACTTCTTTGGTTGAAATCA |
| chr5 | 177238319 | 177238337 | + | 1093 | AATCTTCTTTGGTTGAAATC |
| chr5 | 177238320 | 177238338 | + | 1094 | AACTCTTCTTTGGTTGAAAT |
| chr5 | 177238321 | 177238339 | + | 1095 | AAGCTCTTCTTTGGTTGAAA |
| chr5 | 177238322 | 177238340 | + | 1096 | AAGGCTCTTCTTTGGTTGAA |
| chr5 | 177238323 | 177238341 | + | 1097 | AAGGCTCTTCTTTGGTTGA |
| chr5 | 177238324 | 177238342 | + | 1098 | AAGAGGCTCTTCTTTGGTTG |
| chr5 | 177238325 | 177238343 | + | 1099 | AAGGAGGCTCTTCTTTGGTT |
| chr5 | 177238326 | 177238344 | + | 1100 | AATGGAGGCTCTTCTTTGGT |
| chr5 | 177238327 | 177238345 | + | 1101 | AACTGGAGGCTCTTCTTTGG |
| chr5 | 177238328 | 177238346 | + | 1102 | AACTGGAGGCTCTTCTTTG |
| chr5 | 177238329 | 177238347 | + | 1103 | AACTGGAGGCTCTTCTTT |
| chr5 | 177238330 | 177238348 | + | 1104 | AAGAACTGGAGGCTCTTCTT |
| chr5 | 177238331 | 177238349 | + | 1105 | AAGAACTGGAGGCTCTTCT |
| chr5 | 177238332 | 177238350 | + | 1106 | AAGAACTGGAGGCTCTTC |
| chr5 | 177238333 | 177238351 | + | 1107 | AACAAGAACTGGAGGCTCTT |
| chr5 | 177238334 | 177238352 | + | 1108 | AATCAAGAACTGGAGGCTCT |
| chr5 | 177238335 | 177238353 | + | 1109 | AATTCAAGAACTGGAGGCTC |
| chr5 | 177238336 | 177238354 | + | 1110 | AATTTCAAGAACTGGAGGCT |
| chr5 | 177238337 | 177238355 | + | 1111 | AACTTTCAAGAACTGGAGGC |
| chr5 | 177238338 | 177238356 | + | 1112 | AACCTTTCAAGAACTGGAGG |
| chr5 | 177238339 | 177238357 | + | 1113 | AACCCTTTCAAGAACTGGAG |
| chr5 | 177238340 | 177238358 | + | 1114 | AATCCCTTTCAAGAACTGGA |
| chr5 | 177238341 | 177238359 | + | 1115 | AACTCCCTTTCAAGAACTGG |
| chr5 | 177238342 | 177238360 | + | 1116 | AACCTCCCTTTCAAGAACTG |
| chr5 | 177238343 | 177238361 | + | 1117 | AAGCCTCCCTTTCAAGAACT |
| chr5 | 177238344 | 177238362 | + | 1118 | AAGCCTCCCTTTCAAGAAC |
| chr5 | 177238345 | 177238363 | + | 1119 | AAGAGCCTCCCTTTCAAGAA |
| chr5 | 177238346 | 177238364 | + | 1120 | AAGGAGCCTCCCTTTCAAGA |
| chr5 | 177238347 | 177238365 | + | 1121 | AACGGAGCCTCCCTTTCAAG |
| chr5 | 177238348 | 177238366 | + | 1122 | AACGGAGCCTCCCTTTCAA |
| chr5 | 177238349 | 177238367 | + | 1123 | AACGGAGCCTCCCTTTCA |
| chr5 | 177238350 | 177238368 | + | 1124 | AAACGGAGCCTCCCTTTC |
| chr5 | 177238351 | 177238369 | + | 1125 | AAAACGGAGCCTCCCTTT |
| chr5 | 177238352 | 177238370 | + | 1126 | AAAAACGGAGCCTCCCTT |
| chr5 | 177238353 | 177238371 | + | 1127 | AACAAAAACGGAGCCTCCCT |
| chr5 | 177238354 | 177238372 | + | 1128 | AACCAAAAACGGAGCCTCCC |
| chr5 | 177238355 | 177238373 | + | 1129 | AATCCAAAAACGGAGCCTCC |
| chr5 | 177238356 | 177238374 | + | 1130 | AACTCCAAAAACGGAGCCTC |
| chr5 | 177238357 | 177238375 | + | 1131 | AACCTCCAAAAACGGAGCCT |
| chr5 | 177238358 | 177238376 | + | 1132 | AACCCTCCAAAAACGGAGCC |
| chr5 | 177238359 | 177238377 | + | 1133 | AAGCCCTCCAAAAACGGAGC |
| chr5 | 177238360 | 177238378 | + | 1134 | AAGGCCCTCCAAAAACGGAG |
| chr5 | 177238361 | 177238379 | + | 1135 | AAGGGCCCTCCAAAAACGGA |
| chr5 | 177238362 | 177238380 | + | 1136 | AAGGGGCCCTCCAAAAACGG |
| chr5 | 177238363 | 177238381 | + | 1137 | AAGGGGCCCTCCAAAAACG |
| chr5 | 177238364 | 177238382 | + | 1138 | AAGGGGCCCTCCAAAAAC |
| chr5 | 177238365 | 177238383 | + | 1139 | AACAAGGGGCCCTCCAAAAA |
| chr5 | 177238366 | 177238384 | + | 1140 | AACCAAGGGGCCCTCCAAAA |
| chr5 | 177238367 | 177238385 | + | 1141 | AAGCCAAGGGGCCCTCCAAA |
| chr5 | 177238368 | 177238386 | + | 1142 | AAGCCAAGGGGCCCTCCAA |
| chr5 | 177238369 | 177238387 | + | 1143 | AAGAGCCAAGGGGCCCTCCA |
| chr5 | 177238370 | 177238388 | + | 1144 | AATGAGCCAAGGGGCCCTCC |
| chr5 | 177238371 | 177238389 | + | 1145 | AACTGAGCCAAGGGGCCCTC |
| chr5 | 177238372 | 177238390 | + | 1146 | AACTGAGCCAAGGGGCCCT |
| chr5 | 177238373 | 177238391 | + | 1147 | AAGACTGAGCCAAGGGGCCC |
| chr5 | 177238374 | 177238392 | + | 1148 | AATGACTGAGCCAAGGGGCC |
| chr5 | 177238375 | 177238393 | + | 1149 | AACTGACTGAGCCAAGGGGC |
| chr5 | 177238376 | 177238394 | + | 1150 | AATCTGACTGAGCCAAGGGG |
| chr5 | 177238377 | 177238395 | + | 1151 | AATTCTGACTGAGCCAAGGG |
| chr5 | 177238378 | 177238396 | + | 1152 | AAGTTCTGACTGAGCCAAGG |
| chr5 | 177238379 | 177238397 | + | 1153 | AAGTTCTGACTGAGCCAAG |
| chr5 | 177238380 | 177238398 | + | 1154 | AAGTTCTGACTGAGCCAA |
| chr5 | 177238381 | 177238399 | + | 1155 | AACAAGTTCTGACTGAGCCA |
| chr5 | 177238382 | 177238400 | + | 1156 | AACCAAGTTCTGACTGAGCC |
| chr5 | 177238383 | 177238401 | + | 1157 | AATCCAAGTTCTGACTGAGC |
| chr5 | 177238384 | 177238402 | + | 1158 | AACTCCAAGTTCTGACTGAG |
| chr5 | 177238385 | 177238403 | + | 1159 | AACCTCCAAGTTCTGACTGA |
| chr5 | 177238386 | 177238404 | + | 1160 | AACCTCCAAGTTCTGACTG |
| chr5 | 177238387 | 177238405 | + | 1161 | AACACCTCCAAGTTCTGACT |
| chr5 | 177238388 | 177238406 | + | 1162 | AACCACCTCCAAGTTCTGAC |
| chr5 | 177238389 | 177238407 | + | 1163 | AATCCACCTCCAAGTTCTGA |
| chr5 | 177238390 | 177238408 | + | 1164 | AAGTCCACCTCCAAGTTCTG |
| chr5 | 177238391 | 177238409 | + | 1165 | AATGTCCACCTCCAAGTTCT |
| chr5 | 177238392 | 177238410 | + | 1166 | AATGTCCACCTCCAAGTTC |
| chr5 | 177238393 | 177238411 | + | 1167 | AACATGTCCACCTCCAAGTT |
| chr5 | 177238394 | 177238412 | + | 1168 | AAGCATGTCCACCTCCAAGT |
| chr5 | 177238395 | 177238413 | + | 1169 | AAGCATGTCCACCTCCAAG |
| chr5 | 177238396 | 177238414 | + | 1170 | AACAGCATGTCCACCTCCAA |
| chr5 | 177238397 | 177238415 | + | 1171 | AATCAGCATGTCCACCTCCA |
| chr5 | 177238398 | 177238416 | + | 1172 | AACTCAGCATGTCCACCTCC |
| chr5 | 177238399 | 177238417 | + | 1173 | AACTCAGCATGTCCACCTC |
| chr5 | 177238400 | 177238418 | + | 1174 | AACTCAGCATGTCCACCT |
| chr5 | 177238401 | 177238419 | + | 1175 | AACAACTCAGCATGTCCACC |
| chr5 | 177238402 | 177238420 | + | 1176 | AAGCAACTCAGCATGTCCAC |
| chr5 | 177238403 | 177238421 | + | 1177 | AAGGCAACTCAGCATGTCCA |
| chr5 | 177238404 | 177238422 | + | 1178 | AACGGCAACTCAGCATGTCC |
| chr5 | 177238405 | 177238423 | + | 1179 | AAGCGGCAACTCAGCATGTC |
| chr5 | 177238406 | 177238424 | + | 1180 | AATGCGGCAACTCAGCATGT |
| chr5 | 177238407 | 177238425 | + | 1181 | AACTGCGGCAACTCAGCATG |
| chr5 | 177238408 | 177238426 | + | 1182 | AAGCTGCGGCAACTCAGCAT |
| chr5 | 177238409 | 177238427 | + | 1183 | AAGCTGCGGCAACTCAGCA |
| chr5 | 177238410 | 177238428 | + | 1184 | AACAGCTGCGGCAACTCAGC |
| chr5 | 177238411 | 177238429 | + | 1185 | AATCAGCTGCGGCAACTCAG |
| chr5 | 177238412 | 177238430 | + | 1186 | AAGTCAGCTGCGGCAACTCA |
| chr5 | 177238413 | 177238431 | + | 1187 | AAGGTCAGCTGCGGCAACTC |
| chr5 | 177238414 | 177238432 | + | 1188 | AAGGTCAGCTGCGGCAACT |
| chr5 | 177238415 | 177238433 | + | 1189 | AAGGTCAGCTGCGGCAAC |
| chr5 | 177238416 | 177238434 | + | 1190 | AACAAGGTCAGCTGCGGCAA |
| chr5 | 177238417 | 177238435 | + | 1191 | AACAAGGTCAGCTGCGGCA |
| chr5 | 177238418 | 177238436 | + | 1192 | AAGACAAGGTCAGCTGCGGC |
| chr5 | 177238419 | 177238437 | + | 1193 | AAGACAAGGTCAGCTGCGG |
| chr5 | 177238420 | 177238438 | + | 1194 | AACAGACAAGGTCAGCTGCG |
| chr5 | 177238421 | 177238439 | + | 1195 | AACAGACAAGGTCAGCTGC |
| chr5 | 177238422 | 177238440 | + | 1196 | AACACAGACAAGGTCAGCTG |
| chr5 | 177238423 | 177238441 | + | 1197 | AAGCACAGACAAGGTCAGCT |
| chr5 | 177238424 | 177238442 | + | 1198 | AAGGCACAGACAAGGTCAGC |
| chr5 | 177238425 | 177238443 | + | 1199 | AAGGCACAGACAAGGTCAG |
| chr5 | 177238426 | 177238444 | + | 1200 | AACAGGCACAGACAAGGTCA |
| chr5 | 177238427 | 177238445 | + | 1201 | AACAGGCACAGACAAGGTC |
| chr5 | 177238428 | 177238446 | + | 1202 | AACACAGGCACAGACAAGGT |
| chr5 | 177238429 | 177238447 | + | 1203 | AACCACAGGCACAGACAAGG |
| chr5 | 177238430 | 177238448 | + | 1204 | AAGCCACAGGCACAGACAAG |
| chr5 | 177238431 | 177238449 | + | 1205 | AAGCCACAGGCACAGACAA |
| chr5 | 177238432 | 177238450 | + | 1206 | AAGAGCCACAGGCACAGACA |
| chr5 | 177238433 | 177238451 | + | 1207 | AAGGAGCCACAGGCACAGAC |
| chr5 | 177238434 | 177238452 | + | 1208 | AACGGAGCCACAGGCACAGA |
| chr5 | 177238435 | 177238453 | + | 1209 | AACCGGAGCCACAGGCACAG |
| chr5 | 177238436 | 177238454 | + | 1210 | AATCCGGAGCCACAGGCACA |
| chr5 | 177238437 | 177238455 | + | 1211 | AATTCCGGAGCCACAGGCAC |
| chr5 | 177238438 | 177238456 | + | 1212 | AACTTCCGGAGCCACAGGCA |
| chr5 | 177238439 | 177238457 | + | 1213 | AACTTCCGGAGCCACAGGC |
| chr5 | 177238440 | 177238458 | + | 1214 | AAGACTTCCGGAGCCACAGG |
| chr5 | 177238441 | 177238459 | + | 1215 | AAGACTTCCGGAGCCACAG |
| chr5 | 177238442 | 177238460 | + | 1216 | AAGAGACTTCCGGAGCCACA |
| chr5 | 177238443 | 177238461 | + | 1217 | AAGAGACTTCCGGAGCCAC |
| chr5 | 177238444 | 177238462 | + | 1218 | AAGAGAGACTTCCGGAGCCA |
| chr5 | 177238445 | 177238463 | + | 1219 | AAGGAGAGACTTCCGGAGCC |
| chr5 | 177238446 | 177238464 | + | 1220 | AATGGAGAGACTTCCGGAGC |
| chr5 | 177238447 | 177238465 | + | 1221 | AAGTGGAGAGACTTCCGGAG |
| chr5 | 177238448 | 177238466 | + | 1222 | AACGTGGAGAGACTTCCGGA |
| chr5 | 177238449 | 177238467 | + | 1223 | AACCGTGGAGAGACTTCCGG |
| chr5 | 177238450 | 177238468 | + | 1224 | AAGCCGTGGAGAGACTTCCG |
| chr5 | 177238451 | 177238469 | + | 1225 | AAGGCCGTGGAGAGACTTCC |
| chr5 | 177238452 | 177238470 | + | 1226 | AAGGCCGTGGAGAGACTTC |
| chr5 | 177238453 | 177238471 | + | 1227 | AACAGGCCGTGGAGAGACTT |
| chr5 | 177238454 | 177238472 | + | 1228 | AAGCAGGCCGTGGAGAGACT |
| chr5 | 177238455 | 177238473 | + | 1229 | AAGGCAGGCCGTGGAGAGAC |
| chr5 | 177238456 | 177238474 | + | 1230 | AAGGGCAGGCCGTGGAGAGA |
| chr5 | 177238457 | 177238475 | + | 1231 | AAGGGCAGGCCGTGGAGAG |
| chr5 | 177238458 | 177238476 | + | 1232 | AAGGGCAGGCCGTGGAGA |
| chr5 | 177238459 | 177238477 | + | 1233 | AACAAGGGCAGGCCGTGGAG |
| chr5 | 177238460 | 177238478 | + | 1234 | AATCAAGGGCAGGCCGTGGA |
| chr5 | 177238461 | 177238479 | + | 1235 | AACTCAAGGGCAGGCCGTGG |
| chr5 | 177238462 | 177238480 | + | 1236 | AACTCAAGGGCAGGCCGTG |
| chr5 | 177238463 | 177238481 | + | 1237 | AAGACTCAAGGGCAGGCCGT |
| chr5 | 177238464 | 177238482 | + | 1238 | AAGACTCAAGGGCAGGCCG |
| chr5 | 177238465 | 177238483 | + | 1239 | AACAGACTCAAGGGCAGGCC |
| chr5 | 177238466 | 177238484 | + | 1240 | AATCAGACTCAAGGGCAGGC |
| chr5 | 177238467 | 177238485 | + | 1241 | AACTCAGACTCAAGGGCAGG |
| chr5 | 177238468 | 177238486 | + | 1242 | AACCTCAGACTCAAGGGCAG |
| chr5 | 177238469 | 177238487 | + | 1243 | AATCCTCAGACTCAAGGGCA |
| chr5 | 177238470 | 177238488 | + | 1244 | AATTCCTCAGACTCAAGGGC |
| chr5 | 177238471 | 177238489 | + | 1245 | AATTCCTCAGACTCAAGGG |
| chr5 | 177238472 | 177238490 | + | 1246 | AATTCCTCAGACTCAAGG |
| chr5 | 177238473 | 177238491 | + | 1247 | AACAATTCCTCAGACTCAAG |
| chr5 | 177238474 | 177238492 | + | 1248 | AAGCAATTCCTCAGACTCAA |
| chr5 | 177238475 | 177238493 | + | 1249 | AAGCAATTCCTCAGACTCA |
| chr5 | 177238476 | 177238494 | + | 1250 | AATAGCAATTCCTCAGACTC |
| chr5 | 177238477 | 177238495 | + | 1251 | AACTAGCAATTCCTCAGACT |
| chr5 | 177238478 | 177238496 | + | 1252 | AACTAGCAATTCCTCAGAC |
| chr5 | 177238479 | 177238497 | + | 1253 | AACTAGCAATTCCTCAGA |
| chr5 | 177238480 | 177238498 | + | 1254 | AATAACTAGCAATTCCTCAG |
| chr5 | 177238481 | 177238499 | + | 1255 | AATTAACTAGCAATTCCTCA |
| chr5 | 177238482 | 177238500 | + | 1256 | AATTTAACTAGCAATTCCTC |
| chr5 | 177238483 | 177238501 | + | 1257 | AATTTTAACTAGCAATTCCT |
| chr5 | 177238484 | 177238502 | + | 1258 | AAGTTTTAACTAGCAATTCC |
| chr5 | 177238485 | 177238503 | + | 1259 | AACGTTTTAACTAGCAATTC |
| chr5 | 177238514 | 177238532 | + | 1260 | AATACTGAGACCCCAACCCC |
| chr5 | 177238515 | 177238533 | + | 1261 | AATACTGAGACCCCAACCC |
| chr5 | 177238516 | 177238534 | + | 1262 | AATACTGAGACCCCAACC |
| chr5 | 177238517 | 177238535 | + | 1263 | AAATACTGAGACCCCAAC |
| chr5 | 177238518 | 177238536 | + | 1264 | AACAAATACTGAGACCCCAA |
| chr5 | 177238519 | 177238537 | + | 1265 | AATCAAATACTGAGACCCCA |
| chr5 | 177238520 | 177238538 | + | 1266 | AACTCAAATACTGAGACCCC |
| chr5 | 177238521 | 177238539 | + | 1267 | AAGCTCAAATACTGAGACCC |
| chr5 | 177238522 | 177238540 | + | 1268 | AATGCTCAAATACTGAGACC |
| chr5 | 177238523 | 177238541 | + | 1269 | AACTGCTCAAATACTGAGAC |
| chr5 | 177238524 | 177238542 | + | 1270 | AATCTGCTCAAATACTGAGA |
| chr5 | 177238525 | 177238543 | + | 1271 | AATCTGCTCAAATACTGAG |
| chr5 | 177238526 | 177238544 | + | 1272 | AATATCTGCTCAAATACTGA |
| chr5 | 177238527 | 177238545 | + | 1273 | AATATCTGCTCAAATACTG |
| chr5 | 177238528 | 177238546 | + | 1274 | AACATATCTGCTCAAATACT |
| chr5 | 177238529 | 177238547 | + | 1275 | AATCATATCTGCTCAAATAC |
| chr5 | 177238530 | 177238548 | + | 1276 | AATCATATCTGCTCAAATA |
| chr5 | 177238531 | 177238549 | + | 1277 | AATCATATCTGCTCAAAT |
| chr5 | 177238532 | 177238550 | + | 1278 | AATAATCATATCTGCTCAAA |
| chr5 | 177238533 | 177238551 | + | 1279 | AACTAATCATATCTGCTCAA |
| chr5 | 177238534 | 177238552 | + | 1280 | AATCTAATCATATCTGCTCA |
| chr5 | 177238535 | 177238553 | + | 1281 | AACTCTAATCATATCTGCTC |
| chr5 | 177238536 | 177238554 | + | 1282 | AACCTCTAATCATATCTGCT |
| chr5 | 177238537 | 177238555 | + | 1283 | AATCCTCTAATCATATCTGC |
| chr5 | 177238538 | 177238556 | + | 1284 | AATTCCTCTAATCATATCTG |
| chr5 | 177238539 | 177238557 | + | 1285 | AACTTCCTCTAATCATATCT |
| chr5 | 177238540 | 177238558 | + | 1286 | AAGCTTCCTCTAATCATATC |
| chr5 | 177238541 | 177238559 | + | 1287 | AATGCTTCCTCTAATCATAT |
| chr5 | 177238542 | 177238560 | + | 1288 | AACTGCTTCCTCTAATCATA |
| chr5 | 177238543 | 177238561 | + | 1289 | AACCTGCTTCCTCTAATCAT |
| chr5 | 177238544 | 177238562 | + | 1290 | AATCCTGCTTCCTCTAATCA |
| chr5 | 177238545 | 177238563 | + | 1291 | AACTCCTGCTTCCTCTAATC |
| chr5 | 177238546 | 177238564 | + | 1292 | AATCTCCTGCTTCCTCTAAT |
| chr5 | 177238547 | 177238565 | + | 1293 | AATCTCCTGCTTCCTCTAA |
| chr5 | 177238548 | 177238566 | + | 1294 | AATCTCCTGCTTCCTCTA |
| chr5 | 177238549 | 177238567 | + | 1295 | AAATCTCCTGCTTCCTCT |
| chr5 | 177238550 | 177238568 | + | 1296 | AAAATCTCCTGCTTCCTC |
| chr5 | 177238551 | 177238569 | + | 1297 | AATAAAATCTCCTGCTTCCT |
| chr5 | 177238552 | 177238570 | + | 1298 | AACTAAAATCTCCTGCTTCC |
| chr5 | 177238553 | 177238571 | + | 1299 | AACTAAAATCTCCTGCTTC |
| chr5 | 177238554 | 177238572 | + | 1300 | AATACTAAAATCTCCTGCTT |
| chr5 | 177238555 | 177238573 | + | 1301 | AATACTAAAATCTCCTGCT |
| chr5 | 177238556 | 177238574 | + | 1302 | AACATACTAAAATCTCCTGC |
| chr5 | 177238557 | 177238575 | + | 1303 | AACATACTAAAATCTCCTG |
| chr5 | 177238558 | 177238576 | + | 1304 | AACATACTAAAATCTCCT |
| chr5 | 177238559 | 177238577 | + | 1305 | AAACATACTAAAATCTCC |
| chr5 | 177238560 | 177238578 | + | 1306 | AAAACATACTAAAATCTC |
| chr5 | 177238561 | 177238579 | + | 1307 | AACAAAACATACTAAAATCT |
| chr5 | 177238562 | 177238580 | + | 1308 | AATCAAAACATACTAAAATC |
| chr5 | 177238563 | 177238581 | + | 1309 | AATCAAAACATACTAAAAT |
| chr5 | 177238564 | 177238582 | + | 1310 | AACATCAAAACATACTAAAA |
| chr5 | 177238565 | 177238583 | + | 1311 | AACATCAAAACATACTAAA |
| chr5 | 177238566 | 177238584 | + | 1312 | AATACATCAAAACATACTAA |
| chr5 | 177238567 | 177238585 | + | 1313 | ATTACATCAAAACATACTA |
| chr5 | 177238568 | 177238586 | + | 1314 | ATTTACATCAAAACATACT |
| chr5 | 177238569 | 177238587 | + | 1315 | AACTTTACATCAAAACATAC |
| chr5 | 177238570 | 177238588 | + | 1316 | AAGCTTTACATCAAAACATA |
| chr5 | 177238571 | 177238589 | + | 1317 | AAGGCTTTACATCAAAACAT |
| chr5 | 177238572 | 177238590 | + | 1318 | AATGGCTTTACATCAAAACA |
| chr5 | 177238573 | 177238591 | + | 1319 | AATTGGCTTTACATCAAAAC |
| chr5 | 177238574 | 177238592 | + | 1320 | AAGTTGGCTTTACATCAAAA |
| chr5 | 177238575 | 177238593 | + | 1321 | AATGTTGGCTTTACATCAAA |
| chr5 | 177238576 | 177238594 | + | 1322 | AATGTTGGCTTTACATCAA |
| chr5 | 177238577 | 177238595 | + | 1323 | AATGTTGGCTTTACATCA |
| chr5 | 177238578 | 177238596 | + | 1324 | AACAATGTTGGCTTTACATC |
| chr5 | 177238579 | 177238597 | + | 1325 | AACAATGTTGGCTTTACAT |
| chr5 | 177238580 | 177238598 | + | 1326 | AATACAATGTTGGCTTTACA |
| chr5 | 177238581 | 177238599 | + | 1327 | AATACAATGTTGGCTTTAC |
| chr5 | 177238582 | 177238600 | + | 1328 | AAGATACAATGTTGGCTTTA |
| chr5 | 177238583 | 177238601 | + | 1329 | AAGATACAATGTTGGCTTT |
| chr5 | 177238584 | 177238602 | + | 1330 | AATAGATACAATGTTGGCTT |
| chr5 | 177238585 | 177238603 | + | 1331 | AATAGATACAATGTTGGCT |
| chr5 | 177238586 | 177238604 | + | 1332 | AATATAGATACAATGTTGGC |
| chr5 | 177238587 | 177238605 | + | 1333 | AATATAGATACAATGTTGG |
| chr5 | 177238588 | 177238606 | + | 1334 | AATATATAGATACAATGTTG |
| chr5 | 177238589 | 177238607 | + | 1335 | AAGTATATAGATACAATGTT |
| chr5 | 177238590 | 177238608 | + | 1336 | AATGTATATAGATACAATGT |
| chr5 | 177238591 | 177238609 | + | 1337 | AATTGTATATAGATACAATG |
| chr5 | 177238592 | 177238610 | + | 1338 | AATTGTATATAGATACAAT |
| chr5 | 177238593 | 177238611 | + | 1339 | AATATTGTATATAGATACAA |
| chr5 | 177238594 | 177238612 | + | 1340 | AATTATTGTATATAGATACA |
| chr5 | 177238595 | 177238613 | + | 1341 | AATTTATTGTATATAGATAC |
| chr5 | 177238596 | 177238614 | + | 1342 | AAGTTTATTGTATATAGATA |
| chr5 | 177238597 | 177238615 | + | 1343 | AAGTTTATTGTATATAGAT |
| chr5 | 177238598 | 177238616 | + | 1344 | AATAGTTTATTGTATATAGA |
| chr5 | 177238599 | 177238617 | + | 1345 | AAGTAGTTTATTGTATATAG |
| chr5 | 177238600 | 177238618 | + | 1346 | AAGGTAGTTTATTGTATATA |
| chr5 | 177238601 | 177238619 | + | 1347 | AAGGGTAGTTTATTGTATAT |
| chr5 | 177238602 | 177238620 | + | 1348 | AAGGGGTAGTTTATTGTATA |
| chr5 | 177238603 | 177238621 | + | 1349 | AAGGGGGTAGTTTATTGTAT |
| chr5 | 177238604 | 177238622 | + | 1350 | AAGGGGGTAGTTTATTGTA |
| chr5 | 177238605 | 177238623 | + | 1351 | AAGGGGGTAGTTTATTGT |
| chr5 | 177238606 | 177238624 | + | 1352 | AAAGGGGGTAGTTTATTG |
| chr5 | 177238607 | 177238625 | + | 1353 | AAAAGGGGGTAGTTTATT |
| chr5 | 177238608 | 177238626 | + | 1354 | AACAAAAGGGGGTAGTTTAT |
| chr5 | 177238609 | 177238627 | + | 1355 | AACAAAAGGGGGTAGTTTA |

TABLE 5C
Exemplary U7 Vector Sequence

| | | SEQ |
|---|---|---|
| | | ID |
| Region | Sequence | NO: |
| Promoter | cccacatcgcctgccactacttaagtccgattcacttcggctttagctccaagcctttaatctcgcgaagct | 1751 |
| sequence | ctttttttttttttaacaacataggagctgtgattggctgttttcagccaatcagcactgactcatttgcat | |
| (Mouse U7 | agcctttacaagcggtcacaaactcaagaaacgagcggttttaatagtcttttagaatattgtttatcgaac | |
| promoter) | cgaataaggaactgtgctttgtgattcacatatcagtggaggggtgtggaaatggcaccttgatctcaccct | |
| | catcgaaagtggagttgatgtccttccctggctcgctacagacgcacttccgc | |
| Wild-type U7 | AAGTGTTACAGCTCTTTTAG | 1752 |
| Antisense | | |
| sequence | | |
| Wild-type U7 | AAGTGTTACAGCTCTTTTAGAATTTGTCTAGCAGGTTTTCTGAC | 1753 |
| non-coding | TTCGGTCGGAAAACCCCT | |
| RNA sequence | | |
| ASO sequences | Any of the ASO sequence from Table 4, Table 5A, Table 5A-1, Table 5B, | |
| replacing the | Table 5B-1, Table 5G, and Table 5G-1 | |
| Wild-type U7 | | |
| Antisense | | |
| sequence | | |
| Modified U7 | AAGTGTTACAGCTCTTTTAGAATTTTTGGAGCAGGTTTTCTGAC | 1754 |
| snRNA | TTCGGTCGGAAAACCCCT | |
| (smOPT) non- | | |
| coding RNA | | |
| sequence | | |
| smOPT | AATTTTTGGAG | 1755 |
| sequence | | |
| 3′ regulatory | cccaatttcactggtctacaatgaaagcaaaacagttctcttccccgctccccggtgtgtgagaggggct | 1756 |
| sequence | ttgatccttctctggtttcctaggaaacgcgtatgtgctagagccacgctctgagacttccgcctcgtgc | |
| | ggtcccgcttcctttctgcctcctctgg | |
| Exemplary Full | cccacatcgcctgccactacttaagtccgattcacttcggctttagctccaagcctttaatctcgcgaag | 1757 |
| sequence of | ctctttttttttttttaacaacataggagctgtgattggctgttttcagccaatcagcactgactcattt | |
| wild-type U7 | gcatagcctttacaagcggtcacaaactcaagaaacgagcggttttaatagtcttttagaatattgttta | |
| snRNA | tcgaaccgaataaggaactgtgctttgtgattcacatatcagtggaggggtgtggaaatggcaccttgat | |
| | ctcaccctcatcgaaagtggagttgatgtccttccctggctcgctacagacgcacttccgcAAGTGTTAC | |
| | AGCTCTTTTAGAATTTGTCTAGCAGGTTTTCTGACTTCGGTCGGAAAACCCCTcccaattt | |
| | cactggtctacaatgaaagcaaaacagttctcttccccgctccccggtgtgtgagaggggctttgatcct | |
| | tctctggtttcctaggaaacgcgtatgtgctagagccacgctctgagacttccgcctcgtgcggtcccgc | |
| | ttcctttctgcctcctctgg | |
| Exemplary Full | cccacatcgcctgccactacttaagtccgattcacttcggctttagctccaagcctttaatctcgcgaag | 1758 |
| sequence of | ctctttttttttttttaacaacataggagctgtgattggctgttttcagccaatcagcactgactcattt | |
| modified U7 | gcatagcctttacaagcggtcacaaactcaagaaacgagcggttttaatagtcttttagaatattgttta | |
| snRNA | tcgaaccgaataaggaactgtgctttgtgattcacatatcagtggaggggtgtggaaatggcaccttgat | |
| (smOPT) | ctcaccctcatcgaaagtggagttgatgtccttccctggctcgctacagacgcacttccgcAAGTGTTAC | |
| | AGCTCTTTTAGAATTTTTGGAGCAGGTTTTCTGACTTCGGTCGGAAAACCCCTcccaattt | |
| | cactggtctacaatgaaagcaaaacagttctcttccccgctccccggtgtgtgagaggggctttgatcct | |
| | tctctggtttcctaggaaacgcgtatgtgctagagccacgctctgagacttccgcctcgtgcggtcccgc | |
| | ttcctttctgcctcctctgg | |
| Full sequence | cccacatcgcctgccactacttaagtccgattcacttcggctttagctccaagcctttaatctcgcgaag | 1751 |
| of modified U7 | ctctttttttttttttaacaacataggagctgtgattggctgttttcagccaatcagcactgactcattt | |
| snRNA | gcatagcctttacaagcggtcacaaactcaagaaacgagcggttttaatagtcttttagaatattgttta | |
| (smOPT) | tcgaaccgaataaggaactgtgctttgtgattcacatatcagtggaggggtgtggaaatggcaccttgat | |
| containing an | ctcaccctcatcgaaagtggagttgatgtccttccctggctcgctacagacgcacttccgc[ASO | 1759 |
| ASO sequence | sequence]AATTTTTGGAGCAGGTTTTCTGACTTCGGTCGGAAAACCCCTccca | |
| replacing the | atttcactggtctacaatgaaagcaaaacagttctcttccccgctccccggtgtgtgagaggggctttga | |
| antisense | tccttctctggtttcctaggaaacgcgtatgtgctagagccacgctctgagacttccgcctcgtgcggtc | |
| sequence of U7 | ccgcttcctttctgcctcctctgg | |
| snRNA | | |
| Exemplary full | cccacatcgcctgccactacttaagtccgattcactteggctttagctccaagcctttaatctcgcgaag | 1760 |
| sequence of | ctctttttttttttttaacaacataggagctgtgattggctgttttcagccaatcagcactgactcattt | |
| modified U7 | gcatagcctttacaagcggtcacaaactcaagaaacgagcggttttaatagtcttttagaatattgttta | |
| snRNA | tcgaaccgaataaggaactgtgctttgtgattcacatatcagtggaggggtgtggaaatggcaccttgat | |
| (smOPT) | ctcaccctcatcgaaagtggagttgatgtccttccctggctcgctacagacgcacttccgcAACAAAGGC | |
| containing an | TACAAAAAGTAATTTTTGGAGCAGGTTTTCTGACTTCGGTCGGAAAACCCCTcccaat | |
| ASO sequence | ttcactggtctacaatgaaagcaaaacagttctcttccccgctccccggtgtgtgagaggggctttgatc | |
| replacing the | cttctctggtttcctaggaaacgcgtatgtgctagagccacgctctgagacttccgcctcgtgcggtccc | |
| antisense | gcttcctttctgcctcctctgg | |
| sequence of U7 | | |
| snRNA | | |

TABLE 5D
Exemplary ASO Sequences

| | | | | | SEQ | |
|---|---|---|---|---|---|---|
| chr# | Start | End | Name | Strand | ID NO: | ASO Sequence |
| chr5 | 177238265 | 177238283 | NSD1: NM_001365684: | + | 1356 | GATCGACCTCGGG |
| | | | U1: Ex8: 30 | | | CTAGA |
| chr5 | 177238270 | 177238288 | NSD1: NM_001365684: | + | 1357 | CACTAGATCGACCT |
| | | | U1: Ex8: 35 | | | CGGG |
| chr5 | 177238275 | 177238293 | NSD1: NM_001365684: | + | 1358 | CTGAGCACTAGATC |
| | | | U1: Ex8: 40 | | | GACC |
| chr5 | 177238280 | 177238298 | NSD1: NM_001365684: | + | 1359 | TTGTTCTGAGCACT |
| | | | U1: Ex8: 45 | | | AGAT |
| chr5 | 177238285 | 177238303 | NSD1: NM_001365684: | + | 1360 | CCTGCTTGTTCTGA |
| | | | U1: Ex8: 50 | | | GCAC |
| chr5 | 177238290 | 177238308 | NSD1: NM_001365684: | + | 1361 | GTCCACCTGCTTGT |
| | | | U1: Ex8: 55 | | | TCTG |
| chr5 | 177238295 | 177238313 | NSD1: NM_001365684: | + | 1362 | TTCTCGTCCACCTG |
| | | | U1: Ex8: 60 | | | CTTG |
| chr5 | 177238300 | 177238318 | NSD1: NM_001365684: | + | 1363 | AAGAATTCTCGTCC |
| | | | U1: Ex8: 65 | | | ACCT |
| chr5 | 177238305 | 177238323 | NSD1: NM_001365684: | + | 1364 | AATCAAAGAATTCT |
| | | | U1: Ex8: 70 | | | CGTC |
| chr5 | 177238310 | 177238328 | NSD1: NM_001365684: | + | 1365 | GTTGAAATCAAAG |
| | | | U1: Ex8: 75 | | | AATTC |
| chr5 | 177238315 | 177238333 | NSD1: NM_001365684: | + | 1366 | CTTTGGTTGAAATC |
| | | | U1: Ex8: 80 | | | AAAG |
| chr5 | 177238320 | 177238338 | NSD1: NM_001365684: | + | 1367 | CTCTTCTTTGGTTG |
| | | | U1: Ex8: 85 | | | AAAT |
| chr5 | 177238325 | 177238343 | NSD1: NM_001365684: | + | 1368 | GGAGGCTCTTCTTT |
| | | | U1: Ex8: 90 | | | GGTT |
| chr5 | 177238330 | 177238348 | NSD1: NM_001365684: | + | 1369 | GAACTGGAGGCTC |
| | | | U1: Ex8: 95 | | | TTCTT |
| chr5 | 177238335 | 177238353 | NSD1: NM_001365684: | + | 1370 | TTCAAGAACTGGA |
| | | | U1: Ex8: 100 | | | GGCTC |
| chr5 | 177238340 | 177238358 | NSD1: NM_001365684: | + | 1371 | TCCCTTTCAAGAAC |
| | | | U1: Ex8: 105 | | | TGGA |
| chr5 | 177238345 | 177238363 | NSD1: NM_001365684: | + | 1372 | GAGCCTCCCTTTCA |
| | | | U1: Ex8: 110 | | | AGAA |
| chr5 | 177238350 | 177238368 | NSD1: NM_001365684: | + | 1373 | AAACGGAGCCTCC |
| | | | U1: Ex8: 115 | | | CTTTC |
| chr5 | 177238355 | 177238373 | NSD1: NM_001365684: | + | 1374 | TCCAAAAACGGAG |
| | | | U1: Ex8: 120 | | | CCTCC |
| chr5 | 177238360 | 177238378 | NSD1: NM_001365684: | + | 1375 | GGCCCTCCAAAAA |
| | | | U1: Ex8: 125 | | | CGGAG |
| chr5 | 177238365 | 177238383 | NSD1: NM_001365684: | + | 1376 | CAAGGGGCCCTCC |
| | | | U1: Ex8: 130 | | | AAAAA |
| chr5 | 177238370 | 177238388 | NSD1: NM_001365684: | + | 1377 | TGAGCCAAGGGGC |
| | | | U1: Ex8: 135 | | | CCTCC |
| chr5 | 177238371 | 177238389 | NSD1: NM_001365684: | + | 1378 | CTGAGCCAAGGGG |
| | | | U1: Ex8: −119 | | | CCCTC |
| chr5 | 177238376 | 177238394 | NSD1: NM_001365684: | + | 1379 | TCTGACTGAGCCAA |
| | | | U1: Ex8: −114 | | | GGGG |
| chr5 | 177238381 | 177238399 | NSD1: NM_001365684: | + | 1380 | CAAGTTCTGACTGA |
| | | | U1: Ex8: −109 | | | GCCA |
| chr5 | 177238386 | 177238404 | NSD1: NM_001365684: | + | 1381 | ACCTCCAAGTTCTG |
| | | | U1: Ex8: −104 | | | ACTG |
| chr5 | 177238391 | 177238409 | NSD1: NM_001365684: | + | 1382 | TGTCCACCTCCAAG |
| | | | U1: Ex8: −99 | | | TTCT |
| chr5 | 177238396 | 177238414 | NSD1: NM_001365684: | + | 1383 | CAGCATGTCCACCT |
| | | | U1: Ex8: −94 | | | CCAA |
| chr5 | 177238401 | 177238419 | NSD1: NM_001365684: | + | 1384 | CAACTCAGCATGTC |
| | | | U1: Ex8: −89 | | | CACC |
| chr5 | 177238406 | 177238424 | NSD1: NM_001365684: | + | 1385 | TGCGGCAACTCAG |
| | | | U1: Ex8: −84 | | | CATGT |
| chr5 | 177238411 | 177238429 | NSD1: NM_001365684: | + | 1386 | TCAGCTGCGGCAA |
| | | | U1:Ex8:− 79 | | | CTCAG |
| chr5 | 177238416 | 177238434 | NSD1: NM_001365684: | + | 1387 | CAAGGTCAGCTGC |
| | | | U1: Ex8: −74 | | | GGCAA |
| chr5 | 177238421 | 177238439 | NSD1: NM_001365684: | + | 1388 | ACAGACAAGGTCA |
| | | | U1: Ex8: −69 | | | GCTGC |
| chr5 | 177238426 | 177238444 | NSD1: NM_001365684: | + | 1389 | CAGGCACAGACAA |
| | | | U1: Ex8: −64 | | | GGTCA |
| chr5 | 177238431 | 177238449 | NSD1: NM_001365684: | + | 1390 | AGCCACAGGCACA |
| | | | U1: Ex8: −59 | | | GACAA |
| chr5 | 177238436 | 177238454 | NSD1:NM_001365684: | + | 1391 | TCCGGAGCCACAG |
| | | | U1: Ex8: −54 | | | GCACA |
| chr5 | 177238441 | 177238459 | NSD1:NM_001365684: | + | 1392 | AGACTTCCGGAGC |
| | | | U1: Ex8: −49 | | | CACAG |
| chr5 | 177238446 | 177238464 | NSD1:NM_001365684: | + | 1393 | TGGAGAGACTTCC |
| | | | U1: Ex8: −44 | | | GGAGC |
| chr5 | 177238451 | 177238469 | NSD1:NM_001365684: | + | 1394 | GGCCGTGGAGAGA |
| | | | U1: Ex8: −39 | | | CTTCC |
| chr5 | 177238456 | 177238474 | NSD1:NM_001365684: | + | 1395 | GGGCAGGCCGTGG |
| | | | U1: Ex8: −34 | | | AGAGA |
| chr5 | 177238461 | 177238479 | NSD1: NM_001365684: | + | 1396 | CTCAAGGGCAGGC |
| | | | U1: Ex8:− 29 | | | CGTGG |
| chr5 | 177238466 | 177238484 | NSD1: NM_001365684: | + | 1397 | TCAGACTCAAGGG |
| | | | U1: Ex8:− 24 | | | CAGGC |
| chr5 | 177238471 | 177238489 | NSD1: NM_001365684: | + | 1398 | ATTCCTCAGACTCA |
| | | | U1: Ex8:− 19 | | | AGGG |
| chr5 | 177238476 | 177238494 | NSD1: NM_001365684: | + | 1399 | TAGCAATTCCTCAG |
| | | | U1: Ex8:− 14 | | | ACTC |
| chr5 | 177238481 | 177238499 | NSD1: NM_001365684: | + | 1400 | TTAACTAGCAATTC |
| | | | U1: Ex8:− 9 | | | CTCA |
| chr5 | 177238486 | 177238504 | NSD1: NM_001365684: | + | 1401 | GCGTTTTAACTAGC |
| | | | U1: Ex8:− 4 | | | AATT |
| chr5 | 177238504 | 177238522 | NSD1: NM_001365684: | + | 1402 | CCAACCCCACCTTA |
| | | | U1: Ex8−IVS8: − 3 | | | CCTG |
| chr5 | 177238509 | 177238527 | NSD1: NM_001365684: | + | 1403 | AGACCCCAACCCC |
| | | | U1: IVS8: 3 | | | ACCTT |
| chr5 | 177238514 | 177238532 | NSD1: NM_001365684: | + | 1404 | TACTGAGACCCCA |
| | | | U1: IVS8: 8 | | | ACCCC |
| chr5 | 177238519 | 177238537 | NSD1: NM_001365684: | + | 1405 | TCAAATACTGAGA |
| | | | U1: IVS8: 13 | | | CCCCA |
| chr5 | 177238524 | 177238542 | NSD1: NM_001365684: | + | 1406 | TCTGCTCAAATACT |
| | | | U1: IVS8: 18 | | | GAGA |
| chr5 | 177238529 | 177238547 | NSD1: NM_001365684: | + | 1407 | TCATATCTGCTCAA |
| | | | U1: IVS8: 23 | | | ATAC |
| chr5 | 177238534 | 177238552 | NSD1: NM_001365684: | + | 1408 | TCTAATCATATCTG |
| | | | U1: IVS8: 28 | | | CTCA |
| chr5 | 177238539 | 177238557 | NSD1:NM_001365684: | + | 1409 | CTTCCTCTAATCAT |
| | | | U1: IVS8: 33 | | | ATCT |
| chr5 | 177238544 | 177238562 | NSD1:NM_001365684: | + | 1410 | TCCTGCTTCCTCTA |
| | | | U1: IVS8: 38 | | | ATCA |
| chr5 | 177238549 | 177238567 | NSD1:NM_001365684: | + | 1411 | AAATCTCCTGCTTC |
| | | | U1: IVS8: 43 | | | CTCT |
| chr5 | 177238554 | 177238572 | NSD1:NM_001365684: | + | 1412 | TACTAAAATCTCCT |
| | | | U1: IVS8: 48 | | | GCTT |
| chr5 | 177238559 | 177238577 | NSD1:NM_001365684: | + | 1413 | AAACATACTAAAA |
| | | | U1: IVS8: 53 | | | TCTCC |
| chr5 | 177238564 | 177238582 | NSD1:NM_001365684: | + | 1414 | CATCAAAACATACT |
| | | | U1: IVS8: 58 | | | AAAA |
| chr5 | 177238569 | 177238587 | NSD1:NM_001365684: | + | 1415 | CTTTACATCAAAAC |
| | | | U1: IVS8: 63 | | | ATAC |
| chr5 | 177238574 | 177238592 | NSD1:NM_001365684: | + | 1416 | GTTGGCTTTACATC |
| | | | U1: IVS8: 68 | | | AAAA |
| chr5 | 177238579 | 177238597 | NSD1:NM_001365684: | + | 1417 | ACAATGTTGGCTTT |
| | | | U1: IVS8: 73 | | | ACAT |
| chr5 | 177238584 | 177238602 | NSD1:NM_001365684: | + | 1418 | TAGATACAATGTTG |
| | | | U1: IVS8: 78 | | | GCTT |
| chr5 | 177238589 | 177238607 | NSD1: NM_001365684: | + | 1419 | GTATATAGATACA |
| | | | U1: IVS8: 83 | | | ATGTT |
| chr5 | 177238594 | 177238612 | NSD1: NM_001365684: | + | 1420 | TTATTGTATATAGA |
| | | | U1: IVS8: 88 | | | TACA |
| chr5 | 177238599 | 177238617 | NSD1: NM_001365684: | + | 1421 | GTAGTTTATTGTAT |
| | | | U1: IVS8: 93 | | | ATAG |
| chr5 | 177238604 | 177238622 | NSD1: NM_001365684: | + | 1422 | AGGGGGTAGTTTAT |
| | | | U1: IVS8: 98 | | | TGTA |
| chr5 | 177238606 | 177238624 | NSD1: NM_001365684: | + | 1423 | AAAGGGGGTAGTT |
| | | | U1: IVS8: 100 | | | TATTG |

TABLE 5E
Exemplary ASO Sequences

| | | | | | SEQ | |
|---|---|---|---|---|---|---|
| | | | | | ID | |
| chr | Start | End | Name | Strand | NO: | ASO sequence |
| chr5 | 177238265 | 177238283 | NSD1:NM_001365684: | + | 1424 | GATCGACCTCGGGC |
| | | | U1:Ex8:30 | | | TAGA |
| chr5 | 17723 | 1772382 | NSD1:NM_001365684: | + | 1425 | AGATCGACCTCGGG |
| | 8266 | 84 | U1:Ex8:31 | | | CTAG |
| chr5 | 17723 | 1772382 | NSD1:NM_001365684: | + | 1426 | TAGATCGACCTCGG |
| | 8267 | 85 | U1:Ex8:32 | | | GCTA |
| chr5 | 17723 | 1772382 | NSD1:NM_001365684: | + | 1427 | CTAGATCGACCTCG |
| | 8268 | 86 | U1:Ex8:33 | | | GGCT |
| chr5 | 17723 | 1772382 | NSD1:NM_001365684: | + | 1428 | ACTAGATCGACCTC |
| | 8269 | 87 | U1:Ex8:34 | | | GGGC |
| chr5 | 17723 | 1772382 | NSD1:NM_001365684: | + | 1429 | CACTAGATCGACCTC |
| | 8270 | 88 | U1:Ex8:35 | | | GGG |
| chr5 | 17723 | 1772382 | NSD1:NM_001365684: | + | 1430 | GCACTAGATCGACC |
| | 8271 | 89 | U1:Ex8:36 | | | TCGG |
| chr5 | 17723 | 1772382 | NSD1:NM_001365684: | + | 1431 | AGCACTAGATCGAC |
| | 8272 | 90 | U1:Ex8:37 | | | CTCG |
| chr5 | 17723 | 1772382 | NSD1:NM_001365684: | + | 1432 | GAGCACTAGATCGA |
| | 8273 | 91 | U1:Ex8:38 | | | CCTC |
| chr5 | 17723 | 1772382 | NSD1:NM_001365684: | + | 1433 | TGAGCACTAGATCG |
| | 8274 | 92 | U1:Ex8:39 | | | ACCT |
| chr5 | 17723 | 1772382 | NSD1:NM_001365684: | + | 1434 | CTGAGCACTAGATC |
| | 8275 | 93 | U1:Ex8:40 | | | GACC |
| chr5 | 17723 | 1772382 | NSD1:NM_001365684: | + | 1435 | TCTGAGCACTAGATC |
| | 8276 | 94 | U1:Ex8:41 | | | GAC |
| chr5 | 17723 | 1772382 | NSD1:NM_001365684: | + | 1436 | TTCTGAGCACTAGAT |
| | 8277 | 95 | U1:Ex8:42 | | | CGA |
| chr5 | 17723 | 1772382 | NSD1:NM_001365684: | + | 1437 | GTTCTGAGCACTAG |
| | 8278 | 96 | U1:Ex8:43 | | | ATCG |
| chr5 | 17723 | 1772382 | NSD1:NM_001365684: | + | 1438 | TGTTCTGAGCACTAG |
| | 8279 | 97 | U1:Ex8:44 | | | ATC |
| chr5 | 17723 | 1772382 | NSD1:NM_001365684: | + | 1439 | TTGTTCTGAGCACTA |
| | 8280 | 98 | U1:Ex8:45 | | | GAT |
| chr5 | 17723 | 1772382 | NSD1:NM_001365684: | + | 1440 | CTTGTTCTGAGCACT |
| | 8281 | 99 | U1:Ex8:46 | | | AGA |
| chr5 | 17723 | 1772383 | NSD1:NM_001365684: | + | 1441 | GCTTGTTCTGAGCAC |
| | 8282 | 00 | U1:Ex8:47 | | | TAG |
| chr5 | 17723 | 1772383 | NSD1:NM_001365684: | + | 1442 | TGCTTGTTCTGAGCA |
| | 8283 | 01 | U1:Ex8:48 | | | CTA |
| chr5 | 17723 | 1772383 | NSD1:NM_001365684: | + | 1443 | CTGCTTGTTCTGAGC |
| | 8284 | 02 | U1:Ex8:49 | | | ACT |
| chr5 | 17723 | 1772383 | NSD1:NM_001365684: | + | 1444 | CCTGCTTGTTCTGAG |
| | 8285 | 03 | U1:Ex8:50 | | | CAC |
| chr5 | 17723 | 1772383 | NSD1:NM_001365684: | + | 1445 | ACCTGCTTGTTCTGA |
| | 8286 | 04 | U1:Ex8:51 | | | GCA |
| chr5 | 17723 | 1772383 | NSD1:NM_001365684: | + | 1446 | CACCTGCTTGTTCTG |
| | 8287 | 05 | U1:Ex8:52 | | | AGC |
| chr5 | 17723 | 1772383 | NSD1:NM_001365684: | + | 1447 | CCACCTGCTTGTTCT |
| | 8288 | 06 | U1:Ex8:53 | | | GAG |
| chr5 | 17723 | 1772383 | NSD1:NM_001365684: | + | 1448 | TCCACCTGCTTGTTC |
| | 8289 | 07 | U1:Ex8:54 | | | TGA |
| chr5 | 17723 | 1772383 | NSD1:NM_001365684: | + | 1449 | GTCCACCTGCTTGTT |
| | 8290 | 08 | U1:Ex8:55 | | | CTG |
| chr5 | 17723 | 1772383 | NSD1:NM_001365684: | + | 1450 | CGTCCACCTGCTTGT |
| | 8291 | 09 | U1:Ex8:56 | | | TCT |
| chr5 | 17723 | 1772383 | NSD1:NM_001365684: | + | 1451 | TCGTCCACCTGCTTG |
| | 8292 | 10 | U1:Ex8:57 | | | TTC |
| chr5 | 17723 | 1772383 | NSD1:NM_001365684: | + | 1452 | CTCGTCCACCTGCTT |
| | 8293 | 11 | U1:Ex8:58 | | | GTT |
| chr5 | 17723 | 1772383 | NSD1:NM_001365684: | 1 | 1453 | TCTCGTCCACCTGCT |
| | 8294 | 12 | U1:Ex8:59 | | | TGT |
| chr5 | 17723 | 1772383 | NSD1:NM_001365684: | + | 1454 | TTCTCGTCCACCTGC |
| | 8295 | 13 | U1:Ex8:60 | | | TTG |
| chr5 | 17723 | 1772383 | NSD1:NM_001365684: | + | 1455 | ATTCTCGTCCACCTG |
| | 8296 | 14 | U1:Ex8:61 | | | CTT |
| chr5 | 17723 | 1772383 | NSD1:NM_001365684: | + | 1456 | AATTCTCGTCCACCT |
| | 8297 | 15 | U1:Ex8:62 | | | GCT |
| chr5 | 17723 | 1772383 | NSD1:NM_001365684: | + | 1457 | GAATTCTCGTCCACC |
| | 8298 | 16 | U1:Ex8:63 | | | TGC |
| chr5 | 17723 | 1772383 | NSD1:NM_001365684: | + | 1458 | AGAATTCTCGTCCAC |
| | 8299 | 17 | U1:Ex8:64 | | | CTG |
| chr5 | 17723 | 1772383 | NSD1:NM_001365684: | + | 1459 | AAGAATTCTCGTCCA |
| | 8300 | 18 | U1:Ex8:65 | | | CCT |
| chr5 | 17723 | 1772383 | NSD1:NM_001365684: | + | 1460 | AAAGAATTCTCGTCC |
| | 8301 | 19 | U1:Ex8:66 | | | ACC |
| chr5 | 17723 | 1772383 | NSD1:NM_001365684: | + | 1461 | CAAAGAATTCTCGTC |
| | 8302 | 20 | U1:Ex8:67 | | | CAC |
| chr5 | 17723 | 1772383 | NSD1:NM_001365684: | + | 1462 | TCAAAGAATTCTCGT |
| | 8303 | 21 | U1:Ex8:68 | | | CCA |
| chr5 | 17723 | 1772383 | NSD1:NM_001365684: | + | 1463 | ATCAAAGAATTCTC |
| | 8304 | 22 | U1:Ex8:69 | | | GTCC |
| chr5 | 17723 | 1772383 | NSD1:NM_001365684: | + | 1464 | AATCAAAGAATTCT |
| | 8305 | 23 | U1:Ex8:70 | | | CGTC |
| chr5 | 17723 | 1772383 | NSD1:NM_001365684: | + | 1465 | AAATCAAAGAATTC |
| | 8306 | 24 | U1:Ex8:71 | | | TCGT |
| chr5 | 17723 | 1772383 | NSD1:NM_001365684: | + | 1466 | GAAATCAAAGAATT |
| | 8307 | 25 | U1:Ex8:72 | | | CTCG |
| chr5 | 17723 | 1772383 | NSD1:NM_001365684: | + | 1467 | TGAAATCAAAGAAT |
| | 8308 | 26 | U1:Ex8:73 | | | TCTC |
| chr5 | 17723 | 1772383 | NSD1:NM_001365684: | + | 1468 | TTGAAATCAAAGAA |
| | 8309 | 27 | U1:Ex8:74 | | | TTCT |
| chr5 | 17723 | 1772383 | NSD1:NM_001365684: | + | 1469 | GTTGAAATCAAAGA |
| | 8310 | 28 | U1:Ex8:75 | | | ATTC |
| chr5 | 17723 | 1772383 | NSD1:NM_001365684: | + | 1470 | GGTTGAAATCAAAG |
| | 8311 | 29 | U1:Ex8:76 | | | AATT |
| chr5 | 17723 | 1772383 | NSD1:NM_001365684: | + | 1471 | TGGTTGAAATCAAA |
| | 8312 | 30 | U1:Ex8:77 | | | GAAT |
| chr5 | 17723 | 1772383 | NSD1:NM_001365684: | + | 1472 | TTGGTTGAAATCAA |
| | 8313 | 31 | U1:Ex8:78 | | | AGAA |
| chr5 | 17723 | 1772383 | NSD1:NM_001365684: | + | 1473 | TTTGGTTGAAATCAA |
| | 8314 | 32 | U1:Ex8:79 | | | AGA |
| chr5 | 17723 | 1772383 | NSD1:NM_001365684: | + | 1474 | CTTTGGTTGAAATCA |
| | 8315 | 33 | U1:Ex8:80 | | | AAG |
| chr5 | 17723 | 1772383 | NSD1:NM_001365684: | + | 1475 | TCTTTGGTTGAAATC |
| | 8316 | 34 | U1:Ex8:81 | | | AAA |
| chr5 | 17723 | 1772383 | NSD1:NM_001365684: | + | 1476 | TTCTTTGGTTGAAAT |
| | 8317 | 35 | U1:Ex8:82 | | | CAA |
| chr5 | 17723 | 1772383 | NSD1:NM_001365684: | + | 1477 | CTTCTTTGGTTGAAA |
| | 8318 | 36 | U1:Ex8:83 | | | TCA |
| chr5 | 17723 | 1772383 | NSD1:NM_001365684: | + | 1478 | TCTTCTTTGGTTGAA |
| | 8319 | 37 | U1:Ex8:84 | | | ATC |
| chr5 | 17723 | 1772383 | NSD1:NM_001365684: | + | 1479 | CTCTTCTTTGGTTGA |
| | 8320 | 38 | U1:Ex8:85 | | | AAT |
| chr5 | 17723 | 1772383 | NSD1:NM_001365684: | + | 1480 | GCTCTTCTTTGGTTG |
| | 8321 | 39 | U1:Ex8:86 | | | AAA |
| chr5 | 17723 | 1772383 | NSD1:NM_001365684: | + | 1481 | GGCTCTTCTTTGGTT |
| | 8322 | 40 | U1:Ex8:87 | | | GAA |
| chr5 | 17723 | 1772383 | NSD1:NM_001365684: | + | 1482 | AGGCTCTTCTTTGGT |
| | 8323 | 41 | U1:Ex8:88 | | | TGA |
| chr5 | 17723 | 1772383 | NSD1:NM_001365684: | + | 1483 | GAGGCTCTTCTTTGG |
| | 8324 | 42 | U1:Ex8:89 | | | TTG |
| chr5 | 17723 | 1772383 | NSD1:NM_001365684: | + | 1484 | GGAGGCTCTTCTTTG |
| | 8325 | 43 | U1:Ex8:90 | | | GTT |
| chr5 | 17723 | 1772383 | NSD1:NM_001365684: | + | 1485 | TGGAGGCTCTTCTTT |
| | 8326 | 44 | U1:Ex8:91 | | | GGT |
| chr5 | 17723 | 1772383 | NSD1:NM_001365684: | + | 1486 | CTGGAGGCTCTTCTT |
| | 8327 | 45 | U1:Ex8:92 | | | TGG |
| chr5 | 17723 | 1772383 | NSD1:NM_001365684: | + | 1487 | ACTGGAGGCTCTTCT |
| | 8328 | 46 | U1:Ex8:93 | | | TTG |
| chr5 | 17723 | 1772383 | NSD1:NM_001365684: | + | 1488 | AACTGGAGGCTCTTC |
| | 8329 | 47 | U1:Ex8:94 | | | TTT |
| chr5 | 17723 | 1772383 | NSD1:NM_001365684: | 1 | 1489 | GAACTGGAGGCTCT |
| | 8330 | 48 | U1:Ex8:95 | | | TCTT |
| chr5 | 17723 | 1772383 | NSD1:NM_001365684: | + | 1490 | AGAACTGGAGGCTC |
| | 8331 | 49 | U1:Ex8:96 | | | TTCT |
| chr5 | 17723 | 1772383 | NSD1:NM_001365684: | + | 1491 | AAGAACTGGAGGCT |
| | 8332 | 50 | U1:Ex8:97 | | | CTTC |
| chr5 | 17723 | 1772383 | NSD1:NM_001365684: | + | 1492 | CAAGAACTGGAGGC |
| | 8333 | 51 | U1:Ex8:98 | | | TCTT |
| chr5 | 17723 | 1772383 | NSD1:NM_001365684: | + | 1493 | TCAAGAACTGGAGG |
| | 8334 | 52 | U1:Ex8:99 | | | CTCT |
| chr5 | 17723 | 1772383 | NSD1:NM_001365684: | + | 1494 | TTCAAGAACTGGAG |
| | 8335 | 53 | U1:Ex8:100 | | | GCTC |
| chr5 | 17723 | 1772383 | NSD1:NM_001365684: | + | 1495 | TTTCAAGAACTGGA |
| | 8336 | 54 | U1:Ex8:101 | | | GGCT |
| chr5 | 17723 | 1772383 | NSD1:NM_001365684: | + | 1496 | CTTTCAAGAACTGG |
| | 8337 | 55 | U1:Ex8:102 | | | AGGC |
| chr5 | 17723 | 1772383 | NSD1:NM_001365684: | + | 1497 | CCTTTCAAGAACTGG |
| | 8338 | 56 | U1:Ex8:103 | | | AGG |
| chr5 | 17723 | 1772383 | NSD1:NM_001365684: | + | 1498 | CCCTTTCAAGAACTG |
| | 8339 | 57 | U1:Ex8:104 | | | GAG |
| chr5 | 17723 | 1772383 | NSD1:NM_001365684: | + | 1499 | TCCCTTTCAAGAACT |
| | 8340 | 58 | U1:Ex8:105 | | | GGA |
| chr5 | 17723 | 1772383 | NSD1:NM_001365684: | + | 1500 | CTCCCTTTCAAGAAC |
| | 8341 | 59 | U1:Ex8:106 | | | TGG |
| chr5 | 17723 | 1772383 | NSD1:NM_001365684: | + | 1501 | CCTCCCTTTCAAGAA |
| | 8342 | 60 | U1:Ex8:107 | | | CTG |
| chr5 | 17723 | 1772383 | NSD1:NM_001365684: | + | 1502 | GCCTCCCTTTCAAGA |
| | 8343 | 61 | U1:Ex8:108 | | | ACT |
| chr5 | 17723 | 1772383 | NSD1:NM_001365684: | + | 1503 | AGCCTCCCTTTCAAG |
| | 8344 | 62 | U1:Ex8:109 | | | AAC |
| chr5 | 17723 | 1772383 | NSD1:NM_001365684: | + | 1504 | GAGCCTCCCTTTCAA |
| | 8345 | 63 | U1:Ex8:110 | | | GAA |
| chr5 | 17723 | 1772383 | NSD1:NM_001365684: | + | 1505 | GGAGCCTCCCTTTCA |
| | 8346 | 64 | U1:Ex8:111 | | | AGA |
| chr5 | 17723 | 1772383 | NSD1:NM_001365684: | + | 1506 | CGGAGCCTCCCTTTC |
| | 8347 | 65 | U1:Ex8:112 | | | AAG |
| chr5 | 17723 | 1772383 | NSD1:NM_001365684: | + | 1507 | ACGGAGCCTCCCTTT |
| | 8348 | 66 | U1:Ex8:113 | | | CAA |
| chr5 | 17723 | 1772383 | NSD1:NM_001365684: | + | 1508 | AACGGAGCCTCCCTT |
| | 8349 | 67 | U1:Ex8:114 | | | TCA |
| chr5 | 17723 | 1772383 | NSD1:NM_001365684: | + | 1509 | AAACGGAGCCTCCC |
| | 8350 | 68 | U1:Ex8:115 | | | TTTC |
| chr5 | 17723 | 1772383 | NSD1:NM_001365684: | + | 1510 | AAAACGGAGCCTCC |
| | 8351 | 69 | U1:Ex8:116 | | | CTTT |
| chr5 | 17723 | 1772383 | NSD1:NM_001365684: | + | 1511 | AAAAACGGAGCCTC |
| | 8352 | 70 | U1:Ex8:117 | | | CCTT |
| chr5 | 17723 | 1772383 | NSD1:NM_001365684: | + | 1512 | CAAAAACGGAGCCT |
| | 8353 | 71 | U1:Ex8:118 | | | CCCT |
| chr5 | 17723 | 1772383 | NSD1:NM_001365684: | + | 1513 | CCAAAAACGGAGCC |
| | 8354 | 72 | U1:Ex8:119 | | | TCCC |
| chr5 | 17723 | 1772383 | NSD1:NM_001365684: | + | 1514 | TCCAAAAACGGAGC |
| | 8355 | 73 | U1:Ex8:120 | | | CTCC |
| chr5 | 17723 | 1772383 | NSD1:NM_001365684: | + | 1515 | CTCCAAAAACGGAG |
| | 8356 | 74 | U1:Ex8:121 | | | CCTC |
| chr5 | 17723 | 1772383 | NSD1:NM_001365684:U | + | 1516 | CCTCCAAAAACGGA |
| | 8357 | 75 | U1:Ex8:122 | | | GCCT |
| chr5 | 17723 | 1772383 | NSD1:NM_001365684: | + | 1517 | CCCTCCAAAAACGG |
| | 8358 | 76 | U1:Ex8:123 | | | AGCC |
| chr5 | 17723 | 1772383 | NSD1:NM_001365684: | + | 1518 | GCCCTCCAAAAACG |
| | 8359 | 77 | U1:Ex8:124 | | | GAGC |
| chr5 | 17723 | 1772383 | NSD1:NM_001365684: | + | 1519 | GGCCCTCCAAAAAC |
| | 8360 | 78 | U1:Ex8:125 | | | GGAG |
| chr5 | 17723 | 1772383 | NSD1:NM_001365684: | + | 1520 | GGGCCCTCCAAAAA |
| | 8361 | 79 | U1:Ex8:126 | | | CGGA |
| chr5 | 17723 | 1772383 | NSD1:NM_001365684: | + | 1521 | GGGGCCCTCCAAAA |
| | 8362 | 80 | U1:Ex8:127 | | | ACGG |
| chr5 | 17723 | 1772383 | NSD1:NM_001365684: | + | 1522 | AGGGGCCCTCCAAA |
| | 8363 | 81 | U1:Ex8:128 | | | AACG |
| chr5 | 17723 | 1772383 | NSD1:NM_001365684: | + | 1523 | AAGGGGCCCTCCAA |
| | 8364 | 82 | U1:Ex8:129 | | | AAAC |
| chr5 | 17723 | 1772383 | NSD1:NM_001365684: | + | 1524 | CAAGGGGCCCTCCA |
| | 8365 | 83 | U1:Ex8:130 | | | AAAA |
| chr5 | 17723 | 1772383 | NSD1:NM_001365684: | + | 1525 | CCAAGGGGCCCTCC |
| | 8366 | 84 | U1:Ex8:131 | | | AAAA |
| chr5 | 17723 | 1772383 | NSD1:NM_001365684: | + | 1526 | GCCAAGGGGCCCTC |
| | 8367 | 85 | U1:Ex8:132 | | | CAAA |
| chr5 | 17723 | 1772383 | NSD1:NM_001365684: | + | 1527 | AGCCAAGGGGCCCT |
| | 8368 | 86 | U1:Ex8:133 | | | CCAA |
| chr5 | 17723 | 1772383 | NSD1:NM_001365684: | + | 1528 | GAGCCAAGGGGCCC |
| | 8369 | 87 | U1:Ex8:134 | | | TCCA |
| chr5 | 17723 | 1772383 | NSD1:NM_001365684: | + | 1529 | TGAGCCAAGGGGCC |
| | 8370 | 88 | U1:Ex8:135 | | | CTCC |
| chr5 | 17723 | 1772383 | NSD1:NM_001365684: | + | 1530 | CTGAGCCAAGGGGC |
| | 8371 | 89 | U1:Ex8:−119 | | | CCTC |
| chr5 | 17723 | 1772383 | NSD1:NM_001365684: | + | 1531 | ACTGAGCCAAGGGG |
| | 8372 | 90 | U1:Ex8:−118 | | | CCCT |
| chr5 | 17723 | 1772383 | NSD1:NM_001365684: | + | 1532 | GACTGAGCCAAGGG |
| | 8373 | 91 | U1:Ex8:−117 | | | GCCC |
| chr5 | 17723 | 1772383 | NSD1:NM_001365684: | + | 1533 | TGACTGAGCCAAGG |
| | 8374 | 92 | U1:Ex8:−116 | | | GGCC |
| chr5 | 17723 | 1772383 | NSD1:NM_001365684: | + | 1534 | CTGACTGAGCCAAG |
| | 8375 | 93 | U1:Ex8:−115 | | | GGGC |
| chr5 | 17723 | 1772383 | NSD1:NM_001365684: | + | 1535 | TCTGACTGAGCCAA |
| | 8376 | 94 | U1:Ex8:−114 | | | GGGG |
| chr5 | 17723 | 1772383 | NSD1:NM_001365684: | + | 1536 | TTCTGACTGAGCCAA |
| | 8377 | 95 | U1:Ex8:−113 | | | GGG |
| chr5 | 17723 | 1772383 | NSD1:NM_001365684: | + | 1537 | GTTCTGACTGAGCCA |
| | 8378 | 96 | U1:Ex8:−112 | | | AGG |
| chr5 | 17723 | 1772383 | NSD1:NM_001365684: | + | 1538 | AGTTCTGACTGAGCC |
| | 8379 | 97 | U1:Ex8:−111 | | | AAG |
| chr5 | 17723 | 1772383 | NSD1:NM_001365684: | + | 1539 | AAGTTCTGACTGAG |
| | 8380 | 98 | U1:Ex8:−110 | | | CCAA |
| chr5 | 17723 | 1772383 | NSD1:NM_001365684: | + | 1540 | CAAGTTCTGACTGA |
| | 8381 | 99 | U1:Ex8:−109 | | | GCCA |
| chr5 | 17723 | 1772384 | NSD1:NM_001365684: | + | 1541 | CCAAGTTCTGACTGA |
| | 8382 | 00 | U1:Ex8:−108 | | | GCC |
| chr5 | 17723 | 1772384 | NSD1:NM_001365684: | + | 1542 | TCCAAGTTCTGACTG |
| | 8383 | 01 | U1:Ex8:−107 | | | AGC |
| chr5 | 17723 | 1772384 | NSD1:NM_001365684: | + | 1543 | CTCCAAGTTCTGACT |
| | 8384 | 02 | U1:Ex8:−106 | | | GAG |
| chr5 | 17723 | 1772384 | NSD1:NM_001365684: | + | 1544 | CCTCCAAGTTCTGAC |
| | 8385 | 03 | U1:Ex8:−105 | | | TGA |
| chr5 | 17723 | 1772384 | NSD1:NM_001365684: | + | 1545 | ACCTCCAAGTTCTGA |
| | 8386 | 04 | U1:Ex8:−104 | | | CTG |
| chr5 | 17723 | 1772384 | NSD1:NM_001365684: | + | 1546 | CACCTCCAAGTTCTG |
| | 8387 | 05 | U1:Ex8:−103 | | | ACT |
| chr5 | 17723 | 1772384 | NSD1:NM_001365684: | + | 1547 | CCACCTCCAAGTTCT |
| | 8388 | 06 | U1:Ex8:−102 | | | GAC |
| chr5 | 17723 | 1772384 | NSD1:NM_001365684: | + | 1548 | TCCACCTCCAAGTTC |
| | 8389 | 07 | U1:Ex8:−101 | | | TGA |
| chr5 | 17723 | 1772384 | NSD1:NM_001365684: | + | 1549 | GTCCACCTCCAAGTT |
| | 8390 | 08 | U1:Ex8:−100 | | | CTG |
| chr5 | 17723 | 1772384 | NSD1:NM_001365684: | + | 1550 | TGTCCACCTCCAAGT |
| | 8391 | 09 | U1:Ex8:−99 | | | TCT |
| chr5 | 17723 | 1772384 | NSD1:NM_001365684: | + | 1551 | ATGTCCACCTCCAAG |
| | 8392 | 10 | U1:Ex8:−98 | | | TTC |
| chr5 | 17723 | 1772384 | NSD1:NM_001365684: | + | 1552 | CATGTCCACCTCCAA |
| | 8393 | 11 | U1:Ex8:−97 | | | GTT |
| chr5 | 17723 | 1772384 | NSD1:NM_001365684: | + | 1553 | GCATGTCCACCTCCA |
| | 8394 | 12 | U1:Ex8:−96 | | | AGT |
| chr5 | 17723 | 1772384 | NSD1:NM_001365684: | + | 1554 | AGCATGTCCACCTCC |
| | 8395 | 13 | U1:Ex8:−95 | | | AAG |
| chr5 | 17723 | 1772384 | NSD1:NM_001365684: | + | 1555 | CAGCATGTCCACCTC |
| | 8396 | 14 | U1:Ex8:−94 | | | CAA |
| chr5 | 17723 | 1772384 | NSD1:NM_001365684: | + | 1556 | TCAGCATGTCCACCT |
| | 8397 | 15 | U1:Ex8:−93 | | | CCA |
| chr5 | 17723 | 1772384 | NSD1:NM_001365684: | + | 1557 | CTCAGCATGTCCACC |
| | 8398 | 16 | U1:Ex8:−92 | | | TCC |
| chr5 | 17723 | 1772384 | NSD1:NM_001365684: | + | 1558 | ACTCAGCATGTCCAC |
| | 8399 | 17 | U1:Ex8:−91 | | | CTC |
| chr5 | 17723 | 1772384 | NSD1:NM_001365684: | + | 1559 | AACTCAGCATGTCC |
| | 8400 | 18 | U1:Ex8:−90 | | | ACCT |
| chr5 | 17723 | 1772384 | NSD1:NM_001365684: | + | 1560 | CAACTCAGCATGTCC |
| | 8401 | 19 | U1:Ex8:−89 | | | ACC |
| chr5 | 17723 | 1772384 | NSD1:NM_001365684: | + | 1561 | GCAACTCAGCATGT |
| | 8402 | 20 | U1:Ex8:−88 | | | CCAC |
| chr5 | 17723 | 1772384 | NSD1:NM_001365684: | + | 1562 | GGCAACTCAGCATG |
| | 8403 | 21 | U1:Ex8:−87 | | | TCCA |
| chr5 | 17723 | 1772384 | NSD1:NM_001365684: | + | 1563 | CGGCAACTCAGCAT |
| | 8404 | 22 | U1:Ex8:−86 | | | GTCC |
| chr5 | 17723 | 1772384 | NSD1:NM_001365684: | + | 1564 | GCGGCAACTCAGCA |
| | 8405 | 23 | U1:Ex8:−85 | | | TGTC |
| chr5 | 17723 | 1772384 | NSD1:NM_001365684: | + | 1565 | TGCGGCAACTCAGC |
| | 8406 | 24 | U1:Ex8:−84 | | | ATGT |
| chr5 | 17723 | 1772384 | NSD1:NM_001365684: | + | 1566 | CTGCGGCAACTCAG |
| | 8407 | 25 | U1:Ex8:−83 | | | CATG |
| chr5 | 17723 | 1772384 | NSD1:NM_001365684: | + | 1567 | GCTGCGGCAACTCA |
| | 8408 | 26 | U1:Ex8:−82 | | | GCAT |
| chr5 | 17723 | 1772384 | NSD1:NM_001365684: | + | 1568 | AGCTGCGGCAACTC |
| | 8409 | 27 | U1:Ex8:−81 | | | AGCA |
| chr5 | 17723 | 1772384 | NSD1:NM_001365684: | + | 1569 | CAGCTGCGGCAACT |
| | 8410 | 28 | U1:Ex8:−80 | | | CAGC |
| chr5 | 17723 | 1772384 | NSD1:NM_001365684: | + | 1570 | TCAGCTGCGGCAAC |
| | 8411 | 29 | U1:Ex8:−79 | | | TCAG |
| chr5 | 17723 | 1772384 | NSD1:NM_001365684: | + | 1571 | GTCAGCTGCGGCAA |
| | 8412 | 30 | U1:Ex8:−78 | | | CTCA |
| chr5 | 17723 | 1772384 | NSD1:NM_001365684: | + | 1572 | GGTCAGCTGCGGCA |
| | 8413 | 31 | U1:Ex8:−77 | | | ACTC |
| chr5 | 17723 | 1772384 | NSD1:NM_001365684: | + | 1573 | AGGTCAGCTGCGGC |
| | 8414 | 32 | U1:Ex8:−76 | | | AACT |
| chr5 | 17723 | 1772384 | NSD1:NM_001365684: | + | 1574 | AAGGTCAGCTGCGG |
| | 8415 | 33 | U1:Ex8:−75 | | | CAAC |
| chr5 | 17723 | 1772384 | NSD1:NM_001365684: | + | 1575 | CAAGGTCAGCTGCG |
| | 8416 | 34 | U1:Ex8:−74 | | | GCAA |
| chr5 | 17723 | 1772384 | NSD1:NM_001365684: | + | 1576 | ACAAGGTCAGCTGC |
| | 8417 | 35 | Ex8:−73 | | | GGCA |
| chr5 | 17723 | 1772384 | NSD1:NM_001365684: | + | 1577 | GACAAGGTCAGCTG |
| | 8418 | 36 | U1:Ex8:−72 | | | CGGC |
| chr5 | 17723 | 1772384 | NSD1:NM_001365684: | + | 1578 | AGACAAGGTCAGCT |
| | 8419 | 37 | U1:Ex8:−71 | | | GCGG |
| chr5 | 17723 | 1772384 | NSD1:NM_001365684: | + | 1579 | CAGACAAGGTCAGC |
| | 8420 | 38 | U1:Ex8:−70 | | | TGCG |
| chr5 | 17723 | 1772384 | NSD1:NM_001365684: | + | 1580 | ACAGACAAGGTCAG |
| | 8421 | 39 | U1:Ex8:−69 | | | CTGC |
| chr5 | 17723 | 1772384 | NSD1:NM_001365684: | + | 1581 | CACAGACAAGGTCA |
| | 8422 | 40 | U1:Ex8:−68 | | | GCTG |
| chr5 | 17723 | 1772384 | NSD1:NM_001365684: | + | 1582 | GCACAGACAAGGTC |
| | 8423 | 41 | U1:Ex8:−67 | | | AGCT |
| chr5 | 17723 | 1772384 | NSD1:NM_001365684: | + | 1583 | GGCACAGACAAGGT |
| | 8424 | 42 | U1:Ex8:−66 | | | CAGC |
| chr5 | 17723 | 1772384 | NSD1:NM_001365684: | + | 1584 | AGGCACAGACAAGG |
| | 8425 | 43 | U1:Ex8:−65 | | | TCAG |
| chr5 | 17723 | 1772384 | NSD1:NM_001365684: | 1 | 1585 | CAGGCACAGACAAG |
| | 8426 | 44 | U1:Ex8:−64 | | | GTCA |
| chr5 | 17723 | 1772384 | NSD1:NM_001365684: | + | 1586 | ACAGGCACAGACAA |
| | 8427 | 45 | U1:Ex8:−63 | | | GGTC |
| chr5 | 17723 | 1772384 | NSD1:NM_001365684: | + | 1587 | CACAGGCACAGACA |
| | 8428 | 46 | U1:Ex8:−62 | | | AGGT |
| chr5 | 17723 | 1772384 | NSD1:NM_001365684: | + | 1588 | CCACAGGCACAGAC |
| | 8429 | 47 | U1:Ex8:−61 | | | AAGG |
| chr5 | 17723 | 1772384 | NSD1:NM_001365684: | + | 1589 | GCCACAGGCACAGA |
| | 8430 | 48 | U1:Ex8:−60 | | | CAAG |
| chr5 | 17723 | 1772384 | NSD1:NM_001365684: | + | 1590 | AGCCACAGGCACAG |
| | 8431 | 49 | U1:Ex8:−59 | | | ACAA |
| chr5 | 17723 | 1772384 | NSD1:NM_001365684: | + | 1591 | GAGCCACAGGCACA |
| | 8432 | 50 | U1:Ex8:−58 | | | GACA |
| chr5 | 17723 | 1772384 | NSD1:NM_001365684: | + | 1592 | GGAGCCACAGGCAC |
| | 8433 | 51 | U1:Ex8:−57 | | | AGAC |
| chr5 | 17723 | 1772384 | NSD1:NM_001365684: | + | 1593 | CGGAGCCACAGGCA |
| | 8434 | 52 | U1:Ex8:−56 | | | CAGA |
| chr5 | 17723 | 1772384 | NSD1:NM_001365684: | + | 1594 | CCGGAGCCACAGGC |
| | 8435 | 53 | U1:Ex8:−55 | | | ACAG |
| chr5 | 17723 | 1772384 | NSD1:NM_001365684: | + | 1595 | TCCGGAGCCACAGG |
| | 8436 | 54 | U1:Ex8:−54 | | | CACA |
| chr5 | 17723 | 1772384 | NSD1:NM_001365684: | + | 1596 | TTCCGGAGCCACAG |
| | 8437 | 55 | U1:Ex8:−53 | | | GCAC |
| chr5 | 17723 | 1772384 | NSD1:NM_001365684: | + | 1597 | CTTCCGGAGCCACA |
| | 8438 | 56 | U1:Ex8:−52 | | | GGCA |
| chr5 | 17723 | 1772384 | NSD1:NM_001365684: | + | 1598 | ACTTCCGGAGCCAC |
| | 8439 | 57 | U1:Ex8:−51 | | | AGGC |
| chr5 | 17723 | 1772384 | NSD1:NM_001365684: | + | 1599 | GACTTCCGGAGCCA |
| | 8440 | 58 | U1:Ex8:−50 | | | CAGG |
| chr5 | 17723 | 1772384 | NSD1:NM_001365684: | + | 1600 | AGACTTCCGGAGCC |
| | 8441 | 59 | U1:Ex8:−49 | | | ACAG |
| chr5 | 17723 | 1772384 | NSD1:NM_001365684: | + | 1601 | GAGACTTCCGGAGC |
| | 8442 | 60 | U1:Ex8:−48 | | | CACA |
| chr5 | 17723 | 1772384 | NSD1:NM_001365684: | + | 1602 | AGAGACTTCCGGAG |
| | 8443 | 61 | U1:Ex8:−47 | | | CCAC |
| chr5 | 17723 | 1772384 | NSD1:NM_001365684: | + | 1603 | GAGAGACTTCCGGA |
| | 8444 | 62 | U1:Ex8:−46 | | | GCCA |
| chr5 | 17723 | 1772384 | NSD1:NM_001365684: | + | 1604 | GGAGAGACTTCCGG |
| | 8445 | 63 | U1:Ex8:−45 | | | AGCC |
| chr5 | 17723 | 1772384 | NSD1:NM_001365684: | + | 1605 | TGGAGAGACTTCCG |
| | 8446 | 64 | U1:Ex8:−44 | | | GAGC |
| chr5 | 17723 | 1772384 | NSD1:NM_001365684: | + | 1606 | GTGGAGAGACTTCC |
| | 8447 | 65 | U1:Ex8:−43 | | | GGAG |
| chr5 | 17723 | 1772384 | NSD1:NM_001365684: | + | 1607 | CGTGGAGAGACTTC |
| | 8448 | 66 | Ex8:−42 | | | CGGA |
| chr5 | 17723 | 1772384 | NSD1:NM_001365684: | + | 1608 | CCGTGGAGAGACTT |
| | 8449 | 67 | U1:Ex8:−41 | | | CCGG |
| chr5 | 17723 | 1772384 | NSD1:NM_001365684: | + | 1609 | GCCGTGGAGAGACT |
| | 8450 | 68 | U1:Ex8:−40 | | | TCCG |
| chr5 | 17723 | 1772384 | NSD1:NM_001365684: | + | 1610 | GGCCGTGGAGAGAC |
| | 8451 | 69 | U1:Ex8:−39 | | | TTCC |
| chr5 | 17723 | 1772384 | NSD1:NM_001365684: | + | 1611 | AGGCCGTGGAGAGA |
| | 8452 | 70 | U1:Ex8:−38 | | | CTTC |
| chr5 | 17723 | 1772384 | NSD1:NM_001365684: | + | 1612 | CAGGCCGTGGAGAG |
| | 8453 | 71 | U1:Ex8:−37 | | | ACTT |
| chr5 | 17723 | 1772384 | NSD1:NM_001365684: | + | 1613 | GCAGGCCGTGGAGA |
| | 8454 | 72 | U1:Ex8:−36 | | | GACT |
| chr5 | 17723 | 1772384 | NSD1:NM_001365684: | + | 1614 | GGCAGGCCGTGGAG |
| | 8455 | 73 | U1:Ex8:−35 | | | AGAC |
| chr5 | 17723 | 1772384 | NSD1:NM_001365684: | + | 1615 | GGGCAGGCCGTGGA |
| | 8456 | 74 | U1:Ex8:−34 | | | GAGA |
| chr5 | 17723 | 1772384 | NSD1:NM_001365684: | + | 1616 | AGGGCAGGCCGTGG |
| | 8457 | 75 | U1:Ex8:−33 | | | AGAG |
| chr5 | 17723 | 1772384 | NSD1:NM_001365684: | + | 1617 | AAGGGCAGGCCGTG |
| | 8458 | 76 | U1:Ex8:−32 | | | GAGA |
| chr5 | 17723 | 1772384 | NSD1:NM_001365684: | 1 | 1618 | CAAGGGCAGGCCGT |
| | 8459 | 77 | U1:Ex8:−31 | | | GGAG |
| chr5 | 17723 | 1772384 | NSD1:NM_001365684: | + | 1619 | TCAAGGGCAGGCCG |
| | 8460 | 78 | U1:Ex8:−30 | | | TGGA |
| chr5 | 17723 | 1772384 | NSD1:NM_001365684: | 1 | 1620 | CTCAAGGGCAGGCC |
| | 8461 | 79 | U1:Ex8:−29 | | | GTGG |
| chr5 | 17723 | 1772384 | NSD1:NM_001365684: | + | 1621 | ACTCAAGGGCAGGC |
| | 8462 | 80 | U1:Ex8:−28 | | | CGTG |
| chr5 | 17723 | 1772384 | NSD1:NM_001365684: | + | 1622 | GACTCAAGGGCAGG |
| | 8463 | 81 | U1:Ex8:−27 | | | CCGT |
| chr5 | 17723 | 1772384 | NSD1:NM_001365684: | 1 | 1623 | AGACTCAAGGGCAG |
| | 8464 | 82 | U1:Ex8:−26 | | | GCCG |
| chr5 | 17723 | 1772384 | NSD1:NM_001365684: | 1 | 1624 | CAGACTCAAGGGCA |
| | 8465 | 83 | U1:Ex8:−25 | | | GGCC |
| chr5 | 17723 | 1772384 | NSD1:NM_001365684: | + | 1625 | TCAGACTCAAGGGC |
| | 8466 | 84 | U1:Ex8:−24 | | | AGGC |
| chr5 | 17723 | 1772384 | NSD1:NM_001365684: | + | 1626 | CTCAGACTCAAGGG |
| | 8467 | 85 | U1:Ex8:−23 | | | CAGG |
| chr5 | 17723 | 1772384 | NSD1:NM_001365684: | 1 | 1627 | CCTCAGACTCAAGG |
| | 8468 | 86 | U1:Ex8:−22 | | | GCAG |
| chr5 | 17723 | 1772384 | NSD1:NM_001365684: | + | 1628 | TCCTCAGACTCAAG |
| | 8469 | 87 | U1:Ex8:−21 | | | GGCA |
| chr5 | 17723 | 1772384 | NSD1:NM_001365684: | + | 1629 | TTCCTCAGACTCAAG |
| | 8470 | 88 | U1:Ex8:−20 | | | GGC |
| chr5 | 17723 | 1772384 | NSD1:NM_001365684: | + | 1630 | ATTCCTCAGACTCAA |
| | 8471 | 89 | U1:Ex8:−19 | | | GGG |
| chr5 | 17723 | 1772384 | NSD1:NM_001365684: | + | 1631 | AATTCCTCAGACTCA |
| | 8472 | 90 | U1:Ex8:−18 | | | AGG |
| chr5 | 17723 | 1772384 | NSD1:NM_001365684: | + | 1632 | CAATTCCTCAGACTC |
| | 8473 | 91 | U1:Ex8:−17 | | | AAG |
| chr5 | 17723 | 1772384 | NSD1:NM_001365684: | + | 1633 | GCAATTCCTCAGACT |
| | 8474 | 92 | U1:Ex8:−16 | | | CAA |
| chr5 | 17723 | 1772384 | NSD1:NM_001365684: | 1 | 1634 | AGCAATTCCTCAGA |
| | 8475 | 93 | U1:Ex8:−15 | | | CTCA |
| chr5 | 17723 | 1772384 | NSD1:NM_001365684: | + | 1635 | TAGCAATTCCTCAGA |
| | 8476 | 94 | U1:Ex8:−14 | | | CTC |
| chr5 | 17723 | 1772384 | NSD1:NM_001365684: | + | 1636 | CTAGCAATTCCTCAG |
| | 8477 | 95 | U1:Ex8:−13 | | | ACT |
| chr5 | 17723 | 1772384 | NSD1:NM_001365684: | + | 1637 | ACTAGCAATTCCTCA |
| | 8478 | 96 | U1:Ex8:−12 | | | GAC |
| chr5 | 17723 | 1772384 | NSD1:NM_001365684: | + | 1638 | AACTAGCAATTCCTC |
| | 8479 | 97 | U1:Ex8:−11 | | | AGA |
| chr5 | 17723 | 1772384 | NSD1:NM_001365684: | + | 1639 | TAACTAGCAATTCCT |
| | 8480 | 98 | U1:Ex8:−10 | | | CAG |
| chr5 | 17723 | 1772384 | NSD1:NM_001365684: | + | 1640 | TTAACTAGCAATTCC |
| | 8481 | 99 | U1:Ex8:−9 | | | TCA |
| chr5 | 17723 | 1772385 | NSD1:NM_001365684: | + | 1641 | TTTAACTAGCAATTC |
| | 8482 | 00 | U1:Ex8:−8 | | | CTC |
| chr5 | 17723 | 1772385 | NSD1:NM_001365684: | + | 1642 | TTTTAACTAGCAATT |
| | 8483 | 01 | U1:Ex8:−7 | | | CCT |
| chr5 | 17723 | 1772385 | NSD1:NM_001365684: | + | 1643 | GTTTTAACTAGCAAT |
| | 8484 | 02 | U1:Ex8:−6 | | | TCC |
| chr5 | 17723 | 1772385 | NSD1:NM_001365684: | + | 1644 | CGTTTTAACTAGCAA |
| | 8485 | 03 | U1:Ex8:−5 | | | TTC |
| chr5 | 17723 | 1772385 | NSD1:NM_001365684: | + | 1645 | GCGTTTTAACTAGCA |
| | 8486 | 04 | U1:Ex8:−4 | | | ATT |
| chr5 | 17723 | 1772385 | NSD1:NM_001365684: | + | 1646 | CCAACCCCACCTTAC |
| | 8504 | 22 | U1:Ex8-IVS8:−3 | | | CTG |
| chr5 | 17723 | 1772385 | NSD1:NM_001365684: | + | 1647 | CCCAACCCCACCTTA |
| | 8505 | 23 | U1:Ex8-IVS8:−2 | | | CCT |
| chr5 | 17723 | 1772385 | NSD1:NM_001365684: | + | 1648 | CCCCAACCCCACCTT |
| | 8506 | 24 | U1:Ex8-IVS8:−1 | | | ACC |
| chr5 | 17723 | 1772385 | NSD1:NM_001365684: | + | 1649 | ACCCCAACCCCACCT |
| | 8507 | 25 | U1:IVS8:1 | | | TAC |
| chr5 | 17723 | 1772385 | NSD1:NM_001365684: | + | 1650 | GACCCCAACCCCAC |
| | 8508 | 26 | U1:IVS8:2 | | | CTTA |
| chr5 | 17723 | 1772385 | NSD1:NM_001365684: | + | 1651 | AGACCCCAACCCCA |
| | 8509 | 27 | U1:IVS8:3 | | | CCTT |
| chr5 | 17723 | 1772385 | NSD1:NM_001365684: | + | 1652 | GAGACCCCAACCCC |
| | 8510 | 28 | U1:IVS8:4 | | | ACCT |
| chr5 | 17723 | 1772385 | NSD1:NM_001365684: | + | 1653 | TGAGACCCCAACCC |
| | 8511 | 29 | U1:IVS8:5 | | | CACC |
| chr5 | 17723 | 1772385 | NSD1:NM_001365684: | + | 1654 | CTGAGACCCCAACC |
| | 8512 | 30 | U1:IVS8:6 | | | CCAC |
| chr5 | 17723 | 1772385 | NSD1:NM_001365684: | + | 1655 | ACTGAGACCCCAAC |
| | 8513 | 31 | U1:IVS8:7 | | | CCCA |
| chr5 | 17723 | 1772385 | NSD1:NM_001365684: | + | 1656 | TACTGAGACCCCAA |
| | 8514 | 32 | U1:IVS8:8 | | | CCCC |
| chr5 | 17723 | 1772385 | NSD1:NM_001365684: | + | 1657 | ATACTGAGACCCCA |
| | 8515 | 33 | U1:IVS8:9 | | | ACCC |
| chr5 | 17723 | 1772385 | NSD1:NM_001365684: | + | 1658 | AATACTGAGACCCC |
| | 8516 | 34 | U1:IVS8:10 | | | AACC |
| chr5 | 17723 | 1772385 | NSD1:NM_001365684: | + | 1659 | AAATACTGAGACCC |
| | 8517 | 35 | U1:IVS8:11 | | | CAAC |
| chr5 | 17723 | 1772385 | NSD1:NM_001365684: | + | 1660 | CAAATACTGAGACC |
| | 8518 | 36 | U1:IVS8:12 | | | CCAA |
| chr5 | 17723 | 1772385 | NSD1:NM_001365684: | + | 1661 | TCAAATACTGAGAC |
| | 8519 | 37 | U1:IVS8:13 | | | CCCA |
| chr5 | 17723 | 1772385 | NSD1:NM_001365684: | + | 1662 | CTCAAATACTGAGA |
| | 8520 | 38 | U1:IVS8:14 | | | CCCC |
| chr5 | 17723 | 1772385 | NSD1:NM_001365684: | + | 1663 | GCTCAAATACTGAG |
| | 8521 | 39 | U1:IVS8:15 | | | ACCC |
| chr5 | 17723 | 1772385 | NSD1:NM_001365684: | + | 1664 | TGCTCAAATACTGA |
| | 8522 | 40 | U1:IVS8:16 | | | GACC |
| chr5 | 17723 | 1772385 | NSD1:NM_001365684: | + | 1665 | CTGCTCAAATACTGA |
| | 8523 | 41 | U1:IVS8:17 | | | GAC |
| chr5 | 17723 | 1772385 | NSD1:NM_001365684: | + | 1666 | TCTGCTCAAATACTG |
| | 8524 | 42 | U1:IVS8:18 | | | AGA |
| chr5 | 17723 | 1772385 | NSD1:NM_001365684: | + | 1667 | ATCTGCTCAAATACT |
| | 8525 | 43 | U1:IVS8:19 | | | GAG |
| chr5 | 17723 | 1772385 | NSD1:NM_001365684: | + | 1668 | TATCTGCTCAAATAC |
| | 8526 | 44 | U1:IVS8:20 | | | TGA |
| chr5 | 17723 | 1772385 | NSD1:NM_001365684: | + | 1669 | ATATCTGCTCAAATA |
| | 8527 | 45 | U1:IVS8:21 | | | CTG |
| chr5 | 17723 | 1772385 | NSD1:NM_001365684: | + | 1670 | CATATCTGCTCAAAT |
| | 8528 | 46 | U1:IVS8:22 | | | ACT |
| chr5 | 17723 | 1772385 | NSD1:NM_001365684: | + | 1671 | TCATATCTGCTCAAA |
| | 8529 | 47 | U1:IVS8:23 | | | TAC |
| chr5 | 17723 | 1772385 | NSD1:NM_001365684: | + | 1672 | ATCATATCTGCTCAA |
| | 8530 | 48 | U1:IVS8:24 | | | ATA |
| chr5 | 17723 | 1772385 | NSD1:NM_001365684: | + | 1673 | AATCATATCTGCTCA |
| | 8531 | 49 | U1:IVS8:25 | | | AAT |
| chr5 | 17723 | 1772385 | NSD1:NM_001365684: | + | 1674 | TAATCATATCTGCTC |
| | 8532 | 50 | U1:IVS8:26 | | | AAA |
| chr5 | 17723 | 1772385 | NSD1:NM_001365684: | + | 1675 | CTAATCATATCTGCT |
| | 8533 | 51 | U1:IVS8:27 | | | CAA |
| chr5 | 17723 | 1772385 | NSD1:NM_001365684: | + | 1676 | TCTAATCATATCTGC |
| | 8534 | 52 | U1:IVS8:28 | | | TCA |
| chr5 | 17723 | 1772385 | NSD1:NM_001365684: | + | 1677 | CTCTAATCATATCTG |
| | 8535 | 53 | U1:IVS8:29 | | | CTC |
| chr5 | 17723 | 1772385 | NSD1:NM_001365684: | + | 1678 | CCTCTAATCATATCT |
| | 8536 | 54 | U1:IVS8:30 | | | GCT |
| chr5 | 17723 | 1772385 | NSD1:NM_001365684: | + | 1679 | TCCTCTAATCATATC |
| | 8537 | 55 | U1:IVS8:31 | | | TGC |
| chr5 | 17723 | 1772385 | NSD1:NM_001365684: | + | 1680 | TTCCTCTAATCATAT |
| | 8538 | 56 | U1:IVS8:32 | | | CTG |
| chr5 | 17723 | 1772385 | NSD1:NM_001365684: | + | 1681 | CTTCCTCTAATCATA |
| | 8539 | 57 | U1:IVS8:33 | | | TCT |
| chr5 | 17723 | 1772385 | NSD1:NM_001365684: | + | 1682 | GCTTCCTCTAATCAT |
| | 8540 | 58 | U1:IVS8:34 | | | ATC |
| chr5 | 17723 | 1772385 | NSD1:NM_001365684: | + | 1683 | TGCTTCCTCTAATCA |
| | 8541 | 59 | U1:IVS8:35 | | | TAT |
| chr5 | 17723 | 1772385 | NSD1:NM_001365684: | + | 1684 | CTGCTTCCTCTAATC |
| | 8542 | 60 | U1:IVS8:36 | | | ATA |
| chr5 | 17723 | 1772385 | NSD1:NM_001365684: | + | 1685 | CCTGCTTCCTCTAAT |
| | 8543 | 61 | U1:IVS8:37 | | | CAT |
| chr5 | 17723 | 1772385 | NSD1:NM_001365684: | + | 1686 | TCCTGCTTCCTCTAA |
| | 8544 | 62 | U1:IVS8:38 | | | TCA |
| chr5 | 17723 | 1772385 | NSD1:NM_001365684: | 1 | 1687 | CTCCTGCTTCCTCTA |
| | 8545 | 63 | U1:IVS8:39 | | | ATC |
| chr5 | 17723 | 1772385 | NSD1:NM_001365684: | + | 1688 | TCTCCTGCTTCCTCT |
| | 8546 | 64 | U1:IVS8:40 | | | AAT |
| chr5 | 17723 | 1772385 | NSD1:NM_001365684: | + | 1689 | ATCTCCTGCTTCCTC |
| | 8547 | 65 | U1:IVS8:41 | | | TAA |
| chr5 | 17723 | 1772385 | NSD1:NM_001365684: | + | 1690 | AATCTCCTGCTTCCT |
| | 8548 | 66 | U1:IVS8:42 | | | CTA |
| chr5 | 17723 | 1772385 | NSD1:NM_001365684: | + | 1691 | AAATCTCCTGCTTCC |
| | 8549 | 67 | U1:IVS8:43 | | | TCT |
| chr5 | 17723 | 1772385 | NSD1:NM_001365684: | + | 1692 | AAAATCTCCTGCTTC |
| | 8550 | 68 | U1:IVS8:44 | | | CTC |
| chr5 | 17723 | 1772385 | NSD1:NM_001365684: | + | 1693 | TAAAATCTCCTGCTT |
| | 8551 | 69 | U1:IVS8:45 | | | CCT |
| chr5 | 17723 | 1772385 | NSD1:NM_001365684: | + | 1694 | CTAAAATCTCCTGCT |
| | 8552 | 70 | U1:IVS8:46 | | | TCC |
| chr5 | 17723 | 1772385 | NSD1:NM_001365684: | + | 1695 | ACTAAAATCTCCTGC |
| | 8553 | 71 | U1:IVS8:47 | | | TTC |
| chr5 | 17723 | 1772385 | NSD1:NM_001365684: | + | 1696 | TACTAAAATCTCCTG |
| | 8554 | 72 | U1:IVS8:48 | | | CTT |
| chr5 | 17723 | 1772385 | NSD1:NM_001365684: | + | 1697 | ATACTAAAATCTCCT |
| | 8555 | 73 | U1:IVS8:49 | | | GCT |
| chr5 | 17723 | 1772385 | NSD1:NM_001365684: | + | 1698 | CATACTAAAATCTCC |
| | 8556 | 74 | U1:IVS8:50 | | | TGC |
| chr5 | 17723 | 1772385 | NSD1:NM_001365684: | + | 1699 | ACATACTAAAATCTC |
| | 8557 | 75 | U1:IVS8:51 | | | CTG |
| chr5 | 17723 | 1772385 | NSD1:NM_001365684: | + | 1700 | AACATACTAAAATC |
| | 8558 | 76 | U1:IVS8:52 | | | TCCT |
| chr5 | 17723 | 1772385 | NSD1:NM_001365684: | + | 1701 | AAACATACTAAAAT |
| | 8559 | 77 | U1:IVS8:53 | | | CTCC |
| chr5 | 17723 | 1772385 | NSD1:NM_001365684: | + | 1702 | AAAACATACTAAAA |
| | 8560 | 78 | U1:IVS8:54 | | | TCTC |
| chr5 | 17723 | 1772385 | NSD1:NM_001365684: | + | 1703 | CAAAACATACTAAA |
| | 8561 | 79 | U1:IVS8:55 | | | ATCT |
| chr5 | 17723 | 1772385 | NSD1:NM_001365684: | + | 1704 | TCAAAACATACTAA |
| | 8562 | 80 | U1:IVS8:56 | | | AATC |
| chr5 | 17723 | 1772385 | NSD1:NM_001365684: | + | 1705 | ATCAAAACATACTA |
| | 8563 | 81 | U1:IVS8:57 | | | AAAT |
| chr5 | 17723 | 1772385 | NSD1:NM_001365684: | + | 1706 | CATCAAAACATACT |
| | 8564 | 82 | U1:IVS8:58 | | | AAAA |
| chr5 | 17723 | 1772385 | NSD1:NM_001365684: | + | 1707 | ACATCAAAACATAC |
| | 8565 | 83 | U1:IVS8:59 | | | TAAA |
| chr5 | 17723 | 1772385 | NSD1:NM_001365684: | + | 1708 | TACATCAAAACATA |
| | 8566 | 84 | U1:IVS8:60 | | | CTAA |
| chr5 | 17723 | 1772385 | NSD1:NM_001365684: | + | 1709 | TTACATCAAAACAT |
| | 8567 | 85 | U1:IVS8:61 | | | ACTA |
| chr5 | 17723 | 1772385 | NSD1:NM_001365684: | + | 1710 | TTTACATCAAAACAT |
| | 8568 | 86 | U1:IVS8:62 | | | ACT |
| chr5 | 17723 | 1772385 | NSD1:NM_001365684: | + | 1711 | CTTTACATCAAAACA |
| | 8569 | 87 | U1:IVS8:63 | | | TAC |
| chr5 | 17723 | 1772385 | NSD1:NM_001365684: | + | 1712 | GCTTTACATCAAAAC |
| | 8570 | 88 | U1:IVS8:64 | | | ATA |
| chr5 | 17723 | 1772385 | NSD1:NM_001365684: | + | 1713 | GGCTTTACATCAAA |
| | 8571 | 89 | U1:IVS8:65 | | | ACAT |
| chr5 | 17723 | 1772385 | NSD1:NM_001365684: | + | 1714 | TGGCTTTACATCAAA |
| | 8572 | 90 | U1:IVS8:66 | | | ACA |
| chr5 | 17723 | 1772385 | NSD1:NM_001365684: | + | 1715 | TTGGCTTTACATCAA |
| | 8573 | 91 | U1:IVS8:67 | | | AAC |
| chr5 | 17723 | 1772385 | NSD1:NM_001365684: | + | 1716 | GTTGGCTTTACATCA |
| | 8574 | 92 | U1:IVS8:68 | | | AAA |
| chr5 | 17723 | 1772385 | NSD1:NM_001365684: | + | 1717 | TGTTGGCTTTACATC |
| | 8575 | 93 | U1:IVS8:69 | | | AAA |
| chr5 | 17723 | 1772385 | NSD1:NM_001365684: | + | 1718 | ATGTTGGCTTTACAT |
| | 8576 | 94 | U1:IVS8:70 | | | CAA |
| chr5 | 17723 | 1772385 | NSD1:NM_001365684: | + | 1719 | AATGTTGGCTTTACA |
| | 8577 | 95 | U1:IVS8:71 | | | TCA |
| chr5 | 17723 | 1772385 | NSD1:NM_001365684: | + | 1720 | CAATGTTGGCTTTAC |
| | 8578 | 96 | U1:IVS8:72 | | | ATC |
| chr5 | 17723 | 1772385 | NSD1:NM_001365684: | + | 1721 | ACAATGTTGGCTTTA |
| | 8579 | 97 | U1:IVS8:73 | | | CAT |
| chr5 | 17723 | 1772385 | NSD1:NM_001365684: | + | 1722 | TACAATGTTGGCTTT |
| | 8580 | 98 | U1:IVS8:74 | | | ACA |
| chr5 | 17723 | 1772385 | NSD1:NM_001365684: | + | 1723 | ATACAATGTTGGCTT |
| | 8581 | 99 | U1:IVS8:75 | | | TAC |
| chr5 | 17723 | 1772386 | NSD1:NM_001365684: | + | 1724 | GATACAATGTTGGCT |
| | 8582 | 00 | U1:IVS8:76 | | | TTA |
| chr5 | 17723 | 1772386 | NSD1:NM_001365684: | + | 1725 | AGATACAATGTTGG |
| | 8583 | 01 | U1:IVS8:77 | | | CTTT |
| chr5 | 17723 | 1772386 | NSD1:NM_001365684: | + | 1726 | TAGATACAATGTTG |
| | 8584 | 02 | U1:IVS8:78 | | | GCTT |
| chr5 | 17723 | 1772386 | NSD1:NM_001365684: | + | 1727 | ATAGATACAATGTT |
| | 8585 | 03 | U1:IVS8:79 | | | GGCT |
| chr5 | 17723 | 1772386 | NSD1:NM_001365684: | + | 1728 | TATAGATACAATGTT |
| | 8586 | 04 | U1:IVS8:80 | | | GGC |
| chr5 | 17723 | 1772386 | NSD1:NM_001365684: | + | 1729 | ATATAGATACAATG |
| | 8587 | 05 | U1:IVS8:81 | | | TTGG |
| chr5 | 17723 | 1772386 | NSD1:NM_001365684: | + | 1730 | TATATAGATACAAT |
| | 8588 | 06 | U1:IVS8:82 | | | GTTG |
| chr5 | 17723 | 1772386 | NSD1:NM_001365684: | + | 1731 | GTATATAGATACAA |
| | 8589 | 07 | U1:IVS8:83 | | | TGTT |
| chr5 | 17723 | 1772386 | NSD1:NM_001365684: | + | 1732 | TGTATATAGATACA |
| | 8590 | 08 | U1:IVS8:84 | | | ATGT |
| chr5 | 17723 | 1772386 | NSD1:NM_001365684: | + | 1733 | TTGTATATAGATACA |
| | 8591 | 09 | U1:IVS8:85 | | | ATG |
| chr5 | 17723 | 1772386 | NSD1:NM_001365684: | + | 1734 | ATTGTATATAGATAC |
| | 8592 | 10 | U1:IVS8:86 | | | AAT |
| chr5 | 17723 | 1772386 | NSD1:NM_001365684: | + | 1735 | TATTGTATATAGATA |
| | 8593 | 11 | U1:IVS8:87 | | | CAA |
| chr5 | 17723 | 1772386 | NSD1:NM_001365684: | + | 1736 | TTATTGTATATAGAT |
| | 8594 | 12 | U1:IVS8:88 | | | ACA |
| chr5 | 17723 | 1772386 | NSD1:NM_001365684: | + | 1737 | TTTATTGTATATAGA |
| | 8595 | 13 | U1:IVS8:89 | | | TAC |
| chr5 | 17723 | 1772386 | NSD1:NM_001365684: | + | 1738 | GTTTATTGTATATAG |
| | 8596 | 14 | U1:IVS8:90 | | | ATA |
| chr5 | 17723 | 1772386 | NSD1:NM_001365684: | + | 1739 | AGTTTATTGTATATA |
| | 8597 | 15 | U1:IVS8:91 | | | GAT |
| chr5 | 17723 | 1772386 | NSD1:NM_001365684: | + | 1740 | TAGTTTATTGTATAT |
| | 8598 | 16 | U1:IVS8:92 | | | AGA |
| chr5 | 17723 | 1772386 | NSD1:NM_001365684: | + | 1741 | GTAGTTTATTGTATA |
| | 8599 | 17 | U1:IVS8:93 | | | TAG |
| chr5 | 17723 | 1772386 | NSD1:NM_001365684: | + | 1742 | GGTAGTTTATTGTAT |
| | 8600 | 18 | U1:IVS8:94 | | | ATA |
| chr5 | 17723 | 1772386 | NSD1:NM_001365684: | + | 1743 | GGGTAGTTTATTGTA |
| | 8601 | 19 | U1:IVS8:95 | | | TAT |
| chr5 | 17723 | 1772386 | NSD1:NM_001365684: | + | 1744 | GGGGTAGTTTATTGT |
| | 8602 | 20 | U1:IVS8:96 | | | ATA |
| chr5 | 17723 | 1772386 | NSD1:NM_001365684: | + | 1745 | GGGGGTAGTTTATTG |
| | 8603 | 21 | U1:IVS8:97 | | | TAT |
| chr5 | 17723 | 1772386 | NSD1:NM_001365684: | + | 1746 | AGGGGGTAGTTTATT |
| | 8604 | 22 | U1:IVS8:98 | | | GTA |
| chr5 | 17723 | 1772386 | NSD1:NM_001365684: | + | 1747 | AAGGGGGTAGTTTA |
| | 8605 | 23 | U1:IVS8:99 | | | TTGT |
| chr5 | 17723 | 1772386 | NSD1:NM_001365684: | + | 1748 | AAAGGGGGTAGTTT |
| | 8606 | 24 | U1:IVS8:100 | | | ATTG |

TABLE 5F
Exemplary U1 Vector Sequences

| | | SEQ ID |
|---|---|---|
| Region | Sequence | NO: |
| Promoter | gctccatctggccaccgaaaggttgctccttaacacaggctaaggaccagcttctttgggagagaaca | 1761 |
| sequence | gacgcaggggcgggagggaaaaagggagaggcagacgtcacttccccttggcggctctggcagcagatt | |
| (Human U1 | ggtcggttgagtggcagaaaggcagacggggactgggcaaggcactgtcggtgacatcacggacagg | |
| promoter) | gcgacttctatgtagatgaggcagcgcagaggctgctgcttcgccacttgctgcttcaccacgaagga | |
| | gttcccgtgccctgggagcgggttcaggaccgctgatcggaagtgagaatcccagctgtgtgtcaggg | |
| | ctggaaagggctcgggagtgcgcggggcaagtgaccgtgtgtgtaaagagtgaggcgtatgaggctgt | |
| | gtcggggcagaggcccaagatctc | |
| Wild-type U1 | acttacctg | |
| Antisense | | |
| sequence | | |
| Wild-type U1 | atacttacctggcaggggagataccatgatcacgaaggtggttttcccagggcgaggcttatccattg | 1762 |
| non-coding | cactccggatgtgctgacccctgcgatttccccaaatgtgggaaactcgactgcataatttgtggtag | |
| RNA | tgggggactgcgttcgcgctttcccctg | |
| sequence | Any of the ASO sequences from Table 4, Table 5A, Table 5B, Table 5D, | |
| ASO | Table 5E, and Table 5G | |
| sequences | | |
| replacing the | | |
| Wild-type U1 | | |
| Antisense | | |
| sequence | | |
| 3′ regulatory | actttctggagtttcaaaagtagactgtacgctaagggtcatatctttttttgttttggtttgtgtcttgg | 1763 |
| sequence | ttggcgtcttaaatgttaatcctacagtggagggctgcggaataggaagtaacatgtcgcctgcacgccat | |
| | aggagaaaaagcgagcatcagccgtatcggctttgtaacacaaattagctatcgtgaagtccgctcag | |
| Exemplary | gctccatctggccaccgaaaggttgctccttaacacaggctaaggaccagcttctttgggagagaacaga | 1764 |
| Full sequence | cgcaggggcgggagggaaaaagggagaggcagacgtcacttccccttggcggctctggcagcagattgg | |
| of wild-type | tcggttgagtggcagaaaggcagacggggactgggcaaggcactgtcggtgacatcacggacagggcg | |
| U1 snRNA | acttctatgtagatgaggcagcgcagaggctgctgcttcgccacttgctgcttcaccacgaaggagtt | |
| | cccgtgccctgggagcgggttcaggaccgctgatcggaagtgagaatcccagctgtgtgtcagggctg | |
| | gaaagggctcgggagtgcgcggggcaagtgaccgtgtgtgtaaagagtgaggcgtatgaggctgtgtc | |
| | ggggcagaggcccaagatctcatacttacctggcaggggagataccatgatcacgaaggtggttttcc | |
| | cagggcgaggcttatccattgcactccggatgtgctgacccctgcgatttccccaaatgtgggaaact | |
| | cgactgcataatttgtggtagtgggggactgcgttcgcgctttcccctgactttctggagtttcaaaa | |
| | gtagactgtacgctaagggtcatatctttttttgttttggtttgtgtcttggttggcgtcttaaatgt | |
| | taatcctacagtggagggctgcggaataggaagtaacatgtcgcctgcacgccataggagaaaaagcg | |
| | agcatcagccgtatcggctttgtaacacaaattagctatcgtgaagtccgctcag | |
| Full sequence | Gctccatctggccaccgaaaggttgctccttaacacaggctaaggaccagcttctttgggagagaacag | 1765 |
| of U1 snRNA | acgcaggggcgggagggaaaaagggagaggcagacgtcacttccccttggcggctctggcagcagat | |
| containing an | tggtcggttgagtggcagaaaggcagacggggactgggcaaggcactgtcggtgacatcacggacag | |
| ASO | ggcgacttctatgtagatgaggcagcgcagaggctgctgcttcgccacttgctgcttcaccacgaaggag | |
| sequence | ttcccgtgccctgggagcgggttcaggaccgctgatcggaagtgagaatcccagctgtgtgtcagggct | 1766 |
| replacing the | ggaaagggctcgggagtgcgcggggcaagtgaccgtgtgtgtaaagagtgaggcgtatgaggctgtgt | |
| antisense | cggggcagaggcccaagatctcat[ASO sequence] | |
| sequence of | gcaggggagataccatgatcacgaaggtggttttcccagggcgaggcttatccattgcactccggatgtg | |
| U1 snRNA | ctgacccctgcgatttccccaaatgtgggaaactcgactgcataatttgtggtagtgggggactgcgttcg | |
| | cgctttcccctgactttctggagtttcaaaagtagactgtacgctaagggtcatatctttttttgttttgg | |
| | tttgtgtcttggttggcgtcttaaatgttaatcctacagtggagggctgcggaataggaagtaacatgtcg | |
| | cctgcacgccataggagaaaaagcgagcatcagccgtatcggctttgtaacacaaattagctatcgtgaag | |
| | tccgctcag | |
| Exemplary | gctccatctggccaccgaaaggttgctccttaacacaggctaaggaccagcttctttgggagagaacaga | 1767 |
| full sequence | cgcaggggcgggagggaaaaagggagaggcagacgtcacttccccttggcggctctggcagcagatt | |
| of U1 snRNA | ggtcggttgagtggcagaaaggcagacggggactgggcaaggcactgtcggtgacatcacggacagg | |
| containing an | gcgacttctatgtagatgaggcagcgcagaggctgctgcttcgccacttgctgcttcaccacgaaggagtt | |
| ASO | cccgtgccctgggagcgggttcaggaccgctgatcggaagtgagaatcccagctgtgtgtcagggctg | |
| sequence | gaaagggctcgggagtgcgcggggcaagtgaccgtgtgtgtaaagagtgaggcgtatgaggctgtgtc | |
| replacing the | ggggcagaggcccaagatctcat | |
| antisense | ACAAAGGCTACAAAAAGTgcaggggagataccatgatcacgaaggtggttttcccag | |
| sequence of | ggcgaggcttatccattgcactccggatgtgctgacccctgcgatttccccaaatgtgggaaactcgactg | |
| U1 snRNA | cataatttgtggtagtgggggactgcgttcgcgctttcccctgactttctggagtttcaaaagtagactgt | |
| | acgctaagggtcatatctttttttgttttggtttgtgtcttggttggcgtcttaaatgttaatcctacagt | |
| | ggagggctgcggaataggaagtaacatgtcgcctgcacgccataggagaaaaagcgagcatcagccgtatc | |
| | ggctttgtaacacaaattagctatcgtgaagtccgctcag | |

TABLE 5G
Exemplary ASO Sequences

| | | | SEQ | |
|---|---|---|---|---|
| | | | ID | |
| chr | Start | End | NO: | ASO Sequence |
| chr5 | 177238123 | 177238141 | 110 | ACAAAGGCTACAAAAAGT |
| chr5 | 177238128 | 177238146 | 111 | TTCTGACAAAGGCTACAA |
| chr5 | 177238133 | 177238151 | 112 | TGAAATTCTGACAAAGGC |
| chr5 | 177238138 | 177238156 | 113 | AGGAATGAAATTCTGACA |
| chr5 | 177238143 | 177238161 | 114 | TTAAAAGGAATGAAATTC |
| chr5 | 177238148 | 177238166 | 115 | ACACTTTAAAAGGAATGA |
| chr5 | 177238153 | 177238171 | 116 | ATAACACACTTTAAAAGG |
| chr5 | 177238158 | 177238176 | 117 | AAAGAATAACACACTTTA |
| chr5 | 177238163 | 177238181 | 118 | GTCAAAAAGAATAACACA |
| chr5 | 177238168 | 177238186 | 119 | TAAGTGTCAAAAAGAATA |
| chr5 | 177238173 | 177238191 | 120 | TAATTTAAGTGTCAAAAA |
| chr5 | 177238178 | 177238196 | 121 | TGTTGTAATTTAAGTGTC |
| chr5 | 177238183 | 177238201 | 122 | AAAATTGTTGTAATTTAA |
| chr5 | 177238188 | 177238206 | 123 | AGGCCAAAATTGTTGTAA |
| chr5 | 177238193 | 177238211 | 124 | TCCACAGGCCAAAATTGT |
| chr5 | 177238198 | 177238216 | 125 | TAGAGTCCACAGGCCAAA |
| chr5 | 177238203 | 177238221 | 126 | AAAAATAGAGTCCACAGG |
| chr5 | 177238208 | 177238226 | 127 | AAAATAAAAATAGAGTCC |
| chr5 | 177238213 | 177238231 | 128 | AACAAAAAATAAAAATAG |
| chr5 | 177238218 | 177238236 | 129 | CTAAGAACAAAAAATAAA |
| chr5 | 177238223 | 177238241 | 130 | CTTACCTAAGAACAAAAA |
| chr5 | 177238228 | 177238246 | 131 | GGGAACTTACCTAAGAAC |
| chr5 | 177238233 | 177238251 | 132 | ACAGCGGGAACTTACCTA |
| chr5 | 177238236 | 177238254 | 133 | TTCACAGCGGGAACTTAC |
| chr5 | 177238237 | 177238255 | 134 | CTTCACAGCGGGAACTTA |
| chr5 | 177238241 | 177238259 | 135 | TCCTCTTCACAGCGGGAA |
| chr5 | 177238246 | 177238264 | 136 | GGCTTTCCTCTTCACAGC |
| chr5 | 177238251 | 177238269 | 137 | TAGAAGGCTTTCCTCTTC |
| chr5 | 177238256 | 177238274 | 138 | CGGGCTAGAAGGCTTTCC |
| chr5 | 177238261 | 177238279 | 139 | GACCTCGGGCTAGAAGGC |
| chr5 | 177238266 | 177238284 | 140 | AGATCGACCTCGGGCTAG |
| chr5 | 177238271 | 177238289 | 141 | GCACTAGATCGACCTCGG |
| chr5 | 177238276 | 177238294 | 142 | TCTGAGCACTAGATCGAC |
| chr5 | 177238281 | 177238299 | 143 | CTTGTTCTGAGCACTAGA |
| chr5 | 177238286 | 177238304 | 144 | ACCTGCTTGTTCTGAGCA |
| chr5 | 177238291 | 177238309 | 145 | CGTCCACCTGCTTGTTCT |
| chr5 | 177238296 | 177238314 | 146 | ATTCTCGTCCACCTGCTT |
| chr5 | 177238301 | 177238319 | 147 | AAAGAATTCTCGTCCACC |
| chr5 | 177238306 | 177238324 | 148 | AAATCAAAGAATTCTCGT |
| chr5 | 177238311 | 177238329 | 149 | GGTTGAAATCAAAGAATT |
| chr5 | 177238316 | 177238334 | 150 | TCTTTGGTTGAAATCAAA |
| chr5 | 177238321 | 177238339 | 151 | GCTCTTCTTTGGTTGAAA |
| chr5 | 177238326 | 177238344 | 152 | TGGAGGCTCTTCTTTGGT |
| chr5 | 177238331 | 177238349 | 153 | AGAACTGGAGGCTCTTCT |
| chr5 | 177238336 | 177238354 | 154 | TTTCAAGAACTGGAGGCT |
| chr5 | 177238341 | 177238359 | 155 | CTCCCTTTCAAGAACTGG |
| chr5 | 177238346 | 177238364 | 156 | GGAGCCTCCCTTTCAAGA |
| chr5 | 177238351 | 177238369 | 157 | AAAACGGAGCCTCCCTTT |
| chr5 | 177238356 | 177238374 | 158 | CTCCAAAAACGGAGCCTC |
| chr5 | 177238361 | 177238379 | 159 | GGGCCCTCCAAAAACGGA |
| chr5 | 177238366 | 177238384 | 160 | CCAAGGGGCCCTCCAAAA |
| chr5 | 177238374 | 177238392 | 161 | TGACTGAGCCAAGGGGCC |
| chr5 | 177238379 | 177238397 | 162 | AGTTCTGACTGAGCCAAG |
| chr5 | 177238384 | 177238402 | 163 | CTCCAAGTTCTGACTGAG |
| chr5 | 177238389 | 177238407 | 164 | TCCACCTCCAAGTTCTGA |
| chr5 | 177238394 | 177238412 | 165 | GCATGTCCACCTCCAAGT |
| chr5 | 177238399 | 177238417 | 166 | ACTCAGCATGTCCACCTC |
| chr5 | 177238404 | 177238422 | 167 | CGGCAACTCAGCATGTCC |
| chr5 | 177238409 | 177238427 | 168 | AGCTGCGGCAACTCAGCA |
| chr5 | 177238414 | 177238432 | 169 | AGGTCAGCTGCGGCAACT |
| chr5 | 177238419 | 177238437 | 170 | AGACAAGGTCAGCTGCGG |
| chr5 | 177238424 | 177238442 | 171 | GGCACAGACAAGGTCAGC |
| chr5 | 177238429 | 177238447 | 172 | CCACAGGCACAGACAAGG |
| chr5 | 177238434 | 177238452 | 173 | CGGAGCCACAGGCACAGA |
| chr5 | 177238439 | 177238457 | 174 | ACTTCCGGAGCCACAGGC |
| chr5 | 177238444 | 177238462 | 175 | GAGAGACTTCCGGAGCCA |
| chr5 | 177238449 | 177238467 | 176 | CCGTGGAGAGACTTCCGG |
| chr5 | 177238454 | 177238472 | 177 | GCAGGCCGTGGAGAGACT |
| chr5 | 177238459 | 177238477 | 178 | CAAGGGCAGGCCGTGGAG |
| chr5 | 177238464 | 177238482 | 179 | AGACTCAAGGGCAGGCCG |
| chr5 | 177238469 | 177238487 | 180 | TCCTCAGACTCAAGGGCA |
| chr5 | 177238474 | 177238492 | 181 | GCAATTCCTCAGACTCAA |
| chr5 | 177238479 | 177238497 | 182 | AACTAGCAATTCCTCAGA |
| chr5 | 177238484 | 177238502 | 183 | GTTTTAACTAGCAATTCC |
| chr5 | 177238487 | 177238505 | 184 | GGCGTTTTAACTAGCAAT |
| chr5 | 177238489 | 177238507 | 185 | CTGGCGTTTTAACTAGCA |
| chr5 | 177238492 | 177238510 | 186 | TACCTGGCGTTTTAACTA |
| chr5 | 177238497 | 177238515 | 187 | CACCTTACCTGGCGTTTT |
| chr5 | 177238502 | 177238520 | 188 | AACCCCACCTTACCTGGC |
| chr5 | 177238507 | 177238525 | 189 | ACCCCAACCCCACCTTAC |
| chr5 | 177238512 | 177238530 | 190 | CTGAGACCCCAACCCCAC |
| chr5 | 177238517 | 177238535 | 191 | AAATACTGAGACCCCAAC |
| chr5 | 177238522 | 177238540 | 192 | TGCTCAAATACTGAGACC |
| chr5 | 177238527 | 177238545 | 193 | ATATCTGCTCAAATACTG |
| chr5 | 177238532 | 177238550 | 194 | TAATCATATCTGCTCAAA |
| chr5 | 177238537 | 177238555 | 195 | TCCTCTAATCATATCTGC |
| chr5 | 177238542 | 177238560 | 196 | CTGCTTCCTCTAATCATA |
| chr5 | 177238547 | 177238565 | 197 | ATCTCCTGCTTCCTCTAA |
| chr5 | 177238552 | 177238570 | 198 | CTAAAATCTCCTGCTTCC |
| chr5 | 177238557 | 177238575 | 199 | ACATACTAAAATCTCCTG |
| chr5 | 177238562 | 177238580 | 200 | TCAAAACATACTAAAATC |
| chr5 | 177238567 | 177238585 | 201 | TTACATCAAAACATACTA |
| chr5 | 177238572 | 177238590 | 202 | TGGCTTTACATCAAAACA |
| chr5 | 177238577 | 177238595 | 203 | AATGTTGGCTTTACATCA |
| chr5 | 177238582 | 177238600 | 204 | GATACAATGTTGGCTTTA |
| chr5 | 177238587 | 177238605 | 205 | ATATAGATACAATGTTGG |
| chr5 | 177238592 | 177238610 | 206 | ATTGTATATAGATACAAT |
| chr5 | 177238597 | 177238615 | 207 | AGTTTATTGTATATAGAT |
| chr5 | 177238602 | 177238620 | 208 | GGGGTAGTTTATTGTATA |
| chr5 | 177238504 | 177238522 | 209 | CCAACCCCACCTTACCTG |
| chr5 | 177238538 | 177238556 | 210 | TTCCTCTAATCATATCTG |
| chr5 | 177238537 | 177238556 | 211 | TTCCTCTAATCATATCTGC |
| chr5 | 177238536 | 177238556 | 212 | TTCCTCTAATCATATCTGCT |
| chr5 | 177238538 | 177238558 | 213 | GCTTCCTCTAATCATATCTG |
| chr5 | 177238536 | 177238554 | 214 | CCTCTAATCATATCTGCT |
| chr5 | 177238535 | 177238555 | 215 | TCCTCTAATCATATCTGCTC |

TABLE 5G-1
Exemplary ASO Sequences

| | | | SEQ | |
|---|---|---|---|---|
| | | | ID | |
| chr | Start | End | NO: | ASO Sequence |
| chr5 | 177238123 | 177238141 | 216 | AACAAAGGCTACAAAAAGT |
| chr5 | 177238128 | 177238146 | 217 | AATTCTGACAAAGGCTACAA |
| chr5 | 177238133 | 177238151 | 218 | AATGAAATTCTGACAAAGGC |
| chr5 | 177238138 | 177238156 | 219 | AAGGAATGAAATTCTGACA |
| chr5 | 177238143 | 177238161 | 220 | AATTAAAAGGAATGAAATTC |
| chr5 | 177238148 | 177238166 | 221 | AACACTTTAAAAGGAATGA |
| chr5 | 177238153 | 177238171 | 222 | ATAACACACTTTAAAAGG |
| chr5 | 177238158 | 177238176 | 223 | AAAGAATAACACACTTTA |
| chr5 | 177238163 | 177238181 | 224 | AAGTCAAAAAGAATAACACA |
| chr5 | 177238168 | 177238186 | 225 | AATAAGTGTCAAAAAGAATA |
| chr5 | 177238173 | 177238191 | 226 | AATAATTTAAGTGTCAAAAA |
| chr5 | 177238178 | 177238196 | 227 | AATGTTGTAATTTAAGTGTC |
| chr5 | 177238183 | 177238201 | 228 | AAAATTGTTGTAATTTAA |
| chr5 | 177238188 | 177238206 | 229 | AAGGCCAAAATTGTTGTAA |
| chr5 | 177238193 | 177238211 | 230 | AATCCACAGGCCAAAATTGT |
| chr5 | 177238198 | 177238216 | 231 | AATAGAGTCCACAGGCCAAA |
| chr5 | 177238203 | 177238221 | 232 | AAAAATAGAGTCCACAGG |
| chr5 | 177238208 | 177238226 | 233 | AAAATAAAAATAGAGTCC |
| chr5 | 177238213 | 177238231 | 234 | AACAAAAAATAAAAATAG |
| chr5 | 177238218 | 177238236 | 235 | AACTAAGAACAAAAAATAAA |
| chr5 | 177238223 | 177238241 | 236 | AACTTACCTAAGAACAAAAA |
| chr5 | 177238228 | 177238246 | 237 | AAGGGAACTTACCTAAGAAC |
| chr5 | 177238233 | 177238251 | 238 | AACAGCGGGAACTTACCTA |
| chr5 | 177238236 | 177238254 | 239 | AATTCACAGCGGGAACTTAC |
| chr5 | 177238237 | 177238255 | 240 | AACTTCACAGCGGGAACTTA |
| chr5 | 177238241 | 177238259 | 241 | AATCCTCTTCACAGCGGGAA |
| chr5 | 177238246 | 177238264 | 242 | AAGGCTTTCCTCTTCACAGC |
| chr5 | 177238251 | 177238269 | 243 | AATAGAAGGCTTTCCTCTTC |
| chr5 | 177238256 | 177238274 | 244 | AACGGGCTAGAAGGCTTTCC |
| chr5 | 177238261 | 177238279 | 245 | AAGACCTCGGGCTAGAAGGC |
| chr5 | 177238266 | 177238284 | 246 | AAGATCGACCTCGGGCTAG |
| chr5 | 177238271 | 177238289 | 247 | AAGCACTAGATCGACCTCGG |
| chr5 | 177238276 | 177238294 | 248 | AATCTGAGCACTAGATCGAC |
| chr5 | 177238281 | 177238299 | 249 | AACTTGTTCTGAGCACTAGA |
| chr5 | 177238286 | 177238304 | 250 | AACCTGCTTGTTCTGAGCA |
| chr5 | 177238291 | 177238309 | 251 | AACGTCCACCTGCTTGTTCT |
| chr5 | 177238296 | 177238314 | 252 | AATTCTCGTCCACCTGCTT |
| chr5 | 177238301 | 177238319 | 253 | AAAGAATTCTCGTCCACC |
| chr5 | 177238306 | 177238324 | 254 | AAATCAAAGAATTCTCGT |
| chr5 | 177238311 | 177238329 | 255 | AAGGTTGAAATCAAAGAATT |
| chr5 | 177238316 | 177238334 | 256 | AATCTTTGGTTGAAATCAAA |
| chr5 | 177238321 | 177238339 | 257 | AAGCTCTTCTTTGGTTGAAA |
| chr5 | 177238326 | 177238344 | 258 | AATGGAGGCTCTTCTTTGGT |
| chr5 | 177238331 | 177238349 | 259 | AAGAACTGGAGGCTCTTCT |
| chr5 | 177238336 | 177238354 | 260 | AATTTCAAGAACTGGAGGCT |
| chr5 | 177238341 | 177238359 | 261 | AACTCCCTTTCAAGAACTGG |
| chr5 | 177238346 | 177238364 | 262 | AAGGAGCCTCCCTTTCAAGA |
| chr5 | 177238351 | 177238369 | 263 | AAAACGGAGCCTCCCTTT |
| chr5 | 177238356 | 177238374 | 264 | AACTCCAAAAACGGAGCCTC |
| chr5 | 177238361 | 177238379 | 265 | AAGGGCCCTCCAAAAACGGA |
| chr5 | 177238366 | 177238384 | 266 | AACCAAGGGGCCCTCCAAAA |
| chr5 | 177238374 | 177238392 | 267 | AATGACTGAGCCAAGGGGCC |
| chr5 | 177238379 | 177238397 | 268 | AAGTTCTGACTGAGCCAAG |
| chr5 | 177238384 | 177238402 | 269 | AACTCCAAGTTCTGACTGAG |
| chr5 | 177238389 | 177238407 | 270 | AATCCACCTCCAAGTTCTGA |
| chr5 | 177238394 | 177238412 | 271 | AAGCATGTCCACCTCCAAGT |
| chr5 | 177238399 | 177238417 | 272 | AACTCAGCATGTCCACCTC |
| chr5 | 177238404 | 177238422 | 273 | AACGGCAACTCAGCATGTCC |
| chr5 | 177238409 | 177238427 | 274 | AAGCTGCGGCAACTCAGCA |
| chr5 | 177238414 | 177238432 | 275 | AAGGTCAGCTGCGGCAACT |
| chr5 | 177238419 | 177238437 | 276 | AAGACAAGGTCAGCTGCGG |
| chr5 | 177238424 | 177238442 | 277 | AAGGCACAGACAAGGTCAGC |
| chr5 | 177238429 | 177238447 | 278 | AACCACAGGCACAGACAAGG |
| chr5 | 177238434 | 177238452 | 279 | AACGGAGCCACAGGCACAGA |
| chr5 | 177238439 | 177238457 | 280 | AACTTCCGGAGCCACAGGC |
| chr5 | 177238444 | 177238462 | 281 | AAGAGAGACTTCCGGAGCCA |
| chr5 | 177238449 | 177238467 | 282 | AACCGTGGAGAGACTTCCGG |
| chr5 | 177238454 | 177238472 | 283 | AAGCAGGCCGTGGAGAGACT |
| chr5 | 177238459 | 177238477 | 284 | AACAAGGGCAGGCCGTGGAG |
| chr5 | 177238464 | 177238482 | 285 | AAGACTCAAGGGCAGGCCG |
| chr5 | 177238469 | 177238487 | 286 | AATCCTCAGACTCAAGGGCA |
| chr5 | 177238474 | 177238492 | 287 | AAGCAATTCCTCAGACTCAA |
| chr5 | 177238479 | 177238497 | 288 | AACTAGCAATTCCTCAGA |
| chr5 | 177238484 | 177238502 | 289 | AAGTTTTAACTAGCAATTCC |
| chr5 | 177238487 | 177238505 | 290 | AAGGCGTTTTAACTAGCAAT |
| chr5 | 177238489 | 177238507 | 291 | AACTGGCGTTTTAACTAGCA |
| chr5 | 177238492 | 177238510 | 292 | AATACCTGGCGTTTTAACTA |
| chr5 | 177238497 | 177238515 | 293 | AACACCTTACCTGGCGTTTT |
| chr5 | 177238502 | 177238520 | 294 | AACCCCACCTTACCTGGC |
| chr5 | 177238507 | 177238525 | 295 | AACCCCAACCCCACCTTAC |
| chr5 | 177238512 | 177238530 | 296 | AACTGAGACCCCAACCCCAC |
| chr5 | 177238517 | 177238535 | 297 | AAATACTGAGACCCCAAC |
| chr5 | 177238522 | 177238540 | 298 | AATGCTCAAATACTGAGACC |
| chr5 | 177238527 | 177238545 | 299 | AATATCTGCTCAAATACTG |
| chr5 | 177238532 | 177238550 | 300 | AATAATCATATCTGCTCAAA |
| chr5 | 177238537 | 177238555 | 301 | AATCCTCTAATCATATCTGC |
| chr5 | 177238542 | 177238560 | 302 | AACTGCTTCCTCTAATCATA |
| chr5 | 177238547 | 177238565 | 303 | AATCTCCTGCTTCCTCTAA |
| chr5 | 177238552 | 177238570 | 304 | AACTAAAATCTCCTGCTTCC |
| chr5 | 177238557 | 177238575 | 305 | AACATACTAAAATCTCCTG |
| chr5 | 177238562 | 177238580 | 306 | AATCAAAACATACTAAAATC |
| chr5 | 177238567 | 177238585 | 307 | AATTACATCAAAACATACTA |
| chr5 | 177238572 | 177238590 | 308 | AATGGCTTTACATCAAAACA |
| chr5 | 177238577 | 177238595 | 309 | AATGTTGGCTTTACATCA |
| chr5 | 177238582 | 177238600 | 310 | AAGATACAATGTTGGCTTTA |
| chr5 | 177238587 | 177238605 | 311 | AATATAGATACAATGTTGG |
| chr5 | 177238592 | 177238610 | 312 | AATTGTATATAGATACAAT |
| chr5 | 177238597 | 177238615 | 313 | AAGTTTATTGTATATAGAT |
| chr5 | 177238602 | 177238620 | 314 | AAGGGGTAGTTTATTGTATA |
| chr5 | 177238504 | 177238522 | 315 | AACCAACCCCACCTTACCTG |
| chr5 | 177238538 | 177238556 | 316 | AATTCCTCTAATCATATCTG |
| chr5 | 177238537 | 177238556 | 317 | AATTCCTCTAATCATATCTGC |
| chr5 | 177238536 | 177238556 | 318 | AATTCCTCTAATCATATCTGCT |
| chr5 | 177238538 | 177238558 | 319 | AAGCTTCCTCTAATCATATCTG |
| chr5 | 177238536 | 177238554 | 320 | AACCTCTAATCATATCTGCT |
| chr5 | 177238535 | 177238555 | 321 | AATCCTCTAATCATATCTGCTC |

Alternative splicing events in PKD1, ABCA4, FUS, CEL, or NSD1 gene can lead to non-productive mRNA transcripts which in turn can lead to aberrant protein expression, and therapeutic agents which can target the alternative splicing events in PKD1, ABCA4, FUS, CEL, or NSD1 gene can modulate the expression level of functional proteins in DS patients and/or inhibit aberrant protein expression. Such therapeutic agents can be used to treat a condition caused by polycystin-1, ATP binding cassette subfamily A member 4, FUS RNA binding protein, carboxyl ester lipase, or nuclear receptor binding SET domain protein 1 protein deficiency.

One of the alternative splicing events that can lead to non-productive mRNA transcripts is the inclusion of an extra exon in the mRNA transcript that can induce non-sense mediated mRNA decay. The present disclosure provides compositions and methods for modulating alternative splicing of PKD1, ABCA4, FUS, CEL, or NSD1 to increase the production of protein-coding mature mRNA, and thus, translated functional polycystin-1, ATP binding cassette subfamily A member 4, FUS RNA binding protein, carboxyl ester lipase, or nuclear receptor binding SET domain protein 1 protein. These compositions and methods include antisense oligomers (ASOs) that can cause exon skipping, e.g., pseudoexon skipping, and promote constitutive splicing of PKD1, ABCA4, FUS, CEL, or NSD1 pre-mRNA. In various embodiments, functional polycystin-1, ATP binding cassette subfamily A member 4, FUS RNA binding protein, carboxyl ester lipase, or nuclear receptor binding SET domain protein 1 protein can be increased using the methods of the disclosure to treat a condition caused by polycystin-1, ATP binding cassette subfamily A member 4, FUS RNA binding protein, carboxyl ester lipase, or nuclear receptor binding SET domain protein 1 protein deficiency.

### Target Transcripts

In some embodiments, the methods of the present disclosure exploit the presence of ASCE in the pre-mRNA transcribed from PKD1, ABCA4, FUS, CEL, or NSD1 genes. Splicing of the identified PKD1, ABCA4, FUS, CEL, or NSD1 ASCE pre-mRNA species to produce functional mature PKD1, ABCA4, FUS, CEL, or NSD1 mRNA may be induced using a therapeutic agent such as an ASO that stimulates exon skipping of an ASCE. Induction of exon skipping may result in inhibition of an NMD pathway. The resulting mature PKD1, ABCA4, FUS, CEL, or NSD1 mRNA can be translated normally without activating NMD pathway, thereby increasing the amount of polycystin-1, ATP binding cassette subfamily A member 4, FUS RNA binding protein, carboxyl ester lipase, or nuclear receptor binding SET domain protein 1 protein in the patient's cells and alleviating symptoms of a condition or disease associated with polycystin-1, ATP binding cassette subfamily A member 4, FUS RNA binding protein, carboxyl ester lipase, or nuclear receptor binding SET domain protein 1 deficiency, such as Polycystic Kidney Disease 1 with or without Polycystic Liver Disease; Autosomal Dominant Polycystic Kidney Disease; Age-related macular degeneration-2; Stargardt Disease 1; Amyotrophic Lateral Sclerosis; Amyotrophic Lateral Sclerosis 6 with or without Frontotemporal Dementia; Tremor, Hereditary Essential, 4; Frontotemporal Dementia; Maturity-Onset Diabetes Of The Young, Type 8, with Exocrine Dysfunction; Maturity-Onset Diabetes Of The Young; Sotos Syndrome 1; or Beckwith-Wiedemann Syndrome.

In various embodiments, the present disclosure provides a therapeutic agent which can target PKD1, ABCA4, FUS, CEL, or NSD1 mRNA transcripts to modulate splicing or protein expression level. The therapeutic agent can be a small molecule, polynucleotide, or polypeptide. In some embodiments, the therapeutic agent is an ASO. Various regions or sequences on the PKD1, ABCA4, FUS, CEL, or NSD1 pre-mRNA can be targeted by a therapeutic agent, such as an ASO. In some embodiments, the ASO targets a PKD1, ABCA4, FUS, CEL, or NSD1 pre-mRNA transcript containing an ASCE. In some embodiments, the ASO targets a sequence within an ASCE of a PKD1, ABCA4, FUS, CEL, or NSD1 pre-mRNA transcript. In some embodiments, the ASO targets a sequence upstream (or 5′) from the 5′ end of an ASCE (3′ss) of a PKD1, ABCA4, FUS, CEL, or NSD1 pre-mRNA transcript. In some embodiments, the ASO targets a sequence downstream (or 3′) from the 3′ end of an ASCE (5′ss) of a PKD1, ABCA4, FUS, CEL, or NSD1 pre-mRNA transcript. In some embodiments, the ASO targets a sequence that is within an intron flanking on the 5′ end of the ASCE of a PKD1, ABCA4, FUS, CEL, or NSD1 pre-mRNA transcript. In some embodiments, the ASO targets a sequence that is within an intron flanking the 3′ end of the ASCE of a PKD1, ABCA4, FUS, CEL, or NSD1 pre-mRNA transcript. In some embodiments, the ASO targets a sequence comprising an ASCE-intron boundary of a PKD1, ABCA4, FUS, CEL, or NSD1 pre-mRNA transcript. An ASCE-intron boundary can refer to the junction of an intron sequence and an ASCE region. The intron sequence can flank the 5′ end of the ASCE, or the 3′ end of the ASCE. In some embodiments, the ASO targets a sequence within an exon of a PKD1, ABCA4, FUS, CEL, or NSD1 pre-mRNA transcript. In some embodiments, the ASO targets a sequence within an intron of a PKD1, ABCA4, FUS, CEL, or NSD1 pre-mRNA transcript. In some embodiments, the ASO targets a sequence comprising both a portion of an intron and a portion of an exon of a PKD1, ABCA4, FUS, CEL, or NSD1 pre-mRNA transcript.

In some embodiments, the ASO targets a sequence about 4 to about 300 nucleotides upstream (or 5′) from the 5′ end of the ASCE. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 5′ end of the ASCE region. In some embodiments, the ASO may target a sequence more than 300 nucleotides upstream from the 5′ end of the ASCE. In some embodiments, the ASO targets a sequence about 4 to about 300 nucleotides downstream (or 3′) from the 3′ end of the ASCE. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 3′ end of the ASCE. In some embodiments, the ASO targets a sequence more than 300 nucleotides downstream from the 3′ end of the ASCE.

In some embodiments, the PKD1, ABCA4, FUS, CEL, or NSD1 ASCE-containing pre-mRNA transcript is encoded by a genetic sequence with at least about 80%, 85%, 90%, 95%, 96%, 97%, 98%, 99% or 100% sequence identity to any one of SEQ ID NOS: 1-5. In some embodiments, the PKD1, ABCA4, FUS, CEL, or NSD1 ASCE pre-mRNA transcript comprises a sequence with at least about 80%, 85%, 90%, 95%, 96%, 97%, 98%, 99% or 100% sequence identity to any one of SEQ ID NOS: 6-10.

In some embodiments, the PKD1, ABCA4, FUS, CEL, or NSD1 ASCE-containing pre-mRNA transcript comprises a sequence with at least about 80%, 85%, 90%, 95%, 97%, or 100% sequence identity to any one of SEQ ID NOS: 6-10. In some embodiments, PKD1, ABCA4, FUS, CEL, or NSD1 ASCE-containing pre-mRNA transcript is encoded by a sequence with at least about 80%, 85%, 90%, 95%, 97%, or 100% sequence identity to any one of SEQ ID NOS: 1-5. In some embodiments, the targeted portion of the PKD1, ABCA4, FUS, CEL, or NSD1 ASCE-containing pre-mRNA comprises a sequence with at least 80%, 85%, 90%, 95%, 97%, or 100% sequence identity to a region comprising at least 8 contiguous nucleic acids of any one of SEQ ID NOS: 6-10.

In some embodiments, the ASO targets an intron upstream of the ASCE, an intron downstream of the ASCE, an exon upstream of the ASCE, an exon downstream of the ASCE or within the ASCE of ASCE-containing pre-mRNA. In some embodiments, the ASO targets an intron upstream of the ASCE, an intron downstream of the ASCE, an exon upstream of the ASCE, an exon downstream of the ASCE or within the ASCE of a PKD1, ABCA4, FUS, CEL or NSD1 ASCE-containing pre-mRNA.

In some embodiments, the ASO targets a sequence at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of the ASCE. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of the ASCE. In some embodiments, the ASO targets a sequence at about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of the ASCE.

In some embodiments, the ASO targets a sequence at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of the ASCE. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of the ASCE. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of the ASCE.

In some embodiments, the ASO targets a PKD1 ASCE-containing pre-mRNA, wherein the ASCE is exon 38 of PKD1. In some embodiments, the ASO targets a PKD1 ASCE-containing pre-mRNA, wherein the ASCE is exon GRCh38/hg38: chr16 2092954 2093093 of PKD1. In some embodiments, the ASO targets a PKD1 ASCE-containing pre-mRNA, wherein the ASCE comprises a sequence with at least about 80%, 85%, 90%, 95%, 97%, or 100% sequence identity to SEQ ID NO: 11. In some embodiments, the ASO targets an intron upstream of the ASCE, an intron downstream of the ASCE, an exon upstream of the ASCE, an exon downstream of the ASCE or within the ASCE of a PKD1 ASCE-containing pre-mRNA. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr16 2092954 of PKD1. In some embodiments, the ASO targets a sequence at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr16 2093093 of PKD1. In some embodiments, the ASO targets a sequence within GRCh38/hg38: chr16 2092954 2093093 of PKD1.

In some embodiments, the ASO targets a ABCA4 ASCE-containing pre-mRNA, wherein the ASCE is exon 3 of ABCA4. In some embodiments, the ASO targets a ABCA4 ASCE-containing pre-mRNA, wherein the ASCE is exon GRCh38/hg38: chr1 94111438 94111579 of ABCA4. In some embodiments, the ASO targets a ABCA4 ASCE-containing pre-mRNA, wherein the ASCE comprises a sequence with at least about 80%, 85%, 90%, 95%, 97%, or 100% sequence identity to SEQ ID NO: 12. In some embodiments, the ASO targets an intron upstream of the ASCE, an intron downstream of the ASCE, an exon upstream of the ASCE, an exon downstream of the ASCE or within the ASCE of a ABCA4 ASCE-containing pre-mRNA. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr1 94111438 of ABCA4. In some embodiments, the ASO targets a sequence at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr1 94111579 of ABCA4. In some embodiments, the ASO targets a sequence within GRCh38/hg38: chr1 94111438 94111579 of ABCA4.

In some embodiments, the ASO targets a FUS ASCE-containing pre-mRNA, wherein the ASCE is exon 7 of FUS. In some embodiments, the ASO targets a FUS ASCE-containing pre-mRNA, wherein the ASCE is exon GRCh38/hg38: chr16 31186802 31186836 of FUS. In some embodiments, the ASO targets a FUS ASCE-containing pre-mRNA, wherein the ASCE comprises a sequence with at least about 80%, 85%, 90%, 95%, 97%, or 100% sequence identity to SEQ ID NO: 13. In some embodiments, the ASO targets an intron upstream of the ASCE, an intron downstream of the ASCE, an exon upstream of the ASCE, an exon downstream of the ASCE or within the ASCE of a FUS ASCE-containing pre-mRNA. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr16 31186802 of FUS. In some embodiments, the ASO targets a sequence at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr16 31186836 of FUS. In some embodiments, the ASO targets a sequence within GRCh38/hg38: chr16 31186802 31186836 of FUS.

In some embodiments, the ASO targets a CEL ASCE-containing pre-mRNA, wherein the ASCE is exon 5 of CEL. In some embodiments, the ASO targets a CEL ASCE-containing pre-mRNA, wherein the ASCE is exon GRCh38/hg38: chr9 133066530 133066660 of CEL. In some embodiments, the ASO targets a CEL ASCE-containing pre-mRNA, wherein the ASCE comprises a sequence with at least about 80%, 85%, 90%, 95%, 97%, or 100% sequence identity to SEQ ID NO: 14. In some embodiments, the ASO targets an intron upstream of the ASCE, an intron downstream of the ASCE, an exon upstream of the ASCE, an exon downstream of the ASCE or within the ASCE of a CEL ASCE-containing pre-mRNA. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr9 133066530 of CEL. In some embodiments, the ASO targets a sequence at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr9 133066660 of CEL. In some embodiments, the ASO targets a sequence within GRCh38/hg38: chr9 133066530 133066660 of CEL.

In some embodiments, the ASO targets a NSD1 ASCE-containing pre-mRNA, wherein the ASCE is exon 8 of NSD1. In some embodiments, the ASO targets a NSD1 ASCE-containing pre-mRNA, wherein the ASCE is exon GRCh38/hg38: chr5 177238237 177238507 of NSD1. In some embodiments, the ASO targets a NSD1 ASCE-containing pre-mRNA, wherein the ASCE comprises a sequence with at least about 80%, 85%, 90%, 95%, 97%, or 100% sequence identity to SEQ ID NO: 15. In some embodiments, the ASO targets an intron upstream of the ASCE, an intron downstream of the ASCE, an exon upstream of the ASCE, an exon downstream of the ASCE or within the ASCE of a NSD1 ASCE-containing pre-mRNA. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr5 177238237 of NSD1. In some embodiments, the ASO targets a sequence at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr5 177238507 of NSD. In some embodiments, the ASO targets a sequence within GRCh38/hg38: chr5 177238237 177238507 of NSD1.

In some embodiments, the ASO comprises a sequence complementary to the targeted portion of the ASCE-containing pre-mRNA encoded by a gene having a sequence with at least about 80%, 85%, 90%, 95%, 97%, or 100% sequence identity to any one of SEQ ID NOS: 1-5. In some embodiments, the ASO comprises a sequence complementary to the targeted portion of the ASCE-containing pre-mRNA having a sequence with at least about 80%, 85%, 90%, 95%, 97%, or 100% sequence identity to any one of SEQ ID NOS: 6-10. In some embodiments, the ASO comprises a sequence complementary to the targeted portion of the ASCE having a sequence with at least about 80%, 85%, 90%, 95%, 97%, or 100% sequence identity to any one of SEQ ID NOS: 11-15. In some embodiments, the ASO comprises a sequence with at least about 80%, 85%, 90%, 95%, 97%, or 100% sequence identity to any one of SEQ ID NOS: 16-309. In some embodiments, the ASO comprises a sequence with at least about 80%, 85%, 90%, 95%, 97%, or 100% sequence identity to the reverse complement sequence of any one of SEQ ID NOS: 16-309. In some embodiments, the ASO comprises a sequence with at least about 80%, 85%, 90%, 95%, 97%, or 100% sequence identity to the complement sequence of any one of SEQ ID NOS: 16-309. In some embodiments, the ASO comprises a sequence with at least about 80%, 85%, 90%, 95%, 97%, or 100% sequence identity to a sequence of any one of SEQ ID NOS: 16-309 in which each T is U.

In some embodiments, the ASO targets a sequence upstream from the 5′ end of an ASCE.

In some embodiments, the ASOs target a sequence containing an exon-intron boundary (or junction). In some embodiments, the ASOs do not target a sequence containing an exon-intron boundary (or junction). In some embodiments, the ASOs target a sequence downstream from the 3′ end of an ASCE. In some embodiments, ASOs target a sequence within an ASCE.

### Protein Expression

In some embodiments, the methods described herein are used to increase the production of a functional polycystin-1, ATP binding cassette subfamily A member 4, FUS RNA binding protein, carboxyl ester lipase, or nuclear receptor binding SET domain protein 1 protein or RNA. As used herein, the term “functional” refers to the amount of activity or function of a polycystin-1, ATP binding cassette subfamily A member 4, FUS RNA binding protein, carboxyl ester lipase, or nuclear receptor binding SET domain protein 1 protein or RNA that is necessary to eliminate any one or more symptoms of a treated condition or disease, e.g., Polycystic Kidney Disease 1 with or without Polycystic Liver Disease; Autosomal Dominant Polycystic Kidney Disease; Age-related macular degeneration-2; Stargardt Disease 1; Amyotrophic Lateral Sclerosis; Amyotrophic Lateral Sclerosis 6 with or without Frontotemporal Dementia; Tremor, Hereditary Essential, 4; Frontotemporal Dementia; Maturity-Onset Diabetes Of The Young, Type 8, with Exocrine Dysfunction; Maturity-Onset Diabetes Of The Young; Sotos Syndrome 1; or Beckwith-Wiedemann Syndrome. In some embodiments, the methods are used to increase the production of a partially functional polycystin-1, ATP binding cassette subfamily A member 4, FUS RNA binding protein, carboxyl ester lipase, or nuclear receptor binding SET domain protein 1 protein or RNA. As used herein, the term “partially functional” refers to any amount of activity or function of the polycystin-1, ATP binding cassette subfamily A member 4, FUS RNA binding protein, carboxyl ester lipase, or nuclear receptor binding SET domain protein 1 protein or RNA that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the polycystin-1, ATP binding cassette subfamily A member 4, FUS RNA binding protein, carboxyl ester lipase, or nuclear receptor binding SET domain protein 1 protein by cells of a subject having a ASCE-containing pre-mRNA encoding the polycystin-1, ATP binding cassette subfamily A member 4, FUS RNA binding protein, carboxyl ester lipase, or nuclear receptor binding SET domain protein 1 protein, wherein the subject has Polycystic Kidney Disease 1 with or without Polycystic Liver Disease; Autosomal Dominant Polycystic Kidney Disease; Age-related macular degeneration-2; Stargardt Disease 1; Amyotrophic Lateral Sclerosis; Amyotrophic Lateral Sclerosis 6 with or without Frontotemporal Dementia; Tremor, Hereditary Essential, 4; Frontotemporal Dementia; Maturity-Onset Diabetes Of The Young, Type 8, with Exocrine Dysfunction; Maturity-Onset Diabetes Of The Young; Sotos Syndrome 1; or Beckwith-Wiedemann Syndrome caused by a deficient amount of activity of polycystin-1, ATP binding cassette subfamily A member 4, FUS RNA binding protein, carboxyl ester lipase, or nuclear receptor binding SET domain protein 1 protein, and wherein the deficient amount of the polycystin-1, ATP binding cassette subfamily A member 4, FUS RNA binding protein, carboxyl ester lipase, or nuclear receptor binding SET domain protein 1 protein is caused by haploinsufficiency of the polycystin-1, ATP binding cassette subfamily A member 4, FUS RNA binding protein, carboxyl ester lipase, or nuclear receptor binding SET domain protein 1 protein. In such an embodiment, the subject has a first allele encoding a functional polycystin-1, ATP binding cassette subfamily A member 4, FUS RNA binding protein, carboxyl ester lipase, or nuclear receptor binding SET domain protein 1 protein, and a second allele from which the polycystin-1, ATP binding cassette subfamily A member 4, FUS RNA binding protein, carboxyl ester lipase, or nuclear receptor binding SET domain protein 1 protein is not produced. In another such embodiment, the subject has a first allele encoding a functional polycystin-1, ATP binding cassette subfamily A member 4, FUS RNA binding protein, carboxyl ester lipase, or nuclear receptor binding SET domain protein 1 protein, and a second allele encoding a nonfunctional polycystin-1, ATP binding cassette subfamily A member 4, FUS RNA binding protein, carboxyl ester lipase, or nuclear receptor binding SET domain protein 1 protein. In another such embodiment, the subject has a first allele encoding a functional polycystin-1, ATP binding cassette subfamily A member 4, FUS RNA binding protein, carboxyl ester lipase, or nuclear receptor binding SET domain protein 1 protein, and a second allele encoding a partially functional polycystin-1, ATP binding cassette subfamily A member 4, FUS RNA binding protein, carboxyl ester lipase, or nuclear receptor binding SET domain protein 1 protein. In any of these embodiments, the antisense oligomer binds to a targeted portion of the ASCE-containing pre-mRNA transcribed from the second allele, thereby inhibiting or reducing exon skipping of the ASCE from the pre-mRNA or promoting inclusion of the ASCE in a mature RNA processed from the ASCE-containing pre-mRNA, and causing an increase in the level of mature mRNA encoding functional polycystin-1, ATP binding cassette subfamily A member 4, FUS RNA binding protein, carboxyl ester lipase, or nuclear receptor binding SET domain protein 1 protein, and an increase in the expression of the polycystin-1, ATP binding cassette subfamily A member 4, FUS RNA binding protein, carboxyl ester lipase, or nuclear receptor binding SET domain protein 1 protein in the cells of the subject.

In some embodiments, the method is a method of increasing the expression of the polycystin-1, ATP binding cassette subfamily A member 4, FUS RNA binding protein, carboxyl ester lipase, or nuclear receptor binding SET domain protein 1 protein by cells of a subject having a ASCE-containing pre-mRNA encoding the polycystin-1, ATP binding cassette subfamily A member 4, FUS RNA binding protein, carboxyl ester lipase, or nuclear receptor binding SET domain protein 1 protein, wherein the subject has Polycystic Kidney Disease 1 with or without Polycystic Liver Disease; Autosomal Dominant Polycystic Kidney Disease; Age-related macular degeneration-2; Stargardt Disease 1; Amyotrophic Lateral Sclerosis; Amyotrophic Lateral Sclerosis 6 with or without Frontotemporal Dementia; Tremor, Hereditary Essential, 4; Frontotemporal Dementia; Maturity-Onset Diabetes Of The Young, Type 8, with Exocrine Dysfunction; Maturity-Onset Diabetes Of The Young; Sotos Syndrome 1; or Beckwith-Wiedemann Syndrome caused by a deficient amount of activity of polycystin-1, ATP binding cassette subfamily A member 4, FUS RNA binding protein, carboxyl ester lipase, or nuclear receptor binding SET domain protein 1 protein, and wherein the deficient amount of the polycystin-1, ATP binding cassette subfamily A member 4, FUS RNA binding protein, carboxyl ester lipase, or nuclear receptor binding SET domain protein 1 protein is caused by autosomal recessive inheritance.

In some embodiments, the method is a method of increasing the expression of the polycystin-1, ATP binding cassette subfamily A member 4, FUS RNA binding protein, carboxyl ester lipase, or nuclear receptor binding SET domain protein 1 protein by cells of a subject having a ASCE-containing pre-mRNA encoding the polycystin-1, ATP binding cassette subfamily A member 4, FUS RNA binding protein, carboxyl ester lipase, or nuclear receptor binding SET domain protein 1 protein, wherein the subject has Polycystic Kidney Disease 1 with or without Polycystic Liver Disease; Autosomal Dominant Polycystic Kidney Disease; Age-related macular degeneration-2; Stargardt Disease 1; Amyotrophic Lateral Sclerosis; Amyotrophic Lateral Sclerosis 6 with or without Frontotemporal Dementia; Tremor, Hereditary Essential, 4; Frontotemporal Dementia; Maturity-Onset Diabetes Of The Young, Type 8, with Exocrine Dysfunction; Maturity-Onset Diabetes Of The Young; Sotos Syndrome 1; or Beckwith-Wiedemann Syndrome caused by a deficient amount of activity of polycystin-1, ATP binding cassette subfamily A member 4, FUS RNA binding protein, carboxyl ester lipase, or nuclear receptor binding SET domain protein 1 protein, and wherein the deficient amount of the polycystin-1, ATP binding cassette subfamily A member 4, FUS RNA binding protein, carboxyl ester lipase, or nuclear receptor binding SET domain protein 1 protein is caused by autosomal dominant inheritance.

In some embodiments, the method is a method of increasing the expression of the polycystin-1, ATP binding cassette subfamily A member 4, FUS RNA binding protein, carboxyl ester lipase, or nuclear receptor binding SET domain protein 1 protein by cells of a subject having a ASCE-containing pre-mRNA encoding the polycystin-1, ATP binding cassette subfamily A member 4, FUS RNA binding protein, carboxyl ester lipase, or nuclear receptor binding SET domain protein 1 protein, wherein the subject has Polycystic Kidney Disease 1 with or without Polycystic Liver Disease; Autosomal Dominant Polycystic Kidney Disease; Age-related macular degeneration-2; Stargardt Disease 1; Amyotrophic Lateral Sclerosis; Amyotrophic Lateral Sclerosis 6 with or without Frontotemporal Dementia; Tremor, Hereditary Essential, 4; Frontotemporal Dementia; Maturity-Onset Diabetes Of The Young, Type 8, with Exocrine Dysfunction; Maturity-Onset Diabetes Of The Young; Sotos Syndrome 1; or Beckwith-Wiedemann Syndrome caused by a deficient amount of activity of polycystin-1, ATP binding cassette subfamily A member 4, FUS RNA binding protein, carboxyl ester lipase, or nuclear receptor binding SET domain protein 1 protein, and wherein the deficient amount of the polycystin-1, ATP binding cassette subfamily A member 4, FUS RNA binding protein, carboxyl ester lipase, or nuclear receptor binding SET domain protein 1 protein is caused by X-linked dominant inheritance.

In related embodiments, the method is a method of using an ASO to increase the expression of a protein or functional RNA. In some embodiments, an ASO may be used to increase the expression of polycystin-1, ATP binding cassette subfamily A member 4, FUS RNA binding protein, carboxyl ester lipase, or nuclear receptor binding SET domain protein 1 protein in cells of a subject having a ASCE-containing pre-mRNA encoding polycystin-1, ATP binding cassette subfamily A member 4, FUS RNA binding protein, carboxyl ester lipase, or nuclear receptor binding SET domain protein 1 protein, wherein the subject has a deficiency, e.g., Polycystic Kidney Disease 1 with or without Polycystic Liver Disease; Autosomal Dominant Polycystic Kidney Disease; Age-related macular degeneration-2; Stargardt Disease 1; Amyotrophic Lateral Sclerosis; Amyotrophic Lateral Sclerosis 6 with or without Frontotemporal Dementia; Tremor, Hereditary Essential, 4; Frontotemporal Dementia; Maturity-Onset Diabetes Of The Young, Type 8, with Exocrine Dysfunction; Maturity-Onset Diabetes Of The Young; Sotos Syndrome 1; or Beckwith-Wiedemann Syndrome, in the amount or function of a polycystin-1, ATP binding cassette subfamily A member 4, FUS RNA binding protein, carboxyl ester lipase, or nuclear receptor binding SET domain protein 1 protein.

In some embodiments, the ASCE-containing pre-mRNA transcript that encodes the protein that is causative of the disease or condition is targeted by the ASOs described herein. In some embodiments, a ASCE-containing pre-mRNA transcript that encodes a protein that is not causative of the disease is targeted by the ASOs. For example, a disease that is the result of a mutation or deficiency of a first protein in a particular pathway may be ameliorated by targeting a ASCE-containing pre-mRNA that encodes a second protein, thereby increasing production of the second protein. In some embodiments, the function of the second protein is able to compensate for the mutation or deficiency of the first protein (which is causative of the disease or condition).

In some embodiments, the subject has:
(a) a first mutant allele from which
   (i) the polycystin-1, ATP binding cassette subfamily A member 4, FUS RNA binding protein, carboxyl ester lipase, or nuclear receptor binding SET domain protein 1 protein is produced at a reduced level compared to production from a wild-type allele,
   (ii) the polycystin-1, ATP binding cassette subfamily A member 4, FUS RNA binding protein, carboxyl ester lipase, or nuclear receptor binding SET domain protein 1 protein is produced in a form having reduced function compared to an equivalent wild-type protein, or
   (iii) the polycystin-1, ATP binding cassette subfamily A member 4, FUS RNA binding protein, carboxyl ester lipase, or nuclear receptor binding SET domain protein 1 protein or functional RNA is not produced; and
(b) a second mutant allele from which
   (i) the polycystin-1, ATP binding cassette subfamily A member 4, FUS RNA binding protein, carboxyl ester lipase, or nuclear receptor binding SET domain protein 1 protein is produced at a reduced level compared to production from a wild-type allele,
   (ii) the polycystin-1, ATP binding cassette subfamily A member 4, FUS RNA binding protein, carboxyl ester lipase, or nuclear receptor binding SET domain protein 1 protein is produced in a form having reduced function compared to an equivalent wild-type protein, or
   (iii) the polycystin-1, ATP binding cassette subfamily A member 4, FUS RNA binding protein, carboxyl ester lipase, or nuclear receptor binding SET domain protein 1 protein is not produced, and

wherein the ASCE-containing pre-mRNA is transcribed from the first allele and/or the second allele. In these embodiments, the ASO binds to a targeted portion of the ASCE-containing pre-mRNA transcribed from the first allele or the second allele, thereby promoting exon inclusion of the ASCE in a processed mRNA processed from the ASCE-containing pre-mRNA, and causing an increase in the level of mRNA encoding polycystin-1, ATP binding cassette subfamily A member 4, FUS RNA binding protein, carboxyl ester lipase, or nuclear receptor binding SET domain protein 1 protein and an increase in the expression of the target protein or functional RNA in the cells of the subject. In these embodiments, the target protein or functional RNA having an increase in expression level resulting from the reduction or inhibition of exon skipping of the ASCE from the ASCE-containing pre-mRNA may be either in a form having reduced function compared to the equivalent wild-type protein (partially functional), or having full function compared to the equivalent wild-type protein (fully functional).

In some embodiments, the level of mRNA encoding polycystin-1, ATP binding cassette subfamily A member 4, FUS RNA binding protein, carboxyl ester lipase, or nuclear receptor binding SET domain protein 1 protein is increased 1.1 to 10-fold, when compared to the amount of mRNA encoding polycystin-1, ATP binding cassette subfamily A member 4, FUS RNA binding protein, carboxyl ester lipase, or nuclear receptor binding SET domain protein 1 protein that is produced in a control cell, e.g., one that is not treated with the antisense oligomer or one that is treated with an antisense oligomer that does not bind to the targeted portion of the PKD1, ABCA4, FUS, CEL, or NSD1 ASCE-containing pre-mRNA.

In some embodiments, a subject treated using the methods of the present disclosure expresses a partially functional polycystin-1, ATP binding cassette subfamily A member 4, FUS RNA binding protein, carboxyl ester lipase, or nuclear receptor binding SET domain protein 1 protein from one allele, wherein the partially functional polycystin-1, ATP binding cassette subfamily A member 4, FUS RNA binding protein, carboxyl ester lipase, or nuclear receptor binding SET domain protein 1 protein may be caused by a frameshift mutation, a nonsense mutation, a missense mutation, or a partial gene deletion. In some embodiments, a subject treated using the methods of the disclosure expresses a nonfunctional polycystin-1, ATP binding cassette subfamily A member 4, FUS RNA binding protein, carboxyl ester lipase, or nuclear receptor binding SET domain protein 1 protein from one allele, wherein the nonfunctional polycystin-1, ATP binding cassette subfamily A member 4, FUS RNA binding protein, carboxyl ester lipase, or nuclear receptor binding SET domain protein 1 protein may be caused by a frameshift mutation, a nonsense mutation, a missense mutation, a partial gene deletion, in one allele. In some embodiments, a subject treated using the methods of the disclosure has a PKD1, ABCA4, FUS, CEL, or NSD1 whole gene deletion, in one allele.

### Exon Inclusion

As used herein, an “ASCE-containing pre-mRNA” is a pre-mRNA transcript that contains at least one alternatively-spliced coding exon. Alternative or aberrant splicing can result in exclusion of the at least one ASC in the mature mRNA transcripts. The terms “mature mRNA,” and “fully spliced mRNA,” are used interchangeably herein to describe a fully processed mRNA. Inclusion of the at least one pseudo-exon can be non-productive mRNA and lead to NMD of the mature mRNA. ASCE-containing mature mRNA may sometimes lead to aberrant protein expression.

In some embodiments, the included pseudo-exon is the most abundant pseudo-exon in a population of ASCE-containing pre-mRNAs transcribed from the gene encoding the target protein in a cell. In some embodiments, the included pseudo-exon is the most abundant pseudo-exon in a population of ASCE-containing pre-mRNAs transcribed from the gene encoding the target protein in a cell, wherein the population of ASCE-containing pre-mRNAs comprises two or more included pseudo-exons. In some embodiments, an antisense oligomer targeted to the most abundant pseudo-exon in the population of ASCE-containing pre-mRNAs encoding the target protein induces exon skipping of one or two or more pseudo-exons in the population, including the pseudo-exon to which the antisense oligomer is targeted or binds. In some embodiments, the targeted region is in a pseudo-exon that is the most abundant pseudo-exon in an ASCE-containing pre-mRNA encoding the polycystin-1, ATP binding cassette subfamily A member 4, FUS RNA binding protein, carboxyl ester lipase, or nuclear receptor binding SET domain protein 1 protein.

The degree of exon inclusion can be expressed as percent exon inclusion, e.g., the percentage of transcripts in which a given pseudo-exon is included. In brief, percent exon inclusion can be calculated as the percentage of the amount of RNA transcripts with the exon inclusion, over the sum of the average of the amount of RNA transcripts with exon inclusion plus the average of the amount of RNA transcripts with exon exclusion.

In some embodiments, an ASCE is an exon that is identified as an ASCE based on a determination of at least about 5%, at least about 10%, at least about 15%, at least about 20%, at least about 25%, at least about 30%, at least about 35%, at least about 40%, at least about 45%, or at least about 50%, exclusion. In embodiments, an ASCE is an exon that is identified as an ASCE based on a determination of about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 15% to about 100%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, or about 25% to about 35%, exclusion. ENCODE data (described by, e.g., Tilgner, et al., 2012, “Deep sequencing of subcellular RNA fractions shows splicing to be predominantly co-transcriptional in the human genome but inefficient for Inc RNAs,” Genome Research 22(9):1616-25) can be used to aid in identifying exon inclusion or exclusion.

In some embodiments, contacting cells with an ASO that is complementary to a targeted portion of a PKD1, ABCA4, FUS, CEL, or NSD1 pre-mRNA transcript results in an increase in the amount of polycystin-1, ATP binding cassette subfamily A member 4, FUS RNA binding protein, carboxyl ester lipase, or nuclear receptor binding SET domain protein 1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of polycystin-1, ATP binding cassette subfamily A member 4, FUS RNA binding protein, carboxyl ester lipase, or nuclear receptor binding SET domain protein 1 protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of polycystin-1, ATP binding cassette subfamily A member 4, FUS RNA binding protein, carboxyl ester lipase, or nuclear receptor binding SET domain protein 1 protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a PKD1, ABCA4, FUS, CEL, or NSD1 pre-mRNA transcript results in an increase in the amount of PKD1, ABCA4, FUS, CEL, or NSD1 mRNA including the mature mRNA encoding the target protein. In some embodiments, the amount of mRNA encoding polycystin-1, ATP binding cassette subfamily A member 4, FUS RNA binding protein, carboxyl ester lipase, or nuclear receptor binding SET domain protein 1 protein, or the mature mRNA encoding the polycystin-1, ATP binding cassette subfamily A member 4, FUS RNA binding protein, carboxyl ester lipase, or nuclear receptor binding SET domain protein 1 protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding polycystin-1, ATP binding cassette subfamily A member 4, FUS RNA binding protein, carboxyl ester lipase, or nuclear receptor binding SET domain protein 1 protein, or the mature mRNA encoding polycystin-1, ATP binding cassette subfamily A member 4, FUS RNA binding protein, carboxyl ester lipase, or nuclear receptor binding SET domain protein 1 protein produced in the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding polycystin-1, ATP binding cassette subfamily A member 4, FUS RNA binding protein, carboxyl ester lipase, or nuclear receptor binding SET domain protein 1 protein, or the mature mRNA encoding polycystin-1, ATP binding cassette subfamily A member 4, FUS RNA binding protein, carboxyl ester lipase, or nuclear receptor binding SET domain protein 1 protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the PKD1, ABCA4, FUS, CEL, or NSD1 ASCE-containing pre-mRNA.

The ASCE can be in any length. The ASCE can comprise a canonical exon. The ASCE can comprise a full sequence of a canonical exon. In some embodiments, the ASCE can be from 5 nucleotides to 10 nucleotides in length, from 10 nucleotides to 15 nucleotides in length, from 15 nucleotides to 20 nucleotides in length, from 20 nucleotides to 25 nucleotides in length, from 25 nucleotides to 30 nucleotides in length, from 30 nucleotides to 35 nucleotides in length, from 35 nucleotides to 40 nucleotides in length, from 40 nucleotides to 45 nucleotides in length, from 45 nucleotides to 50 nucleotides in length, from 50 nucleotides to 55 nucleotides in length, from 55 nucleotides to 60 nucleotides in length, from 60 nucleotides to 65 nucleotides in length, from 65 nucleotides to 70 nucleotides in length, from 70 nucleotides to 75 nucleotides in length, from 75 nucleotides to 80 nucleotides in length, from 80 nucleotides to 85 nucleotides in length, from 85 nucleotides to 90 nucleotides in length, from 90 nucleotides to 95 nucleotides in length, or from 95 nucleotides to 100 nucleotides in length. In some embodiments, the ASCE can be at least 10 nucleotides, at least 20 nucleotides, at least 30 nucleotides, at least 40 nucleotides, at least 50 nucleotides, at least 60 nucleoids, at least 70 nucleotides, at least 80 nucleotides in length, at least 90 nucleotides, or at least 100 nucleotides in length. In some embodiments, the ASCE can be from 100 to 200 nucleotides in length, from 200 to 300 nucleotides in length, from 300 to 400 nucleotides in length, from 400 to 500 nucleotides in length, from 500 to 600 nucleotides in length, from 600 to 700 nucleotides in length, from 700 to 800 nucleotides in length, from 800 to 900 nucleotides in length, from 900 to 1,000 nucleotides in length. In some embodiments, the ASCE may be longer than 1,000 nucleotides in length.

Exclusion of a ASCE can lead to a frameshift and the introduction of a premature termination codon (PIC) in the mature mRNA transcript rendering the transcript a target of NMD. Mature mRNA transcript lacking the ASCE can be non-productive mRNA transcript which does not lead to protein expression. The PIC can be present in any position downstream of the exon upstream of the ASCE in the pre-mRNA. In some embodiments, the PIC can be present in any exon downstream of the exon upstream of the ASCE in the pre-mRNA.

### Therapeutic Agents

In various embodiments of the present disclosure, compositions and methods comprising a therapeutic agent are provided to modulate protein expression level of ABCA4, FUS, CEL, or NSD1. In some embodiments, provided herein are compositions and methods to modulate alternative splicing of PKD1, ABCA4, FUS, CEL, or NSD1 pre-mRNA. In some embodiments, provided herein are compositions and methods to promote ASCE inclusion in the splicing of PKD1, ABCA4, FUS, CEL, or NSD1 pre-mRNA, e.g., to inhibit ASCE skipping of a ASCE during splicing of PKD1, ABCA4, FUS, CEL, or NSD1 pre-mRNA.

A therapeutic agent disclosed herein can be an NMD repressor agent. A therapeutic agent may comprise a polynucleic acid polymer.

According to one aspect of the present disclosure, provided herein is a method of treatment or prevention of a condition or disease associated with a functional polycystin-1, ATP binding cassette subfamily A member 4, FUS RNA binding protein, carboxyl ester lipase, or nuclear receptor binding SET domain protein 1 protein deficiency, comprising administering a ASCE repressor agent to a subject to increase levels of functional polycystin-1, ATP binding cassette subfamily A member 4, FUS RNA binding protein, carboxyl ester lipase, or nuclear receptor binding SET domain protein 1 protein, wherein the agent binds to a region of the pre-mRNA transcript to decrease inclusion of the ASCE in the mature transcript. For example, provided herein is a method of treatment or prevention of a condition associated with a functional polycystin-1, ATP binding cassette subfamily A member 4, FUS RNA binding protein, carboxyl ester lipase, or nuclear receptor binding SET domain protein 1 protein deficiency, comprising administering a ASCE repressor agent to a subject to increase levels of functional polycystin-1, ATP binding cassette subfamily A member 4, FUS RNA binding protein, carboxyl ester lipase, or nuclear receptor binding SET domain protein 1 protein, wherein the agent binds to a region of a pre-mRNA containing an ASCE. For example, provided herein is a method of treatment or prevention of a condition associated with a functional polycystin-1, ATP binding cassette subfamily A member 4, FUS RNA binding protein, carboxyl ester lipase, or nuclear receptor binding SET domain protein 1 protein deficiency, comprising administering a ASCE repressor agent to a subject to increase levels of functional polycystin-1, ATP binding cassette subfamily A member 4, FUS RNA binding protein, carboxyl ester lipase, or nuclear receptor binding SET domain protein 1 protein, wherein the agent binds to a region of a pre-mRNA containing an ASCE (e.g., ASCE (GRCh38/hg38: chr16 2092954 2093093) of PKD1; ASCE (GRCh38/hg38: chr1 94111438 94111579) of ABC4; ASCE (GRCh38/hg38: chr16 31186802 31186836) of FUS; ASCE (GRCh38/hg38: chr9 133066530 133066660) of CEL; ASCE (GRCh38/hg38: chr5 177238237 177238507) of NSD1).

Where reference is made to promoting ASCE inclusion in the mature mRNA, the promotion may be complete, e.g., 100%, or may be partial. The promotion may be clinically significant. The promotion/correction may be relative to the level of ASCE inclusion in the subject without treatment, or relative to the amount of ASCE inclusion in a population of similar subjects. The promotion/correction may be at least 10% more ASCE inclusion relative to the average subject, or the subject prior to treatment. The promotion may be at least 20% more ASCE inclusion relative to an average subject, or the subject prior to treatment. The promotion may be at least 40% more ASCE inclusion relative to an average subject, or the subject prior to treatment. The promotion may be at least 50% more ASCE inclusion relative to an average subject, or the subject prior to treatment. The promotion may be at least 60% more ASCE inclusion relative to an average subject, or the subject prior to treatment. The promotion may be at least 80% more ASCE inclusion relative to an average subject, or the subject prior to treatment. The promotion may be at least 90% more ASCE inclusion relative to an average subject, or the subject prior to treatment.

Where reference is made to increasing active polycystin-1, ATP binding cassette subfamily A member 4, FUS RNA binding protein, carboxyl ester lipase, or nuclear receptor binding SET domain protein 1 protein levels, the increase may be clinically significant. The increase may be relative to the level of active polycystin-1, ATP binding cassette subfamily A member 4, FUS RNA binding protein, carboxyl ester lipase, or nuclear receptor binding SET domain protein 1 protein in the subject without treatment, or relative to the amount of active polycystin-1, ATP binding cassette subfamily A member 4, FUS RNA binding protein, carboxyl ester lipase, or nuclear receptor binding SET domain protein 1 protein in a population of similar subjects. The increase may be at least 10% more active polycystin-1, ATP binding cassette subfamily A member 4, FUS RNA binding protein, carboxyl ester lipase, or nuclear receptor binding SET domain protein 1 protein relative to the average subject, or the subject prior to treatment. The increase may be at least 20% more active polycystin-1, ATP binding cassette subfamily A member 4, FUS RNA binding protein, carboxyl ester lipase, or nuclear receptor binding SET domain protein 1 protein relative to the average subject, or the subject prior to treatment. The increase may be at least 40% more active polycystin-1, ATP binding cassette subfamily A member 4, FUS RNA binding protein, carboxyl ester lipase, or nuclear receptor binding SET domain protein 1 protein relative to the average subject, or the subject prior to treatment. The increase may be at least 50% more active polycystin-1, ATP binding cassette subfamily A member 4, FUS RNA binding protein, carboxyl ester lipase, or nuclear receptor binding SET domain protein 1 protein relative to the average subject, or the subject prior to treatment. The increase may be at least 80% more active polycystin-1, ATP binding cassette subfamily A member 4, FUS RNA binding protein, carboxyl ester lipase, or nuclear receptor binding SET domain protein 1 protein relative to the average subject, or the subject prior to treatment. The increase may be at least 100% more active polycystin-1, ATP binding cassette subfamily A member 4, FUS RNA binding protein, carboxyl ester lipase, or nuclear receptor binding SET domain protein 1 protein relative to the average subject, or the subject prior to treatment. The increase may be at least 200% more active polycystin-1, ATP binding cassette subfamily A member 4, FUS RNA binding protein, carboxyl ester lipase, or nuclear receptor binding SET domain protein 1 protein relative to the average subject, or the subject prior to treatment. The increase may be at least 500% more active polycystin-1, ATP binding cassette subfamily A member 4, FUS RNA binding protein, carboxyl ester lipase, or nuclear receptor binding SET domain protein 1 protein relative to the average subject, or the subject prior to treatment.

In embodiments wherein the ASCE repressor agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

The sequence of the polynucleic acid polymer may have 4 or fewer mismatches to a target sequence of the pre-mRNA transcript. The sequence of the polynucleic acid polymer may have 3 or fewer mismatches to a target sequence of the pre-mRNA transcript. The sequence of the polynucleic acid polymer may have 2 or fewer mismatches to a target sequence of the pre-mRNA transcript. The sequence of the polynucleic acid polymer may have 1 or fewer mismatches to a target sequence of the pre-mRNA transcript. The sequence of the polynucleic acid polymer may have no mismatches to a target sequence of the pre-mRNA transcript.

The polynucleic acid polymer may specifically hybridize to a target sequence of the pre-mRNA transcript. For example, the polynucleic acid polymer may have 91%, 92%, 93%, 94%, 95%, 96%, 97%, 98%, 99%, 99.5% or 100% sequence complementarity to a target sequence of the pre-mRNA transcript. The hybridization may be under high stringent hybridization conditions.

The polynucleic acid polymer comprising a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOS: 16-309. The polynucleic acid polymer may comprise a sequence with 100% sequence identity to a sequence selected from the group consisting of SEQ ID NOS: 16-309.

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence. References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

### Antisense Oligomers

Provided herein is a composition comprising an antisense oligomer that induces exon skipping by binding to a targeted portion of a PKD1, ABCA4, FUS, CEL, or NSD1 ASCE-containing pre-mRNA. As used herein, the terms “ASO” and “antisense oligomer” are used interchangeably and refer to an oligomer such as a polynucleotide, comprising nucleobases that hybridizes to a target nucleic acid (e.g., a PKD1, ABCA4, FUS, CEL, or NSD1 ASCE-containing pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art, for example in PCT Application No. PCT/US2014/054151, published as WO 2015/035091, titled “Reducing Nonsense-Mediated mRNA Decay,” incorporated by reference herein, can be used to practice the methods described herein.

In some embodiments, ASOs “specifically hybridize” to or are “specific” to a target nucleic acid or a targeted portion of an ASCE-containing pre-mRNA. Typically, such hybridization occurs with a Tₘ substantially greater than 37° C., preferably at least 50° C., and typically between 60° C. to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a target portion of an ASCE-containing pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally-occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. Nos. 8,258,109 B2, 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphorothioate linkage. In some embodiments, the backbone modification is a phosphoramidate linkage.

In some embodiments, the stereochemistry at each of the phosphorus internucleotide linkages of the ASO backbone is random. In some embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In some embodiments, an ASO used in the methods of the disclosure, including, but not limited to, any of the ASOs set forth herein in Tables 4, 5A, 5A-1, 5B, 5B-1, 5D, 5E, 5G, and 5G-1, comprises an ASO having phosphorus internucleotide linkages that are not random. In some embodiments, a composition used in the methods of the disclosure comprises a pure diastereomeric ASO. In some embodiments, a composition used in the methods of the disclosure comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second-generation antisense oligonucleotides containing chiral phosphorothioate linkages,” Nucleic Acids Research 42(22): 13456-13468, incorporated herein by reference). In some embodiments, an ASO used in the methods of the disclosure, including, but not limited to, any of the ASOs set forth herein in SEQ ID NOS: 16-309, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOS: 16-309, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In some embodiments, an ASO used in the methods of the disclosure, including, but not limited to, any of the ASOs set forth herein comprise a sequence that is complementary to a sequence with at least about 80%, 85%, 90%, 95%, 97%, or 100% sequence identity to a region comprising at least 8 contiguous nucleic acids of any one of SEQ ID NOS: 6-10, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In embodiments, an ASO used in the methods of the disclosure, including, but not limited to, any of the ASOs set forth herein comprise a sequence that is complementary to a sequence with at least about 80%, 85%, 90%, 95%, 97%, or 100% sequence identity to a region comprising at least 8 contiguous nucleic acids of any one of SEQ ID NOS: 6-10, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′F; N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-O ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a PKD1, ABCA4, FUS, CEL, or NSD1 ASCE-containing pre-mRNA that is downstream (in the 3′ direction) of the 5′ splice site (or 3′ end of the ASCE) of the ASCE in a PKD1, ABCA4, FUS, CEL, or NSD1 ASCE-containing pre-mRNA (e.g., the direction designated by positive numbers relative to the 5′ splice site). In some embodiments, the ASOs are complementary to a targeted portion of the PKD1, ABCA4, FUS, CEL, or NSD1 ASCE-containing pre-mRNA that is within the region about +1 to about +500 relative to the 5′ splice site (or 3′ end) of the ASCE. In some embodiments, the ASOs may be complementary to a targeted portion of a PKD1, ABCA4, FUS, CEL, or NSD1 ASCE-containing pre-mRNA that is within the region between nucleotides +6 and +40,000 relative to the 5′ splice site (or 3′ end) of the ASCE. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20 relative to 5′ splice site (or 3′ end) of the ASCE. In some aspects, the ASOs are complementary to a targeted portion that is within the region from about +1 to about +100, from about +100 to about +200, from about +200 to about +300, from about +300 to about +400, or from about +400 to about +500 relative to 5′ splice site (or 3′ end) of the ASCE.

In some embodiments, the ASOs are complementary to (and bind to) a targeted portion of a PKD1, ABCA4, FUS, CEL, or NSD1 ASCE-containing pre-mRNA that is upstream (in the 5′ direction) of the 5′ splice site (or 3′ end) of the ASCE in a PKD1, ABCA4, FUS, CEL, or NSD1 ASCE-containing pre-mRNA (e.g., the direction designated by negative numbers relative to the 5′ splice site). In some embodiments, the ASOs are complementary to a targeted portion of the PKD1, ABCA4, FUS, CEL, or NSD1 ASCE-containing pre-mRNA that is within the region about −4 to about −270 relative to the 5′ splice site (or 3′end) of the ASCE. In some embodiments, the ASOs may be complementary to a targeted portion of a PKD1, ABCA4, FUS, CEL, or NSD1 ASCE-containing pre-mRNA that is within the region between nucleotides −1 and −40,000 relative to the 5′ splice site (or 3′ end) of the ASCE. In some aspects, the ASOs are complementary to a targeted portion that is within the region about −1 to about −40,000, about −1 to about −30,000, about −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 5′ splice site (or 3′ end) of the ASCE.

In some embodiments, the ASOs are complementary to a targeted region of a PKD1, ABCA4, FUS, CEL, or NSD1 ASCE-containing pre-mRNA that is upstream (in the 5′ direction) of the 3′ splice site (or 5′ end) of the ASCE in a PKD1, ABCA4, FUS, CEL, or NSD1 ASCE-containing pre-mRNA (e.g., in the direction designated by negative numbers). In some embodiments, the ASOs are complementary to a targeted portion of the PKD1, ABCA4, FUS, CEL, or NSD1 ASCE-containing pre-mRNA that is within the region about −1 to about −500 relative to the 3′ splice site (or 5′ end) of the ASCE. In some embodiments, the ASOs are complementary to a targeted portion of the PKD1, ABCA4, FUS, CEL, or NSD1 ASCE-containing pre-mRNA that is within the region −1 to −40,000 relative to the 3′ splice site of the ASCE. In some aspects, the ASOs are complementary to a targeted portion that is within the region about −1 to about −40,000, about −1 to about −30,000, −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 3′ splice site of the ASCE. In some aspects, the ASOs are complementary to a targeted portion that is within the region from about −1 to about −100, from about −100 to about −200, from about −200 to about −300, from about −300 to about −400, or from about −400 to about −500 relative to 3′ splice site of the ASCE.

In some embodiments, the ASOs are complementary to a targeted region of a PKD1, ABCA4, FUS, CEL, or NSD1 ASCE-containing pre-mRNA that is downstream (in the 3′ direction) of the 3′ splice site (5′ end) of the ASCE in a PKD1, ABCA4, FUS, CEL, or NSD1 ASCE-containing pre-mRNA (e.g., in the direction designated by positive numbers). In some embodiments, the ASOs are complementary to a targeted portion of the PKD1, ABCA4, FUS, CEL, or NSD1 ASCE-containing pre-mRNA that is within the region of about +1 to about +40,000 relative to the 3′ splice site of the ASCE. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20, or about +1 to about +10 relative to 3′ splice site of the ASCE.

In some embodiments, the targeted portion of the PKD1, ABCA4, FUS, CEL, or NSD1 ASCE-containing pre-mRNA is within the region +100 relative to the 5′ splice site (3′ end) of the ASCE to −100 relative to the 3′ splice site (5′ end) of the ASCE. In some embodiments, the targeted portion of the PKD1, ABCA4, FUS, CEL, or NSD1 ASCE-containing pre-mRNA is within the ASCE. In some embodiments, the target portion of the PKD1, ABCA4, FUS, CEL, or NSD1 ASCE-containing pre-mRNA comprises a ASCE and intron boundary. In some embodiments, the target portion of the PKD1, ABCA4, FUS, CEL, or NSD1 ASCE-containing pre-mRNA does not comprise a ASCE and intron boundary.

The ASOs may be of any length suitable for specific binding and effective reduction of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted portion of the ASCE-containing pre-mRNA are used. In some embodiments, two or more ASOs that are complementary to different targeted portions of the ASCE-containing pre-mRNA are used.

In some embodiments, the antisense oligonucleotides of the disclosure are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptide, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a PKD1, ABCA4, FUS, CEL, or NSD1 ASCE-containing pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

### Pharmaceutical Compositions

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent, or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present disclosure includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In embodiments, the present disclosure employs a penetration enhancer to effect the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple antisense oligonucleotides. In embodiments, the antisense oligonucleotide is administered in combination with another drug or therapeutic agent.

### Combination Therapies

In some embodiments, the ASOs disclosed in the present disclosure can be used in combination with one or more additional therapeutic agents. In some embodiments, the one or more additional therapeutic agents can comprise a small molecule. For example, the one or more additional therapeutic agents can comprise a small molecule described in WO2016128343A1, WO2017053982A1, WO2016196386A1, WO201428459A1, WO201524876A2, WO2013119916A2, and WO2014209841A2, which are incorporated by reference herein in their entirety.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In embodiments, a fetus is treated in utero, e.g., by administering the ASO composition to the fetus directly or indirectly (e.g., via the mother).

Suitable routes for administration of ASOs of the present disclosure may vary depending on cell type to which delivery of the ASOs is desired. Multiple tissues and organs are affected by Dravet syndrome, with the brain being the most significantly affected tissue. The ASOs of the present disclosure may be administered to patients parenterally, for example, by intrathecal injection, intracerebroventricular injection, intraperitoneal injection, intramuscular injection, subcutaneous injection, or intravenous injection.

In embodiments, the antisense oligonucleotide is administered with one or more agents capable of promoting penetration of the subject antisense oligonucleotide across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the antisense oligonucleotides are linked or conjugated with agents that provide desirable pharmaceutical or pharmacodynamic properties. In embodiments, the antisense oligonucleotide is coupled to a substance, known in the art to promote penetration or transport across the blood-brain barrier, e.g., an antibody to the transferrin receptor. In embodiments, the antisense oligonucleotide is linked with a viral vector, e.g., to render the antisense compound more effective or increase transport across the blood-brain barrier. In embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 9,193,969, “Compositions and methods for selective delivery of oligonucleotide molecules to specific neuron types,” U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each incorporated herein by reference.

In some embodiments, an ASO of the disclosure is coupled to a dopamine reuptake inhibitor (DRI), a selective serotonin reuptake inhibitor (SSRI), a noradrenaline reuptake inhibitor (NRI), a norepinephrine-dopamine reuptake inhibitor (NDRI), and a serotonin-norepinephrine-dopamine reuptake inhibitor (SNDRI), using methods described in, e.g., U.S. Pat. No. 9,193,969, incorporated herein by reference.

In some embodiments, subjects treated using the methods and compositions are evaluated for improvement in condition using any methods known and described in the art.

Methods of Identifying Additional ASOs that Promote Inclusion of an ASCE

Also within the scope of the present disclosure are methods for identifying or determining ASOs that promote inclusion of an ASCE in a processed mRNA that is processed from a pre-mRNA comprising the ASCE. Also within the scope of the present disclosure are methods for identifying or determining ASOs that promote inclusion of an ASCE of a PKD1, ABCA4, FUS, CEL, or NSD1 ASCE-containing pre-mRNA. Also within the scope of the present disclosure are methods for identifying or determining ASOs that promote inclusion of an ASCE in a processed mRNA that is processed from a pre-mRNA comprising the ASCE.

For example, a method can comprise identifying or determining ASOs that promote inclusion of an ASCE of a PKD1, ABCA4, FUS, CEL, or NSD1 ASCE-containing pre-mRNA. ASOs that specifically hybridize to different nucleotides within the target region of the pre-mRNA may be screened to identify or determine ASOs that improve the rate and/or extent of splicing of the target intron. In some embodiments, the ASO may block or interfere with the binding site(s) of a splicing repressor(s)/silencer. Any method known in the art may be used to identify (determine) an ASO that when hybridized to the target region of the exon results in the desired effect (e.g., exon inclusion, protein or functional RNA production). These methods also can be used for identifying ASOs that promote exon inclusion of the excluded exon by binding to a targeted region in an intron flanking the excluded exon, or in a non-excluded exon. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ splice site flanking the ASCE (e.g., a portion of sequence of the intron located upstream of the target/ASCE) to approximately 100 nucleotides downstream of the 3′ splice site flanking the target/ASCE and/or from approximately 100 nucleotides upstream of the 5′ splice site flanking the ASCE to approximately 100 nucleotides downstream of the 5′ splice site flanking the target/ASCE (e.g., a portion of sequence of the intron located downstream of the target/ASCE). For example, a first ASO of 15 nucleotides in length may be designed to specifically hybridize to nucleotides −6 to −20 relative to the 3′ splice site flanking the target/ASCE. A second ASO may be designed to specifically hybridize to nucleotides −1 to −15 relative to the 3′ splice site flanking the target/ASCE. ASOs are designed as such spanning the target region of the pre-mRNA. In embodiments, the ASOs can be tiled more closely, e.g., every 1, 2, 3, or 4 nucleotides. Further, the ASOs can be tiled from 100 nucleotides downstream of the 5′ splice site, to 100 nucleotides upstream of the 3′ splice site. In some embodiments, the ASOs can be tiled from about 1000 or 500 nucleotides upstream of the 3′ splice site, to about 1000 or 500 nucleotides downstream of the 5′ splice site. In some embodiments, the ASOs can be tiled from about 1000 or 500 nucleotides upstream of the 3′ splice site, to about 1000 or 500 nucleotides downstream of the 3′ splice site.

One or more ASOs, or a control ASO (an ASO with a scrambled sequence, sequence that is not expected to hybridize to the target region) are delivered, for example by transfection, into a disease-relevant cell line that expresses the target pre-mRNA (e.g., a ASCE-containing pre-mRNA described herein). The exon inclusion effects of each of the ASOs may be assessed by any method known in the art, for example by reverse transcriptase (RT)-PCR using primers that span the splice junction, as described in Example 3. An increase or presence of a longer RT-PCR product produced using the primers spanning the region containing the ASCE (e.g., including the flanking introns of the ASCE) in ASO-treated cells as compared to in control ASO-treated cells indicates that splicing out of the target ASCE has been reduced. In some embodiments, the exon inclusion efficiency, the ratio of unspliced to spliced pre-mRNA, the rate of splicing, or the extent of splicing may be modulated using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, Jess blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in exon inclusion (or reduced splicing of the ASCE).

Regions defined by ASOs that reduce splicing of the target exon are explored in greater detail by means of an ASO “micro-walk,” involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the ASCE, as described herein (see, e.g., Example 5). An increase or presence of a longer RT-PCR product produced using the primers spanning the ASCE in ASO-treated cells as compared to in control ASO-treated cells indicates that exon inclusion has been enhanced. In some embodiments, the exon inclusion efficiency, the ratio of unspliced to spliced pre-mRNA, the rate of splicing, or the extent of splicing may be modulated using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, Jess blotting, flow cytometry, immunofluorescence microscopy, and ELISA, can be used.

ASOs that when hybridized to a region of a pre-mRNA result in exon inclusion and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intrathecal injection, intracerebroventricular injection, intraperitoneal injection, intramuscular injection, subcutaneous injection, or intravenous injection. Following administration, the cells, tissues, and/or organs of the model animals may be assessed to determine the effect of the ASO treatment by for example evaluating splicing (e.g., efficiency, rate, extent) and protein production by methods known in the art and described herein. The animal models may also be any phenotypic or behavioral indication of the disease or disease severity.

Also within the scope of the present disclosure is a method to identify or validate an ASCE in the presence of an NMD inhibitor, for example, cycloheximide. An exemplary method is provided in Example 2.

### EXAMPLES

The present disclosure will be more specifically illustrated by the following Examples. However, it should be understood that the present disclosure is not limited by these examples in any manner.

### Example 1. Identification of NMD-Inducing Exon Inclusion Events in Transcripts by RNAseq Using Next-Generation Sequencing

Whole transcriptome shotgun sequencing is carried out using next generation sequencing to reveal a snapshot of transcripts produced by the genes described herein to identify ASCE inclusion events. For this purpose, polyA+ RNA from nuclear and cytoplasmic fractions of human cells is isolated and cDNA libraries are constructed using Illumina's TruSeq Stranded mRNA library Prep Kit. The libraries are pair-end sequenced resulting in 100-nucleotide reads that are mapped to the human genome (GRCh38/hg38 assembly).

### Example 2. Confirmation of ASCE via Cycloheximide Treatment

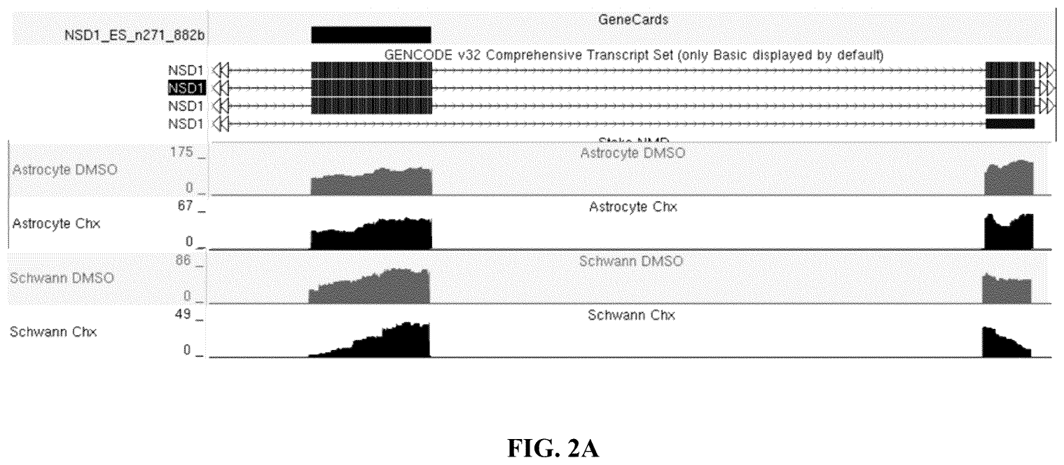

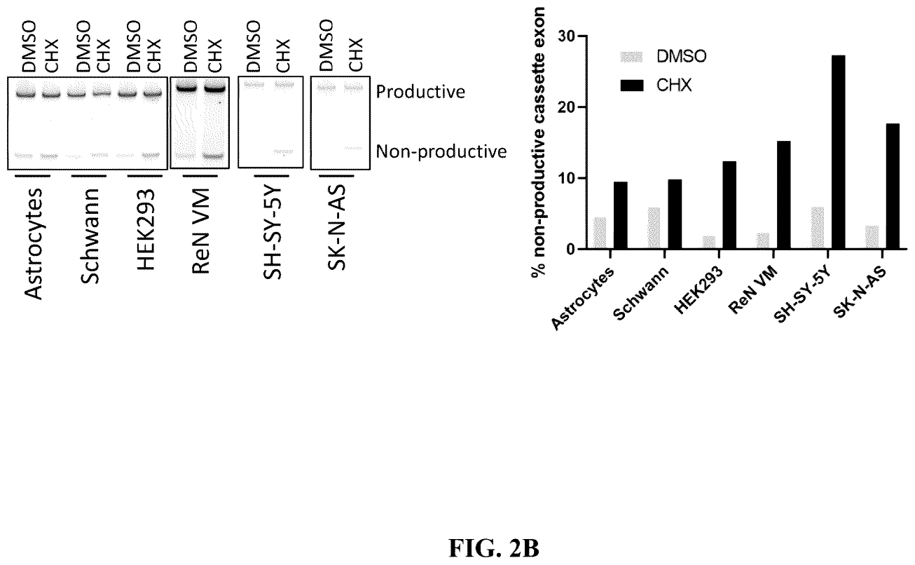

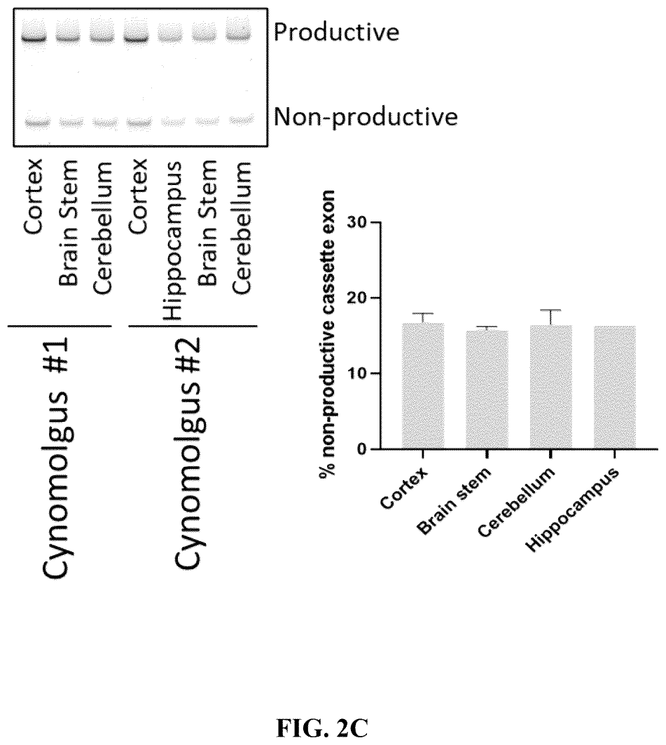

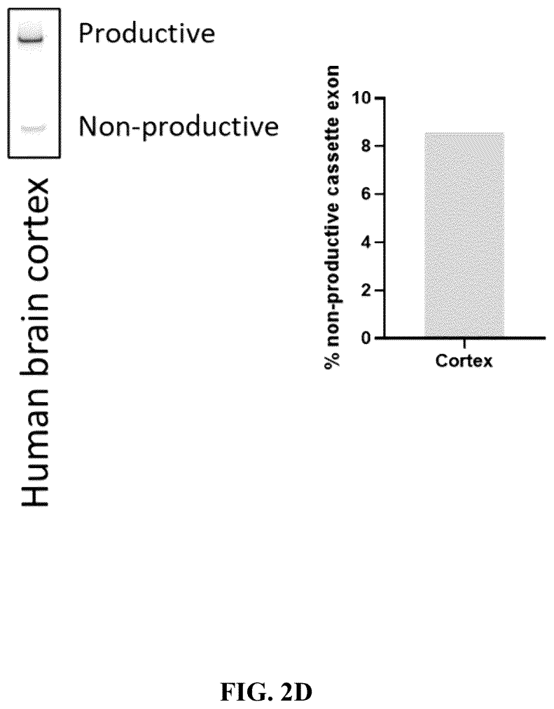

RT-PCR analysis using RNA extracts from DMSO-treated or cycloheximide-treated human and mouse cells and primers in exons (e.g., a forward primer complimentary to exon 7 and a reverse primer complimentary to exon 9) confirmed the presence of a band corresponding to an NMD-inducing exon exclusion event. Treatment of cells with cycloheximide to inhibit NMD can lead to an increase of the product corresponding to the NMD-inducing exon exclusion event in the cytoplasmic fraction. RT-PCR and quantification of the cassette exon of NSD1 RNA (exon 8: GRCh38/hg38: chr5 177238237:177238507) were conducted. Densitometry analysis of the bands on the image of the RT-PCR products was performed to calculate percent ASCE inclusion of total transcript. FIGS. 2A-2D depict confirmation of exemplary alternative splicing events of an ASCE in the NSD1 gene via cycloheximide treatment in various human cells, as well as confirmation of existence of non-productive NSD1 mRNA transcripts in cynomolgus monkey brain regions and human cortex. FIG. 2A depicts a schematic in which peaks corresponding to RNA sequencing reads were identified in exon 8 of NSD1 (GRCh38/hg38: chr5 177238237:177238507). FIG. 2B depicts gel images and a graph showing that cycloheximide treatment led to increase in the amount of non-productive mature NSD1 mRNA transcripts (processed NSD1 mRNA containing a premature termination codon rendering the transcript a target of NMD) in various human cells, including astrocytes, Schwann cells, HEK293 cells, SH-SY-5Y (neuroblastoma cell line) cells, and SK-N-AS (neuroblastoma cell line) cells. FIG. 2C depicts a gel image and a graph showing the existence of non-productive mature NSD1 mRNA transcript in various cynomolgus brain regions, including cortex, brain stem, hippocampus, and cerebellum. FIG. 2D depicts a gel image and a graph showing the existence of non-productive mature NSD1 mRNA transcript in human cortex.

### Example 3. Confirmation of ASCE Via Cycloheximide Treatment in Mice

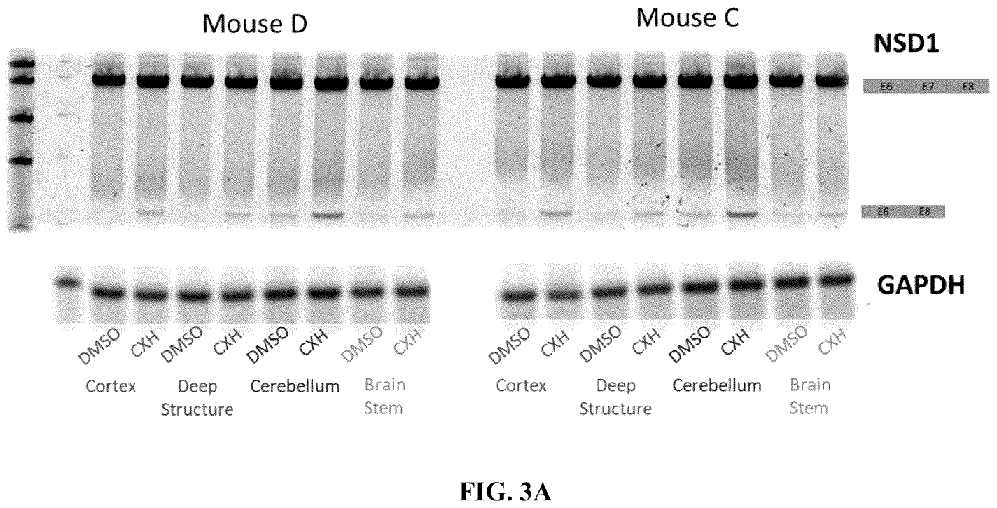

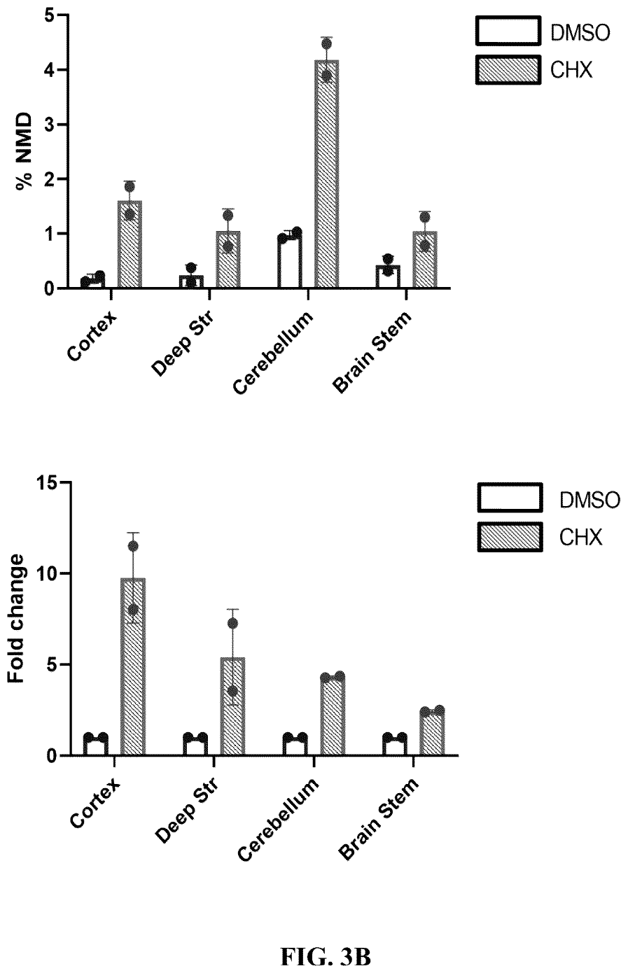

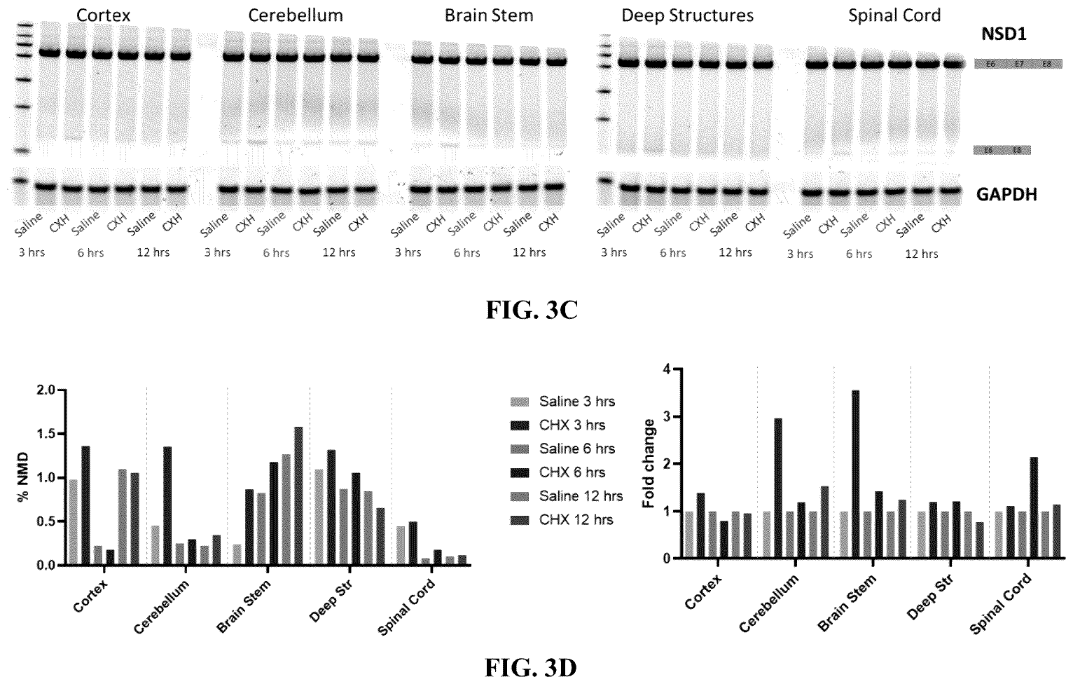

RT-PCR analysis using total RNA from in vivo or ex vivo DMSO-treated or cycloheximide-treated mouse brain regions (cortex, deep structure, cerebellum and brain stem) and primers in exons (e.g., a forward primer complimentary to mouse exon 6 and a reverse primer complimentary to mouse exon 8) confirmed the presence of a band corresponding to an ASCE exclusion event (FIGS. 3A-3D). FIG. 3A depicts gel images showing that, in ex vivo cycloheximide- or DMSO-treated mouse brains, exclusion of an ASCE of mouse NSD1 (mouse exon 7, corresponding to human exon 8) leads to formation of a processed mRNA containing a premature termination codon rendering the transcript a target of NMD. FIG. 3B depicts graphs of the percentage of NMD (top) and fold-change (bottom) of the NMD event of the non-productive NSD1 mRNA product relative to NSD1 productive NSD1 mRNA product according to densitometry analysis of the bands from the gel images of FIG. 3A to calculate percent ASCE. The fold-change in the bottom panel of FIG. 3B was calculated as fold change in percentage NMD between DMSO- and cycloheximide-treated samples, i.e., the percentage NMD in cycloheximide-treated samples divided by the percentage NMD in the corresponding DMSO-treated sample for each indicated brain region. FIG. 3C depicts gel images showing that, in in vivo cycloheximide-treated mouse brains that were treated for 3 or 6 or 12 hours, exclusion of an ASCE of mouse NSD1 (mouse exon 7, corresponding to human exon 8) leads to formation of a processed mRNA containing a premature termination codon rendering the transcript a target of NMD. FIG. 3D depicts graphs of the percentage of NMD (left) and fold-change (right) of the NMD event of the non-productive NSD1 mRNA product relative to NSD1 productive NSD1 mRNA product according to densitometry analysis of the bands from the gel images of FIG. 3C to calculate percent ASCE. The fold-change in the right panel of FIG. 3D was calculated as fold change in percentage NMD between saline and cycloheximide-treated samples, i.e., the percentage NMD in cycloheximide-treated samples divided by the percentage NMD in the corresponding saline treated sample for each indicated brain region.

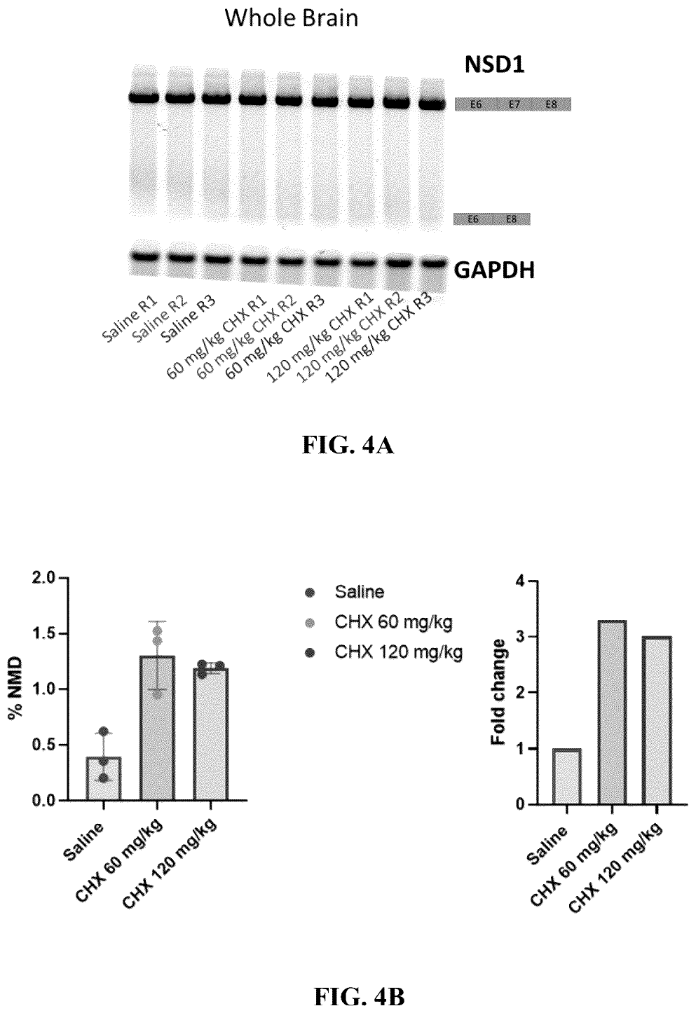

FIGS. 4A-4B depict confirmation of inclusion or exclusion an ASCE of mouse NSD1 (mouse exon 7, corresponding to human exon 8) in NSD1 mRNA products processed from NSD1 pre-mRNA in mouse brains via in vivo cycloheximide treatment. FIG. 4A depicts a gel image showing that, in in vivo cycloheximide-treated mouse brains, exclusion of an ASCE of mouse NSD1 (mouse exon 7, corresponding to human exon 8) leads to formation of a processed mRNA containing a premature termination codon rendering the transcript a target of NMD. FIG. 4B depicts graphs of the percentage of NMD (left) and fold-change (right) of the NMD event of the non-productive NSD1 mRNA product relative to NSD1 productive NSD1 mRNA product according to densitometry analysis of the bands from the gel images of FIG. 4A to calculate percent ASCE. The fold-change in the right panel of FIG. 4B was calculated as fold change in percentage NMD between saline and cycloheximide-treated samples, i.e., the percentage NMD in the 60 mg/kg or 120 mg/kg cycloheximide-treated samples divided by the percentage NMD in the corresponding saline treated samples.

### Example 4. ASCE Region ASO Walk

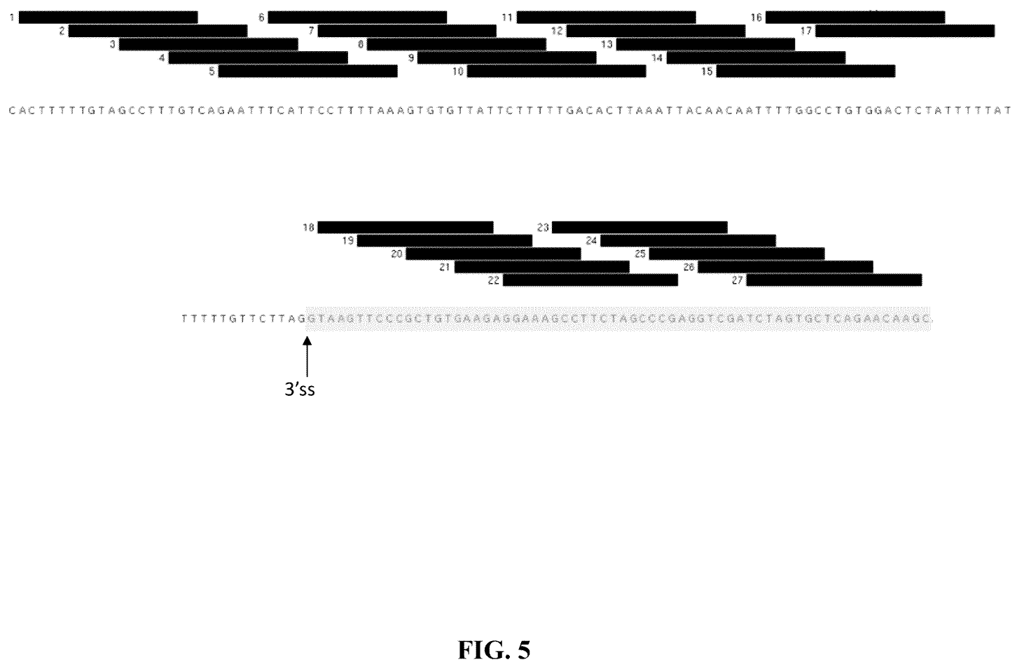

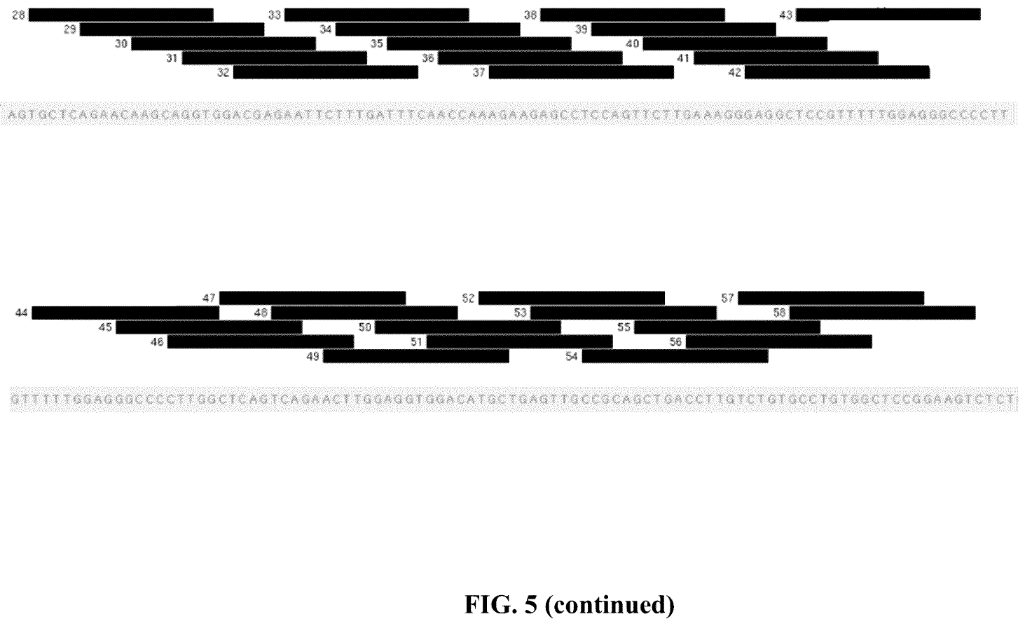

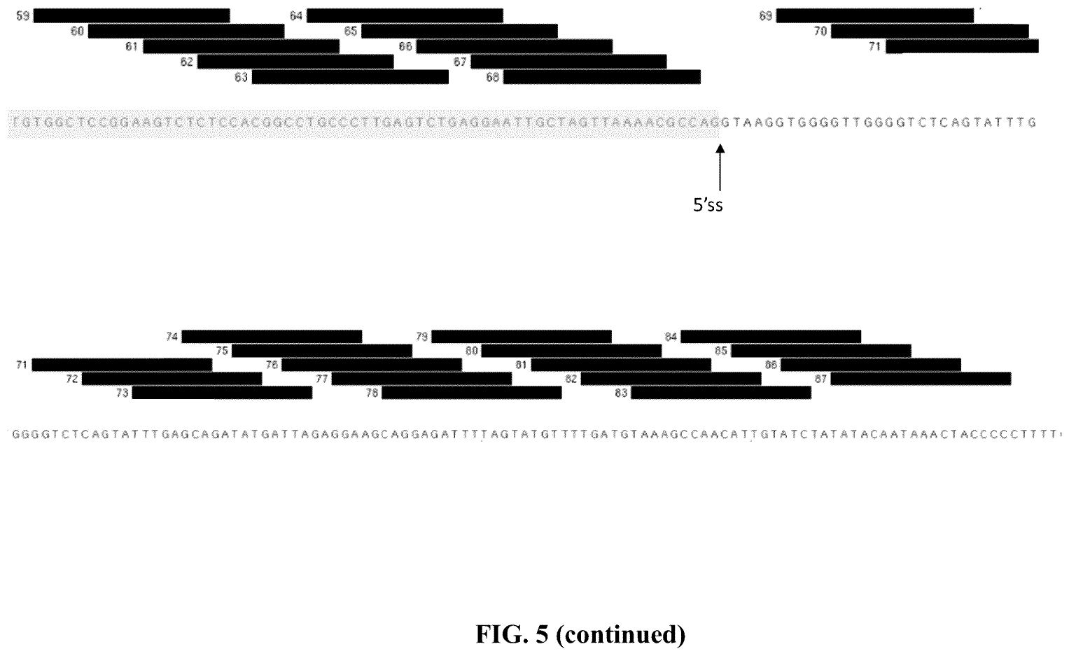

An ASO walk can be performed for ASCE region targeting sequences upstream of the canonical 3′ splice site, across the 3′splice sited, the skipped exon (exon 8 for instance), across the 5′ splice site, and downstream of the 5′ splice site using 2′-MOE ASOs, PS backbone. ASOs can be designed to cover these regions by shifting 5 nucleotides at a time or by shifting any predetermined number of nucleotides at a time. In some embodiments, ASO walk can be performed for ASCE region targeting sequences that are not across the 3′splice site and/or not across the 5′ splice site. FIG. 5 depicts an exemplary ASO walk for an exemplary ASCE region. The shaded nucleotides in FIG. 5 correspond to the exon skipping event and arrows point to canonical splice sites.

### Example 5. ASCE Region ASO Walk Evaluated by RT-PCR

ASO walk sequences can be evaluated by for example RT-PCR. PAGE can be used to show SYBR-safe-stained RT-PCR products of mock-treated or ASO-treated cells targeting the ASCE regions as described herein at 20-NM concentration in human/mouse cells by gymnotic uptake. Products corresponding to exon exclusion and full-length can be quantified and percent MD can be plotted. Full-length products can be normalized to internal controls.

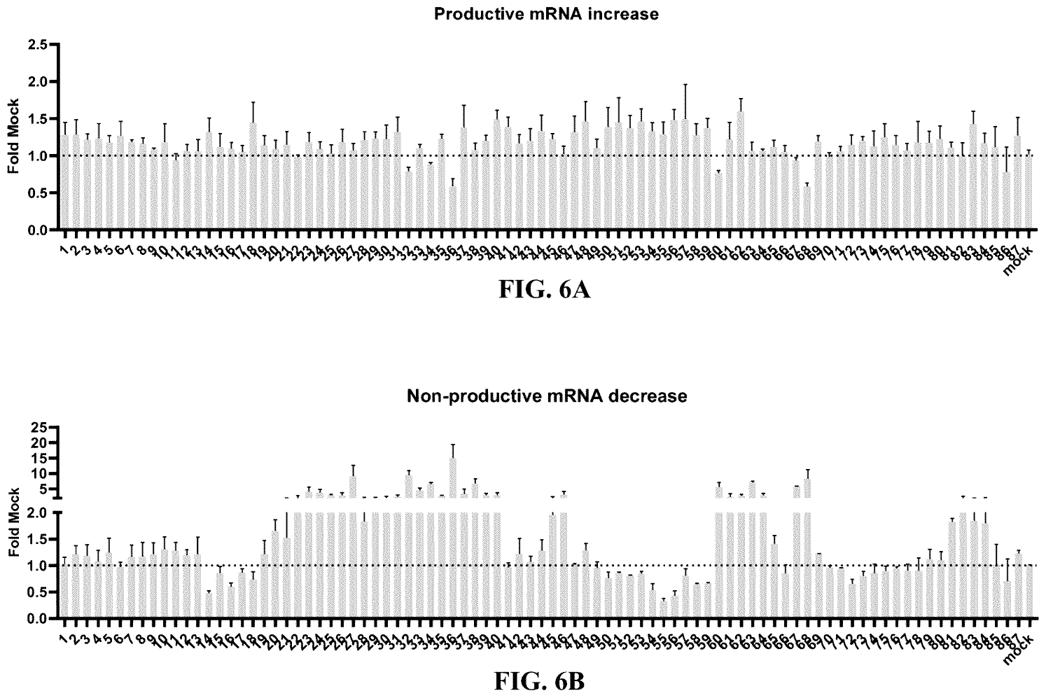

In one experiment, HEK293 cells were transfected for 24 hours with exemplary ASOs according to some embodiments of the present disclosure at an 80-nM concentration. FIG. 6A shows a graph summarizing the changes in the level of productive NSD1 mRNA in one ASO walk around the cassette exon (exon 8). FIG. 6B shows a graph summarizing the changes in the level of non-productive NSD1 mRNA in one ASO walk around the cassette exon (exon 8).

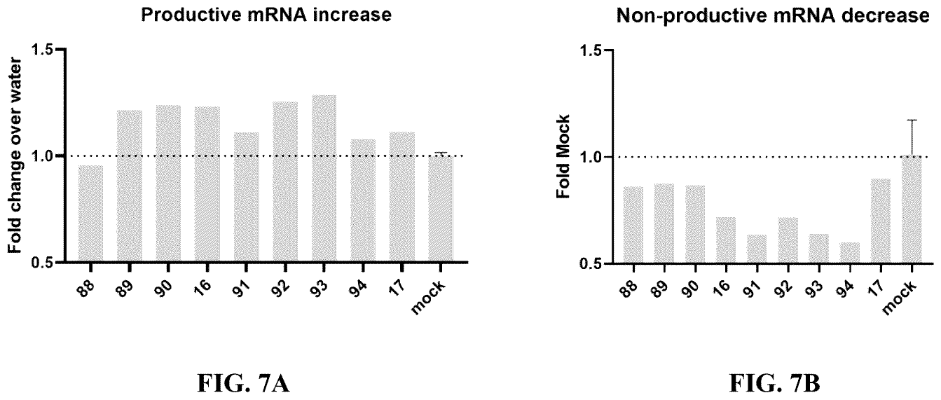

In another experiment, ASO micro-walk was conducted by nucleofecting SH-SY-5Y cells for 24 hours with exemplary ASOs according to some embodiments of the present disclosure at a 5 mM concentration. FIG. 7A shows a graph summarizing the changes in the level of productive NSD1 mRNA in one ASO walk around the cassette exon (exon 8). FIG. 7B shows a graph summarizing the changes in the level of non-productive NSD1 mRNA in one ASO walk around the cassette exon (exon 8).

### Example 6. NSD1 ASCE Region Vectorized ASO Walk

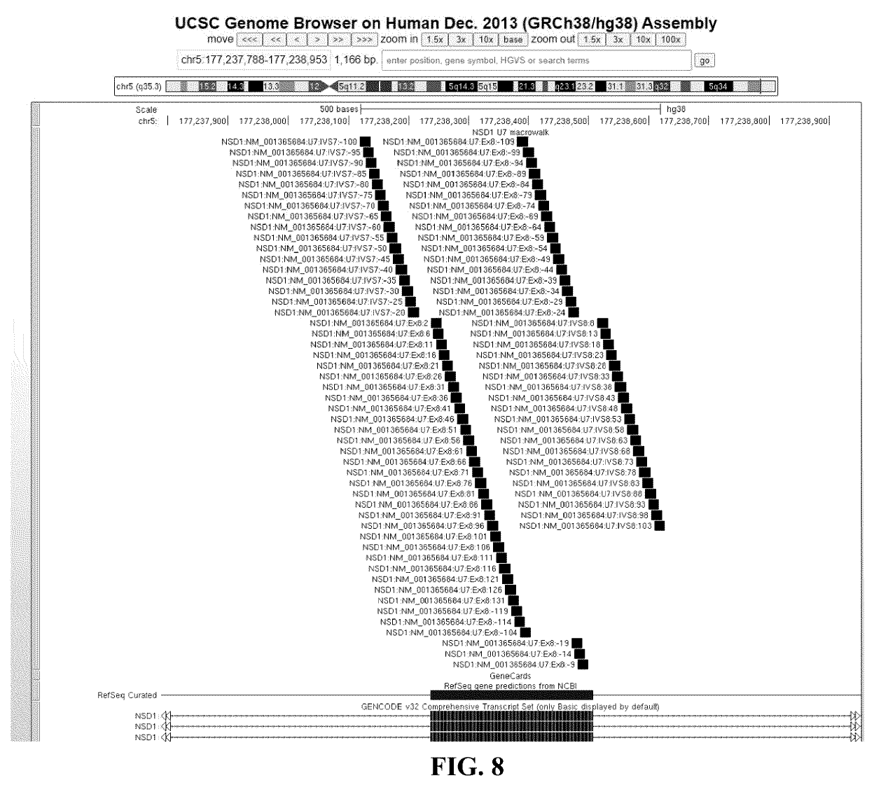

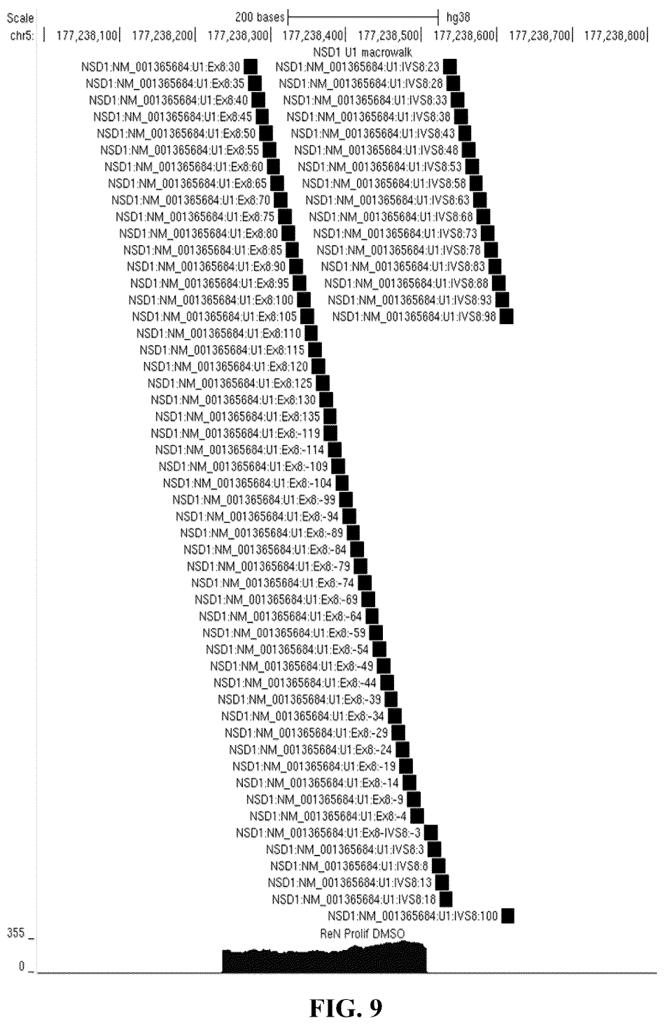

An ASO walk can be performed for ASCE region targeting sequences upstream of the canonical 3′ splice site, across the 3′ splice site, the skipped exon (exon 8 for instance), across the 5′ splice site, and downstream of the 5′ splice site to identify vectorized ASOs that can prevent a non-productive AS event (e.g., promote inclusion of an ASCE in a processed mRNA), a systematic vectorized ASO walk can be performed in 5-nt or 2-nt steps along the AS event of interest. These vectorized ASOs can be expressed from a vector as a modified U1 snRNA or U7 snRNA, which contains an ASO sequence as its targeting sequence. FIG. 8 shows a systematic vectorized ASO walk along the AS event of NSD1 pre-mRNA for a vectorized ASO that is expressed as a modified U7 snRNA. FIG. 9 shows a systematic vectorized ASO walk along the AS event of NSD1 pre-mRNA for a vectorized ASO that is expressed as a U1 snRNA. RT-PCR analysis from transfected cell lines can identify several vectorized ASOs that lead to reduced AS (e.g., promote inclusion of an ASCE in a processed mRNA) in the NSD1 mRNA and increase in productive mRNA. The observed increase in NSD1 productive mRNA can be confirmed by TaqMan qPCR. The fold change of AS may be plotted against the increase in productive mRNA (as measured by qPCR) to demonstrate that the vectorized ASOs are mechanistically functioning.

### Example 7. NSD1 Non-Productive mRNA Levels in Various Cell Lines

Alternative NMD inhibitors (i.e., non-ASOs) were tested in different cell lines to assess the baseline differences in non-productive RNA levels across various cell types.

The cell lines used were SH-SY5Y, U-87 MG, HEK293, and SK-N-AS. SH-SY5Y is a subcloned cell line from a neuroblastoma cell line originating from metastatic bone tumors. U-87 MG is a cell line isolated from malignant gliomas displaying epithelial morphology. Human Embryonic Kidney (HEK) 293 is a cell line routinely used for basic biotechnology research. SK-N-AS cells are human neuroblasts originating from neuroblastoma cells.

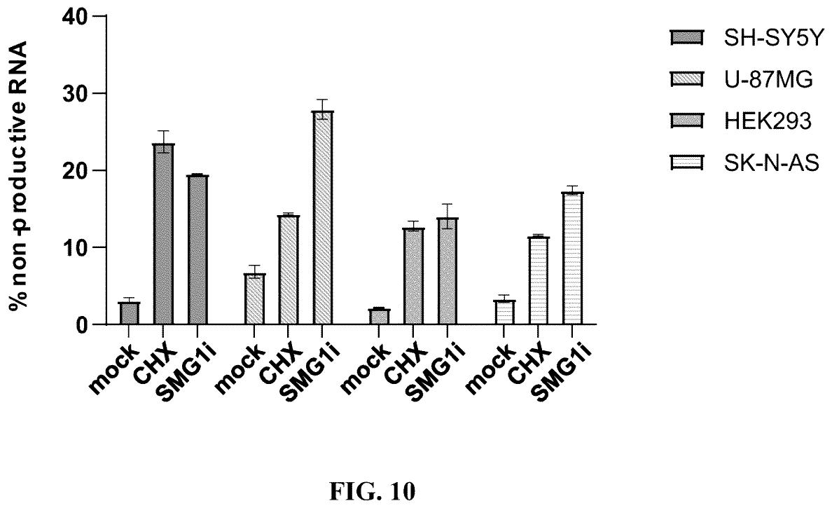

The NMD inhibitors tested include SMG1 Nonsense-Mediated MRNA Decay Associated PI3K Related Kinase inhibitor (SMG1i) and cycloheximide (CHX). SMG1i is an inhibitor of nonsense-mediated mRNA decay (NMD) regulator SMG1 and was originally designed to target multiple myeloma. SMG1i was used as an NMD inhibitor and tested alongside CHX, a standard mRNA translation inhibitor also known to inhibit NMD. The effects of NMD inhibitors were measured in different cell lines to determine whether cell lines had different baseline levels of non-productive RNA, interpreted to be equivalent to NMD events. Each of the four cell lines (SH-SY5Y, U-87 MG, HEK293, and SK-N-AS) was incubated with a negative control (mock) and NMD inhibitors (CHX at a final concentration of 50 μg/ml and SMG1i at a final concentration of 1 μM), respectively, for three hours to evaluate the baseline levels of non-productive NSD1 mRNA (FIG. 10). After treatment with either water-only mock control, CHX, or SMG1i, cells from each cell line were harvested, and RNA was extracted and quantitated. Treatment with SMG1i resulted in ˜28% NSD1 non-productive mRNA (percentage of the level of non-productive NSD1 mRNA transcript in the total level of all NSD1 mRNA transcripts) in U-87 MG cells, ˜19% NSD1 non-productive mRNA levels in SH-SY5Y cells, and <˜18% NSD1 non-productive mRNA levels in HEK293 and SK-N-AS cells. Treatment with CHX resulted in ˜23% NSD1 non-productive mRNA in SH-SY5Y cells, ˜15% NSD1 non-productive mRNA in U-87 MG cells, and ˜13% NSD1 non-productive mRNA in HEK293 and SK-N-AS cells. In cells treated only with water (mock), the percentage of non-productive RNA remained low.

### Example 8. Effects of Exemplary Chemically Modified ASOs

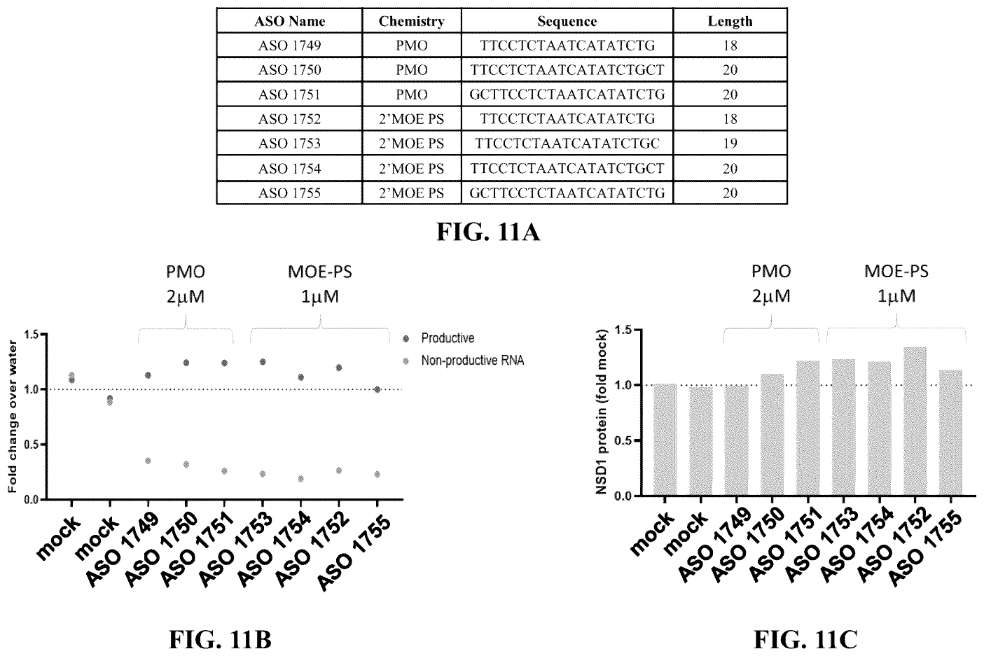

The effect of ASOs with modified backbone chemistry in U-87 MG cells was determined. U-87 MG cells were treated either with (1) ASOs that had phosphorodiamidate morpholino (PMO) modifications, or (2) ASOs that had 2′-O-methoxyethyl modifications and phosphorothioate backbones (2′MOE-PS) (FIG. 11A, Table 6).

NSD1 mRNA levels were assessed 24 hours after nucleofecting U-87 MG cells with either 2 μM ASOs with PMO modifications or 1 μM ASOs with 2′MOE-PS modifications, and the fold change was quantitated for productive and non-productive mRNA transcripts compared to mock controls (FIG. 11B). All results are normalized to the mock controls. PMO-containing ASO 1749 corresponds in sequence to 2′MOE-PS-containing ASO 1752. Both chemically modified ASO 1749 and ASO 1752 resulted in at least about a 1.1-fold increase in productive NSD1 mRNA compared to mock controls. ASO 1749 resulted in a decrease to about 0.4-fold of non-productive NSD1 mRNA and ASO 1752 resulted in a decrease to about 0.3-fold of non-productive NSD1 mRNA compared to water-only mock controls. PMO-containing ASO 1750 corresponds in sequence to 2′MOE-PS-containing ASO 1754. PMO-containing ASO 1750 resulted in at least about 1.2-fold increase in productive NSD1 mRNA and decreased non-productive NSD1 mRNA to about 0.3-fold compared to mock controls. 2′MOE-PS-containing ASO 1754 resulted in at least about 1.1-fold increase in productive NSD1 mRNA and decreased non-productive NSD1 mRNA to about 0.2-fold compared to mock controls. PMO-containing ASO 1751 corresponds in sequence to 2′MOE-PS-containing ASO 1755. PMO-containing ASO 1751 resulted in at least 1.2-fold increase in productive NSD1 mRNA and decreased non-productive NSD1 mRNA to about 0.3-fold compared to mock controls. 2′MOE-PS-containing ASO 1755 resulted in no change in productive NSD1 mRNA but decreased non-productive NSD1 mRNA to about 0.3-fold compared to mock controls. 2′MOE-PS-containing ASO 1753 resulted in at least about 1.2-fold increase in productive NSD1 mRNA and decreased non-productive NSD1 mRNA to about 0.3-fold compared to mock controls. In general, productive NSD1 mRNA levels increased and non-productive NSD1 mRNA decreased relative to mock controls when cells were treated with either 2 μM ASOs with PMO modifications or 1 M ASOs with 2′MOE-PS modifications.

NSD1 protein levels were assessed 72 hours after nucleofecting U-87 MG cells with either 2 μM ASOs with PMO modifications or 1 μM ASOs with 2′MOE-PS modifications and compared to mock controls (FIG. 11C). All results are normalized to the mock controls. 2′MOE-PS-containing ASO 1752 resulted in at least about a 1.3-fold increase in NSD1 protein compared to mock controls. PMO-containing ASO 1750 resulted in at least about a 1.1-fold increase in NSD1 protein compared to water-only mock controls. 2′MOE-PS-containing ASO 1754 resulted in at least about a 1.2-fold increase in NSD1 protein compared to mock controls. PMO-containing ASO 1751 resulted in at least about a 1.2-fold increase in NSD1 protein compared to mock controls. 2′MOE-PS-containing ASO 1755 resulted in at least about a 1.1-fold increase in NSD1 protein compared to mock controls. 2′MOE-PS-containing ASO 1753 resulted in at least about a 1.2-fold increase in NSD1 protein compared to mock controls. In general, NSD1 protein levels increased relative to mock controls when cells were treated with either 2 μM ASOs with PMO modifications or 1 μM ASOs with 2′MOE-PS modifications. As such, the effect of MOE-PS ASO hits translates to (i.e., behaves similarly to) ASOs with alternative backbones such as those modified with PMO.

TABLE 6
Exemplary ASOs with Modified
Backbone Chemistry

| | | | SEQ ID |
|---|---|---|---|
| ASO Name | Chemistry | Sequence | NO: |
| ASO 1749 | PMO | TTCCTCTAATCATATCTG | 1768 |
| ASO 1750 | PMO | TTCCTCTAATCATATCTGCT | 1769 |
| ASO 1751 | PMO | GCTTCCTCTAATCATATCTG | 1770 |
| ASO 1752 | 2′MOE PS | TTCCTCTAATCATATCTG | 1771 |
| ASO 1753 | 2′MOE PS | TTCCTCTAATCATATCTGC | 1772 |
| ASO 1754 | 2′MOE PS | TTCCTCTAATCATATCTGCT | 1773 |
| ASO 1755 | 2′MOE PS | GCTTCCTCTAATCATATCTG | 1774 |

### Example 9. Upregulation of NSD1 Protein by Exemplary ASO Resulted in Globally Elevated H3K36me2 Levels

H3K36me2 is an epigenetic modification on Histone H3, and NSD1 is a histone methyltransferase that can mediate the dimethylation of Histone H3 at residue K36 (H3K36me2). NSD1-mediated H3K36me2 can contribute to the recruitment of DNA methyltransferases and the maintenance of DNA methylation at intergenic regions. Thus, levels of H3K36me2 were examined to determine whether the upregulation of NSD1 protein by ASOs would facilitate the elevation of H3K36me2 levels in U-87 MG cells. All results in this Example comprise data drawn from 2-3 independent experiments and all results for each assay are normalized to the water-only controls, mean±SEM.

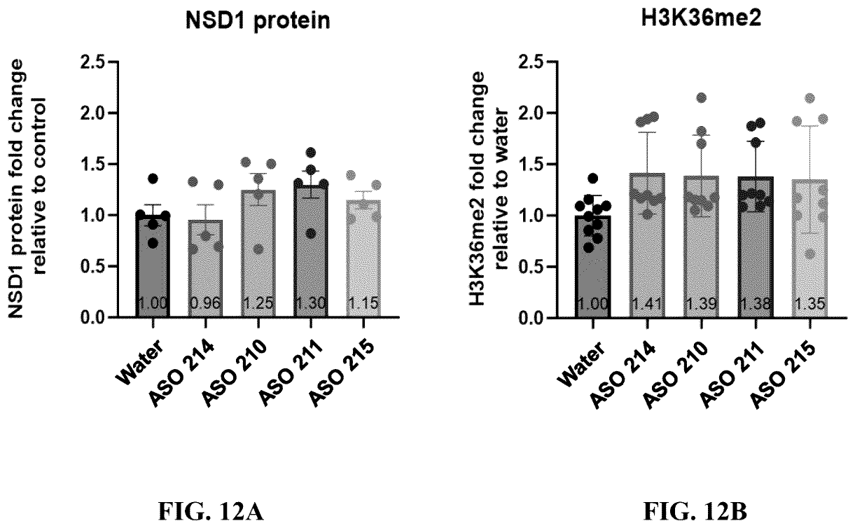

ASOs were evaluated in U-87 MG cells to assess their potency in elevating NSD1 protein expression and H3K36me2 levels. U-87 MG cells were nucleofected with 1 M of one of four exemplary ASOs (ASO 214, ASO 210, ASO 211, or ASO 215) and harvested 72 hours after nucleofection. NSD1 protein levels for each of the four exemplary ASOs were measured by immuno-capillary electrophoresis (JESS) and compared with water-only controls (FIG. 12A). NSD1 protein levels were higher than the water-only controls when U-87 MG cells were treated with ASO 210, ASO 211, or ASO 215, while NSD1 protein levels were slightly lower than those of water-only controls when U-87 MG cells were treated with ASO 214. Treatment with ASO 210 resulted in about a 1.25-fold increase in NSD1 protein levels. Treatment with ASO 211 resulted in about a 1.3-fold increase in NSD1 protein levels. Treatment with ASO 215 resulted in about a 1.15-fold increase in NSD1 protein levels. Treatment with ASO 214 resulted in a decrease in NSD1 protein levels to about 0.96-fold of the water-only controls.

Cellular H3K36me2 levels, as measured by AlphaLISA® assay, were elevated after treatment with all four ASOs (FIG. 12B). H3K36me2 levels were about 1.41-fold higher in cells treated with ASO 214. When compared to H3K36me2 levels in cells treated with water only, H3K36me2 levels were about 1.39-fold higher in cells treated with ASO 210, about 1.38-fold higher in cells treated with ASO 211, and about 1.35-fold higher in cells treated with ASO 215. Considered together, the four ASOs tested were found to both increase NSD1 protein levels and elevate cellular H3K36me2 levels in U-87 MG cells.

### Example 10. ASO 211 Resulted in a Dose-Dependent Increase of Global H3K36me2

ASO 211 was evaluated further to determine whether changes in dosage would affect NSD1 protein expression and H3K36me2 levels in U-87 MG cells.

U-87 MG cells were nucleofected with one of four doses (0.25 μM, 0.5 μM, 1.0 μM, or 2.0 μM) of a hit ASO (ASO 211) or a water-only control. Cells were harvested 72 hours after nucleofection and their NSD1 protein, H3K36me2, and total Histone 3 (H3) levels were quantitated. All results in this Example comprise data drawn from 2-3 independent experiments and all results for each assay are normalized to the water-only controls, mean±SEM; one-way ANOVA; * pval<0.05, ***pval<0.01; ****pval<0.001.

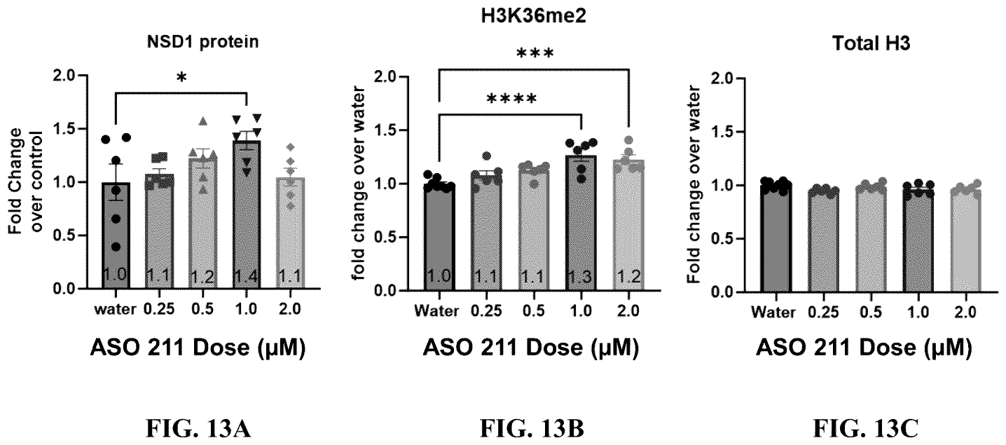

NSD1 protein levels were measured by immuno-capillary electrophoresis (JESS). NSD1 protein levels were higher than the water-only controls at all tested concentrations of ASO 211 (FIG. 13A). In particular, a 0.25-μM concentration of ASO 211 resulted in about a 1.1-fold increase, 0.5 μM resulted in about a 1.2-fold increase, 1.0 μM resulted in about a 1.4-fold increase, and 2.0 μM resulted in about a 1.1-fold increase of NSD1 protein levels relative to the water-only controls. Treatment of U-87 cells with 1.0 μM of ASO 211 yielded the highest-fold increase (about 1.4-fold) of NSD1 protein relative to the remaining three concentrations of ASO. Treatment of U-87 cells with 0.5 μM of ASO 211 yielded the second highest-fold increase (about 1.2-fold) of NSD1 protein relative to the three other concentrations of ASO. Treatment of U-87 cells with either the highest or the lowest concentrations (0.25 μM and 2.0 μM) of ASO 211 yielded the smallest-fold increase (about 1.1-fold) of NSD1 protein relative to the other tested mid-level concentrations of ASO.

Cellular H3K36me2 levels, as measured by AlphaLISA® assay, were elevated after treatment with all tested concentrations of ASO 211 (FIG. 13B). H3K36me2 levels increased about 1.3-fold in cells treated with 1.0 μM of ASO 211 compared to those treated with water only. H3K36me2 levels were about 1.1-fold higher than in water-only cells when the cells were treated with ASO 211 concentrations of either 0.25 μM or 0.5 μM. H3K36me2 levels were about 1.2-fold higher than in water-only cells, when the cells were treated with 2.0 μM of ASO 211. Lower dosage concentrations of the ASO generally resulted in a smaller-fold change in cellular H3K36me2 levels.

To rule out the possibility that the effects observed on NSD1 protein expression and H3K36me2 levels were due to changes in cellular levels of Histone H3, total H3 levels were also measured by AlphaLISA® assay (FIG. 13C). Histone H3 levels were found to be approximately the same across all experimental conditions, whether water or any concentration of ASO 211 was used. As such, the modulation of NSD1 protein expression and H3K36me2 levels by ASO 211 were likely not caused by changes in total Histone H3 levels, but rather, by the presence of the ASO itself and the experimental concentrations tested. In summary, ASO 211 was found to increase global H3K36me2 levels in a dose-dependent manner.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1. A method of modulating expression of a target protein in a cell comprising a pre-mRNA that is transcribed from a target gene and that encodes the target protein, the pre-mRNA comprising an alternatively-spliced coding exon (ASCE), wherein an alternative processed mRNA that is produced by splicing out of the ASCE during processing of the pre-mRNA undergoes non-sense mediated RNA decay, the method comprising contacting a therapeutic agent or a vector encoding the therapeutic agent to the cell, wherein the therapeutic agent promotes inclusion of the ASCE during the processing of the pre-mRNA, thereby increasing a level of a processed mRNA that is processed from the pre-mRNA and that comprises the ASCE.

2. A method of treating or reducing the likelihood of developing a disease or condition in a subject in need thereof by modulating expression of a target protein in a cell of the subject, the method comprising: contacting the cell of the subject with a therapeutic agent or a vector encoding the therapeutic agent, wherein the cell comprises a pre-mRNA that is transcribed from a target gene and that encodes the target protein, the pre-mRNA comprising an alternatively-spliced coding exon (ASCE), wherein an alternative processed mRNA that is produced by splicing out of the ASCE during processing of the pre-mRNA undergoes non-sense mediated RNA decay, wherein the therapeutic agent promotes inclusion of the ASCE during the processing of the pre-mRNA, thereby increasing a level of a processed mRNA that is processed from the pre-mRNA and that comprises the ASCE.

3. The method of claim 1, wherein the expression of the target protein is increased in the cell.

4. The method of claim 1, wherein the target gene is selected from the group consisting of: PKD1, ABCA4, FUS, CEL, and NSD1.

5. (canceled)

6. The method of claim 1, wherein the therapeutic agent
(a) binds to a targeted portion of the pre-mRNA encoding the target protein;
(b) modulates binding of a factor involved in splicing of the ASCE; or
(c) a combination of (a) and (b).

7. The method of claim 6, wherein the therapeutic agent interferes with binding of the factor involved in splicing of the ASCE to a region of the targeted portion.

8. The method of claim 6, wherein the targeted portion is proximal to the ASCE.

9-16. (canceled)

17. The method of claim 6, wherein the targeted portion is located in an intronic region between the ASCE and a canonical exonic region upstream of the ASCE of the pre-mRNA encoding the target protein.

18. The method of claim 6, wherein the targeted portion is located in an intronic region between the ASCE and a canonical exonic region downstream of the ASCE of the pre-mRNA encoding the target protein.

19. The method of claim 6, wherein the targeted portion comprises at least a portion of the ASCE.

20. The method of claim 6, wherein the targeted portion at least a portion of an intronic region upstream or downstream of the ASCE.

21. The method of claim 6, wherein the targeted portion does not comprise a 5′ exon-intron junction of the ASCE or a 3′ exon-intron junction of the ASCE.

22. The method of claim 6, wherein the targeted portion is within the ASCE.

23. The method of claim 6, wherein the targeted portion comprises 5 or more consecutive nucleotides of the ASCE.

24-26. (canceled)

27. The method of claim 1, wherein the targeted portion of the pre-mRNA is within the ASCE selected from the group consisting of: GRCh38/hg38: chr16 2092954 2093093; GRCh38/hg38: chr1 94111438 94111579; GRCh38/hg38: chr16 31186802 31186836; GRCh38/hg38: chr9 133066530 133066660; and GRCh38/hg38: chr5 177238237 177238507.

28. The method of claim 1, wherein the targeted portion of the pre-mRNA is upstream or downstream of the ASCE selected from the group consisting of: GRCh38/hg38: chr16 2092954 2093093; GRCh38/hg38: chr1 94111438 94111579; GRCh38/hg38: chr16 31186802 31186836; GRCh38/hg38: chr9 133066530 133066660; and GRCh38/hg38: chr5 177238237 177238507.

29-34. (canceled)

35. The method of claim 1, wherein the target protein is NSD1, and wherein the method causes a modification of a histone protein in the cell.

36. The method of claim 35, wherein the histone protein is Histone H3.

37-66. (canceled)

67. The method of claim 1, wherein the alternative processed mRNA that is produced by splicing out of the ASCE during processing of the pre-mRNA comprises a premature termination codon (PTC).

68. The method of claim 1, wherein the agent is an antisense oligomer (ASO).

69-164. (canceled)
